SEQUENCE LISTING (168 sequences)

SEQ ID NO: 1 - protein, 262 aa, Artificial Sequence
  Synthetic Polypeptide
MAEVQLLESRGGLVQPGRSLRLSCAASGFTFDDYAMHWVRQAPGKGLEWVSGISWNSGSIGYADSVKGRFTISRDNTKNSLYLQMNSLRAEDTGVYYCAREVTGDLDYWGQGTLVTVSSGSASAPTLGGGGSGGGGSAAAEIVMTQSPGTLSLSPGERATLSCRASQFLSSTYLAWYQQRPGQAPRLLIYSASNRATGVPDRFSGSGSGTDFTLKISRVEAEDVGVYYCMQATHWPYTFGQGTKLEIKRTVAAPSVFKASGA

SEQ ID NO: 2 - protein, 271 aa, Artificial Sequence
  Synthetic Polypeptide
MAQIQLVQSGAEVKKPGASVKVSCKASGYTFTGYYMHWVRQAPGQGLEWMGWINPNSGGTNYAQKFQGWVTMTRDTSISTAYMELSRLRSDDTAVYYCARGGSIAVAGTLVDYYGMDVWGQGTTVTVSSGSASAPTLGGGGSGGGGSAAADIQMTQSPSSLSASVGDRVTITCQASQDISNYLNWYQQKPGKAPKLLIYDASNLETGVPSRFSGSGSGTDFTLTISSLQPEDVATYYCQKYSTAPLTFGGGTKVEIKRTVAAPSVFKASGA

SEQ ID NO: 3 - protein, 267 aa, Artificial Sequence
  Synthetic Polypeptide
MAQVQLVQSGGGVVQPGRSLRLSCAASGFTFSSYAMHWVRQAPGKGLEWVAVISYDGSNEYYADSVKGRFTISRDNSKNTLYLQMNSLRAEDTAVYYCAKDFWSGYPQYNWFDPWGQGTLVTVSSGSASAPTLGGGGSGGGGSAAAEIVMTQSPATLSLSAGERATLSCRASQSVSNYLAWYQQKPGQAPRLLIYDASNRATGIPARFSGSGSGTDFTLTISSLEPEDFAVYYCQQYGSAPLTFGGGTNVEIKRTVAAPSVFKASGA

SEQ ID NO: 4 - protein, 267 aa, Artificial Sequence
  Synthetic Polypeptide
MAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARTPPLWFGEYGAFDIWGQGAMVTVSSGSASAPTLGGGGSGGGGSAAADIQMTQSPSSLSASVGDRVTITCQASQDISNYLNWYQQKPGKAPKLLIYDASNLETGVPSRFSGSGSGTDFTLTISSLQPDDFATYYCQQSYTTPITFGQGTRLEIKRTVAAPSVFKASGA

SEQ ID NO: 5 - protein, 264 aa, Artificial Sequence
  Synthetic Polypeptide
MAEVQLVQSGAEVKKPGASVKVSCKASGYTFTGYYIYWVRQAPGQGLEWMGWIRPNSGDTNYAQKFQDRLTMTRDTSISTAYMELSRLRSDDTAVYYCAGNYDILTGYQAPLGYWGQGALVTVSSGSASAPTLGGGGSGGGGSAAADIVLTQSPATLSVSPGERATLSCRASQRVISNYLAWYQQKPGQAPRLLIYGASSRATGIPDRFSGSGSGTDFTLSISRLEPEDFAVYYCQHYGPFGGGTKVEIKRTVAAPSVFKASGA

SEQ ID NO: 6 - protein, 265 aa, Artificial Sequence
  Synthetic Polypeptide
MAQVQLQESGPGLVKPSETLSLTCTVSGGSISSSSYYWGWVRQPPGKGLEWIGEIYHSGSTNYNPSLKSRVTISVDTSKNQFSLKLSSVTAADTAVYYCARVGTSWEYYFDYWGQGTLVTVSSGSASAPTLGGGGSGGGGSAAADIQMTQSPASLSASVGDRVTITCRTSQHIINYLNWYQQRPGKAPNLLIYKASTLQSGVPSRFSGSGSGTHFTLTISSLQPEDFATYYCQQYQSYPITFGQGTRLEIKRTVAAPSVFKASGA

SEQ ID NO: 7 - protein, 269 aa, Artificial Sequence
  Synthetic Polypeptide
MAEVQLVQSGGGLVQPGGSLRLSCAASGFTFSSYSMNWVRQAPGKGLEWVSYISSSSSTIYYADSVKGRFTISRDNAKNSLYLQMNSLRAEDTAVYYCARDGIYYDSSGYYDLFDYWGQGTLVTVSSGSASAPTLGGGGSGGGGSAAADIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKLEIKRTVAAPSVFKASGA

SEQ ID NO: 8 - protein, 126 aa, Artificial Sequence
  Synthetic Polypeptide
MAEVQLLESRGGLVQPGRSLRLSCAASGFTFDDYAMHWVRQAPGKGLEWVSGISWNSGSIGYADSVKGRFTISRDNTKNSLYLQMNSLRAEDTGVYYCAREVTGDLDYWGQGTLVTVSSGSASAPT

SEQ ID NO: 9 - protein, 122 aa, Artificial Sequence
  Synthetic Polypeptide
EIVMTQSPGTLSLSPGERATLSCRASQFLSSTYLAWYQQRPGQAPRLLIYSASNRATGVPDRFSGSGSGTDFTLKISRVEAEDVGVYYCMQATHWPYTFGQGTKLEIKRTVAAPSVFKASGA

SEQ ID NO: 10 - protein, 136 aa, Artificial Sequence
  Synthetic Polypeptide
MAQIQLVQSGAEVKKPGASVKVSCKASGYTFTGYYMHWVRQAPGQGLEWMGWINPNSGGTNYAQKFQGWVTMTRDTSISTAYMELSRLRSDDTAVYYCARGGSIAVAGTLVDYYGMDVWGQGTTVTVSSGSASAPT

SEQ ID NO: 11 - protein, 121 aa, Artificial Sequence
  Synthetic Polypeptide
DIQMTQSPSSLSASVGDRVTITCQASQDISNYLNWYQQKPGKAPKLLIYDASNLETGVPSRFSGSGSGTDFTLTISSLQPEDVATYYCQKYSTAPLTFGGGTKVEIKRTVAAPSVFKASGA

SEQ ID NO: 12 - protein, 132 aa, Artificial Sequence
  Synthetic Polypeptide
MAQVQLVQSGGGVVQPGRSLRLSCAASGFTFSSYAMHWVRQAPGKGLEWVAVISYDGSNEYYADSVKGRFTISRDNSKNTLYLQMNSLRAEDTAVYYCAKDFWSGYPQYNWFDPWGQGTLVTVSSGSASAPT

SEQ ID NO: 13 - protein, 121 aa, Artificial Sequence
  Synthetic Polypeptide
EIVMTQSPATLSLSAGERATLSCRASQSVSNYLAWYQQKPGQAPRLLIYDASNRATGIPARFSGSGSGTDFTLTISSLEPEDFAVYYCQQYGSAPLTFGGGTNVEIKRTVAAPSVFKASGA

SEQ ID NO: 14 - protein, 132 aa, Artificial Sequence
  Synthetic Polypeptide
MAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARTPPLWFGEYGAFDIWGQGAMVTVSSGSASAPT

SEQ ID NO: 15 - protein, 121 aa, Artificial Sequence
  Synthetic Polypeptide
DIQMTQSPSSLSASVGDRVTITCQASQDISNYLNWYQQKPGKAPKLLIYDASNLETGVPSRFSGSGSGTDFTLTISSLQPDDFATYYCQQSYTTPITFGQGTRLEIKRTVAAPSVFKASGA

SEQ ID NO: 16 - protein, 132 aa, Artificial Sequence
  Synthetic Polypeptide
MAEVQLVQSGAEVKKPGASVKVSCKASGYTFTGYYIYWVRQAPGQGLEWMGWIRPNSGDTNYAQKFQDRLTMTRDTSISTAYMELSRLRSDDTAVYYCAGNYDILTGYQAPLGYWGQGALVTVSSGSASAPT

SEQ ID NO: 17 - protein, 118 aa, Artificial Sequence
  Synthetic Polypeptide
DIVLTQSPATLSVSPGERATLSCRASQRVISNYLAWYQQKPGQAPRLLIYGASSRATGIPDRFSGSGSGTDFTLSISRLEPEDFAVYYCQHYGPFGGGTKVEIKRTVAAPSVFKASGA

SEQ ID NO: 18 - protein, 130 aa, Artificial Sequence
  Synthetic Polypeptide
MAQVQLQESGPGLVKPSETLSLTCTVSGGSISSSSYYWGWVRQPPGKGLEWIGEIYHSGSTNYNPSLKSRVTISVDTSKNQFSLKLSSVTAADTAVYYCARVGTSWEYYFDYWGQGTLVTVSSGSASAPT

SEQ ID NO: 19 - protein, 121 aa, Artificial Sequence
  Synthetic Polypeptide
DIQMTQSPASLSASVGDRVTITCRTSQHIINYLNWYQQRPGKAPNLLIYKASTLQSGVPSRFSGSGSGTHFTLTISSLQPEDFATYYCQQYQSYPITFGQGTRLEIKRTVAAPSVFKASGA

SEQ ID NO: 20 - protein, 134 aa, Artificial Sequence
  Synthetic Polypeptide
MAEVQLVQSGGGLVQPGGSLRLSCAASGFTFSSYSMNWVRQAPGKGLEWVSYISSSSSTIYYADSVKGRFTISRDNAKNSLYLQMNSLRAEDTAVYYCARDGIYYDSSGYYDLFDYWGQGTLVTVSSGSASAPT

SEQ ID NO: 21 - protein, 121 aa, Artificial Sequence
  Synthetic Polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKLEIKRTVAAPSVFKASGA

SEQ ID NO: 22 - protein, 5 aa, Artificial Sequence
  Synthetic Polypeptide
DYAMH

SEQ ID NO: 23 - protein, 17 aa, Artificial Sequence
  Synthetic Polypeptide
GISWNSGSIGYADSVKG

SEQ ID NO: 24 - protein, 8 aa, Artificial Sequence
  Synthetic Polypeptide
EVTGDLDY

SEQ ID NO: 25 - protein, 12 aa, Artificial Sequence
  Synthetic Polypeptide
RASQFLSSTYLA

SEQ ID NO: 26 - protein, 7 aa, Artificial Sequence
  Synthetic Polypeptide
SASNRAT

SEQ ID NO: 27 - protein, 9 aa, Artificial Sequence
  Synthetic Polypeptide
MQATHWPYT

SEQ ID NO: 28 - protein, 5 aa, Artificial Sequence
  Synthetic Polypeptide
GYYMH

SEQ ID NO: 29 - protein, 17 aa, Artificial Sequence
  Synthetic Polypeptide
WINPNSGGTNYAQKFQG

SEQ ID NO: 30 - protein, 18 aa, Artificial Sequence
  Synthetic Polypeptide
GGSIAVAGTLVDYYGMDV

SEQ ID NO: 31 - protein, 11 aa, Artificial Sequence
  Synthetic Polypeptide
QASQDISNYLN

SEQ ID NO: 32 - protein, 7 aa, Artificial Sequence
  Synthetic Polypeptide
DASNLET

SEQ ID NO: 33 - protein, 9 aa, Artificial Sequence
  Synthetic Polypeptide
QKYSTAPLT

SEQ ID NO: 34 - protein, 5 aa, Artificial Sequence
  Synthetic Polypeptide
SYAMH

SEQ ID NO: 35 - protein, 17 aa, Artificial Sequence
  Synthetic Polypeptide
VISYDGSNEYYADSVKG

SEQ ID NO: 36 - protein, 14 aa, Artificial Sequence
  Synthetic Polypeptide
DFWSGYPQYNWFDP

SEQ ID NO: 37 - protein, 11 aa, Artificial Sequence
  Synthetic Polypeptide
RASQSVSNYLA

SEQ ID NO: 38 - protein, 7 aa, Artificial Sequence
  Synthetic Polypeptide
DASNRAT

SEQ ID NO: 39 - protein, 9 aa, Artificial Sequence
  Synthetic Polypeptide
QQYGSAPLT

SEQ ID NO: 40 - protein, 5 aa, Artificial Sequence
  Synthetic Polypeptide
SYGIS

SEQ ID NO: 41 - protein, 17 aa, Artificial Sequence
  Synthetic Polypeptide
WISAYNGNTNYAQKLQG

SEQ ID NO: 42 - protein, 14 aa, Artificial Sequence
  Synthetic Polypeptide
TPPLWFGEYGAFDI

SEQ ID NO: 43 - protein, 11 aa, Artificial Sequence
  Synthetic Polypeptide
QASQDISNYLN

SEQ ID NO: 44 - protein, 7 aa, Artificial Sequence
  Synthetic Polypeptide
DASNLET

SEQ ID NO: 45 - protein, 9 aa, Artificial Sequence
  Synthetic Polypeptide
QQSYTTPIT

SEQ ID NO: 46 - protein, 5 aa, Artificial Sequence
  Synthetic Polypeptide
GYYIY

SEQ ID NO: 47 - protein, 17 aa, Artificial Sequence
  Synthetic Polypeptide
WIRPNSGDTNYAQKFQD

SEQ ID NO: 48 - protein, 14 aa, Artificial Sequence
  Synthetic Polypeptide
NYDILTGYQAPLGY

SEQ ID NO: 49 - protein, 12 aa, Artificial Sequence
  Synthetic Polypeptide
RASQRVISNYLA

SEQ ID NO: 50 - protein, 7 aa, Artificial Sequence
  Synthetic Polypeptide
GASSRAT

SEQ ID NO: 51 - protein, 5 aa, Artificial Sequence
  Synthetic Polypeptide
QHYGP

SEQ ID NO: 52 - protein, 5 aa, Artificial Sequence
  Synthetic Polypeptide
SYYWG

SEQ ID NO: 53 - protein, 16 aa, Artificial Sequence
  Synthetic Polypeptide
EIYHSGSTNYNPSLKS

SEQ ID NO: 54 - protein, 11 aa, Artificial Sequence
  Synthetic Polypeptide
VGTSWEYYFDY

SEQ ID NO: 55 - protein, 11 aa, Artificial Sequence
  Synthetic Polypeptide
RTSQHIINYLN

SEQ ID NO: 56 - protein, 7 aa, Artificial Sequence
  Synthetic Polypeptide
KASTLQS

SEQ ID NO: 57 - protein, 9 aa, Artificial Sequence
  Synthetic Polypeptide
QQYQSYPIT

SEQ ID NO: 58 - protein, 5 aa, Artificial Sequence
  Synthetic Polypeptide
SYSMN

SEQ ID NO: 59 - protein, 17 aa, Artificial Sequence
  Synthetic Polypeptide
YISSSSSTIYYADSVKG

SEQ ID NO: 60 - protein, 16 aa, Artificial Sequence
  Synthetic Polypeptide
DGIYYDSSGYYDLFDY

SEQ ID NO: 61 - protein, 11 aa, Artificial Sequence
  Synthetic Polypeptide
RASQSISSYLN

SEQ ID NO: 62 - protein, 7 aa, Artificial Sequence
  Synthetic Polypeptide
AASSLQS

SEQ ID NO: 63 - protein, 9 aa, Artificial Sequence
  Synthetic Polypeptide
QQSYSTPPT

SEQ ID NO: 64 - protein, 5 aa, Artificial Sequence
  Synthetic Polypeptide
  misc_feature (1)..(1): Xaa is Asp, Gly, or Ser
  misc_feature (3)..(3): Xaa is Ala, Tyr, Gly, or Ser
  misc_feature (4)..(4): Xaa is Met, Ile, OR Trp
  misc_feature (5)..(5): Xaa is His, Ser, Tyr, Gly, or Asn
XYXXX

SEQ ID NO: 65 - protein, 17 aa, Artificial Sequence
  Synthetic Polypeptide
  misc_feature (1)..(1): Xaa is Gly, Trp, Val, Tyr or absent
  misc_feature (2)..(2): Xaa is Ile or Glu
  misc_feature (3)..(3): Xaa is Ser, Asn, Arg, or Ile
  misc_feature (4)..(4): Xaa is Trp, Pro, Tyr, Ala, or Ser
  misc_feature (5)..(5): Xaa is Asn, Asp, Tyr, His, or Ser
  misc_feature (6)..(6): Xaa is Ser, Gly, or Asn
  misc_feature (7)..(7): Xaa is Gly or Ser
  misc_feature (8)..(8): Xaa is Ser, Gly, Asn, Asp, or Thr
  misc_feature (9)..(9): Xaa is Ile, Thr, or Glu
  misc_feature (10)..(10): Xaa is Gly, Asn, or Tyr
  misc_feature (12)..(12): Xaa is Ala or Asn
  misc_feature (13)..(13): Xaa is Asp, Gln, or  Pro
  misc_feature (14)..(14): Xaa is Ser or Lys
  misc_feature (15)..(15): Xaa is Val, Phe, or Leu
  misc_feature (16)..(16): Xaa is Lys or Gln
  misc_feature (17)..(17): Xaa is Gly, Asp, or Ser
XXXXXXXXXXYXXXXXX

SEQ ID NO: 66 - protein, 18 aa, Artificial Sequence
  Synthetic Polypeptide
  misc_feature (1)..(1): Xaa is Gly or absent
  misc_feature (2)..(2): Xaa is Gly or absent
  misc_feature (3)..(3): Xaa is Ser, Asp, or absent
  misc_feature (4)..(4): Xaa is Ile, Gly, or absent
  misc_feature (5)..(5): Xaa is Ala, Asp, Thr, Asn, Ile, or absent
  misc_feature (6)..(6): Xaa is Val, Phe, Pro, Tyr, or absent
  misc_feature (7)..(7): Xaa is Ala, Trp, Pro, Asp, Tyr, or absent
  misc_feature (8)..(8): Xaa is Gly, Ser, Leu, Ile, Val, Asp, or absent
  misc_feature (9)..(9): Xaa is Thr, Gly, Trp, Leu, Ser, or absent
  misc_feature (10)..(10): Xaa is Leu, Tyr, Phe, Thr, Ser, or absent
  misc_feature (11)..(11): Xaa is Glu, Val, Pro, Gly, or Ser
  misc_feature (12)..(12): Xaa is Val, Asp, Gln, Glu, Tyr, or Trp
  misc_feature (13)..(13): Xaa is Thr, Tyr, Gln, or Glu
  misc_feature (14)..(14): Xaa is Gly, Tyr, Asn, Ala, or Asp
  misc_feature (15)..(15): Xaa is Asp, Gly, Trp, Ala, Phe, Tyr, or Leu
  misc_feature (16)..(16): Xaa is Leu, Met, or Phe
  misc_feature (17)..(17): Xaa is Asp or Gly
  misc_feature (18)..(18): Xaa is Tyr, Val, Pro, or Ile
XXXXXXXXXXXXXXXXXX

SEQ ID NO: 67 - protein, 12 aa, Artificial Sequence
  Synthetic Polypeptide
  misc_feature (1)..(1): Xaa is Arg or Gln
  misc_feature (2)..(2): Xaa is Ala or Thr
  misc_feature (5)..(5): Xaa is Phe, Asp, Ser, Arg, or His
  misc_feature (6)..(6): Xaa is Leu, Ile, or Val
  misc_feature (7)..(7): Xaa is Ser, Ile, or absent
  misc_feature (8)..(8): Xaa is Ser or absent
  misc_feature (9)..(9): Xaa is Thr, Asn, or absent
  misc_feature (12)..(12): Xaa is Ala or Asn
XXSQXXXXXYLX

SEQ ID NO: 68 - protein, 7 aa, Artificial Sequence
  Synthetic Polypeptide
  misc_feature (1)..(1): Xaa is Ser, Asp, Gly, Lys, or Ala
  misc_feature (4)..(4): Xaa is Asn, Ser, or Thr
  misc_feature (5)..(5): Xaa is Arg or Leu
  misc_feature (6)..(6): Xaa is Ala, Glu, or Gln
  misc_feature (7)..(7): Xaa is Thr or Ser
XASXXXX

SEQ ID NO: 69 - protein, 9 aa, Artificial Sequence
  Synthetic Polypeptide
  misc_feature (1)..(1): Xaa Met or Gln
  misc_feature (2)..(2): Xaa is Gln, Lys, or His
  misc_feature (3)..(3): Xaa is Ala, Tyr, or Ser
  misc_feature (4)..(4): Xaa is Thr, Ser, Gly, Tyr, or Gln
  misc_feature (5)..(5): Xaa is His, Thr, Ser, or absent
  misc_feature (6)..(6): Xaa is Trp, Ala, Thr, Tyr, or absent
  misc_feature (8)..(8): Xaa is Tyr, Leu, Ile, Pro, or absent
  misc_feature (9)..(9): Xaa is Thr or absent
XXXXXXPXX

SEQ ID NO: 70 - protein, 5 aa, Artificial Sequence
  Synthetic Polypeptide
  misc_feature (1)..(1): Xaa is Asp, Gly, or Ser
  misc_feature (3)..(3): Xaa is Ala, Tyr, or Gly
  misc_feature (4)..(4): Xaa is Met or Ile
  misc_feature (5)..(5): Xaa is His or Ser
XYXXX

SEQ ID NO: 71 - protein, 17 aa, Artificial Sequence
  Synthetic Polypeptide
  misc_feature (1)..(1): Xaa is Gly, Trp, or Val
  misc_feature (3)..(3): Xaa is Ser or Asn
  misc_feature (4)..(4): Xaa is Trp, Pro, Tyr, or Ala
  misc_feature (5)..(5): Xaa is Asn, Asp, or Tyr
  misc_feature (6)..(6): Xaa is Ser, Gly, or Asn
  misc_feature (7)..(7): Xaa is Gly or Ser
  misc_feature (8)..(8): Xaa is Ser, Gly, or Asn
  misc_feature (9)..(9): Xaa is Ile, Thr, or Glu
  misc_feature (10)..(10): Xaa is Gly, Asn, or Tyr
  misc_feature (13)..(13): Xaa is Asp or Gln
  misc_feature (14)..(14): Xaa is Ser or Lys
  misc_feature (15)..(15): Xaa is Val, Phe, or Leu
  misc_feature (16)..(16): Xaa is Lys or Gln
XIXXXXXXXXYAXXXXG

SEQ ID NO: 72 - protein, 18 aa, Artificial Sequence
  Synthetic Polypeptide
  misc_feature (1)..(1): Xaa is Gly or absent
  misc_feature (2)..(2): Xaa is Gly or absent
  misc_feature (3)..(3): Xaa is Ser or absent
  misc_feature (4)..(4): Xaa is Ile or absent
  misc_feature (5)..(5): Xaa is Ala, Asp, Thr, or absent
  misc_feature (6)..(6): Xaa is Val, Phe, Pro, or absent
  misc_feature (7)..(7): Xaa is Ala, Trp, Pro or absent
  misc_feature (8)..(8): Xaa is Gly, Ser, Leu, or absent
  misc_feature (9)..(9): Xaa is Thr, Gly, Trp, or absent
  misc_feature (10)..(10): Xaa is Leu, Tyr, Phe, or absent
  misc_feature (11)..(11): Xaa is Glu, Val, Pro, or Gly
  misc_feature (12)..(12): Xaa is Val, Asp, Gln, or Glu
  misc_feature (13)..(13): Xaa is Thr or Tyr
  misc_feature (14)..(14): Xaa is Gly, Tyr, or Asn
  misc_feature (15)..(15): Xaa is Asp, Gly, Trp, or Ala
  misc_feature (16)..(16): Xaa is Leu, Met, or Phe
  misc_feature (18)..(18): Xaa is Tyr, Val, Phe, or Ile
XXXXXXXXXXXXXXXXDX

SEQ ID NO: 73 - protein, 12 aa, Artificial Sequence
  Synthetic Polypeptide
  misc_feature (1)..(1): Xaa is Arg or Gln
  misc_feature (5)..(5): Xaa is Phe, Asp, or Ser
  misc_feature (6)..(6): Xaa is Leu, Ile, or Val
  misc_feature (7)..(7): Xaa is Ser or absent
  misc_feature (9)..(9): Xaa is Thr or Asn
  misc_feature (12)..(12): Xaa is Ala or Asn
XASQXXXSXYLX

SEQ ID NO: 74 - protein, 7 aa, Artificial Sequence
  Synthetic Polypeptide
  misc_feature (1)..(1): Xaa is Ser or Asp
  misc_feature (5)..(5): Xaa is Arg or Leu
  misc_feature (6)..(6): Xaa is Ala or Glu
XASNXXT

SEQ ID NO: 75 - protein, 9 aa, Artificial Sequence
  Synthetic Polypeptide
  misc_feature (1)..(1): Xaa is Met or Gln
  misc_feature (2)..(2): Xaa is Gln or Lys
  misc_feature (3)..(3): Xaa is Ala, Lys, or Ser
  misc_feature (4)..(4): Xaa is Thr, Ser, Gly, or Tyr
  misc_feature (5)..(5): Xaa is His, Thr, or Ser
  misc_feature (6)..(6): Xaa is Trp, Ala, or Thr
  misc_feature (8)..(8): Xaa is Tyr, Leu, or Ile
XXXXXXPXT

SEQ ID NO: 76 - protein, 5 aa, Artificial Sequence
  Synthetic Polypeptide
  misc_feature (1)..(1): Xaa is Gly or Ser
  misc_feature (3)..(3): Xaa is Tyr or Ser
  misc_feature (4)..(4): Xaa is Ile or Met
  misc_feature (5)..(5): Xaa is Tyr or Asn
XYXXX

SEQ ID NO: 77 - protein, 17 aa, Artificial Sequence
  Synthetic Polypeptide
  misc_feature (1)..(1): Xaa is Trp or Tyr
  misc_feature (3)..(3): Xaa is Arg or Ser
  misc_feature (4)..(4): Xaa is Pro or Ser
  misc_feature (5)..(5): Xaa is Asn or Ser
  misc_feature (7)..(7): Xaa is Gly or Ser
  misc_feature (8)..(8): Xaa is Asp or Thr
  misc_feature (9)..(9): Xaa is Thr or Ile
  misc_feature (10)..(10): Xaa is Asn or Tyr
  misc_feature (13)..(13): Xaa is Gln or Asp
  misc_feature (14)..(14): Xaa is Lys or Ser
  misc_feature (15)..(15): Xaa is Phe or Val
  misc_feature (16)..(16): Xaa is Gln or Lys
  misc_feature (17)..(17): Xaa is Asp or Gly
XIXXXSXXXXYAXXXXX

SEQ ID NO: 78 - protein, 16 aa, Artificial Sequence
  Synthetic Polypeptide
  misc_feature (1)..(1): Xaa is Asp or absent
  misc_feature (2)..(2): Xaa is Gly or absent
  misc_feature (3)..(3): Xaa is Asn or Ile
  misc_feature (5)..(5): Xaa is Asp or Tyr
  misc_feature (6)..(6): Xaa is Ile or Asp
  misc_feature (7)..(7): Xaa is Leu or Ser
  misc_feature (8)..(8): Xaa is Thr or Ser
  misc_feature (11)..(11): Xaa is Gln or Tyr
  misc_feature (12)..(12): Xaa is Ala or Asp
  misc_feature (13)..(13): Xaa is Pro or Leu
  misc_feature (14)..(14): Xaa is Leu or Phe
  misc_feature (15)..(15): Xaa is Gly or Asp
XXXYXXXXGYXXXXXY

SEQ ID NO: 79 - protein, 12 aa, Artificial Sequence
  Synthetic Polypeptide
  misc_feature (5)..(5): Xaa is Arg or Ser
  misc_feature (6)..(6): Xaa is Val or Ile
  misc_feature (7)..(7): Xaa is Ile or Ser
  misc_feature (9)..(9): Xaa is Asn or absent
  misc_feature (12)..(12): Xaa is Ala or Asn
RASQXXXSXYLX

SEQ ID NO: 80 - protein, 7 aa, Artificial Sequence
  Synthetic Polypeptide
  misc_feature (1)..(1): Xaa is Gly or Ala
  misc_feature (5)..(5): Xaa is Arg or Leu
  misc_feature (6)..(6): Xaa is Ala or Gln
  misc_feature (7)..(7): Xaa is Thr or Ser
XASSXXX

SEQ ID NO: 81 - protein, 9 aa, Artificial Sequence
  Synthetic Polypeptide
  misc_feature (2)..(2): Xaa is His or Gln
  misc_feature (3)..(3): Xaa is Tyr or Ser
  misc_feature (4)..(4): Xaa is Gly or Tyr
  misc_feature (5)..(5): Xaa is Ser or absent
  misc_feature (6)..(6): Xaa is Thr or absent
  misc_feature (8)..(8): Xaa is Pro or absent
  misc_feature (9)..(9): Xaa is Thr or absent
QXXXXXPXX

SEQ ID NO: 82 - protein, 383 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 83 - protein, 352 aa, Artificial Sequence [sequence omitted]

SEQ ID NO: 84 - protein, 406 aa, Artificial Sequence
  Synthetic Polypeptide
SPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 85 - protein, 243 aa, Artificial Sequence
  Synthetic Polypeptide
NENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 86 - protein, 274 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRR

SEQ ID NO: 87 - protein, 64 aa, Artificial Sequence
  Synthetic Polypeptide
NENSEQKENVEKEGLCNACTWRQNTKSSRIEAIKIQILSKLRLETAPNISKDVIRQLLPKAPPL

SEQ ID NO: 88 - protein, 86 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPL

SEQ ID NO: 89 - protein, 109 aa, Artificial Sequence
  Synthetic Polypeptide
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 90 - protein, 109 aa, Artificial Sequence
  Synthetic Polypeptide
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 91 - protein, 179 aa, Artificial Sequence
  Synthetic Polypeptide
RELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 92 - protein, 188 aa, Artificial Sequence
  Synthetic Polypeptide
QQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRR

SEQ ID NO: 93 - protein, 352 aa, Mus musculus
NEGSEREENVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 94 - protein, 243 aa, Mus musculus
NEGSEREENVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRR

SEQ ID NO: 95 - protein, 352 aa, Macaca fascicularis
NENSEQKENVEKEGLCNACTWRQNTKSSRIEAIKIQILSKLRLETAPNISKDAIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 96 - protein, 243 aa, Macaca fascicularis
NENSEQKENVEKEGLCNACTWRQNTKSSRIEAIKIQILSKLRLETAPNISKDAIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 97 - protein, 381 aa, Mus musculus
AEGPAAAAAAAAAAAGVGGERSSRPAPSAPPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 98 - protein, 272 aa, Mus musculus
AEGPAAAAAAAAAAAGVGGERSSRPAPSAPPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRR

SEQ ID NO: 99 - protein, 75 aa, Artificial Sequence
  Synthetic Polypeptide
NENSEQKENVEKEGLCNACTWRQNTKSSRIEAIKIQILSKLRLETAPNISKDVIRQLLPKAPPLRELIDQYDVQR

SEQ ID NO: 100 - protein, 64 aa, Artificial Sequence; identical to SEQ ID NO: 87
  Synthetic Polypeptide

SEQ ID NO: 101 - protein, 278 aa, Artificial Sequence
  Synthetic Polypeptide
RDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 102 - protein, 288 aa, Artificial Sequence
  Synthetic Polypeptide
RELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 103 - protein, 179 aa, Artificial Sequence; identical to SEQ ID NO: 91
  Synthetic Polypeptide

SEQ ID NO: 104 - protein, 168 aa, Artificial Sequence
  Synthetic Polypeptide
DDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 105 - protein, 109 aa, Artificial Sequence; identical to SEQ ID NO: 89
  Synthetic Polypeptide

SEQ ID NO: 106 - protein, 86 aa, Artificial Sequence; identical to SEQ ID NO: 88
  Synthetic Polypeptide

SEQ ID NO: 107 - protein, 96 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQ

SEQ ID NO: 108 - protein, 108 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEE

SEQ ID NO: 109 - protein, 178 aa, Artificial Sequence
  Synthetic Polypeptide
GDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRR

SEQ ID NO: 110 - protein, 188 aa, Artificial Sequence; identical to SEQ ID NO: 92
  Synthetic Polypeptide

SEQ ID NO: 111 - protein, 109 aa, Artificial Sequence; identical to SEQ ID NO: 90
  Synthetic Polypeptide

SEQ ID NO: 112 - protein, 64 aa, Artificial Sequence
  Synthetic Polypeptide
SPTPGSEGHSAAPDCPSCALAALPKDVPNSQPEMVEAVKKHILNMLHLKKRPDVTQPVPKAALL

SEQ ID NO: 113 - protein, 76 aa, Artificial Sequence
  Synthetic Polypeptide
SPTPGSEGHSAAPDCPSCALAALPKDVPNSQPEMVEAVKKHILNMLHLKKRPDVTQPVPKAALLNAIRKLHVGKVG

SEQ ID NO: 114 - protein, 224 aa, Artificial Sequence
  Synthetic Polypeptide
NAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRR

SEQ ID NO: 115 - protein, 225 aa, Artificial Sequence
  Synthetic Polypeptide
NAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRR

SEQ ID NO: 116 - protein, 226 aa, Artificial Sequence
  Synthetic Polypeptide
NAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRR

SEQ ID NO: 117 - protein, 213 aa, Artificial Sequence
  Synthetic Polypeptide
ENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRR

SEQ ID NO: 118 - protein, 214 aa, Artificial Sequence
  Synthetic Polypeptide
ENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRR

SEQ ID NO: 119 - protein, 330 aa, Artificial Sequence
  Synthetic Polypeptide
ENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 120 - protein, 116 aa, Artificial Sequence [sequence omitted]

SEQ ID NO: 121 - protein, 373 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 122 - protein, 374 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 123 - protein, 264 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 124 - protein, 265 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 125 - protein, 419 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 126 - protein, 421 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 127 - protein, 310 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRR

SEQ ID NO: 128 - protein, 312 aa, Artificial Sequence
  Synthetic Polypeptide
AEGPAAAAAAAAAAAAAGVGGERSSRPAPSVAPEPDGCPVCVWRQHSRELRLESIKSQILSKLRLKEAPNISREVVKQLLPKAPPLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRR

SEQ ID NO: 129 - protein, 362 aa, Artificial Sequence
  Synthetic Polypeptide
NENSEQKENVEKEGLCNACTWRQNTKSSRIEAIKIQILSKLRLETAPNISKDVIRQLLPKAPPLRELIDQYDVQRGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 130 - protein, 361 aa, Artificial Sequence
  Synthetic Polypeptide
NENSEQKENVEKEGLCNACTWRQNTKSSRIEAIKIQILSKLRLETAPNISKDVIRQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 131 - protein, 253 aa, Artificial Sequence
  Synthetic Polypeptide
NENSEQKENVEKEGLCNACTWRQNTKSSRIEAIKIQILSKLRLETAPNISKDVIRQLLPKAPPLRELIDQYDVQRGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRR

SEQ ID NO: 132 - protein, 361 aa, Artificial Sequence; identical to SEQ ID NO: 130
  Synthetic Polypeptide

SEQ ID NO: 133 - protein, 397 aa, Artificial Sequence
  Synthetic Polypeptide
NENSEQKENVEKEGLCNACTWRQNTKSSRIEAIKIQILSKLRLETAPNISKDVIRQLLPKAPPLRELIDQYDVQRENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 134 - protein, 399 aa, Artificial Sequence
  Synthetic Polypeptide
NENSEQKENVEKEGLCNACTWRQNTKSSRIEAIKIQILSKLRLETAPNISKDVIRQLLPKAPPLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 135 - protein, 289 aa, Artificial Sequence
  Synthetic Polypeptide
NENSEQKENVEKEGLCNACTWRQNTKSSRIEAIKIQILSKLRLETAPNISKDVIRQLLPKAPPLRELIDQYDVQRENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRR

SEQ ID NO: 136 - protein, 290 aa, Artificial Sequence
  Synthetic Polypeptide
NENSEQKENVEKEGLCNACTWRQNTKSSRIEAIKIQILSKLRLETAPNISKDVIRQLLPKAPPLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRR

SEQ ID NO: 137 - protein, 360 aa, Artificial Sequence
  Synthetic Polypeptide
SPTPGSEGHSAAPDCPSCALAALPKDVPNSQPEMVEAVKKHILNMLHLKKRPDVTQPVPKAALLNAIRKLHVGKVGDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 138 - protein, 359 aa, Artificial Sequence
  Synthetic Polypeptide
SPTPGSEGHSAAPDCPSCALAALPKDVPNSQPEMVEAVKKHILNMLHLKKRPDVTQPVPKAALLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 139 - protein, 244 aa, Artificial Sequence
  Synthetic Polypeptide
SPTPGSEGHSAAPDCPSCALAALPKDVPNSQPEMVEAVKKHILNMLHLKKRPDVTQPVPKAALLNAIRKLHVGKVGDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 140 - protein, 243 aa, Artificial Sequence
  Synthetic Polypeptide
SPTPGSEGHSAAPDCPSCALAALPKDVPNSQPEMVEAVKKHILNMLHLKKRPDVTQPVPKAALLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 141 - protein, 370 aa, Artificial Sequence
  Synthetic Polypeptide
SPTPGSEGHSAAPDCPSCALAALPKDVPNSQPEMVEAVKKHILNMLHLKKRPDVTQPVPKAALLNAIRKLHVGKVGGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 142 - protein, 368 aa, Artificial Sequence
  Synthetic Polypeptide
SPTPGSEGHSAAPDCPSCALAALPKDVPNSQPEMVEAVKKHILNMLHLKKRPDVTQPVPKAALLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 143 - protein, 254 aa, Artificial Sequence
  Synthetic Polypeptide
SPTPGSEGHSAAPDCPSCALAALPKDVPNSQPEMVEAVKKHILNMLHLKKRPDVTQPVPKAALLNAIRKLHVGKVGGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRR

SEQ ID NO: 144 - protein, 252 aa, Artificial Sequence
  Synthetic Polypeptide
SPTPGSEGHSAAPDCPSCALAALPKDVPNSQPEMVEAVKKHILNMLHLKKRPDVTQPVPKAALLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRR

SEQ ID NO: 145 - protein, 296 aa, Artificial Sequence
  Synthetic Polypeptide
MDMRVPAQLLGLLLLWFSGVLGDYKDDDDKHHHHHHLEVLFQGPNENSEQKENVEKEGLCNACTWRQNTKSSRIEAIKIQILSKLRLETAPNISKDVIRQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRR

SEQ ID NO: 146 - protein, 265 aa, Artificial Sequence; identical to SEQ ID NO: 124
  Synthetic Polypeptide

SEQ ID NO: 147 - protein, 312 aa, Artificial Sequence; identical to SEQ ID NO: 128
  Synthetic Polypeptide

SEQ ID NO: 148 - protein, 252 aa, Artificial Sequence; identical to SEQ ID NO: 144
  Synthetic Polypeptide

SEQ ID NO: 149 - protein, 32 aa, Artificial Sequence
  Synthetic Polypeptide
KAPPLQQILDLHDFQGDALQPEDFLEEDEYHA

SEQ ID NO: 150 - protein, 19 aa, Artificial Sequence
  Synthetic Polypeptide
ASAPTLGGGGSGGGGSAAA

SEQ ID NO: 151 - protein, 4 aa, Artificial Sequence
  Synthetic Polypeptide
RSSR

SEQ ID NO: 152 - protein, 4 aa, Artificial Sequence
  Synthetic Polypeptide
RSRR

SEQ ID NO: 153 - protein, 34 aa, Artificial Sequence
  Synthetic Polypeptide
GLHPFMELRVLENTKRSRRNLGLDCDEHSSESRC

SEQ ID NO: 154 - protein, 34 aa, Artificial Sequence
  Synthetic Polypeptide
PEPDGCPVCVWRQHSRELRLESIKSQILSKLRLK

SEQ ID NO: 155 - protein, 34 aa, Artificial Sequence
  Synthetic Polypeptide
AAAAAAAAAAGVGGERSSRPAPSVAPEPDGCPVC

SEQ ID NO: 156 - protein, 15 aa, Artificial Sequence
  Synthetic Polypeptide
GLNDIFEAQKIEWHE

SEQ ID NO: 157 - protein, 22 aa, Artificial Sequence
  Synthetic Polypeptide
MDMRVPAQLLGLLLLWFSGVLG

SEQ ID NO: 158 - protein, 8 aa, Artificial Sequence
  Synthetic Polypeptide
DYKDDDDK

SEQ ID NO: 159 - protein, 8 aa, Artificial Sequence
  Synthetic Polypeptide
LEVLFQGP

SEQ ID NO: 160 - protein, 6 aa, Artificial Sequence
  Synthetic Polypeptide
HHHHHH

SEQ ID NO: 161 - protein, 4 aa, Artificial Sequence
  Synthetic Polypeptide
RELR

SEQ ID NO: 162 - protein, 9 aa, Artificial Sequence
  Synthetic Polypeptide
FTFDDYAMH

SEQ ID NO: 163 - protein, 9 aa, Artificial Sequence
  Synthetic Polypeptide
YTFTGYYMH

SEQ ID NO: 164 - protein, 9 aa, Artificial Sequence
  Synthetic Polypeptide
FTFSSYAMH

SEQ ID NO: 165 - protein, 9 aa, Artificial Sequence
  Synthetic Polypeptide
YTFTSYGIS

SEQ ID NO: 166 - protein, 9 aa, Artificial Sequence
  Synthetic Polypeptide
YTFTGYYIY

SEQ ID NO: 167 - protein, 11 aa, Artificial Sequence
  Synthetic Polypeptide
GSISSSSYYWG

SEQ ID NO: 168 - protein, 9 aa, Artificial Sequence
  Synthetic Polypeptide
FTFSSYSMN

(19) United States
(12) Patent Application Publication
(10) Pub. No.: US 2018/0208648 A1
(43) Pub. Date: Jul. 26, 2018

(54) GDF11 BINDING PROTEINS AND USES THEREOF

(71) Applicant: Scholar Rock, Inc., Cambridge, MA (US)

(72) Inventors: Michelle STRAUB, Watertown, MA (US); Katherine Jane TURNER, Acton, MA (US); Justin W. JACKSON, Cambridge, MA (US)

(73) Assignee: Scholar Rock, Inc., Cambridge, MA (US)

(21) Appl. No.: 15/746,467

(22) PCT Filed: Jul. 22, 2016
(86) PCT No.: PCT/US2016/043712

§ 371 Date: Jan. 22, 2018

Related U.S. Application Data
(60) Provisional application No. 62/195,504, filed on Jul. 22, 2015.
(60) Provisional application No. 62/275,068, filed on Jan. 5, 2016.

(51) Int. Cl. C07K 16/22

(52) U.S. Cl. CPC C07K 16/22; C07K 2317/32; C07K 2317/76; C07K 2317/94; C07K 2317/92; C07K 2317/24; C07K 2317/565; C07K 2317/33; C07K 2317/21

(57) ABSTRACT

Binding proteins that specifically bind to GD-F11 prodomain complex are disclosed. In some embodiments, antibodies that specifically bind to GDF11 prodomain complex are disclosed. These binding proteins may be used to treat or to prevent diseases caused by aberrant levels or activities of GDF11.

### CROSS REFERENCE TO RELATED APPLICATIONS

This application claims priority under 35 U.S.C. § 119(e) of U.S. Provisional Patent Application No. 62/195,504, filed on Jul. 22, 2015 and entitled “COMPOSITIONS AND METHODS FOR GROWTH FACTOR MODULATION”; and U.S. Provisional Patent Application No. 62/275,068, filed on Jan. 5, 2016 and entitled “GDF11 BINDING PROTEINS AND USES THEREOF. Each of these applications is incorporated herein by reference in its entirety for all purposes.

### FIELD OF THE DISCLOSURE

The present disclosure relates to GDF11 binding proteins, and to their uses, especially as it relates to the prevention and/or treatment of various diseases.

### BACKGROUND

GDF11 (Growth Differentiation Factor 11) is a member of the TGF-beta superfamily. This family of proteins is characterized generally by a polybasic proteolytic processing site which is cleaved from a prodomain-containing precursor to produce a mature protein containing seven conserved cysteine residues.

GDF11 has been shown to play an important role in regulating cell growth and differentiation in both embryonic and adult tissues (McPherron et al 1999). Dussiot et al. have shown that GDF11 is a negative regulator of late-stage erythropoiesis. Dussiot et al., Nature Med. 20:398-409 (2014). It has also been reported that GDF11 administration results in anemia and erythroid hyperplasia, whereas administration of an ActrIIA-Fc or a modified ActRIIB-Fc promotes erythropoiesis. Several studies have suggested that GDF11 may have deleterious effects on skeletal muscle and other tissues. See, e.g., Sinha et al., Science 344 (6184): 649-52 (2014); Katsimpardi et al., Science 344 (6184): 630-34 (2014); Egerman et al., Cell Metabolism 22: 1-11 (2015); and Loffredo et. al., Cell 153(4):828-839 (2013). More recently, increased levels of circulating GDF11 in older adults with cardiovascular disease has been associated with increased prevelance of diabetes and frailty and increased risk for post-operative complications and rehospitalizations. See Schafer M., et al., “Quantification of GDF11 and Myostatin in Human Aging and Cardiovascular Disease,” Cell Metabolism (2016) 23, 1207-1216.

Full-length GDF11 protein is expressed in an inactive state (proGDF11), with an N-terminal prodomain followed by a C-terminal growth factor domain (FIG. 1). proGDF11 is first converted to latent GDF11 via proteolytic cleavage by a proprotein convertase (such as PCSK5) at a dibasic site at the C terminus of the prodomain. Following cleavage between the prodomain and growth factor domain, the two domains remain non-covalently associated, and the mature growth factor is unable to bind to cell surface receptors and initiate signaling events. Full GDF11 maturation is achieved by proteolysis of the prodomain by the tolloid family of proteases to liberate the mature GDF11 growth factor (FIG. 2). Binding proteins capable of binding to the prodomain of GDF11, for example to the ARM region or the straight jacket region, may prevent proteolytic cleavage of GDF11 precursors (keeping GDF11 in its pro- or latent state), or lock the prodomain onto the growth factor domain, and thereby neutralize mature GDF11 functionality.

### SUMMARY

This disclosure pertains to GDF11 binding proteins (e.g., prodomain complex binding proteins). Binding proteins of the disclosure include, but are not limited to antibodies, antigen binding portions, and other antigen binding proteins capable of binding GDF11 (e.g., human prolatent GDF11 complexes). Further, this disclosure provides methods of making and using GDF11 binding proteins (e.g., prodomain complex binding proteins) to treat disorders caused by aberrant levels and/or activities of GDF11.

In one embodiment, the disclosure pertains to a binding protein capable of binding GDF11 prodomain complex. In another embodiment, the binding protein binds human GDF11 prodomain complex. In another embodiment, the binding protein is capable of modulating a biological function of GDF11. In another embodiment, the binding protein is capable of inhibiting the release of mature GDF11 from the prodomain. In another embodiment, the binding protein is capable of inhibiting proteolytic cleavage of a GDF11 prodomain complex by a proprotein convertase or tolloid protease. Accordingly, in some embodiments, the disclosure relates to inhibitors (e.g., binding proteins and other molecules such as small molecules) of GDF11 activation by proteolysis. In one aspect of the disclosure, the binding protein is capable of binding GDF11 prodomain complex, and inhibits the binding of GDF11 to its target.

One embodiment of the disclosure provides an isolated antibody, or antigen binding fragment thereof, wherein said antibody, or antigen binding fragment thereof binds human GDF11 prodomain complex and inhibits the binding of said GDF11 to its binding partner in a cell. In another embodiment, at a concentration of 50 nM proGDF11, the EC50s for the disclosed antibodies is from 10⁻⁶ M to 10⁻¹⁰ M, for example 10⁻⁶ M, 10⁻⁷ M, 10⁻⁸ M, 10 M, or 10⁻¹⁰ M.

In one embodiment, the binding protein is an isolated antibody. In another embodiment, the disclosure provides an isolated antibody, or antigen binding fragment thereof, wherein the antibody, or antigen binding fragment thereof binds human GDF11 prodomain complex and modulates the levels and/or activities of GDF11. In one aspect, the antibody, or antigen binding fragment thereof inhibits the activities of GDF11 by 50%, 60%, 70%, 80%, 90% or 100% after being administered to a subject in a therapeutically effective amount. In another aspect, the antibody, or antigen binding fragment thereof reduces the levels of GDF11 by 50%, 60%, 70%, 80%, 90% or 100% after being administered to a subject in a therapeutically effective amount.

In one embodiment, the binding protein of the disclosure has an on rate constant (kₒₙ) to GDF11 prodomain complex of at least about 10² M⁻¹ s⁻¹, at least about 10³ M⁻¹ s⁻¹, at least about 10⁴ M⁻¹ s⁻¹, at least about 10⁵ M⁻¹s⁻¹, at least about 10⁶ M⁻¹ s⁻¹, at least about 10⁷ M⁻¹ s⁻¹, or at least about 10⁸ M⁻¹ s⁻¹, as measured by surface biolayer interferometry.

In another embodiment, the binding protein of the disclosure has an off rate constant (k_{off}) to GDF11 prodomain complex of at most about 10⁻³ s⁻¹, at most about 10⁴ s⁻¹, at most about 10⁻⁵ s⁻¹, at most about 10⁻⁶ s⁻¹, at most about 10⁻⁷ s⁻¹, or at most about 10⁻⁷ s⁻¹, as measured by surface biolayer interferometry.

In another embodiment, the binding protein of the disclosure has a dissociation constant (K_{D}) to GDF11 prodomain complex of at most about 10⁻⁷ M; at most about 10⁻⁸ M; at most about 10⁻⁹ M; at most about 10⁻¹⁰ M; at most about 10⁻¹¹ M; at most about 10⁻¹² M; or at most 10⁻¹³ M as measured by surface biolayer interferometry.

In another embodiment, the binding protein comprises a binding domain capable of binding to the prodomain, for example to the ARM region or the straight jacket region, of the GDF11 prodomain complex.

In another embodiment, the binding protein comprises a binding domain capable of competitively inhibiting the binding proteins herein. More specifically, binding proteins capable of competitively inhibiting binding proteins are capable of binding to the ARM (e.g., Bin 1, or Bin 3) or the straightjacket (e.g., Bin 2) of the GDF11 prodomain complex (FIG. 3A).

In another embodiment, the binding protein disclosed here further comprises human Fc region. In another embodiment, the binding protein is a human antibody or antigen binding portion thereof capable of binding GDF11 prodomain complex.

In another embodiment, the binding protein disclosed here further comprises a human acceptor framework. In another embodiment, the binding protein is a CDR grafted antibody or antigen binding portion thereof capable of binding GDF11 prodomain complex. In another embodiment, the CDR grafted antibody or antigen binding portion thereof comprises one or more CDRs disclosed herein. In another embodiment, the CDR grafted antibody or antigen binding portion thereof comprises a human acceptor framework.

In another embodiment, the disclosed binding protein is a humanized antibody or antigen binding portion thereof capable of binding GDF11 prodomain complex. In another embodiment, the humanized antibody or antigen binding portion thereof comprise one or more CDRs disclosed above incorporated into a human antibody variable domain of a human acceptor framework. In another embodiment, the human antibody variable domain is a consensus human variable domain.

In another embodiment, the human acceptor framework comprises at least one Framework Region amino acid substitution at a key residue, wherein the key residue is selected from the group consisting of a residue adjacent to a CDR; a glycosylation site residue; a rare residue; a residue capable of interacting with human GDF11 prodomain complex; a residue capable of interacting with a CDR; a canonical residue; a contact residue between heavy chain variable region and light chain variable region; a residue within a Vernier zone; and a residue in a region that overlaps between a Chothia-defined variable heavy chain CDR1 and a Kabat-defined first heavy chain framework.

In an embodiment, the binding protein is a humanized antibody or antigen binding portion thereof capable of binding GDF11 prodomain complex. In another embodiment, the humanized antibody, or antigen binding portion, thereof comprises one or more CDRs disclosed herein. In another embodiment, the humanized antibody, or antigen binding portion, thereof comprises three or more CDRs disclosed herein. In another embodiment, the humanized antibody, or antigen binding portion, thereof comprises six CDRs disclosed herein.

In some aspects, the disclosure provides antibodies that specifically bind to a GDF11 prodomain complex. The disclosure, in some aspects, includes an antibody that specifically binds to human proGDF11, murine proGDF11, human latent GDF11, and murine latent GDF11, but does not specifically bind to human proGDF11 ARM8, human proGDF8, human prodomain GDF11 AMR8, or mature GDF11.

In some embodiments, the human proGDF11 and the human latent GDF11 has an amino acid sequence as set forth in SEQ ID NO: 82. In another embodiment, the murine proGDF11 and the murine latent GDF11 has an amino acid sequence as set forth in SEQ ID NO: 97. In other embodiments, the proGDF11 ARM8 has an amino acid sequence as set forth in SEQ ID NO: 122. In some embodiments, the human proGDF8 has an amino acid sequence as set forth in SEQ ID NO: 83. In other embodiments, the human prodomain GDF11 ARM8 has an amino acid sequence as set forth in SEQ ID NO: 124. In another embodiment, the mature GDF11 has an amino acid sequence as set forth in SEQ ID NO: 90.

In some embodiments, the antibody comprises a CDR-H3 amino acid sequence set forth in SEQ ID NO: 66, 72, 30, 36, or 42. In other embodiments, the antibody comprises a CDR-L3 amino acid sequence as set forth in SEQ ID NO: 69, 75, 33, 39, or 45. In another embodiment, the antibody comprises a CDR-H1 amino acid sequence as set forth in SEQ ID NO: 64, 70, 28, 34, or 40. In some embodiments, the antibody comprises a CDR-L1 amino acid sequence as set forth in SEQ ID NO: 67, 73, 31, 37, or 43. In other embodiments, the antibody comprises a CDR-H2 amino acid sequence as set forth in SEQ ID NO: 65, 71, 29, 35, or 41. In another embodiment, the antibody comprises a CDR-L2 amino acid sequence as set forth in SEQ ID NO: 68, 74, 32, 38, or 44. In some embodiments, the antibody comprises a variable heavy chain amino acid sequence as set forth in SEQ ID NO: 10, 12, or 14. In other embodiments, the antibody comprises a variable heavy chain amino acid sequence as set forth in SEQ ID NO: 11, 13, or 15.

Another aspect of the disclosure includes an antibody that specifically binds to human proGDF11, murine proGDF11, human latent GDF11, murine latent GDF11, human proGDF8, and human prodomain GDF11 AMR8, but does not specifically bind to human proGDF11 ARM8, or mature GDF11.

In some embodiments, the human proGDF11 and the human latent GDF11 has an amino acid sequence as set forth in SEQ ID NO: 82. In other embodiments, the murine proGDF11 and the murine latent GDF11 has an amino acid sequence as set forth in SEQ ID NO: 97. In another embodiment, the proGDF11 ARM8 has an amino acid sequence as set forth in SEQ ID NO: 122. In other embodiments, the human proGDF8 has an amino acid sequence as set forth in SEQ ID NO: 83. In some embodiments, the human prodomain GDF11 ARM8 has an amino acid sequence as set forth in SEQ ID NO: 124. In another embodiment, the mature GDF11 has an amino acid sequence as set forth in SEQ ID NO: 90.

In some embodiments, the antibody comprises a CDR-H3 amino acid sequence set forth in SEQ ID NO: 66, 72, or 24. In other embodiments, the antibody comprises a CDR-L3 amino acid sequence as set forth in SEQ ID NO: 69, 75, or 27. In another embodiment, the antibody comprises a CDR-H1 amino acid sequence as set forth in SEQ ID NO: 64, 70, or 22. In some embodiments, the antibody comprises a CDR-L1 amino acid sequence as set forth in SEQ ID NO: 67, 73, or 25. In another embodiment, the antibody comprises a CDR-H2 amino acid sequence as set forth in SEQ ID NO: 65, 71, or 23. In other embodiments, the antibody comprises a CDR-L2 amino acid sequence as set forth in SEQ ID NO: 68, 74, or 26. In some embodiments, the antibody comprises a variable heavy chain amino acid sequence as set forth in SEQ ID NO: 8. In another embodiment, the antibody comprises a variable heavy chain amino acid sequence as set forth in SEQ ID NO: 9.

In another aspect, the instant disclosure includes an antibody that specifically binds to human proGDF11, murine proGDF11, human latent GDF11, murine latent GDF11, human proGDF8, human prodomain GDF11 AMR8, human proGDF11 ARM8, and mature GDF11.

In some embodiments, the human proGDF11 and the human latent GDF11 has an amino acid sequence as set forth in SEQ ID NO: 82. In another embodiment, the murine proGDF11 and the murine latent GDF11 has an amino acid sequence as set forth in SEQ ID NO: 97. In a further embodiment, the proGDF11 ARM8 has an amino acid sequence as set forth in SEQ ID NO: 122. In one embodiment, the human proGDF8 has an amino acid sequence as set forth in SEQ ID NO: 83. In another embodiment, the human prodomain GDF11 ARM8 has an amino acid sequence as set forth in SEQ ID NO: 124. In some embodiments, the mature GDF11 has an amino acid sequence as set forth in SEQ ID NO: 90.

In some embodiments, the antibody comprises a CDR-H3 amino acid sequence set forth in SEQ ID NO: 66, 78, or 48. In another embodiment, the antibody comprises a CDR-L3 amino acid sequence as set forth in SEQ ID NO: 69, 81, or 51. In one embodiment, the antibody comprises a CDR-H1 amino acid sequence as set forth in SEQ ID NO: 64, 76, or 46. In other embodiments, the antibody comprises a CDR-L1 amino acid sequence as set forth in SEQ ID NO: 67, 79, or 49. In some embodiments, the antibody comprises a CDR-H2 amino acid sequence as set forth in SEQ ID NO: 65, 77, or 47. In another embodiment, the antibody comprises a CDR-L2 amino acid sequence as set forth in SEQ ID NO: 68, 80, or 50. In one embodiment, the antibody comprises a variable heavy chain amino acid sequence as set forth in SEQ ID NO: 16. In other embodiments, the antibody comprises a variable heavy chain amino acid sequence as set forth in SEQ ID NO: 17.

The disclosure, in another aspect, includes an antibody that specifically binds to human proGDF11, murine proGDF11, human latent GDF11, murine latent GDF11, human proGDF8, human prodomain GDF11 AMR8, and human proGDF11 ARM8, but does not specifically bind to mature GDF11.

In some embodiments, the human proGDF11 and the human latent GDF11 has an amino acid sequence as set forth in SEQ ID NO: 82. In other embodiments, the murine proGDF11 and the murine latent GDF11 has an amino acid sequence as set forth in SEQ ID NO: 97. In another embodiment, the proGDF11 ARM8 has an amino acid sequence as set forth in SEQ ID NO: 122. In one embodiment, the human proGDF8 has an amino acid sequence as set forth in SEQ ID NO: 83. In some embodiments, the human prodomain GDF11 ARM8 has an amino acid sequence as set forth in SEQ ID NO: 124. In other embodiments, the mature GDF11 has an amino acid sequence as set forth in SEQ ID NO: 90.

In some embodiments, the antibody comprises a CDR-H3 amino acid sequence set forth in SEQ ID NO: 66, 78, or 60. In another embodiment, the antibody comprises a CDR-L3 amino acid sequence as set forth in SEQ ID NO: 69, 81, or 63. In one embodiment, the antibody comprises a CDR-H1 amino acid sequence as set forth in SEQ ID NO: 64, 76, or 58. In some embodiments, the antibody comprises a CDR-L1 amino acid sequence as set forth in SEQ ID NO: 67, 79, or 61. In another embodiment, the antibody comprises a CDR-H2 amino acid sequence as set forth in SEQ ID NO: 65, 77, or 59. In one embodiment, the antibody comprises a CDR-L2 amino acid sequence as set forth in SEQ ID NO: 68, 80, or 62. In some embodiments, the antibody comprises a variable heavy chain amino acid sequence as set forth in SEQ ID NO: 20. In other embodiments, the antibody comprises a variable heavy chain amino acid sequence as set forth in SEQ ID NO: 21.

The disclosure, in another aspect, includes an antibody that specifically binds to human proGDF11, murine proGDF11, human latent GDF11, murine latent GDF11, and mature GDF11, but does not specifically bind to human proGDF11 ARM8, human proGDF8, or human prodomain GDF11 AMR8.

In some embodiments, the human proGDF11 and the human latent GDF11 has an amino acid sequence as set forth in SEQ ID NO: 82. In another embodiment, the murine proGDF11 and the murine latent GDF11 has an amino acid sequence as set forth in SEQ ID NO: 97. In another embodiment, the proGDF11 ARM8 has an amino acid sequence as set forth in SEQ ID NO: 122. In a further embodiment, the human proGDF8 has an amino acid sequence as set forth in SEQ ID NO: 83. In some embodiments, the human prodomain GDF11 ARM8 has an amino acid sequence as set forth in SEQ ID NO: 124. In other embodiments, the mature GDF11 has an amino acid sequence as set forth in SEQ ID NO: 90.

In some embodiments, the antibody comprises a CDR-H3 amino acid sequence set forth in SEQ ID NO: 66, or 54. In other embodiments, the antibody comprises a CDR-L3 amino acid sequence as set forth in SEQ ID NO: 69, or 57. In a further embodiment, the antibody comprises a CDR-H1 amino acid sequence as set forth in SEQ ID NO: 64, or 52. In some embodiments, the antibody comprises a CDR-L1 amino acid sequence as set forth in SEQ ID NO: 67, or 55. In other embodiments, the antibody comprises a CDR-H2 amino acid sequence as set forth in SEQ ID NO: 65, or 53. In some embodiments, the antibody comprises a CDR-L2 amino acid sequence as set forth in SEQ ID NO: 68, or 56. In other embodiments, the antibody comprises a variable heavy chain amino acid sequence as set forth in SEQ ID NO: 18. In another embodiment, the antibody comprises a variable heavy chain amino acid sequence as set forth in SEQ ID NO: 19.

The disclosure, in another aspect, provides an antibody comprising an antigen binding domain, said antigen binding domain comprising six CDRs: CDR-H1, CDR-H2, CDR-H3, CDR-L1, CDR-L2, and CDR-L3, wherein at least one of the CDR sequences is selected from the group consisting of; SEQ ID NO: 64, SEQ ID NO: 65, SEQ ID NO: 66, SEQ ID NO: 67, SEQ ID NO: 68, and SEQ ID NO: 69.

In some embodiments, at least one of the CDR sequences is selected from the group consisting of:
CDRH1 sequence is X₁ Y X₃ X₄ X₅ (SEQ ID NO: 64);
   Wherein X₁ is D, G, or S
   Wherein X₃ is A, Y, G, or S
   Wherein X₄ is M, I, or W
   Wherein X₅ is H, S, Y, G, or N:
CDRH2 sequence is X₁ X₂ X₃ X₄ X₅ X₆ X₇ X₈ X₉ X₁₀ Y X₁₂ X₁₃ X₁₄ X₁₅ X₁₆ X₁₇ (SEQ ID NO: 65);
   Wherein X₁ is G, W, V, Y, or absent
   Wherein X₂ is I, or E
   Wherein X₃ is S, N, R, or I
   Wherein X₄ is W, P, Y, A, or S
   Wherein X₅ is N, D, Y, H, or S
   Wherein X₆ is S, G, or N,
   Wherein X₇ is G, or S
   Wherein X₈ is S, G, N, D, or T
   Wherein X₉ is I, T, or E
   Wherein X₁₀ is G, N, or Y,
   Wherein X₁₂ is A, or N
   Wherein X₁₃ is D, Q, or P
   Wherein X₁₄ is S, or K
   Wherein X₁₅ is V, F, or L
   Wherein X₁₆ is K or Q
   Wherein X₁₇ is G, D, or S;
CDRH3 sequence is X₁ X₂ X₃ X₄ X₅ X₆ X₇ X₈ X₉ X₁₀ X₁₁ X₁₂ X₁₃ X₁₄ X₁₅ X₁₆ X₁₇ X₁₈ (SEQ ID NO: 66);
   Wherein X₁ is G, or absent
   Wherein X₂ is G, or absent
   Wherein X₃ is S, D, or absent
   Wherein X₄ is I, G, or absent
   Wherein X₅ is A, D, T, N, I, or absent
   Wherein X₆ is V, F, P, Y, or absent
   Wherein X₇ is A, W, P, D, Y, or absent
   Wherein X₈ is G, S, L, I, V, D, or absent
   Wherein X₉ is T, G, W, L, S, or absent
   Wherein X₁₀ is L, Y, F, T, S, or absent
   Wherein X₁₁ is E, V, P, G, or S,
   Wherein X₁₂ is V, D, Q, E, Y, or W
   Wherein X₁₃ is T, Y, Q, or E
   Wherein X₁₄ is G, Y, N, A, or D
   Wherein X₁₅ is D, G, W, A, P, Y, or L
   Wherein X₁₆ is L, M, or F,
   Wherein X₁₇ is D, or G
   Wherein X₁₈ is Y, V, P or I:
CDRL1 sequence is X₁ X₂ S Q X₅ X₆ X₇ X₈ X₉ Y L X₁₂ (SEQ ID NO: 67);
   Wherein X₁ is R, or Q
   Wherein X₂ is A, or T
   Wherein X₅ is F, D, S, R, or H
   Wherein X₆ is L, I, or V
   Wherein X₇ is S, I, or absent
   Wherein X₈ is S, or absent
   Wherein X₉ is T, N, or absent
   Wherein X₁₂ is A, or N:
CDRL2 sequence is X₁ A S X₄ X₅ X₆ X₇ (SEQ ID NO: 68);
   Wherein X₁ is S, D, G, K, or A
   Wherein X₄ is N, S, or T
   Wherein X₅ is R, or L
   Wherein X₆ is A, E, or Q:
   Wherein X₇ is T, or S, and
CDRL3 sequence is X₁ X₂ X₃ X₄ X₅ X₆ P X₈ X₉ (SEQ ID NO: 69);
   Wherein X₁ is M, or Q
   Wherein X₂ is Q, K, or H
   Wherein X₃ is A, Y, or S
   Wherein X₄ is T, S, G, Y, or Q
   Wherein X₅ is H, T, S, or absent
   Wherein X₆ is W, A, T, Y, or absent
   Wherein X₈ is Y, L, I, P, or absent
   Wherein X₉ is T, or absent.

In another embodiment, at least one of the CDR sequences is selected from the group consisting of:
CDRH1 sequence is X₁ Y X₃ X₄ X₅ (SEQ ID NO: 70)
   Wherein X₁ is D, G, or S
   Wherein X₃ is A, Y, or G
   Wherein X₄ is M, or I
   Wherein X₅ is H, or S
CDRH2 sequence is X₁ I X₃ X₄ X₅ X₆ X₇ X₈ X₉ X₁₀ Y A X₁₃ X₁₄ X₁₅ X₁₆G (SEQ ID NO: 71)
   Wherein X1 is G, W, or V
   Wherein X3 is S, or N
   Wherein X4 is W, P, Y, or A,
   Wherein X5 is N, D, or Y
   Wherein X6 is S, G, or N,
   Wherein X7 is G, or S
   Wherein X8 is S, G, or N
   Wherein X9 is I, T, or E
   Wherein X10 is G, N, or Y,
   Wherein X13 is D, or Q
   Wherein X14 is S, or K
   Wherein X15 is V, F, or L
   Wherein X16 is K or Q
CDRH3 sequence is X₁ X₂ X₃ X₄ ^{X5} X₆ X₇ X₈ X₉ X₁₀ X₁₁ X₁₂ X₁₃ X₁₄ X₁₅ X₁₆ D X₁₈ (SEQ ID NO: 72)
   Wherein X₁ is G, or absent
   Wherein X₂ is G, or absent
   Wherein X₃ is S, or absent
   Wherein X₄ is I, or absent
   Wherein X₅ is A, D, T, or absent
   Wherein X₆ is V, F, P, or absent
   Wherein X₇ is A, W, P, or absent
   Wherein X₈ is G, S, L, or absent
   Wherein X₉ is T, G, W, or absent
   Wherein X₁₀ is L, Y, F, or absent
   Wherein X₁₁ is E, V, P, or G
   Wherein X₁₂ is V, D, Q, or E
   Wherein X₁₃ is T, or Y
   Wherein X₁₄ is G, Y, or N
   Wherein X₁₅ is D, G, W, or A
   Wherein X₁₆ is L, M, or F,
   Wherein X₁₈ is Y, V, P or I
CDRL1 sequence is X₁ A S Q X₅ X₆ X₇ S X₉ Y L X₁₂ (SEQ ID NO: 73)
   Wherein X₁ is R, or Q
   Wherein X₅ is F, D, or S
   Wherein X₆ is L, I, or V
   Wherein X₇ is S, or absent
   Wherein X₉ is T, or N
   Wherein X₁₂ is A, or N
CDRL2 sequence is X₁ A S N₄ X₅ X₆ T (SEQ ID NO: 74)
   Wherein X₁ is S, or D
   Wherein X₅ is R, or L
   Wherein X₆ is A, or E
CDRL3 sequence is X₁ X₂ X₃ X₄ X₅ X₆ P X₈ T (SEQ ID NO: 75)
   Wherein X₁ is M, or Q
   Wherein X₂ is Q, or K
   Wherein X₃ is A, Y, or S
   Wherein X₄ is T, S, G, or Y
   Wherein X₅ is H, T, or S
   Wherein X₆ is W, A, or T
   Wherein X₈ is Y, L, or I.

In other embodiments, at least one of the CDR sequences is selected from the group consisting of:
CDRH1 sequence is X₁ Y X₃ X₄ X₅ (SEQ ID NO: 76)
   Wherein X₁ is G, or S
   Wherein X₃ is Y, or S
   Wherein X₄ is I, or M
   Wherein X₅ is Y, or N
CDRH2 sequence is X₁ I X₃ X₄ X₅ S X₇ X₈ X₉ X₁₀ Y A X₁₃ X₁₄ X₁₅ X₁₆ X₁₇ (SEQ ID NO: 77)
   Wherein X1 is W, or Y
   Wherein X3 is R, or S
   Wherein X4 is P, or S
   Wherein X5 is N, or S
   Wherein X7 is G, or S
   Wherein X8 is D, or T
   Wherein X9 is T, or I
   Wherein X10 is N, or Y,
   Wherein X13 is Q, or D
   Wherein X14 is K, or S
   Wherein X15 is F, or V
   Wherein X16 is Q or K
   Wherein X17 is D, or G
CDRH3 sequence is X₁ X₂ X₃ Y X₅ X₆ X₇ X₈ G Y X₁₁ X₁₂ X₁₃ X₁₄ X₁₅ Y (SEQ ID NO: 78)
   Wherein X₁ is D, or absent
   Wherein X₂ is G, or absent
   Wherein X₃ is N, or I
   Wherein X₅ is D, or Y
   Wherein X₆ is I, or D
   Wherein X₇ is L, or S
   Wherein X₈ T, or S
   Wherein X₁₁ is Q, or Y
   Wherein X₁₂ is A, or D
   Wherein X₁₃ is P, or L
   Wherein X₁₄ is L, or F
   Wherein X₁₅ is G, or D
CDRL1 sequence is RA S Q X₅ X₆ X₇ S X₉ Y L X₁₂ (SEQ ID NO: 79)
   Wherein X₅ is R, or S
   Wherein X₆ is V, or I
   Wherein X₇ I, or S
   Wherein X₉ is N, or absent
   Wherein X₁₂ is A, or N
CDRL2 sequence is X₁ A S S X₅ X₆ X₇ (SEQ ID NO: 80)
   Wherein X₁ is G, or A
   Wherein X₅ is R, or L
   Wherein X₆ is A, or Q
   Wherein X₇ is T, or S
CDRL3 sequence is Q X₂ X₃ X₄ X₅ X₆P X₈ X₉ (SEQ ID NO: 81)
   Wherein X₂ is H, or Q
   Wherein X₃ is Y, or S
   Wherein X₄ is G, or Y
   Wherein X₅ is S, or absent
   Wherein X₆ is T, or absent
   Wherein X₈ is P, or absent
   Wherein X₉ is T, or absent

In some embodiments, the antibody provided herein comprises the CDRH3 sequence of X₁ X₂ X₃ X₄ X₅ X₆ X₇ X₈ X₉ X₁₀ X₁₁ X₁₂ X₁₃ X₁₄ X₁₅ X₁₆ X₁₇ X₁₈ (SEQ ID NO: 66);
Wherein X₁ is G, or absent
Wherein X₂ is G, or absent
Wherein X₃ is S, D, or absent
Wherein X₄ is I, G, or absent
Wherein X₅ is A, D, T, N, I, or absent
Wherein X₆ is V, F, P, Y, or absent
Wherein X₇ is A, W, P, D, Y, or absent
Wherein X₈ is G, S, L, I, V, D, or absent
Wherein X₉ is T, G, W, L, S, or absent
Wherein X₁₀ is L, Y, F, T, S, or absent
Wherein X₁₁ is E, V, P, G, or S,
Wherein X₁₂ is V, D, Q, E, Y, or W
Wherein X₁₃ is T, Y, Q, or E
Wherein X₁₄ is G, Y, N, A, or D
Wherein X₁₅ is D, G, W, A, P, Y, or L
Wherein X₁₆ is L, M, or F,
Wherein X₁₇ is D, or G
Wherein X₁₈ is Y, V, P or I; or
X₁ X₂ X₃ X₄ X₅ X₆ X₇ X₈ X₉ X₁₀ X₁₁ X₁₂ X₁₃ X₁₄ X₁₅ X₁₆ D X₁₈ (SEQ ID NO: 72)
   Wherein X₁ is G, or absent
   Wherein X₂ is G, or absent
   Wherein X₃ is S, or absent
   Wherein X₄ is I, or absent
   Wherein X₅ is A, D, T, or absent
   Wherein X₆ is V, F, P, or absent
   Wherein X₇ is A, W, P, or absent
   Wherein X₈ is G, S, L, or absent
   Wherein X₉ is T, G, W, or absent
   Wherein X₁₀ is L, Y, F, or absent
   Wherein X₁₁ is E, V, P, or G
   Wherein X₁₂ is V, D, Q, or E
   Wherein X₁₃ is T, or Y
   Wherein X₁₄ is G, Y, or N
   Wherein X₁₅ is D, G, W, or A
   Wherein X₁₆ is L, M, or F,
   Wherein X₁₈ is Y, V, P or I; or
X₁ X₂ X₃ Y X₅ X₆ X₇ X₈ G Y X₁₁ X₁₂ X₁₃ X₁₄ X₁₅Y (SEQ ID NO: 78)
   Wherein X₁ is D, or absent
   Wherein X₂ is G, or absent
   Wherein X₃ is N, or I
   Wherein X₅ is D, or Y
   Wherein X₆ is I, or D
   Wherein X₇ is L, or S
   Wherein X₈ T, or S
   Wherein X₁₁ is Q, or Y
   Wherein X₁₂ is A, or D
   Wherein X₁₃ is P, or L
   Wherein X₁₄ is L, or F
   Wherein X₁₅ is G, or D.

In another embodiment, the antibody provided herein comprises the CDRL3 sequence of X₁ X₂ X₃ X₄ X₅ X₆ P X₈ X₉ (SEQ ID NO: 69);
Wherein X₁ is M, or Q
Wherein X₂ is Q, K, or H
Wherein X₃ is A, Y, or S
Wherein X₄ is T, S, G, Y, or Q
Wherein X₅ is H, T, S, or absent
Wherein X₆ is W, A, T, Y, or absent
Wherein X₈ is Y, L, I, P, or absent
Wherein X₉ is T, or absent; or
X₁ X₂ X₃ X₄ X₅ X₆ P X₈ T (SEQ ID NO: 75)
   Wherein X₁ is M, or Q
   Wherein X₂ is Q, or K
   Wherein X₃ is A, Y, or S
   Wherein X₄ is T, S, G, or Y
   Wherein X₅ is H, T, or S
   Wherein X₆ is W, A, or T
   Wherein X₈ is Y, L, or I; or
Q X₂ X₃ X₄ X₅ X₆P X₈ X₉ (SEQ ID NO: 81)
   Wherein X₂ is H, or Q
   Wherein X₃ is Y, or S
   Wherein X₄ is G, or Y
   Wherein X₅ is S, or absent
   Wherein X₆ is T, or absent
   Wherein X₈ is P, or absent
   Wherein X₉ is T, or absent.

The present disclosure, in some aspects, includes an antibody comprising an antigen binding domain, said antigen binding domain comprising six CDRs: CDR-H1, CDR-H2, CDR-H3, CDR-L1, CDR-L2, and CDR-L3, wherein:
CDR-H1 is selected from the group consisting of:
SEQ ID NO:22;
SEQ ID NO:28;
SEQ ID NO:34;
SEQ ID NO:40;
SEQ ID NO:46;
SEQ ID NO:52; and
SEQ ID NO:58;
CDR-H2 is selected from the group consisting of:
SEQ ID NO:23;
SEQ ID NO:29;
SEQ ID NO:35;
SEQ ID NO:41;
SEQ ID NO:47;
SEQ ID NO:53; and
SEQ ID NO:59;
CDR-H3 is selected from the group consisting of:
SEQ ID NO:24;
SEQ ID NO:30;
SEQ ID NO:36;
SEQ ID NO:42;
SEQ ID NO:48;
SEQ ID NO:54; and
SEQ ID NO:60;
CDR-L1 is selected from the group consisting of:
SEQ ID NO:25;
SEQ ID NO:31;
SEQ ID NO:37;
SEQ ID NO:43;
SEQ ID NO:49;
SEQ ID NO:55; and
SEQ ID NO:61;
CDR-L2 is selected from the group consisting of:
SEQ ID NO:26;
SEQ ID NO:32;
SEQ ID NO:38;
SEQ ID NO:44;
SEQ ID NO:50;
SEQ ID NO:56; and
SEQ ID NO:62; and
CDR-L3 is selected from the group consisting of:
SEQ ID NO:27;
SEQ ID NO:33;
SEQ ID NO:39;
SEQ ID NO:45;
SEQ ID NO:51;
SEQ ID NO:57; and
SEQ ID NO:63.

In some embodiments, three of the six CDRs are selected from the group of variable domain CDR sets consisting of:

VH GDF11 Inh-1 CDR Set
CDR-H1: SEQ ID NO:22
CDR-H2: SEQ ID NO:23
CDR-H3: SEQ ID NO:24

VL GDF11 Inh-1 CDR Set
CDR-L1: SEQ ID NO:25
CDR-L2: SEQ ID NO:26
CDR-L3: SEQ ID NO:27

VH GDF11 Inh-2 CDR Set
CDR-H1: SEQ ID NO:28
CDR-H2: SEQ ID NO:29
CDR-H3: SEQ ID NO:30

VL GDF11 Inh-2 CDR Set
CDR-L1: SEQ ID NO:31
CDR-L2: SEQ ID NO:32
CDR-L3: SEQ ID NO:33

VH GDF11 Inh-3 CDR Set
CDR-H1: SEQ ID NO:34
CDR-H2: SEQ ID NO:35
CDR-H3: SEQ ID NO:36

VL GDF11 Inh-3 CDR Set
CDR-L1: SEQ ID NO:37
CDR-L2: SEQ ID NO:38
CDR-L3: SEQ ID NO:39

VH GDF11 Inh-4 CDR Set
CDR-H1: SEQ ID NO:40
CDR-H2: SEQ ID NO:41
CDR-H3: SEQ ID NO:42

VL GDF11 Inh-4 CDR Set
CDR-L1: SEQ ID NO:43
CDR-L2: SEQ ID NO:44
CDR-L3: SEQ ID NO:45

VH GDF11 Inh-5 CDR Set
CDR-H1: SEQ ID NO:46
CDR-H2: SEQ ID NO:47
CDR-H3: SEQ ID NO:48

VL GDF11 Inh-5 CDR Set
CDR-L1: SEQ ID NO:49
CDR-L2: SEQ ID NO:50
CDR-L3: SEQ ID NO:51

VH GDF11 Inh-6 CDR Set
CDR-H1: SEQ ID NO:52
CDR-H2: SEQ ID NO:53
CDR-H3: SEQ ID NO:54

VL GDF11 Inh-6 CDR Set
CDR-L1: SEQ ID NO:55
CDR-L2: SEQ ID NO:56
CDR-L3: SEQ ID NO:57

VH GDF11 Inh-7 CDR Set
CDR-H1: SEQ ID NO:58
CDR-H2: SEQ ID NO:59
CDR-H3: SEQ ID NO:60 and

VL GDF11 Inh-7 CDR Set
CDR-L1: SEQ ID NO:61
CDR-L2: SEQ ID NO:62
CDR-L3: SEQ ID NO:63.

In some embodiments, the antibody comprises at least two variable domain CDR sets. In other embodiments, the antibody comprises at least two variable domain CDR sets are selected from a group consisting of:
VH GDF11 Inh-1 CDR Set and VL GDF11 Inh-1 CDR Set,
VH GDF11 Inh-2 CDR Set and VL GDF11 Inh-2 CDR Set,
VH GDF11 Inh-3 CDR Set and VL GDF11 Inh-3 CDR Set,
VH GDF11 Inh-4 CDR Set and VL GDF11 Inh-4 CDR Set,
VH GDF11 Inh-5 CDR Set and VL GDF11 Inh-5 CDR Set,
VH GDF11 Inh-6 CDR Set and VL GDF11 Inh-6 CDR Set, and
VH GDF11 Inh-7 CDR Set and VL GDF11 Inh-7 CDR Set.

In some embodiments, the antibody further comprising a human acceptor framework.

In other embodiments, the isolated antibody, or antigen binding fragment thereof comprises at least one variable domain having amino acid sequence selected from the group consisting of SEQ ID NOs: 8-21. In other embodiments, the antibody comprises at least one heavy chain variable domain and at least one light chain variable domain, said heavy chain variable domain having amino acid sequence selected from the group consisting of SEQ ID NOs: 8, 10, 12, 14, 16, 18 and 20, and said light chain variable domain having amino acid sequence selected from the group consisting of SEQ ID NOs: 9, 11, 13, 15, 17, 19 and 21. In another embodiment, the antibody comprises two variable domains, wherein said two variable domains have amino acid sequences selected from the group consisting of:
SEQ ID NOs:8 and 9,
SEQ ID NOs:10 and 11,
SEQ ID NOs:12 and 13,
SEQ ID NOs:14 and 15,
SEQ ID NOs:16 and 17,
SEQ ID NOs:18 and 19, and
SEQ ID NOs:20 and 21.

In some embodiments, the antibody provided herein further comprises a heavy chain immunoglobulin constant domain selected from the group consisting of: a human IgM constant domain; a human IgG1 constant domain; a human IgG2 constant domain; a human IgG3 constant domain; a human IgG4 constant domain; a human IgE constant domain and a human IgA constant domain. In other embodiments, said heavy chain immunoglobulin constant domain is a human IgG1 constant domain. In other embodiments, the antibody further comprises a light chain immunoglobulin constant domain, wherein said light chain immunoglobulin constant domain is a human Ig kappa constant domain. In another embodiment, the antibody further comprises a light chain immunoglobulin constant domain, wherein said light chain immunoglobulin constant domain is a human Ig lambda constant domain.

In other embodiments, the antibody is selected from the group consisting of: an immunoglobulin molecule, an scFv, a monoclonal antibody, a human antibody, a chimeric antibody, a humanized antibody, a single domain antibody, a Fab fragment, a Fab′ fragment, an F(ab′)₂, an Fv, a disulfide linked Fv, a single domain antibody, a diabody, a multispecific antibody, a bispecific antibody, and a dual specific antibody. In other embodiments, the antibody is a human antibody.

In another embodiment, the antibody is capable of modulating a biological function or levels of GDF11. In some embodiments, the antibody is capable of neutralizing GDF11. In another embodiment, said GDF11 is human GDF11. In one embodiment, said antibody is capable of enhancing erythropoiesis. In another embodiment, said antibody has a dissociation constant (KD) selected from the group consisting of: at most about 10⁻⁷ M; at most about 10⁻⁸ M; at most about 10⁻⁹ M; at most about 10⁻¹⁰ M; at most about 10⁻¹¹ M; at most about 10-¹² M; and at most 10⁻¹³ M to a human GDF11 pro-domain complex. In a further embodiment, said antibody has an on rate selected from the group consisting of: at least about 10²M⁻¹s⁻¹; at least about 10³M^{-l}s⁻¹; at least about 104M⁻¹ s⁻¹; at least about 10⁵ M⁻¹ s⁻¹; and at least about 10⁶ M⁻¹ s⁻¹ to a human GDF11 pro-domain complex.

In some embodiments, said antibody has an off rate selected from the group consisting of: at most about 10⁻³ s⁻¹; at most about 10⁴ s⁻¹; at most about 10⁻⁵ s⁻¹; and at most about 10⁻⁶ s⁻¹ to a human GDF11 pro-domain complex. In another embodiment, the antibody is isolated. In some embodiments, the antibody specifically binds human GDF11 pro-domain complex.

The disclosure, in another aspect, includes an antibody construct comprising the antibody provided herein and further comprising a linker polypeptide or an immunoglobulin constant domain.

In some embodiments, the antibody construct is selected from the group consisting of: an immunoglobulin molecule, a monoclonal antibody, a chimeric antibody, a CDR-grafted antibody, a humanized antibody, a Fab, a Fab′, a F(ab′)₂, a Fv, a disulfide linked Fv, a scFv, a single domain antibody, a diabody, a multispecific antibody, a dual specific antibody, and a bispecific antibody.

In another embodiment, said antibody construct comprises a heavy chain immunoglobulin constant domain selected from the group consisting of: a human IgM constant domain, a human IgG1 constant domain, a human IgG2 constant domain, a human IgG3 constant domain, a human IgG4 constant domain, a human IgE constant domain, a human IgA constant domain, and an IgG constant domain variant with one or more mutations altering binding strength to Fc neonatal receptor, Fc gamma receptors, or C1q.

The disclosure, in some aspects, includes an antibody conjugate comprising the antibody construct provided herein, wherein said antibody construct is conjugated to a therapeutic or cytotoxic agent. In one embodiment, said therapeutic or cytotoxic agent is selected from the group consisting of: an anti-metabolite, an alkylating agent, an antibiotic, a growth factor, a cytokine, an anti-angiogenic agent, an anti-mitotic agent, an anthracycline, toxin, and an apoptotic agent.

The disclosure, in some aspects, includes a pharmaceutical composition comprising the binding proteins (antibody, antibody construct, or antibody construct) provided herein, and a pharmaceutically acceptable carrier. In one embodiment, binding of the antibody, or the antibody construct, to a proGDF11 inhibits proteolytic cleavage of the proGDF11 by a proprotein convertase.

The disclosure, in another aspect, provides a method for reducing human GDF11 activity, comprising contacting human GDF11 prodomain complex with the binding proteins (antibody, antibody construct, or antibody conjugate) provided herein, such that human GDF11 activity is reduced.

Another aspect of the disclosure provides a method for reducing human GDF11 activity in a human subject suffering from a disorder in which GDF11 activity is detrimental, comprising administering to the human subject the binding proteins (antibody, antibody construct, or antibody conjugate) provided herein, such that human GDF11 activity in the human subject is reduced. In one embodiment, said disorder is selected from the group consisting of: anemia, and erythroid hyperplasia.

An additional aspect of the disclosure provides a method of modulating growth factor activity in a biological system comprising contacting said biological system with the binding proteins (antibody, antibody construct, or antibody conjugate) provided herein.

In some embodiments, said growth factor activity comprises GDF11 activity. In another embodiment, the antibody is a stabilizing antibody and wherein contacting said biological system with said stabilizing antibody results in inhibition of release of at least 5% of total GDF11 mature growth factor in said biological system. In other embodiments, binding of the antibody or antigen binding portion thereof, the antibody construct, or the antibody conjugate to a proGDF11 inhibits proteolytic cleavage of the proGDF11 by a proprotein convertase. In a further embodiment, the antibody inhibits proteolytic cleavage of the proGDF11 by a proprotein convertase.

In one aspect, the present disclosure provides a method of treating a TGF-β-related indication in a subject comprising contacting said subject with the binding proteins (antibody, antibody construct, or antibody conjugate) provided herein.

In one embodiment, said TGF-β-related indication comprises a cardiovascular indication selected from the group consisting of cardiac hypertrophy, cardiac atrophy, atherosclerosis and restenosis. In another embodiment, said TGF-β-related indication comprises a GDF11-related indication. In a further embodiment, said GDF11-related indication comprises erythroid hyperplasia anemia and/or β-thalassemia.

Another aspect of the disclosure includes a nucleic acid encoding the binding proteins (antibody, antibody construct, or the antibody conjugate) provided herein.

A further aspect of the disclosure includes vector comprising the nucleic acid provided herein.

An additional aspect of the disclosure includes a cell comprising the nucleic acid provided herein.

Another aspect of the disclosure provides a kit comprising the binding proteins (antibody, antibody construct, or antibody conjugate) provided herein and instructions for use thereof.

One aspect of the disclosure includes an antibody that competes for binding to an epitope with the binding proteins (antibody, antibody construct, or antibody conjugate) provided herein.

Another aspect of the disclosure provides an antibody that binds to the same epitope as the binding proteins (antibody, antibody construct, or antibody conjugate) provided herein.

A further aspect of the disclosure includes an antibody that competes for binding to an epitope of human proGDF11 or an epitope of human latent GDF11 with the binding proteins (antibody, antibody construct, or antibody conjugate) provided herein.

In some embodiments, the antibody specifically binds to an epitope of human proGDF11 or human latent GDF11 at the same epitope as the binding proteins (antibody, antibody construct, or antibody conjugate) provided herein.

In another embodiment, the antibody competes for binding to the epitope with an equilibrium dissociation constant (Kd) between the antibody and the epitope of less than 10⁻⁶ M. In one embodiment, the Kd is in a range of 10⁻¹¹ M to 10⁻⁶ M.

Another aspect of the disclosure includes a composition comprising the binding proteins (antibody, antibody construct, or antibody conjugate) provided herein and a carrier.

In some embodiments, the composition is a pharmaceutical composition comprising a therapeutically effective amount of the binding proteins (antibody, antibody construct, or antibody conjugate) provided herein, and a pharmaceutically acceptable carrier. Another embodiment of the disclosure includes the composition provided herein for use in preventing erythroid hyperplasia anemia and/or β-thalassemia, comprising a therapeutically effective amount of the binding proteins (antibody, isolated antibody, or antigen binding fragment, antibody construct, or antibody conjugate) provided herein.

In some embodiments, the carrier is a pharmaceutically acceptable carrier.

In another embodiment, the antibody and carrier are in a lyophilized form. In one embodiment, the antibody and carrier are in solution. In some embodiments, wherein the antibody and carrier are frozen. In another embodiment, the antibody and carrier are frozen at a temperature less than or equal to −65° C.

In some embodiments, the antibody is a sweeping antibody. In other embodiments, the antibody is a recycling antibody. In another embodiment, the antibody comprises an Fc portion. In other embodiments, the antibody binds the neonatal Fc receptor FcRn. In another embodiment, the Fc portion binds the neonatal Fc receptor FcRn.

In some embodiments, the antibody binds FcRn at a pH greater than 6.0. In other embodiments, the antibody binds FcRn at a pH in a range from 7.0 to 7.5.

In one embodiment, the Kd of binding of the antibody to the FcRN is in a range from 10⁻³ M to 10⁻⁸ M. In another embodiment, the Kd of binding of the antibody to the FcRN is in a range from 10⁻⁴ M to 10⁻⁸ M. In some embodiments, the Kd of binding of the antibody to the FcRN is in a range from 10⁻⁵ M to 10⁻⁸ M. In other embodiments, the Kd of binding of the antibody to the FcRN is in a range from10⁻⁶ M to 10⁻⁸ M.

The disclosure, in another aspect, includes an antibody that specifically binds to a GDF11 prodomain complex and inhibits the release of mature GDF11 from the GDF11 prodomain complex.

An additional aspect of the disclosure provides an antibody that specifically binds to a GDF11 prodomain complex and inhibits proteolytic cleavage of a proGDF11 or a latent GDF11 by a proprotein convertase or a tolloid protease.

In some embodiments, the antibody inhibits proteolytic cleavage of a tolloid protease cleavage site on the proGDF11 or latent GDF11. In another embodiment, the tolloid protease is selected from the group consisting of BMP-1, mammalian tolloid protein (mTLD), mammalian tolloid-like 1 (mTLL1), and mammalian tolloid-like 2 (mTLL2). In other embodiments, the antibody binds within 10 amino acid residues of a tolloid protease cleavage site of proGDF11 or latent GDF11. In some embodiments, the tolloid protease cleavage site comprises the amino acid sequence GD of proGDF11 or latent GDF11. In other embodiments, the proGDF11 or latent GDF11 comprises the amino acid sequence as set forth in SEQ ID NO: 82, 86, 97, or 98. In another embodiment, the antibody binds to the amino acid sequence KAPPLQQILDLHDFQGDALQPEDFLEEDEYHA (SEQ ID NO: 149).

In one embodiment, the antibody inhibits proteolytic cleavage of a proprotein convertase cleavage site on the proGDF11 or latent GDF11. In another embodiment, the proprotein convertase is selected from the group consisting of furin and PCSK5. In a further embodiment, the antibody binds within 10 amino acid residues of a proprotein converrase cleavage site of proGDF11 or latent GDF11. In another embodiment, the proprotein convertase cleavage site comprises the amino acid sequence RSRR (SEQ ID NO: 151), RELR (SEQ ID NO: 161), RSSR (SEQ ID NO: 152) of proGDF11 or latent GDF11.

In other embodiments, the proGDF11 or latent GDF11 comprises the amino acid sequence as set forth in SEQ ID NO: 82, 86, 97, or 98. In some embodiments, the antibody binds to the amino acid sequence GLHPFMELRVLENTKRSRRNLGLDCDEHSSESRC (SEQ ID NO: 153), PEPDGCPVCVWRQHSRELRLESIKSQILSKLRLK (SEQ ID NO: 154), or AAAAAAAAAAGVGGERSSRPAPSVAPEPDGCPVC (SEQ ID NO: 155).

In some embodiments, the antibody inhibits proteolytic cleavage of the proGDF11. In other embodiments, the antibody inhibits proteolytic cleavage of the latent GDF11. In another embodiment, the antibody inhibits the release of mature GDF11 from the GDF11 prodomain complex in a biological system by at least 5%, at least 10%, at least 20%, at least 40%, or at least 60%. In one embodiment, the antibody inhibits proteolytic cleavage of a proGDF11 or a latent GDF11 by a proprotein convertase or a tolloid protease in a biological system by at least 5%, at least 10%, at least 20%, at least 40%, or at least 60%.

In some embodiments, the biological system is a cell or a subject.

In other embodiments, the antibody is a stabilizing antibody.

In another embodiment, any of the binding proteins, antibody constructs or antibody conjugates disclosed herein exists as a crystal. In another embodiment, the crystal is a carrier-free pharmaceutical controlled release crystal. In another embodiment, the crystallized binding protein, crystallized antibody construct or crystallized antibody conjugate has a greater half-life in vivo than its soluble counterpart. In another embodiment, the crystallized binding protein, crystallized antibody construct or crystallized antibody conjugate retains biological activity after crystallization.

In one embodiment, a host cell is transformed with any of the vectors provided herein. In another embodiment, the host cell is a prokaryotic cell. In another embodiment, the host cell is E. coli. In another embodiment, the host cell is a eukaryotic cell. In another embodiment, the eukaryotic cell is selected from the group consisting of protist cell, animal cell, plant cell and fungal cell. In another embodiment, the host cell is a mammalian cell including, but not limited to, HEK293, CHO and COS; or a fungal cell such as Saccharomyces cerevisiae; or an insect cell such as Sf9.

In another embodiment, a method of producing a binding protein that binds GDF11 prodomain complex is provided. The method may comprise culturing a host cell, (e.g., any of the host cells provided herein) in a culture medium under conditions sufficient to produce a binding protein that binds GDF11 prodomain complex. Another embodiment provides a binding protein produced according to the method disclosed above.

In another embodiment, a composition is disclosed for the release of a binding protein, wherein the composition comprises a formulation which in turn comprises a crystallized binding protein, crystallized antibody construct or crystallized antibody conjugate as disclosed above and an ingredient; and at least one polymeric carrier. In another embodiment, the polymeric carrier is a polymer selected from one or more of the group consisting of: poly (acrylic acid), poly (cyanoacrylates), poly (amino acids), poly (anhydrides), poly (depsipeptide), poly (esters), poly (lactic acid), poly (lactic-co-glycolic acid) or PLGA, poly (b-hydroxybutryate), poly (caprolactone), poly (dioxanone); poly (ethylene glycol), poly ((hydroxypropyl)methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends and copolymers thereof. In another embodiment, the ingredient is selected from the group consisting of albumin, sucrose, trehalose, lactitol, gelatin, hydroxypropyl-.beta.-cyclodextrin, methoxypolyethylene glycol and polyethylene glycol.

In another embodiment, a method is disclosed for treating a mammal comprising the step of administering to the mammal an effective amount of the composition disclosed herein.

The disclosure also provides a pharmaceutical composition comprising a binding protein, antibody construct or antibody conjugate as disclosed herein and a pharmaceutically acceptable carrier. In a further embodiment, the pharmaceutical composition comprises at least one additional therapeutic agent for treating a disorder in which GDF11 activity is detrimental. In another embodiment, the additional agent is selected from the group consisting of: therapeutic agent, imaging agent, cytotoxic agent, angiogenesis inhibitors (including but not limited to anti-VEGF antibodies or VEGF-trap); kinase inhibitors (including but not limited to KDR and TIE-2 inhibitors); co-stimulation molecule blockers (including but not limited to anti-B7.1, anti-B7.2, CTLA4-Ig, anti-CD20); adhesion molecule blockers (including but not limited to anti-LFA-1 Abs, anti-E/L selectin Abs, small molecule inhibitors); anti-cytokine antibody or functional fragment thereof (including but not limited to anti-IL-18, anti-TNF, anti-IL-6/cytokine receptor antibodies); methotrexate; cyclosporin; rapamycin; FK506; detectable label or reporter; a TNF antagonist; an antirheumatic; a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist.

In another aspect, the instant disclosure provides a method for inhibiting human GDF11 activity comprising contacting human GDF11 prodomain complex with a binding protein disclosed herein such that human GDF11 activity is inhibited. In another aspect, the disclosure provides a method for inhibiting human GDF11 activity in a human subject suffering from a disorder in which GDF11 activity is detrimental, comprising administering to the human subject a binding protein disclosed herein such that human GDF11 activity in the human subject is inhibited and treatment is achieved.

In another aspect, the disclosure provides a method of treating (e.g., curing, suppressing, ameliorating, delaying or preventing the onset of, or preventing recurrence or relapse of) or preventing a GDF11-associated disorder, in a subject. The method includes: administering to the subject a GDF11 prodomain complex binding agent, e.g., an anti-GDF11 prodomain complex antibody or fragment thereof as described herein, in an amount sufficient to treat or prevent the GDF11-associated disorder. The GDF11 prodomain complex binding protein, e.g., the anti-GDF11 prodomain complex antibody or fragment thereof, may be administered to the subject, alone or in combination with other therapeutic modalities as described herein.

In one embodiment, the subject is a mammal, e.g., a human suffering from one or more GDF11-associated disorders, including, e.g., respiratory disorders (e.g., asthma (e.g., allergic and nonallergic asthma), chronic obstructive pulmonary disease (COPD), and other conditions involving airway inflammation, eosinophilia, fibrosis and excess mucus production; atopic disorders (e.g., atopic dermatitis and allergic rhinitis); inflammatory and/or autoimmune conditions of, the skin, gastrointestinal organs (e.g., inflammatory bowel diseases (IBD), such as ulcerative colitis and/or Crohn's disease), and liver (e.g., cirrhosis, fibrosis); scleroderma; tumors or cancers, e.g., Hodgkin's lymphoma as described herein. Accordingly, the disclosure includes the use of a GDF11 prodomain complex binding agent (such as an anti-GDF11 prodomain complex antibody or fragment thereof described herein) for a treatment described herein and the use of a GDF11 binding prodomain complex agent (such as an anti-GDF11 prodomain complex antibody or fragment thereof described herein) for preparing a medicament for a treatment described herein. Examples of GDF11-associated disorders include, but are not limited to, anemia, erythroid hyperplasia, beta thalassemia, as described herein.

In other embodiments, this disclosure provides a method of treating (e.g., reducing, ameliorating) or preventing one or more symptoms associated with anemia, or erythroid hyperplasia. The method comprises administering to the subject a GDF11 prodomain complex binding protein, e.g., a GDF11 prodomain complex antibody or a fragment thereof, in an amount sufficient to treat (e.g., reduce, ameliorate) or prevent one or more symptoms. The GDF11 prodomain complex antibody can be administered therapeutically or prophylactically, or both. The GDF11 prodomain complex binding protein, e.g., the anti-GDF11 prodomain complex antibody, or fragment thereof, can be administered to the subject, alone or in combination with other therapeutic modalities as described herein. In another aspect, the subject is a mammal, e.g., a human suffering from a GDF11-associated disorder as described herein.

In another aspect, this application provides a method for detecting the presence of GDF11 prodomain complex in a sample in vitro (e.g., a biological sample, such as serum, plasma, tissue, and biopsy). The subject method can be used to diagnose a disorder. The method includes: (i) contacting the sample or a control sample with the anti-GDF11 prodomain complex antibody or fragment thereof as described herein; and (ii) detecting formation of a complex between the anti-GDF11 prodomain complex antibody or fragment thereof, and the sample or the control sample, wherein a statistically significant change in the formation of the complex in the sample relative to the control sample is indicative of the presence of the GDF11 prodomain complex in the sample.

In another aspect, this application provides a method for detecting the presence of GDF11 prodomain complex in vivo (e.g., in viva imaging in a subject). The subject method can be used to diagnose a disorder, e.g., a GDF11-associated disorder. The method includes: (i) administering the anti-GDF11 prodomain complex antibody or fragment thereof as described herein to a subject or a control subject under conditions that allow binding of the antibody or fragment to GDF11 prodomain complex; and (ii) detecting formation of a complex between the antibody or fragment and GDF11 prodomain complex, wherein a statistically significant change in the formation of the complex in the subject relative to the control subject is indicative of the presence of GDF11 prodomain complex.

In another embodiment, the disclosure provides at least one GDF11 prodomain complex anti-idiotype antibody to at least one GDF11 prodomain complex binding protein disclosed herein. The anti-idiotype antibody includes any protein or peptide containing molecule that comprises at least a portion of an immunoglobulin molecule such as, but not limited to, at least one complementarily determining region (CDR) of a heavy or light chain or a ligand binding portion thereof, a heavy chain or light chain variable region, a heavy chain or light chain constant region, a framework region, or; any portion thereof, that can be incorporated into a binding protein of the present disclosure.

### BRIEF DESCRIPTION OF THE DRAWINGS

FIGS. 1A-1B show GDF11 domain structure and proGDF11 assembly. FIG. 1A is a schematic of GDF11's domain structure. GDF11 is secreted as a proprotein, with an inhibitory prodomain followed by a C-terminal growth factor domain, which exists as a disulfide-linked dimer. FIG. 1B shows the precursor protein, which is assembled in an inactive conformation where the prodomain (purple) encloses the growth factor (cyan) with a “straightjacket” assembly comprised of an alpha helix connected to a loop termed the latency lasso. This figure is an adaption from the structure of latent TGFb1 (Shi et. al., 2011).

FIG. 2 is a schematic illustrating that the activation of GDF11 requires two distinct proteolysis events. The biosynthetic precursor protein, proGDF11, is processed by two separate proteases. The first step shown in this schematic is performed by a member of the proprotein convertase family, such as Furin or PCSK5. This cleavage separates the prodomain from the mature growth factor and produces the latent form of GDF11. The second cleavage event, by the tolloid family of proteases, cleaves within the prodomain. Both cleavage events are required for release of GDF11 and subsequent engagement of the GDF11 growth factor with the Type I and Type II signaling receptors.

FIGS. 3A-3B show GDF11 activation blocking antibodies comprise three separate epitope groups which bind to the prodomain of proGDF11. FIG. 3A shows the results from cross-blocking experiments performed on a ForteBio Octet BLI, which identified three epitope groups. Pairwise binding events are indicated by shading in the boxes, where the first antibody added in the experiment is indicated on the Y axis, and the second antibody on the X axis. The extent of binding that was detected in these cross-blocking experiments is indicated as “no binding”, “some binding” and “unimpeded binding”. FIG. 3B shows the combination of cross-blocking results and epitope mapping studies utilizing chimeric GDF11/Myostatin proteins. Antibodies within Bins 1 and 3 bind to the “ARM” region of the prodomain, which comprises the regions shaded in purple in the figure. Antibodies within Bin 2 bind to the “straightjacket” portion of the prodomain shaded in green.

FIGS. 4A-4B show blockers of GDF11 activation. Following an overnight proteolysis reaction with enzymes from both the proprotein-convertase and tolloid protease families, the release of mature growth factor was measured in the presence of different concentrations of antibodies that inhibit GDF11 activation using a CAGA-based reporter assay in 293T cells (FIG. 4A). Calculated EC50 values are indicated in parentheses. FIG. 4B shows that the GDF11 inhibitory Ab, GDF11 Inh-5, does not block activation of human proGDF8. In this assay, the human proGDF8 concentration was 400 nM.

FIG. 5 is a graph showing CAGA promoter-dependent luciferase activity is the presence of GDF-11 or proGDF-11 after treatment with proprotein convertase, Tolloid proteinase or a combination of proprotein convertase and Tolloid proteinase.

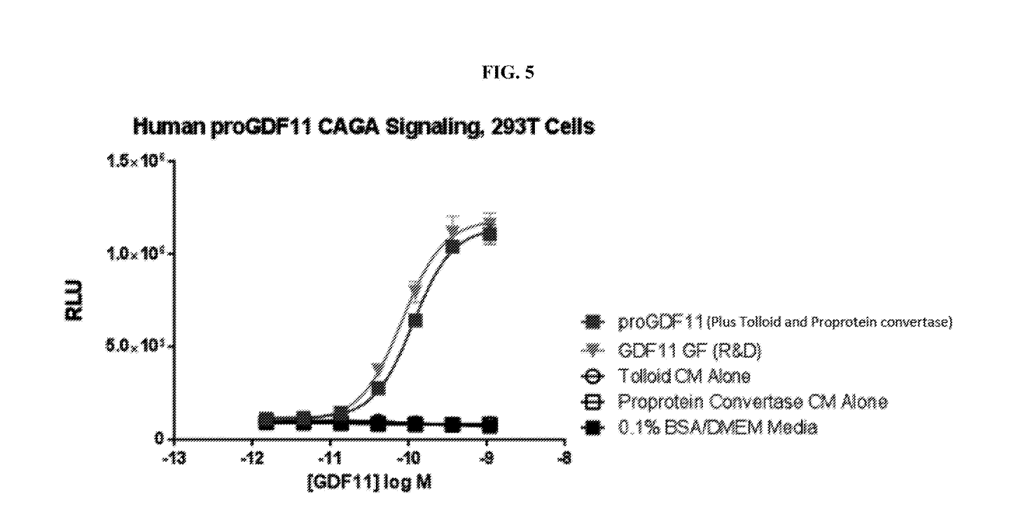

FIG. 6 presents results of a luciferase-based growth factor activity assay.

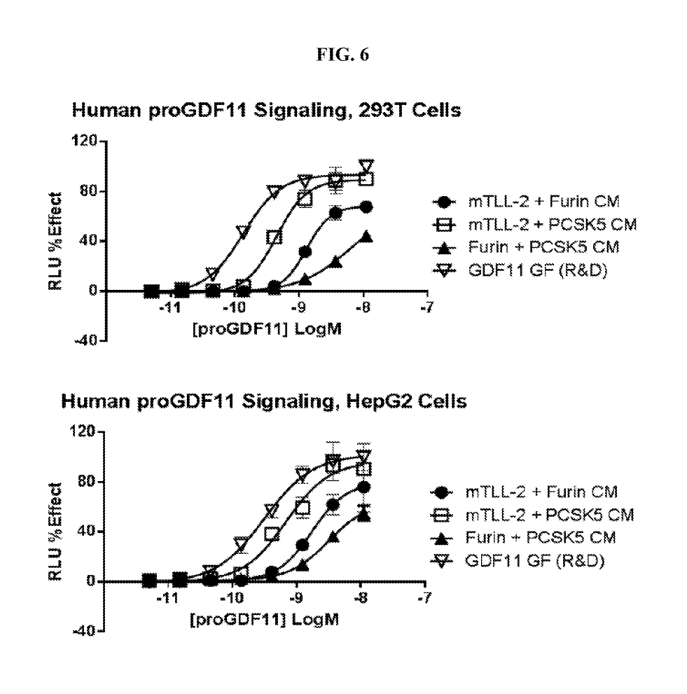

FIG. 7 is a stained gel showing separation of proteinase treated proGDF-11 under reducing and non-reducing conditions.

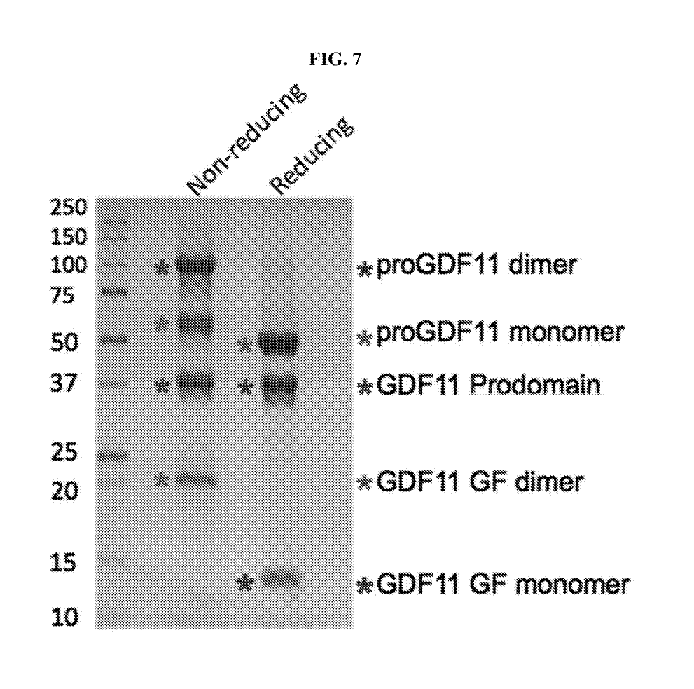

### DETAILED DESCRIPTION

This disclosure pertains to human GDF11 prodomain complex binding proteins, and more particularly to anti-GDF11 prodomain complex antibodies, or antigen-binding portions thereof, that bind GDF11 prodomain complex. Various aspects of the disclosure relate to antibodies and antibody fragments, and pharmaceutical compositions thereof, as well as nucleic acids, recombinant expression vectors and host cells for making such antibodies and fragments. Methods of using the antibodies of the disclosure to detect human GDF11 prodomain complex, to modulate human GDF11 activities and/or levels, either in vitro or in vivo are also disclosed.

Unless otherwise defined herein, scientific and technical terms used in connection with the present disclosure shall have the meanings that are commonly understood by those of ordinary skill in the art. The meaning and scope of the terms are clear, however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the present disclosure may be more readily understood, select terms are defined below.

The term “Polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide (e.g., an antibody) that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art. In some embodiments, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The term “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

The term “human GDF11 prodomain complex” as used herein, refers to the proGDF11 and the latent complex of GDF11 (prodomain complexed with the growth factor domain). In some embodiments, the amino acid sequence of pro and latent GDF11 comprises SEQ ID NO: 82.

“Biological activity” or “activity” of a protein, as used herein, refers to all inherent biological properties of the protein.

The terms “specific binding” or “specifically binding”, as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, including antigen-binding portions, which retains the essential epitope binding features of an Ig molecule. Such mutant, variant, or derivative antibody formats are known in the art. Nonlimiting embodiments of which are discussed below.

In some embodiments, compounds and/or compositions of the present disclosure may comprise antibodies or fragments thereof. In other embodiments, the term “antibody” refers to in the broadest sense and specifically covers various embodiments including, but not limited to monoclonal antibodies, polyclonal antibodies, multispecific antibodies (e.g. bispecific antibodies formed from at least two intact antibodies), and antibody fragments such as diabodies so long as they exhibit a desired biological activity. Antibodies are primarily amino-acid based molecules but may also comprise one or more modifications (including, but not limited to the addition of sugar moieties, fluorescent moieties, chemical tags, etc.).

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding portion” of an antibody (or simply “antibody portion”), as used herein, refers to one or more fragments of an antibody that retain the ability to specifically bind to an antigen (e.g., hGDF11). It has been shown that the antigen-binding function of an antibody can be performed by fragments of a full-length antibody. Such antibody embodiments may also be bispecific, dual specific, or multi-specific formats; specifically binding to two or more different antigens. Multispecific, dual specific, and bispecific antibody constructs are well known in the art and described and characterized in Kontermann (ed.), Bispecific Antibodies, Springer, N.Y. (2011), and Spiess et al., Mol. Immunol. 67(2):96-106 (2015).

Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct” as used herein refers to a polypeptide comprising one or more antigen binding portions of the disclosure linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences and their functional variations are known in the art.

Further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds hGDF11 prodomain complex is substantially free of antibodies that specifically bind antigens other than hGDF11 prodomain complex). An isolated antibody that specifically binds hGDF11 prodomain complex may, however, have cross-reactivity to other antigens, such as GDF11 prodomain complex molecules from other species. Moreover, an isolated antibody may be substantially free of other cellular material and/or chemicals.

The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human GDF11 prodomain complex which can be generated using techniques well known in the art, such as, but not limited to, using human Ig phage libraries such as those disclosed in Jermutus et al., PCT publication No. WO 2005/007699 A2.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti human GDF11 prodomain complex antibodies and antigen binding portions are provided. Such antibodies were generated by obtaining murine anti-hGDF11 prodomain complex monoclonal antibodies using traditional hybridoma technology followed by humanization using in vitro genetic engineering, such as those disclosed in Kasaian et al PCT publication No. WO 2005/123126 A2.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In an embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art. In one embodiment, the acceptor sequences known in the art may be used in the antibodies disclosed herein.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol. 22(3): 183-200 (2002); Marchalonis et al., Adv Exp Med. Biol. 484:13-30 (2001)). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In one embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In another embodiment, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG 1, IgG2, IgG3 and IgG4. The humanized antibody may comprise sequences from more than one class or isotype, and particular constant domains may be selected to optimize desired effector functions using techniques well-known in the art.

In one embodiment, the framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In another embodiment, such mutations, however, will not be extensive. Usually, at least 80%, 85%, 90%, and or 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. In another embodiment, if two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody.

The term “multivalent binding protein” is used in this specification to denote a binding protein comprising two or more antigen binding sites. In another embodiment, a multivalent binding protein may be engineered to have three or more antigen binding sites, and is generally not a naturally occurring antibody.

The term “multispecific binding protein” refers to a binding protein capable of binding two or more related or unrelated targets. As referenced earlier, such antibody constructs are well known in the art, and as described and characterized in Kontermann (ed.), Bispecific Antibodies, Springer, N.Y. (2011), and Spiess et al., Mol. Immunol. 67(2):96-106 (2015). Such bispecific antibody constructs include but are not limited to those commonly known as, Minibodies, Nanobodies, Diabodies, Bites, Duobodies, Tandemabs, Knobs-into-holes Igs, DAFs, CT-Igs, DutamAbs, DVD-Igs, CoDVD-Igs, CoDV-Igs, FIT-Igs, CrossmAbs, CrossfAbs, SEEDbodies, TriomAbs, LUZ-Ys, Zybodies. Multispecific binding proteins as used herein, are binding proteins that comprise two or more antigen binding sites and are tetravalent or multivalent binding proteins. Such DVDs may be monospecific, i.e. capable of binding one antigen or multispecific, i.e. capable of binding two or more antigens. In some embodiments, a multispecific antibody refers to an antibody wherein two or more variable regions bind to different epitopes. The epitopes may be on the same or different targets. In certain embodiments, a multi-specific antibody is a bispecific antibody, which recognizes two different epitopes on the same or different antigens. In some embodiments, bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen. In some embodiments, the binding protein may be a multispecific antibody, a dual specific antibody, and a bispecific antibody. Such antibody constructs are well known in the art, and as described and characterized in Kontermann (ed.), Bispecific Antibodies, Springer, N.Y. (2011), and Spiess et al., Mol. Immunol. 67(2):96-106 (2015). Such bispecific antibody constructs comprise one of more binding domain capable of binding GDF11 prodomain complex and a second target. In one embodiment, the second target is selected from the group consisting of GDF1, GDF3, GDF5, GDF6, GDF7, GDF8, GDF9, GDF10, BMP10, BMP9 (GDF2), nodal, BMP2, BMP4, BMP5, BMP6, BMP8A, BMP8B, BMP15, BMP3, TGFbeta 1, TGF beta 2, TGF beta 3, Inhibin beta A, Inhibin beta B, Inhibin beta C, Inhibin beta E, Lefty 1, Lefty 2, GDF15, Antimullerian hormone, Inhibin alpha. In another embodiment, the second target is selected from the group consisting of CSF1, (MCSF), CSF2 (GM-CSF), CSF3 (GCSF), FGF2, IFN.alpha.1, IFN.beta.1, IFN.gamma., histamine and histamine receptors, IL-1.alpha., IL-1.beta., IL-2, IL-3, IL-4, IL-5, IL-6, IL-7, IL-8, IL-9, IL-10, IL-11, IL-12.alpha., IL-12.beta., IL-14, IL-15, IL-16, IL-17, IL-18, IL-19, KITLG, PDGFB, IL-2R.alpha., IL-4R, IL-5R.alpha., IL-8R.alpha., IL-8R.beta., IL-12R.beta.1, IL-12R.beta.2, GDF11R.alpha.1, GDF11R.alpha.2, IL-18R1, TSLP, CCL1, CCL2, CCL3, CCL4, CCLS, CCL7, CCL8, CCL13, CCL17, CCL18, CCL19, CCL20, CCL22, CCL24, CX3CL1, CXCL1, CXCL2, CXCL3, XCL1, CCR2, CCR3, CCR4, CCRS, CCR6, CCR7, CCR8, CX3CR1, GPR2, XCR1, FOS, GATA3, JAK1, JAK3, STAT6, TBX21, TGFB1, TNFSF6, YY1, CYSLTR1, FCER1A, FCER2, LTB4R, TB4R2, LTBR, and Chitinase. In another embodiment, the multispecifc binding protein is capable of recognizing GDF11 and IL-1.beta., GDF11 and IL-9; GDF11 and L-4; GDF11 and IL-5; GDF11 and IL-25; GDF11 and TARC; GDF11 and MDC; GDF11 and MIF; GDF11 and TGF-.beta.; GDF11 and LHR agonist; GDF11 and CL25; GDF11 and SPRR2a; GDF11 and SPRR2b; or GDF11 and ADAMS. In another embodiment, the multispecifc binding protein is capable of binding GDF11 and TNF-alpha.

As used herein, the term “neutralizing” refers to neutralization of the biological activity of a target protein when a binding protein specifically binds the target protein. In one embodiment, a neutralizing binding protein is a neutralizing antibody whose binding to hGDF11 prodomain complex results in inhibition of a biological activity of hGDF11. In another embodiment, the neutralizing binding protein binds hGDF11 prodomain complex and reduces a biologically activity of GDF11 by at least about 20%, 40%, 60%, 80%, 85% or more. Inhibition of a biological activity of hGDF11 prodomain complex by a neutralizing binding protein can be assessed by measuring one or more indicators of the biological activity of hGDF11 well known in the art.

The term “activity” includes activities such as the binding specificity/affinity of an antibody for an antigen.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “surface biolayer interferometry”, as used herein, refers to an optical phenomenon that allows for the analysis of real-time biospecific interactions by detection of alterations in protein concentrations within a biosensor matrix, for example using the Forte Bio Octet system.

The term “surface plasmon resonance”, as used herein, refers to an optical phenomenon that allows for the analysis of real-time biospecific interactions by detection of alterations in protein concentrations within a biosensor matrix, for example using the BlAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “kₒₙ”, as used herein, is intended to refer to the on rate constant for association of an antibody to the antigen to form the antibody/antigen complex as is known in the art.

The term “k_{off}”, as used herein, is intended to refer to the off rate constant for dissociation of an antibody from the antibody/antigen complex as is known in the art.

The term “K_{D}”, as used herein, is intended to refer to the dissociation constant of a particular antibody-antigen interaction as is known in the art.

The term “labeled binding protein” as used herein, refers to a protein with a label incorporated that provides for the identification of the binding protein. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody conjugate” refers to a binding protein, such as an antibody, chemically linked to a second chemical moiety, such as a therapeutic or cytotoxic agent. The term “agent” is used herein to denote a chemical compound, a mixture of chemical compounds, a biological macromolecule, or an extract made from biological materials. In another embodiment, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The terms “crystal”, and “crystallized” as used herein, refer to an antibody, or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 20 1-16, Oxford University Press, New York, N.Y., (1999).”

The term “polynucleotide” as referred to herein means a polymeric form of two or more nucleotides, either ribonucleotides or deoxynucleotides or a modified form of either type of nucleotide. The term includes single and double stranded forms of DNA

The term “isolated polynucleotide” as used herein shall mean a polynucleotide (e.g., of genomic, cDNA, or synthetic origin, or some combination thereof) that, by virtue of its origin, is not associated with all or a portion of a polynucleotide with which the “isolated polynucleotide” is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences. Protein constructs of the present disclosure may be expressed, and purified using expression vectors and host cells known in the art, including expression cassettes, vectors, recombinant host cells and methods for the recombinant expression and proteolytic processing of recombinant polyproteins and pre-proteins from a single open reading frame (e.g., WO 2007/014162 incorporated herein by reference).

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. In another embodiment, host cells include prokaryotic and eukaryotic cells selected from any of the Kingdoms of life. In another embodiment, eukaryotic cells include protist, fungal, plant and animal cells. In another embodiment, host cells include but are not limited to the prokaryotic cell line E. Coli; mammalian cell lines CHO, HEK 293 and COS; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

“Transgenic organism”, as known in the art and as used herein, refers to an organism having cells that contain a transgene, wherein the transgene introduced into the organism (or an ancestor of the organism) expresses a polypeptide not naturally expressed in the organism. A “transgene” is a DNA construct, which is stably and operably integrated into the genome of a cell from which a transgenic organism develops, directing the expression of an encoded gene product in one or more cell types or tissues of the transgenic organism.

The term “regulate” and “modulate” are used interchangeably, and, as used herein, refers to a change or an alteration in the activity of a molecule of interest (e.g., the biological activity of hGDF11). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator,” as used herein, is a compound capable of changing or altering an activity or function of a molecule of interest (e.g., the biological activity of hGDF11). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in WO01/83525.

The term “agonist”, as used herein, refers to a modulator that, when contacted with a molecule of interest, causes an increase in the magnitude of a certain activity or function of the molecule compared to the magnitude of the activity or function observed in the absence of the agonist. Agonists of GDF11 may include, but are not limited to, proteins (e.g., Ab), nucleic acids, carbohydrates, or any other molecules, which bind to GDF11, or proGDF11.

The term “antagonist” or “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of GDF11. Antagonists and inhibitors of hGDF11 prodomain complex may include, but are not limited to, proteins (e.g., Ab), nucleic acids, carbohydrates, or any other molecules, which bind to latent GDF11, or proGDF11.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, serum, urine, synovial fluid, cells, organs, tissues, bone marrow, lymph nodes and spleen.

### TGFβ Family of Proteins

Aspects of the disclosure provide TGFβ family member proteins. There are 33 different members of the TGF-beta family in humans. Members include, without limitation, the bone morphogenetic proteins (BMPs), inhibin, activin, growth and differentiation factors (GDFs), myostatin, nodal, anti-Mullerian hormone, and lefty proteins. A review of TGF-β family members, related signaling molecules as well as their relationships can be found in Massague., 2000. Nature Reviews Molecular Cell Biology. 1:169-78, the contents of which are herein incorporated by reference in their entirety. In some embodiments, mature growth factors are synthesized along with their prodomains as single polypeptide chains. In some embodiments, such polypeptide chains may comprise cleavage sites for separation of prodomains from mature growth factors. In some embodiments, such cleavage sites are furin cleavage sites recognized and cleaved by proprotein convertases.

In general, homology among TGF-β family member growth factor domains is relatively high. Interestingly, prodomain homology is much lower. This lack of homology may be an important factor in altered growth factor regulation among family members. In some cases, prodomains may guide proper folding and/or dimerization of growth factor domains. Prodomains have very recently been recognized, in some cases, to have important functions in directing growth factors (after secretion) to specific locations in the extracellular matrix (ECM) and/or cellular matrix, until other signals are received that cause growth factor release from latency. Release from latency may occur in highly localized environments whereby growth factors may act over short distances (e.g. from about 1 cell diameter to about a few cell diameters, from about 2 cell diameters to about 100 cell diameters and/or from about 10 cell diameters to about 10,000 cell diameters) and cleared once they reach the circulation. Some growth factor-prodomain complexes are secreted as homodimers. In some embodiments, prodomain-growth factor complexes may be secreted as heterodimers.

As used herein, the term “TGF-β-related protein” refers to a TGF-β isoform, a TGF-β family member or a TGF-β family member-related protein. TGF-β family members may include, but are not limited to, any of those listed in Tables 1-6. These include, but are not limited to TGF-β proteins, BMPs, myostatin, GDFs and inhibins. Aspects of the present invention provide tools and/or methods for characterizing and/or modulating cellular activities related to growth factor signaling. In other embodiments, tools of the present invention may comprise antigens comprising one or more components of one or more TGF-β-related proteins. Some tools may comprise antibodies directed toward antigens of the present invention. In additional embodiments, tools of the present invention may comprise assays for the detection and/or characterization of TGF-β-related proteins, the detection and/or characterization of antibodies directed toward TGF-β-related proteins and/or the detection and/or characterization of cellular activities and/or their cellular signaling related to TGF-β-related proteins.

Described herein are compounds for the modulation of growth factor activity and/or levels. Such growth factors include growth differentiation factor (GDF) proteins which are TGF-β family member proteins involved in a number of cellular and developmental activities. Part of the invention provides GDF-modulatory antibodies as well as methods for generating, optimizing and using such antibodies. Further antibodies include GDF-modulatory antibodies that are capable of distinguishing between various growth factor complexes allowing for growth factor activity modulation that occurs only at sites of specific complex formations.

In some embodiments, the present invention provides GDF-11 modulatory antibodies. Such antibodies may bind GDF-11 GPCs, GDF-11 prodomains, growth factors, or complexes comprising GDF-11 growth factor and activate or inhibit GDF-11 growth factor activity. In some cases, these antibodies are specific for GDF-11, having reduced or no affect on GDF-8 growth factor activity.

Many TGF-β family member proteins are synthesized in conjunction with prodomains. Some prodomains may remain associated with growth factors after cleavage. Such associations may form latent growth factor-prodomain complexes (GPCs) that modulate the availability of growth factors for cell signaling. Growth factors may be released from latency in GPCs through associations with one or more extracellular proteins. In some cases, growth factor release may rely on force applied to GPCs through extracellular protein interactions. Such forces may pull from C-terminal and/or N-terminal regions of GPCs resulting in the release of associated growth factors.

In some TGF-β family members, the prodomain portion of the GPC is responsible for growth factor retention and blocking the interaction of retained growth factors with their receptors. Such GPCs, where the bound growth factor is unable to promote signaling activity, are also referred to herein as “latent complexes.” Prodomain portions of GPCs that function to block growth factor signaling activity are referred to as latency associated peptides (LAPs). TGF-β1, 2 and 3 are know to comprise LAPs. GDF prodomains also function to block growth factor activity. Some prodomains may comprise LAP-like domains. As used herein, the term “LAP-like domain” refers to prodomain portions of GPCs and/or free prodomains that may be structurally similar or synthesized in a similar manner to LAPs, but that may not function to prevent growth factor/receptor interactions.

In some embodiments, growth factor dimers may associate with prodomain modules to form a GPC. In some embodiments, GPCs comprise protein modules necessary for different aspects of growth factor signaling, secretion, latency and/or release from latent GPCs. As used herein, the term “protein module” refers to any component, region and/or feature of a protein. Protein modules may vary in length, comprising one or more amino acids. Protein modules may be from about 2 amino acid residues in length to about 50 amino acid residues in length, from about 5 amino acid residues in length to about 75 amino acid residues in length, from about 10 amino acid residues in length to about 100 amino acid residues in length, from about 25 amino acid residues in length to about 150 amino acid residues in length, from about 125 amino acid residues in length to about 250 amino acid residues in length, from about 175 amino acid residues in length to about 400 amino acid residues in length, from about 200 amino acid residues in length to about 500 amino acid residues in length and/or at least 500 amino acid residues in length.

In some embodiments, protein modules comprise one or more regions with known functional features (e.g. protein binding domain, nucleic acid binding domain, hydrophobic pocket, etc). Protein modules may comprise functional protein domains necessary for different aspects of growth factor signaling, secretion, latency and/or release from latent conformations.

In some embodiments, prodomains may associate with growth factors in GPCs. Some prodomains may sterically prevent growth factor association with one or more cellular receptors. Prodomains may comprise arm regions and/or straight jacket regions. Some prodomains may comprise C-terminal regions referred to herein as “bowtie regions.” In some prodomain dimers, bowtie regions of each monomer may associate and/or interact. Such associations may comprise disulfide bond formation, as is found between monomers of TGF-β isoform LAPs.

In some embodiments, arm regions may comprise trigger loop regions. Trigger loops may comprise regions that associate with integrins. Such regions may comprise amino acid sequences comprising RGD (Arg-Gly-Asp). Regions comprising RGD sequences are referred to herein as RGD sequence regions. In some embodiments, prodomains comprise latency loops (also referred to herein as latency lassos). Some latency loops may maintain associations between prodomains and growth factors present within GPCs. Prodomains may also comprise fastener regions. Such fastener regions may promote associations between prodomains and growth factors present within GPCs by maintaining prodomain conformations that promote growth factor retention.

In some cases, GPCs may require enzymatic cleavage to promote dissociation of bound growth factors and growth factor activity. Such enzymatic cleavage events are referred to herein as “activating cleavage” events. Activating cleavage of GPCs may be carried out in some instances by members of the BMP-1/Tolloid-like proteinase (B/TP) family (Muir et al., 2011. J Biol Chem. 286(49):41905-11, the contents of which are herein incorporated by reference in their entirety). These metaloproteinases may include, but are not limited to BMP-1, mammalian tolloid protein (mTLD), mammalian tolloid-like 1 (mTLL1) and mammalian tolloid-like 2 (mTLL2). Exemplary GPCs that may be subjected to activating cleavage by such metalloproteinases may include, but are not limited to GDF-8 and GDF-11. In some cases, GDF-8 may be cleaved by mTLL2. In some cases, activating cleavages may occur intracellularly. In some cases, activating cleavages may occur extracellularly.

Growth factor release from GPCs may require cleavage by a proprotein convertase enzyme followed by an activating cleavage [e.g. by one or more members of the BMP-1/Tolloid-like proteinase (B/TP) family.] In one example, GDF-8 and GDF-11 GPCs may be transformed by furin cleavage into a latent complex that further requires cleavage by BMP/Tolloid proteases for growth factor release.

In some embodiments, the present invention provides polypeptide inhibitors (e.g., antibodies) that inhibit one or more members of the B/TP family. Such inhibitors may block cleavage of BMP-1/Tolloid cleavage sites, including, but not limited to BMP/Tolloid cleavage sites on one or more latent complexes (e.g., GDF-8 latent complexes and/or GDF-11 latent complexes).

In some cases, activating cleavage may not lead to dissociation of bound growth factor, but instead may promote an active conformation of the GPC. As used herein when referring to a GPC, the term “active conformation” refers to a GPC protein confirmation that allows the growth factor to engage in receptor interaction. Such scenarios have been predicted with proBMP-7 and proBMP-9 (Sengle, G. et al., 2008. JMB. 381: 1025-39 and Mi et al., 2015. PNAS. 112(12): 3710-5, the contents of each of which are herein incorporated by reference in their entirety). In some embodiments, the present invention provides antibodies that specifically target active conformations to modulate growth factor activity.

Active conformations of GDF-11 GPCs are referred to herein as “primed” complexes, and can be produced by the sequential cleavage of GPCs at the furin cleavage site and the BMP/Tolloid cleavage site. Unlike latent GPCs, primed complexes (either the entire complex or portions of the complex) may bind receptors resulting in receptor signaling. In some cases, prodomains may be dissociated from growth factors upon receptor binding and/or signaling activity. In some cases, prodomains may remain associated with growth factors upon receptor binding and/or signaling activity. In some cases, prodomains may become partially dissociated from growth factors during receptor binding and/or signaling activity.

In some cases, primed complexes may bind preferentially to one or more receptors over one or more other receptors. In some cases, receptor activity resulting from primed complex interactions may be quenched or competed for by excess prodomain or fragments thereof.

In some embodiments, the present invention provides polypeptide inhibitors (e.g., inhibiting antibodies) that block the formation of primed complexes from latent complexes. In some cases, such inhibitors bind BMP/Tolloid cleavage sites on latent GPCs (e.g., latent GDF-11). In some embodiments, such inhibitors prevent cleavage of the BMP/Tolloid cleavage site.

Straight jacket regions may comprise alpha 1 helical regions. In some embodiments, alpha 1 helical regions may be positioned between growth factor monomers. Some alpha 1 helical regions comprise N-terminal regions of prodomains. Alpha 1 helical regions may also comprise N-terminal regions for extracellular associations. Such extracellular associations may comprise extracellular matrix proteins and/or proteins associated with the extracellular matrix. Some extracellular associations may comprise associations with proteins that may include, but are not limited to LTBPs (e.g. LTBP1, LTBP2, LTBP3 and/or LTBP4), fibrillins (e.g. fibrillin-1, fibrillin-2, fibrillin-3 and/or fibrillin-4), perlecan, decorin and/or GASPs. N-terminal extracellular associations may comprise disulfide bonds between cysteine residues. In some cases, extracellular matrix proteins and/or proteins associated with the extraceullar matrix may comprise bonds or interactions with one or more regions of prodomains other than N-terminal regions.

In some embodiments, growth factor domains comprise one or more growth factor monomers. Some growth factor domains comprise growth factor dimers. Such growth factor domains may comprise growth factor homodimers or heterodimers (comprising growth factor monomers from different TGF-β-related proteins). Some growth factor domains may comprise fingers regions. Such fingers regions may comprise β-pleated sheets. Fingers regions may associate with prodomains. Some fingers regions may maintain association between growth factor domains and prodomains.

In some embodiments, recombinant proteins of the present invention may comprise protein modules from growth differentiation factor (GDF) proteins. Such GDF protein modules may comprise the protein modules and/or amino acid sequences listed in Table 2 or 4. In some embodiments, protein modules of the present invention may comprise amino acid sequences similar to those in Table 2 or 4, but comprise additional or fewer amino acids than those listed. Some such amino acid sequences may comprise about 1 more or fewer amino acids, about 2 more or fewer amino acids, about 3 more or fewer amino acids, about 4 more or fewer amino acids, about 5 more or fewer amino acids, about 6 more or fewer amino acids, about 7 more or fewer amino acids, about 8 more or fewer amino acids, about 9 more or fewer amino acids, about 10 more or fewer amino acids or greater than 10 more or fewer amino acids on N-terminal and/or C-terminal ends.

In some embodiments, recombinant proteins of the present invention may comprise protein modules from activin subunits. Such protein modules may comprise the protein modules and/or amino acid sequences of the activin subunit inhibin beta A, listed in Table 4. In some embodiments, protein modules of the present invention may comprise amino acid sequences similar to those in Table 4, but comprise additional or fewer amino acids than those listed. Some such amino acid sequences may comprise about 1 more or fewer amino acids, about 2 more or fewer amino acids, about 3 more or fewer amino acids, about 4 more or fewer amino acids, about 5 more or fewer amino acids, about 6 more or fewer amino acids, about 7 more or fewer amino acids, about 8 more or fewer amino acids, about 9 more or fewer amino acids, about 10 more or fewer amino acids or greater than 10 more or fewer amino acids on N-terminal and/or C-terminal ends.

Binding Proteins that Bind Human GDF11 Prodomain Complex

One aspect of the present disclosure provides antibodies, or portions thereof, that are isolated antibodies. One aspect of the present disclosure provides isolated monoclonal antibodies, or antigen-binding portions thereof, that bind to GDF11 prodomain complex with high affinity, a slow off rate and high neutralizing capacity. Another aspect of the disclosure provides antibodies that specifically bind hGDF11 prodomain complex. Another aspect of the disclosure provides fully human antibodies that bind GDF11 prodomain complex. Another aspect of the disclosure provides murine antibodies that bind GDF11 prodomain complex. Another aspect of the disclosure provides chimeric antibodies that bind GDF11 prodomain complex. Another aspect of the disclosure provides humanized antibodies, or antigen-binding portions thereof, that bind GDF11 prodomain complex. In one embodiment, antibodies, or portions thereof, specifically bind hGDF11 prodomain complex. In another embodiment, the antibodies of the disclosure are neutralizing human anti-GDF11 antibodies. More specifically, antibodies of the disclosure are neutralizing human anti-hGDF11 antibodies.

### A. Method of Making Anti GDF11 Antibodies

Antibodies of the present disclosure may be made by any of a number of techniques known in the art.

### Anti-GDF11 Prodomain Complex Monoclonal Antibodies Using Recombinant Antibody Libraries

In vitro methods can be used to make the antibodies of the disclosure, wherein an antibody library is screened to identify an antibody having the desired binding specificity for GDF11. Methods for such screening of recombinant antibody libraries are well known in the art and include methods described in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, US patent application publication 20030186374, and PCT Publication No. WO 97/29131, the contents of each of which are incorporated herein by reference.

The following tables (Tables 1-6) set forth below include the amino acid sequences of TGFβ family member proteins, protein modules of TGFβ family member proteins, non-human TGFβ family member proteins, and chimeric proteins (e.g., of the protein modules provided herein), which may be used in accordance with the disclosure. It should be appreciated that the amino acid sequences provided herein are not meant to be limiting and additional sequences of TGFβ family member proteins are also within the scope of the disclosure. The sequences of additional TGFβ family member proteins, as well as their domains, would be apparent to the skilled artisan in view of this disclosure and knowledge in the art.

TABLE 1
Pro-proteins of the TGF-beta family.

| | | SEQ |
|---|---|---|
| | | ID |
| TGF Member | Prodomain and growth factor Sequence | NO |
| GDF11 | AEGPAAAAAAAAAAAAAGVGGERSSRPAPSVAPEPDGCPV | 82 |
| | CVWRQHSRELRLESIKSQILSKLRLKEAPNISREVVKQLLPKA | |
| | PPLQQILDLHDFQGDALQPEDFLEEDEYHATTETVISMAQET | |
| | DPAVQTDGSPLCCHFHFSPKVMFTKVLKAQLWVYLRPVPRP | |
| | ATVYLQILRLKPLTGEGTAGGGGGGRRHIRIRSLKIELHSRSG | |
| | HWQSIDFKQVLHSWFRQPQSNWGIEINAFDPSGTDLAVTSLG | |
| | PGAEGLHPFMELRVLENTKRSRRNLGLDCDEHSSESRCCRYP | |
| | LTVDFEAFGWDWIIAPKRYKANYCSGQCEYMFMQKYPHTH | |
| | LVQQANPRGSAGPCCTPTKMSPINMLYFNDKQQIIYGKIPGM | |
| | VVDRCGCS | |
| GDF8 | NENSEQKENVEKEGLCNACTWRQNTKSSRIEAIKIQILSKLRL | 83 |
| (myostatin) | ETAPNISKDVIRQLLPKAPPLRELIDQYDVQRDDSSDGSLEDD | |
| | DYHATTETIITMPTESDFLMQVDGKPKCCFFKFSSKIQYNKV | |
| | VKAQLWIYLRPVETPTTVFVQILRLIKPMKDGTRYTGIRSLKL | |
| | DMNPGTGIWQSIDVKTVLQNWLKQPESNLGIEIKALDENGH | |
| | DLAVTFPGPGEDGLNPFLEVKVTDTPKRSRRDFGLDCDEHST | |
| | ESRCCRYPLTVDFEAFGWDWIIAPKRYKANYCSGECEFVFLQ | |
| | KYPHTHLVHQANPRGSAGPCCTPTKMSPINMLYFNGKEQIIY | |
| | GKIPAMVVDRCGCS | |
| Inhibin-beta A | SPTPGSEGHSAAPDCPSCALAALPKDVPNSQPEMVEAVKKHI | 84 |
| | LNMLHLKKRPDVTQPVPKAALLNAIRKLHVGKVGENGYVEI | |
| | EDDIGRRAEMNELMEQTSEIITFAESGTARKTLHFEISKEGSD | |
| | LSVVERAEVWLFLKVPKANRTRTKVTIRLFQQQKHPQGSLD | |
| | TGEEAEEVGLKGERSELLLSEKVVDARKSTWHVFPVSSSIQR | |
| | LLDQGKSSLDVRIACEQCQESGASLVLLGKKKKKEEEGEGK | |
| | KKGGGEGGAGADEEKEQSHRPFLMLQARQSEDHPHRRRRR | |
| | GLECDGKVNICCKKQFFVSFKDIGWNDWIIAPSGYHANYCE | |
| | GECPSHIAGTSGSSLSFHSTVINHYRMRGHSPFANLKSCCVPT | |
| | KLRPMSMLYYDDGQNIIKKDIQNMIVEECGCS | |

Putative proprotein convertase cleavage sites in Table 1 are underlined and in bold.

TABLE 2
GDF protein modules

| TGF-β | | | SEQ |
|---|---|---|---|
| Family | | | ID |
| Member | Protein Module | Prodomain and growth factor Sequence | NO |
| GDF8 | prodomain | NENSEQKENVEKEGLCNACTWRQNTKSSRIEA | 85 |
| | | IKIQILSKLRLETAPNISKDVIRQLLPKAPPLREL | |
| | | IDQYDVQRDDSSDGSLEDDDYHATTETIITMPT | |
| | | ESDFLMQVDGKPKCCFFKFSSKIQYNKVVKAQ | |
| | | LWIYLRPVETPTTVFVQILRLIKPMKDGTRYTG | |
| | | IRSLKLDMNPGTGIWQSIDVKTVLQNWLKQPE | |
| | | SNLGIEIKALDENGHDLAVTFPGPGEDGLNPFL | |
| | | EVKVTDTPKRSRR | |
| GDF11 | prodomain | AEGPAAAAAAAAAAAAAGVGGERSSRPAPSV | 86 |
| | | APEPDGCPVCVWRQHSRELRLESIKSQILSKLR | |
| | | LKEAPNISREVVKQLLPKAPPLQQILDLHDFQG | |
| | | DALQPEDFLEEDEYHATTETVISMAQETDPAV | |
| | | QTDGSPLCCHFHFSPKVMFTKVLKAQLWVYL | |
| | | RPVPRPATVYLQILRLKPLTGEGTAGGGGGGR | |
| | | RHIRIRSLKIELHSRSGHWQSIDFKQVLHSWFR | |
| | | QPQSNWGIEINAFDPSGTDLAVTSLGPGAEGLH | |
| | | PFMELRVLENTKRSRR | |
| GDF8 | straight jacket | NENSEQKENVEKEGLCNACTWRQNTKSSRIEA | 87 |
| | region | IKIQILSKLRLETAPNISKDVIRQLLPKAPPL | |
| GDF11 | straight jacket | AEGPAAAAAAAAAAAAAGVGGERSSRPAPSV | 88 |
| | region | APEPDGCPVCVWRQHSRELRLESIKSQILSKLR | |
| | | LKEAPNISREVVKQLLPKAPPL | |
| GDF8 | growth factor | DFGLDCDEHSTESRCCRYPLTVDFEAFGWDWI | 89 |
| | domain | IAPKRYKANYCSGECEFVFLQKYPHTHLVHQA | |
| | | NPRGSAGPCCTPTKMSPINMLYFNGKEQIIYGK | |
| | | IPAMVVDRCGCS | |
| GDF11 | growth factor | NLGLDCDEHSSESRCCRYPLTVDFEAFGWDWI | 90 |
| | domain | IAPKRYKANYCSGQCEYMFMQKYPHTHLVQQ | |
| | | ANPRGSAGPCCTPTKMSPINMLYFNDKQQIIYG | |
| | | KIPGMVVDRCGCS | |
| GDF8 | BMP/Tolloid | between residues R75 and D76 | — |
| | cleavage site | | |
| GDF11 | BMP/Tolloid | between residues G97 and D98 | — |
| | cleavage site | | |
| GDF8 | arm region | RELIDQYDVQRDDSSDGSLEDDDYHATTETIIT | 91 |
| | | MPTESDFLMQVDGKPKCCFFKFSSKIQYNKVV | |
| | | KAQLWIYLRPVETPTTVFVQILRLIKPMKDGTR | |
| | | YTGIRSLKLDMNPGTGIWQSIDVKTVLQNWLK | |
| | | QPESNLGIEIKALDENGHDLAVTFPGPGEDGLN | |
| | | PFLEVKVTDTPKRSRR | |
| GDF11 | arm region | QQILDLHDFQGDALQPEDFLEEDEYHATTETVI | 92 |
| | | SMAQETDPAVQTDGSPLCCHFHFSPKVMFTKV | |
| | | LKAQLWVYLRPVPRPATVYLQILRLKPLTGEG | |
| | | TAGGGGGGRRHIRIRSLKIELHSRSGHWQSIDF | |
| | | KQVLHSWFRQPQSNWGIEINAFDPSGTDLAVT | |
| | | SLGPGAEGLHPFMELRVLENTKRSRR | |

TABLE 3
Non-human proteins

| | | | SEQ ID |
|---|---|---|---|
| Protein | Species | Sequence | NO |
| GDF8 | Mouse | NEGSEREENVEKEGLCNACAWRQNTRYSRIEAIKIQ | 93 |
| | | ILSKLRLETAPNISKDAIRQLLPRAPPLRELIDQYDV | |
| | | QRDDSSDGSLEDDDYHATTETIITMPTESDFLMQAD | |
| | | GKPKCCFFKFSSKIQYNKVVKAQLWIYLRPVKTPTT | |
| | | VFVQILRLIKPMKDGTRYTGIRSLKLDMSPGTGIWQ | |
| | | SIDVKTVLQNWLKQPESNLGIEIKALDENGHDLAVT | |
| | | FPGPGEDGLNPFLEVKVTDTPKRSRRDFGLDCDEHS | |
| | | TESRCCRYPLTVDFEAFGWDWIIAPKRYKANYCSG | |
| | | ECEFVFLQKYPHTHLVHQANPRGSAGPCCTPTKMS | |
| | | PINMLYFNGKEQIIYGKIPAMVVDRCGCS | |
| GDF8 | Mouse | NEGSEREENVEKEGLCNACAWRQNTRYSRIEAIKIQ | 94 |
| prodomain | | ILSKLRLETAPNISKDAIRQLLPRAPPLRELIDQYDV | |
| | | QRDDSSDGSLEDDDYHATTETIITMPTESDFLMQAD | |
| | | GKPKCCFFKFSSKIQYNKVVKAQLWIYLRPVKTPTT | |
| | | VFVQILRLIKPMKDGTRYTGIRSLKLDMSPGTGIWQ | |
| | | SIDVKTVLQNWLKQPESNLGIEIKALDENGHDLAVT | |
| | | FPGPGEDGLNPFLEVKVTDTPKRSRR | |
| proGDF8 | Cyno | NENSEQKENVEKEGLCNACTWRQNTKSSRIEAIKIQ | 95 |
| | | ILSKLRLETAPNISKDAIRQLLPKAPPLRELIDQYDV | |
| | | QRDDSSDGSLEDDDYHATTETIITMPTESDFLMQVD | |
| | | GKPKCCFFKFSSKIQYNKVVKAQLWIYLRPVETPTT | |
| | | VFVQILRLIKPMKDGTRYTGIRSLKLDMNPGTGIWQ | |
| | | SIDVKTVLQNWLKQPESNLGIEIKALDENGHDLAVT | |
| | | FPGPGEDGLNPFLEVKVTDTPKRSRRDFGLDCDEHS | |
| | | TESRCCRYPLTVDFEAFGWDWIIAPKRYKANYCSG | |
| | | ECEFVFLQKYPHTHLVHQANPRGSAGPCCTPTKMS | |
| | | PINMLYFNGKEQIIYGKIPAMVVDRCGCS | |
| GDF8 | Cyno | NENSEQKENVEKEGLCNACTWRQNTKSSRIEAIKIQ | 96 |
| prodomain | | ILSKLRLETAPNISKDAIRQLLPKAPPLRELIDQYDV | |
| | | QRDDSSDGSLEDDDYHATTETIITMPTESDFLMQVD | |
| | | GKPKCCFFKFSSKIQYNKVVKAQLWIYLRPVETPTT | |
| | | VFVQILRLIKPMKDGTRYTGIRSLKLDMNPGTGIWQ | |
| | | SIDVKTVLQNWLKQPESNLGIEIKALDENGHDLAVT | |
| | | FPGPGEDGLNPFLEVKVTDTPKRSRR | |
| proGDF11 | Mouse | AEGPAAAAAAAAAAAGVGGERSSRPAPSAPPEPDG | 97 |
| | | CPVCVWRQHSRELRLESIKSQILSKLRLKEAPNISRE | |
| | | VVKQLLPKAPPLQQILDLHDFQGDALQPEDFLEEDE | |
| | | YHATTETVISMAQETDPAVQTDGSPLCCHFHFSPKV | |
| | | MFTKVLKAQLWVYLRPVPRPATVYLQILRLKPLTG | |
| | | EGTAGGGGGGRRHIRIRSLKIELHSRSGHWQSIDFK | |
| | | QVLHSWFRQPQSNWGIEINAFDPSGTDLAVTSLGPG | |
| | | AEGLHPFMELRVLENTKRSRRNLGLDCDEHSSESR | |
| | | CCRYPLTVDFEAFGWDWIIAPKRYKANYCSGQCEY | |
| | | MFMQKYPHTHLVQQANPRGSAGPCCTPTKMSPIN | |
| | | MLYFNDKQQIIYGKIPGMVVDRCGCS | |
| GDF11 | Mouse | AEGPAAAAAAAAAAAGVGGERSSRPAPSAPPEPDG | 98 |
| prodomain | | CPVCVWRQHSRELRLESIKSQILSKLRLKEAPNISRE | |
| | | VVKQLLPKAPPLQQILDLHDFQGDALQPEDFLEEDE | |
| | | YHATTETVISMAQETDPAVQTDGSPLCCHFHFSPKV | |
| | | MFTKVLKAQLWVYLRPVPRPATVYLQILRLKPLTG | |
| | | EGTAGGGGGGRRHIRIRSLKIELHSRSGHWQSIDFK | |
| | | QVLHSWFRQPQSNWGIEINAFDPSGTDLAVTSLGPG | |
| | | AEGLHPFMELRVLENTKRSRR | |

TABLE 4
Protein modules

| | | | SEQ |
|---|---|---|---|
| Protein | Residues | Sequence | ID NO |
| GDF8 | 1-75 | NENSEQKENVEKEGLCNACTWRQNTKSSRIEAIKIQIL | 99 |
| | | SKLRLETAPNISKDVIRQLLPKAPPLRELIDQYDVQR | |
| GDF8 | 1-64 | NENSEQKENVEKEGLCNACTWRQNTKSSRIEAIKIQIL | 100 |
| | | SKLRLETAPNISKDVIRQLLPKAPPL | |
| GDF8 | 75-end | RDDSSDGSLEDDDYHATTETIITMPTESDFLMQVDGK | 101 |
| | | PKCCFFKFSSKIQYNKVVKAQLWIYLRPVETPTTVFV | |
| | | QILRLIKPMKDGTRYTGIRSLKLDMNPGTGIWQSIDVK | |
| | | TVLQNWLKQPESNLGIEIKALDENGHDLAVTFPGPGE | |
| | | DGLNPFLEVKVTDTPKRSRRDFGLDCDEHSTESRCCR | |
| | | YPLTVDFEAFGWDWIIAPKRYKANYCSGECEFVFLQK | |
| | | YPHTHLVHQANPRGSAGPCCTPTKMSPINMLYFNGK | |
| | | EQIIYGKIPAMVVDRCGCS | |
| GDF8 | 65-end | RELIDQYDVQRDDSSDGSLEDDDYHATTETIITMPTES | 102 |
| | | DFLMQVDGKPKCCFFKFSSKIQYNKVVKAQLWIYLR | |
| | | PVETPTTVFVQILRLIKPMKDGTRYTGIRSLKLDMNPG | |
| | | TGIWQSIDVKTVLQNWLKQPESNLGIEIKALDENGHD | |
| | | LAVTFPGPGEDGLNPFLEVKVTDTPKRSRRDFGLDCD | |
| | | EHSTESRCCRYPLTVDFEAFGWDWIIAPKRYKANYCS | |
| | | GECEFVFLQKYPHTHLVHQANPRGSAGPCCTPTKMSP | |
| | | INMLYFNGKEQIIYGKIPAMVVDRCGCS | |
| GDF8 | 65-243 | RELIDQYDVQRDDSSDGSLEDDDYHATTETIITMPTES | 103 |
| | | DFLMQVDGKPKCCFFKFSSKIQYNKVVKAQLWIYLR | |
| | | PVETPTTVFVQILRLIKPMKDGTRYTGIRSLKLDMNPG | |
| | | TGIWQSIDVKTVLQNWLKQPESNLGIEIKALDENGHD | |
| | | LAVTFPGPGEDGLNPFLEVKVTDTPKRSRR | |
| GDF8 | 76-243 | DDSSDGSLEDDDYHATTETIITMPTESDFLMQVDGKP | 104 |
| | | KCCFFKFSSKIQYNKVVKAQLWIYLRPVETPTTVFVQI | |
| | | LRLIKPMKDGTRYTGIRSLKLDMNPGTGIWQSIDVKT | |
| | | VLQNWLKQPESNLGIEIKALDENGHDLAVTFPGPGED | |
| | | GLNPFLEVKVTDTPKRSRR | |
| GDF8 | 244-352 | DFGLDCDEHSTESRCCRYPLTVDFEAFGWDWIIAPKR | 105 |
| | | YKANYCSGECEFVFLQKYPHTHLVHQANPRGSAGPC | |
| | | CTPTKMSPINMLYFNGKEQIIYGKIPAMVVDRCGCS | |
| GDF11 | 1-86 | AEGPAAAAAAAAAAAAAGVGGERSSRPAPSVAPEPD | 106 |
| | | GCPVCVWRQHSRELRLESIKSQILSKLRLKEAPNISRE | |
| | | VVKQLLPKAPPL | |
| GDF11 | 1-96 | AEGPAAAAAAAAAAAAAGVGGERSSRPAPSVAPEPD | 107 |
| | | GCPVCVWRQHSRELRLESIKSQILSKLRLKEAPNISRE | |
| | | VVKQLLPKAPPLQQILDLHDFQ | |
| GDF11 | 1-108 | AEGPAAAAAAAAAAAAAGVGGERSSRPAPSVAPEPD | 108 |
| | | GCPVCVWRQHSRELRLESIKSQILSKLRLKEAPNISRE | |
| | | VVKQLLPKAPPLQQILDLHDFQGDALQPEDFLEE | |
| GDF11 | 97-274 | GDALQPEDFLEEDEYHATTETVISMAQETDPAVQTDG | 109 |
| | | SPLCCHFHFSPKVMFTKVLKAQLWVYLRPVPRPATV | |
| | | YLQILRLKPLTGEGTAGGGGGGRRHIRIRSLKIELHSR | |
| | | SGHWQSIDFKQVLHSWFRQPQSNWGIEINAFDPSGTD | |
| | | LAVTSLGPGAEGLHPFMELRVLENTKRSRR | |
| GDF11 | 87-274 | QQILDLHDFQGDALQPEDFLEEDEYHATTETVISMAQ | 110 |
| | | ETDPAVQTDGSPLCCHFHFSPKVMFTKVLKAQLWVY | |
| | | LRPVPRPATVYLQILRLKPLTGEGTAGGGGGGRRHIRI | |
| | | RSLKIELHSRSGHWQSIDFKQVLHSWFRQPQSNWGIEI | |
| | | NAFDPSGTDLAVTSLGPGAEGLHPFMELRVLENTKRS | |
| | | RR | |
| GDF11 | 275-383 | NLGLDCDEHSSESRCCRYPLTVDFEAFGWDWIIAPKR | 111 |
| | | YKANYCSGQCEYMFMQKYPHTHLVQQANPRGSAGP | |
| | | CCTPTKMSPINMLYFNDKQQIIYGKIPGMVVDRCGCS | |
| Inhibin | 1-64 | SPTPGSEGHSAAPDCPSCALAALPKDVPNSQPEMVEA | 112 |
| Beta A | | VKKHILNMLHLKKRPDVTQPVPKAALL | |
| Inhibin | 1-76 | SPTPGSEGHSAAPDCPSCALAALPKDVPNSQPEMVEA | 113 |
| Beta A | | VKKHILNMLHLKKRPDVTQPVPKAALLNAIRKLHVG | |
| | | KVG | |
| Inhibin | 65-288 | NAIRKLHVGKVGENGYVEIEDDIGRRAEMNELMEQT | 114 |
| Beta A | | SEIITFAESGTARKTLHFEISKEGSDLSVVERAEVWLFL | |
| | | KVPKANRTRTKVTIRLFQQQKHPQGSLDTGEEAEEVG | |
| | | LKGERSELLLSEKVVDARKSTWHVFPVSSSIQRLLDQ | |
| | | GKSSLDVRIACEQCQESGASLVLLGKKKKKEEEGEGK | |
| | | KKGGGEGGAGADEEKEQSHRPFLMLQARQSEDHPHR | |
| | | RR | |
| Inhibin | 65-289 | NAIRKLHVGKVGENGYVEIEDDIGRRAEMNELMEQT | 115 |
| Beta A | | SEIITFAESGTARKTLHFEISKEGSDLSVVERAEVWLFL | |
| | | KVPKANRTRTKVTIRLFQQQKHPQGSLDTGEEAEEVG | |
| | | LKGERSELLLSEKVVDARKSTWHVFPVSSSIQRLLDQ | |
| | | GKSSLDVRIACEQCQESGASLVLLGKKKKKEEEGEGK | |
| | | KKGGGEGGAGADEEKEQSHRPFLMLQARQSEDHPHR | |
| | | RRR | |
| Inhibin | 65-290 | NAIRKLHVGKVGENGYVEIEDDIGRRAEMNELMEQT | 116 |
| Beta A | | SEIITFAESGTARKTLHFEISKEGSDLSVVERAEVWLFL | |
| | | KVPKANRTRTKVTIRLFQQQKHPQGSLDTGEEAEEVG | |
| | | LKGERSELLLSEKVVDARKSTWHVFPVSSSIQRLLDQ | |
| | | GKSSLDVRIACEQCQESGASLVLLGKKKKKEEEGEGK | |
| | | KKGGGEGGAGADEEKEQSHRPFLMLQARQSEDHPHR | |
| | | RRRR | |
| Inhibin | 77-289 | ENGYVEIEDDIGRRAEMNELMEQTSEIITFAESGTARK | 117 |
| Beta A | | TLHFEISKEGSDLSVVERAEVWLFLKVPKANRTRTKV | |
| | | TIRLFQQQKHPQGSLDTGEEAEEVGLKGERSELLLSEK | |
| | | VVDARKSTWHVFPVSSSIQRLLDQGKSSLDVRIACEQ | |
| | | CQESGASLVLLGKKKKKEEEGEGKKKGGGEGGAGA | |
| | | DEEKEQSHRPFLMLQARQSEDHPHRRRR | |
| Inhibin | 77-290 | ENGYVEIEDDIGRRAEMNELMEQTSEIITFAESGTARK | 118 |
| Beta A | | TLHFEISKEGSDLSVVERAEVWLFLKVPKANRTRTKV | |
| | | TIRLFQQQKHPQGSLDTGEEAEEVGLKGERSELLLSEK | |
| | | VVDARKSTWHVFPVSSSIQRLLDQGKSSLDVRIACEQ | |
| | | CQESGASLVLLGKKKKKEEEGEGKKKGGGEGGAGA | |
| | | DEEKEQSHRPFLMLQARQSEDHPHRRRRR | |
| Inhibin | 77-end | ENGYVEIEDDIGRRAEMNELMEQTSEIITFAESGTARK | 119 |
| Beta A | | TLHFEISKEGSDLSVVERAEVWLFLKVPKANRTRTKV | |
| | | TIRLFQQQKHPQGSLDTGEEAEEVGLKGERSELLLSEK | |
| | | VVDARKSTWHVFPVSSSIQRLLDQGKSSLDVRIACEQ | |
| | | CQESGASLVLLGKKKKKEEEGEGKKKGGGEGGAGA | |
| | | DEEKEQSHRPFLMLQARQSEDHPHRRRRRGLECDGK | |
| | | VNICCKKQFFVSFKDIGWNDWIIAPSGYHANYCEGEC | |
| | | PSHIAGTSGSSLSFHSTVINHYRMRGHSPFANLKSCCV | |
| | | PTKLRPMSMLYYDDGQNIIKKDIQNMIVEECGCS | |
| Inhibin | 291-406 | GLECDGKVNICCKKQFFVSFKDIGWNDWIIAPSGYHA | 120 |
| Beta A | | NYCEGECPSHIAGTSGSSLSFHSTVINHYRMRGHSPFA | |
| | | NLKSCCVPTKLRPMSMLYYDDGQNIIKKDIQNMIVEE | |
| | | CGCS | |

TABLE 5
Protein module combinations

| | | | | SEQ |
|---|---|---|---|---|
| Protein | Protein | Protein | | ID |
| module 1 | module 2 | module 3 | Chimeric Sequence | NO |
| GDF11 | GDF8 | GDF11 | AEGPAAAAAAAAAAAAAGVGGERSSRPAPSVAP | 121 |
| (1-96) | (76-243) | (275-383) | EPDGCPVCVWRQHSRELRLESIKSQILSKLRLKEA | |
| | | | PNISREVVKQLLPKAPPLQQILDLHDFQDDSSDGS | |
| | | | LEDDDYHATTETIITMPTESDFLMQVDGKPKCCFF | |
| | | | KFSSKIQYNKVVKAQLWIYLRPVETPTTVFVQILR | |
| | | | LIKPMKDGTRYTGIRSLKLDMNPGTGIWQSIDVKT | |
| | | | VLQNWLKQPESNLGIEIKALDENGHDLAVTFPGP | |
| | | | GEDGLNPFLEVKVTDTPKRSRRNLGLDCDEHSSE | |
| | | | SRCCRYPLTVDFEAFGWDWIIAPKRYKANYCSGQ | |
| | | | CEYMFMQKYPHTHLVQQANPRGSAGPCCTPTKM | |
| | | | SPINMLYFNDKQQIIYGKIPGMVVDRCGCS | |
| GDF11 | GDF8 | GDF11 | AEGPAAAAAAAAAAAAAGVGGERSSRPAPSVAP | 122 |
| (1-86) | (65-243) | (275-383) | EPDGCPVCVWRQHSRELRLESIKSQILSKLRLKEA | |
| | | | PNISREVVKQLLPKAPPLRELIDQYDVQRDDSSDG | |
| | | | SLEDDDYHATTETIITMPTESDFLMQVDGKPKCCF | |
| | | | FKFSSKIQYNKVVKAQLWIYLRPVETPTTVFVQIL | |
| | | | RLIKPMKDGTRYTGIRSLKLDMNPGTGIWQSIDV | |
| | | | KTVLQNWLKQPESNLGIEIKALDENGHDLAVTFP | |
| | | | GPGEDGLNPFLEVKVTDTPKRSRRNLGLDCDEHS | |
| | | | SESRCCRYPLTVDFEAFGWDWIIAPKRYKANYCS | |
| | | | GQCEYMFMQKYPHTHLVQQANPRGSAGPCCTPT | |
| | | | KMSPINMLYFNDKQQIIYGKIPGMVVDRCGCS | |
| GDF11 | GDF8 | N/A | AEGPAAAAAAAAAAAAAGVGGERSSRPAPSVAP | 123 |
| (1-96) | (76-243) | | EPDGCPVCVWRQHSRELRLESIKSQILSKLRLKEA | |
| | | | PNISREVVKQLLPKAPPLQQILDLHDFQDDSSDGS | |
| | | | LEDDDYHATTETIITMPTESDFLMQVDGKPKCCFF | |
| | | | KFSSKIQYNKVVKAQLWIYLRPVETPTTVFVQILR | |
| | | | LIKPMKDGTRYTGIRSLKLDMNPGTGIWQSIDVKT | |
| | | | VLQNWLKQPESNLGIEIKALDENGHDLAVTFPGP | |
| | | | GEDGLNPFLEVKVTDTPKRSRR | |
| GDF11 | GDF8 | NA | AEGPAAAAAAAAAAAAAGVGGERSSRPAPSVAP | 124 |
| (1-86) | (65-243) | | EPDGCPVCVWRQHSRELRLESIKSQILSKLRLKEA | |
| | | | PNISREVVKQLLPKAPPLRELIDQYDVQRDDSSDG | |
| | | | SLEDDDYHATTETIITMPTESDFLMQVDGKPKCCF | |
| | | | FKFSSKIQYNKVVKAQLWIYLRPVETPTTVFVQIL | |
| | | | RLIKPMKDGTRYTGIRSLKLDMNPGTGIWQSIDV | |
| | | | KTVLQNWLKQPESNLGIEIKALDENGHDLAVTFP | |
| | | | GPGEDGLNPFLEVKVTDTPKRSRR | |
| GDF11 | Inhibin | GDF11 | AEGPAAAAAAAAAAAAAGVGGERSSRPAPSVAP | 125 |
| (1-96) | Beta A | (275-383) | EPDGCPVCVWRQHSRELRLESIKSQILSKLRLKEA | |
| | (77-290) | | PNISREVVKQLLPKAPPLQQILDLHDFQENGYVEI | |
| | | | EDDIGRRAEMNELMEQTSEIITFAESGTARKTLHF | |
| | | | EISKEGSDLSVVERAEVWLFLKVPKANRTRTKVTI | |
| | | | RLFQQQKHPQGSLDTGEEAEEVGLKGERSELLLS | |
| | | | EKVVDARKSTWHVFPVSSSIQRLLDQGKSSLDVRI | |
| | | | ACEQCQESGASLVLLGKKKKKEEEGEGKKKGGG | |
| | | | EGGAGADEEKEQSHRPFLMLQARQSEDHPHRRR | |
| | | | RRNLGLDCDEHSSESRCCRYPLTVDFEAFGWDWI | |
| | | | IAPKRYKANYCSGQCEYMFMQKYPHTHLVQQAN | |
| | | | PRGSAGPCCTPTKMSPINMLYFNDKQQIIYGKIPG | |
| | | | MVVDRCGCS | |
| GDF11 | Inhibin | GDF11 | AEGPAAAAAAAAAAAAAGVGGERSSRPAPSVAP | 126 |
| (1-86) | Beta A | (275-383) | EPDGCPVCVWRQHSRELRLESIKSQILSKLRLKEA | |
| | (65-290) | | PNISREVVKQLLPKAPPLNAIRKLHVGKVGENGY | |
| | | | VEIEDDIGRRAEMNELMEQTSEIITFAESGTARKTL | |
| | | | HFEISKEGSDLSVVERAEVWLFLKVPKANRTRTK | |
| | | | VTIRLFQQQKHPQGSLDTGEEAEEVGLKGERSELL | |
| | | | LSEKVVDARKSTWHVFPVSSSIQRLLDQGKSSLD | |
| | | | VRIACEQCQESGASLVLLGKKKKKEEEGEGKKKG | |
| | | | GGEGGAGADEEKEQSHRPFLMLQARQSEDHPHR | |
| | | | RRRRNLGLDCDEHSSESRCCRYPLTVDFEAFGWD | |
| | | | WIIAPKRYKANYCSGQCEYMFMQKYPHTHLVQQ | |
| | | | ANPRGSAGPCCTPTKMSPINMLYFNDKQQIIYGKI | |
| | | | PGMVVDRCGCS | |
| GDF11 | Inhibin | N/A | AEGPAAAAAAAAAAAAAGVGGERSSRPAPSVAP | 127 |
| (1-96) | Beta A | | EPDGCPVCVWRQHSRELRLESIKSQILSKLRLKEA | |
| | (77-290) | | PNISREVVKQLLPKAPPLQQILDLHDFQENGYVEI | |
| | | | EDDIGRRAEMNELMEQTSEIITFAESGTARKTLHF | |
| | | | EISKEGSDLSVVERAEVWLFLKVPKANRTRTKVTI | |
| | | | RLFQQQKHPQGSLDTGEEAEEVGLKGERSELLLS | |
| | | | EKVVDARKSTWHVFPVSSSIQRLLDQGKSSLDVRI | |
| | | | ACEQCQESGASLVLLGKKKKKEEEGEGKKKGGG | |
| | | | EGGAGADEEKEQSHRPFLMLQARQSEDHPHRRR | |
| | | | RR | |
| GDF11 | Inhibin | NA | AEGPAAAAAAAAAAAAAGVGGERSSRPAPSVAP | 128 |
| (1-86) | Beta A | | EPDGCPVCVWRQHSRELRLESIKSQILSKLRLKEA | |
| | (65-290) | | PNISREVVKQLLPKAPPLNAIRKLHVGKVGENGY | |
| | | | VEIEDDIGRRAEMNELMEQTSEIITFAESGTARKTL | |
| | | | HFEISKEGSDLSVVERAEVWLFLKVPKANRTRTK | |
| | | | VTIRLFQQQKHPQGSLDTGEEAEEVGLKGERSELL | |
| | | | LSEKVVDARKSTWHVFPVSSSIQRLLDQGKSSLD | |
| | | | VRIACEQCQESGASLVLLGKKKKKEEEGEGKKKG | |
| | | | GGEGGAGADEEKEQSHRPFLMLQARQSEDHPHR | |
| | | | RRRR | |
| GDF8 | GDF11 | GDF8 | NENSEQKENVEKEGLCNACTWRQNTKSSRIEAIKI | 129 |
| (1-75) | (97-274) | (244-352) | QILSKLRLETAPNISKDVIRQLLPKAPPLRELIDQY | |
| | | | DVQRGDALQPEDFLEEDEYHATTETVISMAQETD | |
| | | | PAVQTDGSPLCCHFHFSPKVMFTKVLKAQLWVY | |
| | | | LRPVPRPATVYLQILRLKPLTGEGTAGGGGGGRR | |
| | | | HIRIRSLKIELHSRSGHWQSIDFKQVLHSWFRQPQS | |
| | | | NWGIEINAFDPSGTDLAVTSLGPGAEGLHPFMELR | |
| | | | VLENTKRSRRDFGLDCDEHSTESRCCRYPLTVDFE | |
| | | | AFGWDWIIAPKRYKANYCSGECEFVFLQKYPHTH | |
| | | | LVHQANPRGSAGPCCTPTKMSPINMLYFNGKEQII | |
| | | | YGKIPAMVVDRCGCS | |
| GDF8 | GDF11 | GDF8 | NENSEQKENVEKEGLCNACTWRQNTKSSRIEAIKI | 130 |
| (1-64) | (87-274) | (244-352) | QILSKLRLETAPNISKDVIRQLLPKAPPLQQILDLH | |
| | | | DFQGDALQPEDFLEEDEYHATTETVISMAQETDP | |
| | | | AVQTDGSPLCCHFHFSPKVMFTKVLKAQLWVYL | |
| | | | RPVPRPATVYLQILRLKPLTGEGTAGGGGGGRRHI | |
| | | | RIRSLKIELHSRSGHWQSIDFKQVLHSWFRQPQSN | |
| | | | WGIEINAFDPSGTDLAVTSLGPGAEGLHPFMELRV | |
| | | | LENTKRSRRDFGLDCDEHSTESRCCRYPLTVDFEA | |
| | | | FGWDWIIAPKRYKANYCSGECEFVFLQKYPHTHL | |
| | | | VHQANPRGSAGPCCTPTKMSPINMLYFNGKEQIIY | |
| | | | GKIPAMVVDRCGCS | |
| GDF8 | GDF11 | N/A | NENSEQKENVEKEGLCNACTWRQNTKSSRIEAIKI | 131 |
| (1-75) | (97-274) | | QILSKLRLETAPNISKDVIRQLLPKAPPLRELIDQY | |
| | | | DVQRGDALQPEDFLEEDEYHATTETVISMAQETD | |
| | | | PAVQTDGSPLCCHFHFSPKVMFTKVLKAQLWVY | |
| | | | LRPVPRPATVYLQILRLKPLTGEGTAGGGGGGRR | |
| | | | HIRIRSLKIELHSRSGHWQSIDFKQVLHSWFRQPQS | |
| | | | NWGIEINAFDPSGTDLAVTSLGPGAEGLHPFMELR | |
| | | | VLENTKRSRR | |
| GDF8 | GDF11 | GDF8 | NENSEQKENVEKEGLCNACTWRQNTKSSRIEAIKI | 132 |
| (1-64) | (87-274) | (244-352) | QILSKLRLETAPNISKDVIRQLLPKAPPLQQILDLH | |
| | | | DFQGDALQPEDFLEEDEYHATTETVISMAQETDP | |
| | | | AVQTDGSPLCCHFHFSPKVMFTKVLKAQLWVYL | |
| | | | RPVPRPATVYLQILRLKPLTGEGTAGGGGGGRRHI | |
| | | | RIRSLKIELHSRSGHWQSIDFKQVLHSWFRQPQSN | |
| | | | WGIEINAFDPSGTDLAVTSLGPGAEGLHPFMELRV | |
| | | | LENTKRSRRDFGLDCDEHSTESRCCRYPLTVDFEA | |
| | | | FGWDWIIAPKRYKANYCSGECEFVFLQKYPHTHL | |
| | | | VHQANPRGSAGPCCTPTKMSPINMLYFNGKEQIIY | |
| | | | GKIPAMVVDRCGCS | |
| GDF8 | Inhibin | GDF8 | NENSEQKENVEKEGLCNACTWRQNTKSSRIEAIKI | 133 |
| (1-75) | Beta A | (244-352) | QILSKLRLETAPNISKDVIRQLLPKAPPLRELIDQY | |
| | (77-289) | | DVQRENGYVEIEDDIGRRAEMNELMEQTSEIITFA | |
| | | | ESGTARKTLHFEISKEGSDLSVVERAEVWLFLKVP | |
| | | | KANRTRTKVTIRLFQQQKHPQGSLDTGEEAEEVG | |
| | | | LKGERSELLLSEKVVDARKSTWHVFPVSSSIQRLL | |
| | | | DQGKSSLDVRIACEQCQESGASLVLLGKKKKKEE | |
| | | | EGEGKKKGGGEGGAGADEEKEQSHRPFLMLQAR | |
| | | | QSEDHPHRRRRDFGLDCDEHSTESRCCRYPLTVD | |
| | | | FEAFGWDWIIAPKRYKANYCSGECEFVFLQKYPH | |
| | | | THLVHQANPRGSAGPCCTPTKMSPINMLYFNGKE | |
| | | | QIIYGKIPAMVVDRCGCS | |
| GDF8 | Inhibin | GDF8 | NENSEQKENVEKEGLCNACTWRQNTKSSRIEAIKI | 134 |
| (1-64) | Beta A | (244-352) | QILSKLRLETAPNISKDVIRQLLPKAPPLNAIRKLH | |
| | (65-290) | | VGKVGENGYVEIEDDIGRRAEMNELMEQTSEIITF | |
| | | | AESGTARKTLHFEISKEGSDLSVVERAEVWLFLK | |
| | | | VPKANRTRTKVTIRLFQQQKHPQGSLDTGEEAEE | |
| | | | VGLKGERSELLLSEKVVDARKSTWHVFPVSSSIQR | |
| | | | LLDQGKSSLDVRIACEQCQESGASLVLLGKKKKK | |
| | | | EEEGEGKKKGGGEGGAGADEEKEQSHRPFLMLQ | |
| | | | ARQSEDHPHRRRRRDFGLDCDEHSTESRCCRYPL | |
| | | | TVDFEAFGWDWIIAPKRYKANYCSGECEFVFLQK | |
| | | | YPHTHLVHQANPRGSAGPCCTPTKMSPINMLYFN | |
| | | | GKEQIIYGKIPAMVVDRCGCS | |
| GDF8 | Inhibin | N/A | NENSEQKENVEKEGLCNACTWRQNTKSSRIEAIKI | 135 |
| (1-75) | Beta A | | QILSKLRLETAPNISKDVIRQLLPKAPPLRELIDQY | |
| | (77-290) | | DVQRENGYVEIEDDIGRRAEMNELMEQTSEIITFA | |
| | | | ESGTARKTLHFEISKEGSDLSVVERAEVWLFLKVP | |
| | | | KANRTRTKVTIRLFQQQKHPQGSLDTGEEAEEVG | |
| | | | LKGERSELLLSEKVVDARKSTWHVFPVSSSIQRLL | |
| | | | DQGKSSLDVRIACEQCQESGASLVLLGKKKKKEE | |
| | | | EGEGKKKGGGEGGAGADEEKEQSHRPFLMLQAR | |
| | | | QSEDHPHRRRRR | |
| GDF8 | Inhibin | NA | NENSEQKENVEKEGLCNACTWRQNTKSSRIEAIKI | 136 |
| (1-64) | Beta A | | QILSKLRLETAPNISKDVIRQLLPKAPPLNAIRKLH | |
| | (65-290) | | VGKVGENGYVEIEDDIGRRAEMNELMEQTSEIITF | |
| | | | AESGTARKTLHFEISKEGSDLSVVERAEVWLFLK | |
| | | | VPKANRTRTKVTIRLFQQQKHPQGSLDTGEEAEE | |
| | | | VGLKGERSELLLSEKVVDARKSTWHVFPVSSSIQR | |
| | | | LLDQGKSSLDVRIACEQCQESGASLVLLGKKKKK | |
| | | | EEEGEGKKKGGGEGGAGADEEKEQSHRPFLMLQ | |
| | | | ARQSEDHPHRRRRR | |
| Inhibin | GDF8 | Inhibin | SPTPGSEGHSAAPDCPSCALAALPKDVPNSQPEM | 137 |
| Beta A | (76-243) | Beta A | VEAVKKHILNMLHLKKRPDVTQPVPKAALLNAIR | |
| (1-76) | | (291-406) | KLHVGKVGDDSSDGSLEDDDYHATTETIITMPTES | |
| | | | DFLMQVDGKPKCCFFKFSSKIQYNKVVKAQLWIY | |
| | | | LRPVETPTTVFVQILRLIKPMKDGTRYTGIRSLKLD | |
| | | | MNPGTGIWQSIDVKTVLQNWLKQPESNLGIEIKA | |
| | | | LDENGHDLAVTFPGPGEDGLNPFLEVKVTDTPKR | |
| | | | SRRGLECDGKVNICCKKQFFVSFKDIGWNDWIIAP | |
| | | | SGYHANYCEGECPSHIAGTSGSSLSFHSTVINHYR | |
| | | | MRGHSPFANLKSCCVPTKLRPMSMLYYDDGQNII | |
| | | | KKDIQNMIVEECGCS | |
| Inhibin | GDF8 | Inhibin | SPTPGSEGHSAAPDCPSCALAALPKDVPNSQPEM | 138 |
| Beta A | (65-243) | Beta A | VEAVKKHILNMLHLKKRPDVTQPVPKAALLRELI | |
| (1-64) | | (291-406) | DQYDVQRDDSSDGSLEDDDYHATTETIITMPTES | |
| | | | DFLMQVDGKPKCCFFKFSSKIQYNKVVKAQLWIY | |
| | | | LRPVETPTTVFVQILRLIKPMKDGTRYTGIRSLKLD | |
| | | | MNPGTGIWQSIDVKTVLQNWLKQPESNLGIEIKA | |
| | | | LDENGHDLAVTFPGPGEDGLNPFLEVKVTDTPKR | |
| | | | SRRGLECDGKVNICCKKQFFVSFKDIGWNDWIIAP | |
| | | | SGYHANYCEGECPSHIAGTSGSSLSFHSTVINHYR | |
| | | | MRGHSPFANLKSCCVPTKLRPMSMLYYDDGQNII | |
| | | | KKDIQNMIVEECGCS | |
| Inhibin | GDF8 | N/A | SPTPGSEGHSAAPDCPSCALAALPKDVPNSQPEM | 139 |
| Beta A | (76-243) | | VEAVKKHILNMLHLKKRPDVTQPVPKAALLNAIR | |
| (1-76) | | | KLHVGKVGDDSSDGSLEDDDYHATTETIITMPTES | |
| | | | DFLMQVDGKPKCCFFKFSSKIQYNKVVKAQLWIY | |
| | | | LRPVETPTTVFVQILRLIKPMKDGTRYTGIRSLKLD | |
| | | | MNPGTGIWQSIDVKTVLQNWLKQPESNLGIEIKA | |
| | | | LDENGHDLAVTFPGPGEDGLNPFLEVKVTDTPKR | |
| | | | SRR | |
| Inhibin | GDF8 | NA | SPTPGSEGHSAAPDCPSCALAALPKDVPNSQPEM | 140 |
| Beta A | (65-243) | | VEAVKKHILNMLHLKKRPDVTQPVPKAALLRELI | |
| (1-64) | | | DQYDVQRDDSSDGSLEDDDYHATTETIITMPTES | |
| | | | DFLMQVDGKPKCCFFKFSSKIQYNKVVKAQLWIY | |
| | | | LRPVETPTTVFVQILRLIKPMKDGTRYTGIRSLKLD | |
| | | | MNPGTGIWQSIDVKTVLQNWLKQPESNLGIEIKA | |
| | | | LDENGHDLAVTFPGPGEDGLNPFLEVKVTDTPKR | |
| | | | SRR | |
| Inhibin | GDF11 | Inhibin | SPTPGSEGHSAAPDCPSCALAALPKDVPNSQPEM | 141 |
| Beta A | (97-274) | Beta A | VEAVKKHILNMLHLKKRPDVTQPVPKAALLNAIR | |
| (1-76) | | (291-406) | KLHVGKVGGDALQPEDFLEEDEYHATTETVISMA | |
| | | | QETDPAVQTDGSPLCCHFHFSPKVMFTKVLKAQL | |
| | | | WVYLRPVPRPATVYLQILRLKPLTGEGTAGGGGG | |
| | | | GRRHIRIRSLKIELHSRSGHWQSIDFKQVLHSWFR | |
| | | | QPQSNWGIEINAFDPSGTDLAVTSLGPGAEGLHPF | |
| | | | MELRVLENTKRSRRGLECDGKVNICCKKQFFVSF | |
| | | | KDIGWNDWIIAPSGYHANYCEGECPSHIAGTSGSS | |
| | | | LSFHSTVINHYRMRGHSPFANLKSCCVPTKLRPMS | |
| | | | MLYYDDGQNIIKKDIQNMIVEECGCS | |
| Inhibin | GDF11 | Inhibin | SPTPGSEGHSAAPDCPSCALAALPKDVPNSQPEM | 142 |
| Beta A | (87-274) | Beta A | VEAVKKHILNMLHLKKRPDVTQPVPKAALLQQIL | |
| (1-64) | | (291-406) | DLHDFQGDALQPEDFLEEDEYHATTETVISMAQE | |
| | | | TDPAVQTDGSPLCCHFHFSPKVMFTKVLKAQLW | |
| | | | VYLRPVPRPATVYLQILRLKPLTGEGTAGGGGGG | |
| | | | RRHIRIRSLKIELHSRSGHWQSIDFKQVLHSWFRQ | |
| | | | PQSNWGIEINAFDPSGTDLAVTSLGPGAEGLHPFM | |
| | | | ELRVLENTKRSRRGLECDGKVNICCKKQFFVSFK | |
| | | | DIGWNDWIIAPSGYHANYCEGECPSHIAGTSGSSL | |
| | | | SFHSTVINHYRMRGHSPFANLKSCCVPTKLRPMS | |
| | | | MLYYDDGQNIIKKDIQNMIVEECGCS | |
| Inhibin | GDF11 | N/A | SPTPGSEGHSAAPDCPSCALAALPKDVPNSQPEM | 143 |
| Beta A | (97-274) | | VEAVKKHILNMLHLKKRPDVTQPVPKAALLNAIR | |
| (1-76) | | | KLHVGKVGGDALQPEDFLEEDEYHATTETVISMA | |
| | | | QETDPAVQTDGSPLCCHFHFSPKVMFTKVLKAQL | |
| | | | WVYLRPVPRPATVYLQILRLKPLTGEGTAGGGGG | |
| | | | GRRHIRIRSLKIELHSRSGHWQSIDFKQVLHSWFR | |
| | | | QPQSNWGIEINAFDPSGTDLAVTSLGPGAEGLHPF | |
| | | | MELRVLENTKRSRR | |
| Inhibin | GDF11 | NA | SPTPGSEGHSAAPDCPSCALAALPKDVPNSQPEM | 144 |
| Beta A | (87-274) | | VEAVKKHILNMLHLKKRPDVTQPVPKAALLQQIL | |
| (1-64) | | | DLHDFQGDALQPEDFLEEDEYHATTETVISMAQE | |
| | | | TDPAVQTDGSPLCCHFHFSPKVMFTKVLKAQLW | |
| | | | VYLRPVPRPATVYLQILRLKPLTGEGTAGGGGGG | |
| | | | RRHIRIRSLKIELHSRSGHWQSIDFKQVLHSWFRQ | |
| | | | PQSNWGIEINAFDPSGTDLAVTSLGPGAEGLHPFM | |
| | | | ELRVLENTKRSRR | |

TABLE 6
Recombinant antigens

| | | SEQ |
|---|---|---|
| | | ID |
| Protein | Amino acid sequence | NO |
| GDF8/11 chimera | MDMRVPAQLLGLLLLWFSGVLGDYKDDDDKHHHHHHL | 145 |
| (GDF8 1-64, | EVLFQGPNENSEQKENVEKEGLCNACTWRQNTKSSRIEA | |
| GDF11 87-274) | IKIQILSKLRLETAPNISKDVIRQLLPKAPPLQQILDLHDFQ | |
| | GDALQPEDFLEEDEYHATTETVISMAQETDPAVQTDGSP | |
| | LCCHFHFSPKVMFTKVLKAQLWVYLRPVPRPATVYLQIL | |
| | RLKPLTGEGTAGGGGGGRRHIRIRSLKIELHSRSGHWQSI | |
| | DFKQVLHSWFRQPQSNWGIEINAFDPSGTDLAVTSLGPG | |
| | AEGLHPFMELRVLENTKRSRR | |
| GDF11/8 chimera | AEGPAAAAAAAAAAAAAGVGGERSSRPAPSVAPEPDGC | 146 |
| (GDF11 1-86, | PVCVWRQHSRELRLESIKSQILSKLRLKEAPNISREVVKQ | |
| GDF8 65-243) | LLPKAPPLRELIDQYDVQRDDSSDGSLEDDDYHATTETIIT | |
| | MPTESDFLMQVDGKPKCCFFKFSSKIQYNKVVKAQLWIY | |
| | LRPVETPTTVFVQILRLIKPMKDGTRYTGIRSLKLDMNPG | |
| | TGIWQSIDVKTVLQNWLKQPESNLGIEIKALDENGHDLA | |
| | VTFPGPGEDGLNPFLEVKVTDTPKRSRR | |
| GDF11/Inh Beta A | AEGPAAAAAAAAAAAAAGVGGERSSRPAPSVAPEPDGC | 147 |
| chimera (GDF11 1-86, | PVCVWRQHSRELRLESIKSQILSKLRLKEAPNISREVVKQ | |
| Inh Beta A 65-290) | LLPKAPPLNAIRKLHVGKVGENGYVEIEDDIGRRAEMNE | |
| | LMEQTSEIITFAESGTARKTLHFEISKEGSDLSVVERAEVW | |
| | LFLKVPKANRTRTKVTIRLFQQQKHPQGSLDTGEEAEEV | |
| | GLKGERSELLLSEKVVDARKSTWHVFPVSSSIQRLLDQG | |
| | KSSLDVRIACEQCQESGASLVLLGKKKKKEEEGEGKKKG | |
| | GGEGGAGADEEKEQSHRPFLMLQARQSEDHPHRRRRR | |
| Inhibin Beta | SPTPGSEGHSAAPDCPSCALAALPKDVPNSQPEMVEAVK | 148 |
| A/GDF11 chimera | KHILNMLHLKKRPDVTQPVPKAALLQQILDLHDFQGDAL | |
| (Inh Beta A 1-64, | QPEDFLEEDEYHATTETVISMAQETDPAVQTDGSPLCCHF | |
| GDF11 87-274) | HFSPKVMFTKVLKAQLWVYLRPVPRPATVYLQILRLKPL | |
| | TGEGTAGGGGGGRRHIRIRSLKIELHSRSGHWQSIDFKQV | |
| | LHSWFRQPQSNWGIEINAFDPSGTDLAVTSLGPGAEGLHP | |
| | FMELRVLENTKRSRR | |

Antibodies designed to bind human proGDF11 were tested for binding to human proGDF11 (SEQ ID NO: 82); human latent GDF11 (SEQ ID NO: 82); murine proGDF11 (SEQ ID NO: 97); murine latent GDF11 (SEQ ID NO: 97); human proMyostatin (SEQ ID NO: 83); murine proMyostatin (SEQ ID NO: 93); human latent Myostatin (SEQ ID NO: 83); murine latent myostatin (SEQ ID NO: 93); human GDF11 ARM8 prodomain (SEQ ID NO: 124); human proGDF11ARM8 (SEQ ID NO: 122); human mature GDF11 (SEQ ID NO: 90).

The recombinant antibody library may be from a subject immunized with GDF11 or GDF11 prodomain complex, or a portion of GDF11 or GDF11 prodomain complex. Alternatively, the recombinant antibody library may be from a naive subject, i.e., one who has not been immunized with GDF11 prodomain complex, such as a human antibody library from a human subject who has not been immunized with human GDF11 prodomain complex. Antibodies of the disclosure are selected by screening the recombinant antibody library with the peptide comprising human GDF11 prodomain complex to thereby select those antibodies that recognize GDF11 prodomain complex. Methods for conducting such screening and selection are well known in the art, such as described in the references in the preceding paragraph. To select antibodies of the disclosure having particular binding affinities for hGDF11 prodomain complex, such as those that dissociate from human GDF11 prodomain complex with a particular k_{off} rate constant, the art-known method of surface biolayer interferometry can be used to select antibodies having the desired k_{off} rate constant. To select antibodies of the disclosure having a particular neutralizing activity for hGDF11 prodomain complex, such as those with a particular an IC₅₀, standard methods known in the art for assessing the inhibition of hGDF11 prodomain complex activity may be used.

In one aspect, the disclosure pertains to an isolated antibody, or an antigen-binding portion thereof, that binds human GDF11 prodomain complex. In another embodiment, the antibody is a neutralizing antibody. In various embodiments, the antibody is a recombinant antibody or a monoclonal antibody.

For example, binding proteins (e.g., antibodies or antigen binding portions thereof) of the present disclosure can be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present disclosure include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

Alternative to screening of recombinant antibody libraries by phage display, other methodologies known in the art for screening large combinatorial libraries can be applied to the identification of dual specificity antibodies of the disclosure. One type of alternative expression system is one in which the recombinant antibody library is expressed as RNA-protein fusions, as described in PCT Publication No. WO 98/31700 by Szostak and Roberts, and in Roberts, R. W. and Szostak, J. W. (1997) Proc. Natl. Acad. Sci. USA 94:12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above.

In another approach the antibodies of the present disclosure can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies of the present disclosure include those disclosed Wittrup, et al. U.S. Pat. No. 6,699,658 incorporated herein by reference.

### B. Production of Recombinant GDF11 Prodomain Complex Binding Proteins

Binding proteins (e.g., antibodies or antigen binding portions thereof) of the present disclosure may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. In one embodiment, it is possible to express the binding proteins of the disclosure in either prokaryotic or eukaryotic host cells. In another embodiment, expression of binding proteins is in eukaryotic cells. In another embodiment, expression of binding proteins is in mammalian host cells, because such eukaryotic cells (and in particular mammalian cells) are more likely than prokaryotic cells to assemble and secrete a properly folded and immunologically active binding proteins.

Common mammalian host cells for expressing the recombinant binding proteins include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and HEK293 cells, and SP2 cells. When recombinant expression vectors encoding antibody genes are introduced into mammalian host cells, the binding proteins are produced by culturing the host cells for a period of time sufficient to allow for expression of the antibody in the host cells. In another embodiment, the binding protein is secreted into the culture medium in which the host cells are grown. Binding proteins can be recovered from the culture medium using standard protein purification methods.

Host cells can also be used to produce functional antibody fragments, such as Fab fragments or scFv molecules. It will be understood that variations on the above procedure are within the scope of the present disclosure. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure and the other heavy and light chain are specific for an antigen other than the antigens of interest by cros slinking an antibody of the disclosure to a second antibody by standard chemical cros slinking methods.

In an embodiment, a recombinant expression vector encoding both the antibody heavy chain and the antibody light chain may be introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. In another embodiment, the disclosure provides a method of synthesizing a recombinant antibody of the disclosure by culturing a host cell of the disclosure in a suitable culture medium until a recombinant antibody of the disclosure is synthesized. The method can further comprise isolating the recombinant antibody from the culture medium.

### Anti GDF11 Prodomain Complex Binding Proteins:

In some embodiments, any of the binding proteins (e.g., antibodies or antigen binding portions thereof) provided herein may inhibit GDF11 activity. Exemplary binding proteins and fragments of binding proteins that bind GDF11 are provided below.

Table 7 is an abbreviated sequence list of antibody clones from screening a ScFv library.

TABLE 7
Abbreviated List of Amino Acid Sequences of ScFv candidate
binding protein clones

| | polypeptide | | SEQ ID |
|---|---|---|---|
| clone | region | Amino acid sequence | No. |
| GDF11 | scFv | MAEVQLLESRGGLVQPGRSLRLSCAASGFTFDD | 1 |
| Inh-1 | | YAMHWVRQAPGKGLEWVSGISWNSGSIGYADS | |
| | | VKGRFTISRDNTKNSLYLQMNSLRAEDTGVYYC | |
| | | AREVTGDLDYWGQGTLVTVSSGSASAPTLGGG | |
| | | GSGGGGSAAAEIVMTQSPGTLSLSPGERATLSCR | |
| | | ASQFLSSTYLAWYQQRPGQAPRLLIYSASNRAT | |
| | | GVPDRFSGSGSGTDFTLKISRVEAEDVGVYYCM | |
| | | QATHWPYTFGQGTKLEIKRTVAAPSVFKASGA | |
| GDF11 | scFv | MAQIQLVQSGAEVKKPGASVKVSCKASGYTFT | 2 |
| Inh-2 | | GYYMHWVRQAPGQGLEWMGWINPNSGGTNY | |
| | | AQKFQGWVTMTRDTSISTAYMELSRLRSDDTA | |
| | | VYYCARGGSIAVAGTLVDYYGMDVWGQGTTV | |
| | | TVSSGSASAPTLGGGGSGGGGSAAADIQMTQSP | |
| | | SSLSASVGDRVTITCQASQDISNYLNWYQQKPG | |
| | | KAPKLLIYDASNLETGVPSRFSGSGSGTDFTLTIS | |
| | | SLQPEDVATYYCQKYSTAPLTFGGGTKVEIKRT | |
| | | VAAPSVFKASGA | |
| GDF11 | scFv | MAQVQLVQSGGGVVQPGRSLRLSCAASGFTFSS | 3 |
| Inh-3 | | YAMHWVRQAPGKGLEWVAVISYDGSNEYYAD | |
| | | SVKGRFTISRDNSKNTLYLQMNSLRAEDTAVYY | |
| | | CAKDFWSGYPQYNWFDPWGQGTLVTVSSGSAS | |
| | | APTLGGGGSGGGGSAAAEIVMTQSPATLSLSAG | |
| | | ERATLSCRASQSVSNYLAWYQQKPGQAPRLLIY | |
| | | DASNRATGIPARFSGSGSGTDFTLTISSLEPEDFA | |
| | | VYYCQQYGSAPLTFGGGTNVEIKRTVAAPSVFK | |
| | | ASGA | |
| GDF11 | scFv | MAQVQLVQSGAEVKKPGASVKVSCKASGYTFT | 4 |
| Inh-4 | | SYGISWVRQAPGQGLEWMGWISAYNGNTNYA | |
| | | QKLQGRVTMTTDTSTSTAYMELRSLRSDDTAV | |
| | | YYCARTPPLWFGEYGAFDIWGQGAMVTVSSGS | |
| | | ASAPTLGGGGSGGGGSAAADIQMTQSPSSLSAS | |
| | | VGDRVTITCQASQDISNYLNWYQQKPGKAPKLL | |
| | | IYDASNLETGVPSRFSGSGSGTDFTLTISSLQPDD | |
| | | FATYYCQQSYTTPITFGQGTRLEIKRTVAAPSVF | |
| | | KASGA | |
| GDF11 | scFv | MAEVQLVQSGAEVKKPGASVKVSCKASGYTFT | 5 |
| Inh-5 | | GYYIYWVRQAPGQGLEWMGWIRPNSGDTNYA | |
| | | QKFQDRLTMTRDTSISTAYMELSRLRSDDTAVY | |
| | | YCAGNYDILTGYQAPLGYWGQGALVTVSSGSA | |
| | | SAPTLGGGGSGGGGSAAADIVLTQSPATLSVSP | |
| | | GERATLSCRASQRVISNYLAWYQQKPGQAPRLL | |
| | | IYGASSRATGIPDRFSGSGSGTDFTLSISRLEPEDF | |
| | | AVYYCQHYGPFGGGTKVEIKRTVAAPSVFKAS | |
| | | GA | |
| GDF11 | scFv | MAQVQLQESGPGLVKPSETLSLTCTVSGGSISSS | 6 |
| Inh-6 | | SYYWGWVRQPPGKGLEWIGEIYHSGSTNYNPSL | |
| | | KSRVTISVDTSKNQFSLKLSSVTAADTAVYYCA | |
| | | RVGTSWEYYFDYWGQGTLVTVSSGSASAPTLG | |
| | | GGGSGGGGSAAADIQMTQSPASLSASVGDRVTI | |
| | | TCRTSQHIINYLNWYQQRPGKAPNLLIYKASTL | |
| | | QSGVPSRFSGSGSGTHFTLTISSLQPEDFATYYC | |
| | | QQYQSYPITFGQGTRLEIKRTVAAPSVFKASGA | |
| GDF11 | scFv | MAEVQLVQSGGGLVQPGGSLRLSCAASGFTFSS | 7 |
| Inh-7 | | YSMNWVRQAPGKGLEWVSYISSSSSTIYYADSV | |
| | | KGRFTISRDNAKNSLYLQMNSLRAEDTAVYYC | |
| | | ARDGIYYDSSGYYDLFDYWGQGTLVTVSSGSA | |
| | | SAPTLGGGGSGGGGSAAADIQMTQSPSSLSASV | |
| | | GDRVTITCRASQSISSYLNWYQQKPGKAPKLLIY | |
| | | AASSLQSGVPSRFSGSGSGTDFTLTISSLQPEDFA | |
| | | TYYCQQSYSTPPTFGQGTKLEIKRTVAAPSVFKA | |
| | | SGA | |

TABLE 8
Abbreviated List of Amino Acid Sequences of VH and
VL regions of candidate binding protein clones

| | polypeptide | | SEQ ID |
|---|---|---|---|
| clone | region | Amino acid sequence | No. |
| GDF11 | VH | MAEVQLLESRGGLVQPGRSLRLSCAASGFTFDD | 8 |
| Inh-1 | | YAMHWVRQAPGKGLEWVSGISWNSGSIGYADS | |
| | | VKGRFTISRDNTKNSLYLQMNSLRAEDTGVYYC | |
| | | AREVTGDLDYWGQGTLVTVSSGSASAPT | |
| GDF11 | VL | EIVMTQSPGTLSLSPGERATLSCRASQFLSSTYL | 9 |
| Inh-1 | | AWYQQRPGQAPRLLIYSASNRATGVPDRFSGSG | |
| | | SGTDFTLKISRVEAEDVGVYYCMQATHWPYTF | |
| | | GQGTKLEIKRTVAAPSVFKASGA | |
| GDF11 | VH | MAQIQLVQSGAEVKKPGASVKVSCKASGYTFT | 10 |
| Inh-2 | | GYYMHWVRQAPGQGLEWMGWINPNSGGTNY | |
| | | AQKFQGWVTMTRDTSISTAYMELSRLRSDDTA | |
| | | VYYCARGGSIAVAGTLVDYYGMDVWGQGTTV | |
| | | TVSSGSASAPT | |
| GDF11 | VL | DIQMTQSPSSLSASVGDRVTITCQASQDISNYLN | 11 |
| Inh-2 | | WYQQKPGKAPKLLIYDASNLETGVPSRFSGSGS | |
| | | GTDFTLTISSLQPEDVATYYCQKYSTAPLTFGGG | |
| | | TKVEIKRTVAAPSVFKASGA | |
| GDF11 | VH | MAQVQLVQSGGGVVQPGRSLRLSCAASGFTFSS | 12 |
| Inh-3 | | YAMHWVRQAPGKGLEWVAVISYDGSNEYYAD | |
| | | SVKGRFTISRDNSKNTLYLQMNSLRAEDTAVYY | |
| | | CAKDFWSGYPQYNWFDPWGQGTLVTVSSGSAS | |
| | | APT | |
| GDF11 | VL | EIVMTQSPATLSLSAGERATLSCRASQSVSNYLA | 13 |
| Inh-3 | | WYQQKPGQAPRLLIYDASNRATGIPARFSGSGS | |
| | | GTDFTLTISSLEPEDFAVYYCQQYGSAPLTFGGG | |
| | | TNVEIKRTVAAPSVFKASGA | |
| GDF11 | VH | MAQVQLVQSGAEVKKPGASVKVSCKASGYTFT | 14 |
| Inh-4 | | SYGISWVRQAPGQGLEWMGWISAYNGNTNYA | |
| | | QKLQGRVTMTTDTSTSTAYMELRSLRSDDTAV | |
| | | YYCARTPPLWFGEYGAFDIWGQGAMVTVSSGS | |
| | | ASAPT | |
| GDF11 | VL | DIQMTQSPSSLSASVGDRVTITCQASQDISNYLN | 15 |
| Inh-4 | | WYQQKPGKAPKLLIYDASNLETGVPSRFSGSGS | |
| | | GTDFTLTISSLQPDDFATYYCQQSYTTPITFGQG | |
| | | TRLEIKRTVAAPSVFKASGA | |
| GDF11 | VH | MAEVQLVQSGAEVKKPGASVKVSCKASGYTFT | 16 |
| Inh-5 | | GYYTYWVRQAPGQGLEWMGWIRPNSGDTNYA | |
| | | QKFQDRLTMTRDTSISTAYMELSRLRSDDTAVY | |
| | | YCAGNYDILTGYQAPLGYWGQGALVTVSSGSA | |
| | | SAPT | |
| GDF11 | VL | DIVLTQSPATLSVSPGERATLSCRASQRVISNYL | 17 |
| Inh-5 | | AWYQQKPGQAPRLLIYGASSRATGIPDRFSGSG | |
| | | SGTDFTLSISRLEPEDFAVYYCQHYGPFGGGTKV | |
| | | EIKRTVAAPSVFKASGA | |
| GDF11 | VH | MAQVQLQESGPGLVKPSETLSLTCTVSGGSISSS | 18 |
| Inh-6 | | SYYWGWVRQPPGKGLEWIGEIYHSGSTNYNPSL | |
| | | KSRVTISVDTSKNQFSLKLSSVTAADTAVYYCA | |
| | | RVGTSWEYYFDYWGQGTLVTVSSGSASAPT | |
| GDF11 | VL | DIQMTQSPASLSASVGDRVTITCRTSQHIINYLN | 19 |
| Inh-6 | | WYQQRPGKAPNLLIYKASTLQSGVPSRFSGSGS | |
| | | GTHFTLTISSLQPEDFATYYCQQYQSYPITFGQG | |
| | | TRLEIKRTVAAPSVFKASGA | |
| GDF11 | VH | MAEVQLVQSGGGLVQPGGSLRLSCAASGFTFSS | 20 |
| Inh-7 | | YSMNWVRQAPGKGLEWVSYISSSSSTIYYADSV | |
| | | KGRFTISRDNAKNSLYLQMNSLRAEDTAVYYC | |
| | | ARDGIYYDSSGYYDLFDYWGQGTLVTVSSGSA | |
| | | SAPT | |
| GDF11 | VL | DIQMTQSPSSLSASVGDRVTITCRASQSISSYLN | 21 |
| Inh-7 | | WYQQKPGKAPKLLIYAASSLQSGVPSRFSGSGS | |
| | | GTDFTLTISSLQPEDFATYYCQQSYSTPPTFGQG | |
| | | TKLEIKRTVAAPSVFKASGA | |

The CDR sequences of each clone are listed in Tables 3-9:

TABLE 9
CDR sequences of Clone GDF11 Inh-1

| | | SEQ ID |
|---|---|---|
| Clone | Amino acid sequence | No. |
| CDRH1 | DYAMH | 22 |
| | FTFDDYAMH | 162 |
| CDRH2 | GISWNSGSIGYADSVKG | 23 |
| CDRH3 | EVTGDLDY | 24 |
| CDRL1 | RASQFLSSTYLA | 25 |
| CDRL2 | SASNRAT | 26 |
| CDRL3 | MQATHWPYT | 27 |

TABLE 10
CDR sequences of Clone GDF11 Inh-2

| | | SEQ ID |
|---|---|---|
| Clone | Amino acid sequence | No. |
| CDRH1 | GYYMH | 28 |
| | YTFTGYYMH | 163 |
| CDRH2 | WINPNSGGTNYAQKFQG | 29 |
| CDRH3 | GGSIAVAGTLVDYYGMDV | 30 |
| CDRL1 | QASQDISNYLN | 31 |
| CDRL2 | DASNLET | 32 |
| CDRL3 | QKYSTAPLT | 33 |

TABLE 11
CDR sequences of Clone GDF11 Inh-3

| | | SEQ ID |
|---|---|---|
| Clone | Amino acid sequence | No. |
| CDRH1 | SYAMH | 34 |
| | FTFSSYAMH | 164 |
| CDRH2 | VISYDGSNEYYADSVKG | 35 |
| CDRH3 | DFWSGYPQYNWFDP | 36 |
| CDRL1 | RASQSVSNYLA | 37 |
| CDRL2 | DASNRAT | 38 |
| CDRL3 | QQYGSAPLT | 39 |

TABLE 12
CDR sequences of Clone GDF11 Inh-4

| | | SEQ ID |
|---|---|---|
| Clone | Amino acid sequence | No. |
| CDRH1 | SYGIS | 40 |
| | YTFTSYGIS | 165 |
| CDRH2 | WISAYNGNTNYAQKLQG | 41 |
| CDRH3 | TPPLWFGEYGAFDI | 42 |
| CDRL1 | QASQDISNYLN | 43 |
| CDRL2 | DASNLET | 44 |
| CDRL3 | QQSYTTPIT | 45 |

TABLE 13
CDR sequences of Clone GDF11 Inh-5

| | | SEQ ID |
|---|---|---|
| Clone | Amino acid sequence | No. |
| CDRH1 | GYYIY | 46 |
| | YTFTGYYIY | 166 |
| CDRH2 | WIRPNSGDTNYAQKFQD | 47 |
| CDRH3 | NYDILTGYQAPLGY | 48 |
| CDRL1 | RASQRVISNYLA | 49 |
| CDRL2 | GASSRAT | 50 |
| CDRL3 | QHYGP | 51 |

TABLE 14
CDR sequences of Clone GDF11 Inh-6

| | | SEQ ID |
|---|---|---|
| Clone | Amino acid sequence | No. |
| CDRH1 | SYYWG | 52 |
| | GSISSSSYYWG | 167 |
| CDRH2 | EIYHSGSTNYNPSLKS | 53 |
| CDRH3 | VGTSWEYYFDY | 54 |
| CDRL1 | RTSQHIINYLN | 55 |
| CDRL2 | KASTLQS | 56 |
| CDRL3 | QQYQSYPIT | 57 |

TABLE 15
CDR sequences of Clone GDF11 Inh-7

| | | SEQ ID |
|---|---|---|
| Clone | Amino acid sequence | No. |
| CDRH1 | SYSMN | 58 |
| | FTFSSYSMN | 168 |
| CDRH2 | YISSSSSTIYYADSVKG | 59 |
| CDRH3 | DGIYYDSSGYYDLFDY | 60 |
| CDRL1 | RASQSISSYLN | 61 |
| CDRL2 | AASSLQS | 62 |
| CDRL3 | QQSYSTPPT | 63 |

It should be appreciated that in some embodiments, the CDRH1 sequences of SEQ ID NOs: 22, 28, 34, 40, 46, 52, and 58 may alternatively be the CDRH1 sequences of SEQ ID NOs: 162, 163, 164, 165, 166, 167, and 168, respectively.

The consensus sequence of each CDR region is identified in Tables 16-21. In Tables 16-21, amino acid residues absent from the consensus sequence are indicated with an underscore “_“:

TABLE 16
CDRH1 consensus sequence

| | | SEQ ID |
|---|---|---|
| Clone | Amino acid sequence | NO. |
| GDF11 | DYAMH | 22 |
| Inh-1 | | |
| GDF11 | GYYMH | 28 |
| Inh-2 | | |
| GDF11 | SYAMH | 34 |
| Inh-3 | | |
| GDF11 | SYGIS | 40 |
| Inh-4 | | |
| GDF11 | GYYIY | 46 |
| Inh-5 | | |
| GDF11 | SYYWG | 52 |
| Inh-6 | | |
| GDF11 | SYSMN | 58 |
| Inh-7 | | |

CDRH1 Consensus sequence X₁ Y X₃ X₄ X₅ (SEQ ID NO: 64)

Wherein X₁ is D, G, or S

Wherein X₃ is A, Y, G, or S

Wherein X₄ is M, I, or W

Wherein X₅ is H, S, Y, G, or N

CDRH1 Consensus sequence of Binl, including GDF11 Inh-1, GDF11 Inh-2, GDF11 Inh-3, and GDF11 Inh-4, having the amino acid sequence X₁ Y X₃ X₄ X₅ (SEQ ID NO: 70)

Wherein X₁ is D, G, or S

Wherein X₃ is A, Y, or G

Wherein X₄ is M, or I

Wherein X₅ is H, or S

CDRH1 Consensus sequence of Bin2, including GDF11 Inh-5, and GDF11 Inh-7, having the amino acid sequence X₁ Y X₃ X₄ X₅ (SEQ ID NO: 76)

Wherein X₁ is G, or S

Wherein X₃ is Y, or S

Wherein X₄ is I, or M

Wherein X₅ is Y, or N

TABLE 17
CDRH2 consensus sequence

| | | SEQ ID |
|---|---|---|
| Clone | Amino acid sequence | NO. |
| GDF11 | GISWNSGSIGYADSVKG | 23 |
| Inh-1 | | |
| GDF11 | WINPNSGGTNYAQKFQG | 29 |
| Inh-2 | | |
| GDF11 | VISYDGSNEYYADSVKG | 35 |
| Inh-3 | | |
| GDF11 | WISAYNGNTNYAQKLQG | 41 |
| Inh-4 | | |
| GDF11 | WIRPNSGDTNYAQKFQD | 47 |
| Inh-5 | | |
| GDF11 | _EIYHSGSTNYNPSLKS | 53 |
| Inh-6 | | |
| GDF11 | YISSSSSTIYYADSVKG | 59 |
| Inh-7 | | |

CDRH2 Consensus sequence X₁ X₂ X₃ X₄ X₅ X₆ X₇ X₈ X₉ X₁₀ Y X₁₂ X₁₃ X₁₄ X₁₅ X₁₆ X₁₇ (SEQ ID NO: 65)

Wherein X₁ is G, W, V, Y, or absent

Wherein X₂ is I, or E

Wherein X₃ is S, N, R, or I

Wherein X₄ is W, P, Y, A, or S

Wherein X₅ is N, D, Y, H, or S

Wherein X₆ is S, G, or N,

Wherein X₇ is G, or S

Wherein X₈ is S, G, N, D, or T

Wherein X₉ is I, T, or E

Wherein X₁₀ is G, N, or Y,

Wherein X₁₂ is A, or N

Wherein X₁₃ is D, Q, or P

Wherein X₁₄ is S, or K

Wherein X₁₅ is V, F, or L

Wherein X₁₆ is K or Q

Wherein _{X17} is G, D, or S

CDRH2 Consensus sequence of Binl, including GDF11 Inh-1, GDF11 Inh-2, GDF11 Inh-3, and GDF11 Inh-4, having the amino acid sequence X₁ I X₃ X₄ X₅ X₆ X₇ X₈ X₉ X₁₀ Y A X₁₃ X₁₄ X₁₅ X₁₆ G (SEQ ID NO: 71)

Wherein X1 is G, W, or V

Wherein X3 is S, or N

Wherein X4 is W, P, Y, or A,

Wherein X5 is N, D, or Y

Wherein X6 is S, G, or N,

Wherein X7 is G, or S

Wherein X8 is S, G, or N

Wherein X9 is I, T, or E

Wherein X10 is G, N, or Y,

Wherein X13 is D, or Q

Wherein X14 is S, or K

Wherein X15 is V, F, or L

Wherein X16 is K or Q

CDRH2 Consensus sequence of Bin2, including GDF11 Inh-5, and GDF11 Inh-7, having the amino acid sequence X₁ I X₃ X₄ X₅ S X₇ X₈ X₉ X₁₀ Y A X₁₃ X₁₄ X₁₅ X₁₆ X₁₇ (SEQ ID NO: 77)

Wherein X1 is W, or Y

Wherein X3 is R, or S

Wherein X4 is P, or S

Wherein X5 is N, or S

Wherein X7 is G, or S

Wherein X8 is D, or T

Wherein X9 is T, or I

Wherein X10 is N, or Y,

Wherein X13 is Q, or D

Wherein X14 is K, or S

Wherein X15 is F, or V

Wherein X16 is Q or K

Wherein X17 is D, or G

TABLE 18
CDRH3 consensus sequence

| | | SEQ ID |
|---|---|---|
| Clone | Amino acid sequence | NO. |
| GDF11 | _EVTGDLDY | 24 |
| Inh-1 | | |
| GDF11 | GGSIAVAGTLVDYYGMDV | 30 |
| Inh-2 | | |
| GDF11 | _DFWSGYPQYNWFDP | 36 |
| Inh-3 | | |
| GDF11 | _TPPLWFGEYGAFDI | 42 |
| Inh-4 | | |
| GDF11 | _NYDILTGYQAPLGY | 48 |
| Inh-5 | | |
| GDF11 | _VGTSWEYYFDY | 54 |
| Inh-6 | | |
| GDF11 | _DGIYYDSSGYYDLFDY | 60 |
| Inh-7 | | |

CDRH3 Consensus sequence X₁ X₂ X₃ X₄ X₅ X₆ X₇ X₈ X₉ X₁₀ X₁₁ X₁₂ X₁₃ X₁₄ X₁₅ X₁₆ X₁₇ X₁₈ (SEQ ID NO: 66)

Wherein X₁ is G, or absent

Wherein X₂ is G, or absent

Wherein X₃ is S, D, or absent

Wherein X₄ is I, G, or absent

Wherein X₅ is A, D, T, N, I, or absent

Wherein X₆ is V, F, P, Y, or absent

Wherein X₇ is A, W, P, D, Y, or absent

Wherein X₈ is G, S, L, I, V, D, or absent

Wherein X₉ is T, G, W, L, S, or absent

Wherein X₁₀ is L, Y, F, T, S, or absent

Wherein X₁₁ is E, V, P, G, or S,

Wherein X₁₂ is V, D, Q, E, Y, or W

Wherein X₁₃ is T, Y, Q, or E

Wherein X₁₄ is G, Y, N, A, or D

Wherein X₁₅ is D, G, W, A, P, Y, or L

Wherein X₁₆ is L, M, or F,

Wherein X₁₇ is D, or G

Wherein X₁₈ is Y, V, P or I

CDRH3 Consensus sequence of Binl, including GDF11 Inh-1, GDF11 Inh-2, GDF11 Inh-3, and GDF11 Inh-4, having the amino acid sequence X₁ X₂ X₃ X₄ X₅ X₆ X₇ X₈ X₉ X₁₀ X₁₁ X₁₂ X₁₃ X₁₄

X₁₅ X₁₆ D X₁₈ (SEQ ID NO: 72)

Wherein X₁ is G, or absent

Wherein X₂ is G, or absent

Wherein X₃ is S, or absent

Wherein X₄ is I, or absent

Wherein X₅ is A, D, T, or absent

Wherein X₆ is V, F, P, or absent

Wherein X₇ is A, W, P, or absent

Wherein X₈ is G, S, L, or absent

Wherein X₉ is T, G, W, or absent

Wherein X₁₀ is L, Y, F, or absent

Wherein X₁₁ is E, V, P, or G

Wherein X₁₂ is V, D, Q, or E

Wherein X₁₃ is T, or Y

Wherein X₁₄ is G, Y, or N

Wherein X₁₅ is D, G, W, or A

Wherein X₁₆ is L, M, or F,

Wherein X₁₈ is Y, V, P or I

CDRH3 Consensus sequence of Bin2, including GDF11 Inh-5, and GDF11 Inh-7, having the amino acid sequence X₁ X₂ X₃ Y X₅ X₆ X₇ X₈ G Y X₁₁ X₁₂ X₁₃ X₁₄ X₁₅ Y (SEQ ID NO: 78)

Wherein X₁ is D, or absent

Wherein X₂ is G, or absent

Wherein X₃ is N, or I

Wherein X₅ is D, or Y

Wherein X₆ is I, or D

Wherein X₇ is L, or S

Wherein X₈ T, or S

Wherein X₁₁ is Q, or Y

Wherein X₁₂ is A, or D

Wherein X₁₃ is P, or L

Wherein X₁₄ is L, or F

Wherein X₁₅ is G, or D

TABLE 19
CDRL1 consensus sequence

| | | SEQ ID |
|---|---|---|
| Clone | Amino acid sequence | NO. |
| GDF11 | RASQFLSSTYLA | 25 |
| Inh-1 | | |
| GDF11 | QASQDI_SNYLN | 31 |
| Inh-2 | | |
| GDF11 | RASQSV_SNYLA | 37 |
| Inh-3 | | |
| GDF11 | QASQDI_SNYLN | 43 |
| Inh-4 | | |
| GDF11 | RASQRVISNYLA | 49 |
| Inh-5 | | |
| GDF11 | RTSQHII_NYLN | 55 |
| Inh-6 | | |
| GDF11 | RASQSISS_YLN | 61 |
| Inh-7 | | |

CDRL1 Consensus sequence X₁ A S Q X₅ X₆ X₇ S X₉ Y L X₁₂ (SEQ ID NO: 67)

Wherein X₁ is R, or Q

Wherein X₅ is F, D, S, R, or H

Wherein X₆ is L, I, or V

Wherein X₇ is 5, I, or absent

Wherein X₈ is S, or absent

Wherein X₉ is T, N, or absent

Wherein X₁₂ is A, or N

CDRL1 Consensus sequence of Bin1, including GDF11 Inh-1, GDF11 Inh-2, GDF11 Inh-3, and GDF11 Inh-4, having the amino acid sequence X₁ A S Q X₅ X₆ X₇ S X₉ Y L X₁₂ (SEQ ID NO: 73)

Wherein X₁ is R, or Q

Wherein X₅ is F, D, or S

Wherein X₆ is L, I, or V

Wherein X₇ is S, or absent

Wherein X₉ is T, or N

Wherein X₁₂ is A, or N

CDRL1 Consensus sequence of Bin2, including GDF11 Inh-5, and GDF11 Inh-7, having the amino acid sequence RA S Q X₅ X₆ X₇ S X₉ Y L X₁₂ (SEQ ID NO: 79)

Wherein X₅ is R, or S

Wherein X₆ is V, or I

Wherein X₇ I, or S

Wherein X₉ is N, or absent

Wherein X₁₂ is A, or N

TABLE 20
CDRL2 consensus sequence

| | | SEQ ID |
|---|---|---|
| Clone | Amino acid sequence | NO. |
| GDF11 | SASNRAT | 26 |
| Inh-1 | | |
| GDF11 | DASNLET | 32 |
| Inh-2 | | |
| GDF11 | DASNRAT | 38 |
| Inh-3 | | |
| GDF11 | DASNLET | 44 |
| Inh-4 | | |
| GDF11 | GASSRAT | 50 |
| Inh-5 | | |
| GDF11 | KASTLQS | 56 |
| Inh-6 | | |
| GDF11 | AASSLQS | 62 |
| Inh-7 | | |

CDRL2 Consensus sequence X₁ A S X₄ X₅ X₆ X₇ (SEQ ID NO: 68)

Wherein X₁ is S, D, G, K, or A

Wherein X₄ is N, S, or T

Wherein X₅ is R, or L

Wherein X₆ is A, E, or Q

Wherein X₇ is T, or S

CDRL2 Consensus sequence of Binl, including GDF11 Inh-1, GDF11 Inh-2, GDF11 Inh-3, and GDF11 Inh-4, having the amino acid sequence X₁ A S N₄ X₅ X₆ T (SEQ ID NO: 74)

Wherein X₁ is S, or D

Wherein X₅ is R, or L

Wherein X₆ is A, or E

CDRL2 Consensus sequence of Bin2, including GDF11 Inh-5, and GDF11 Inh-7, having the amino acid sequence X₁ A S S X₅ X₆ X₇ (SEQ ID NO: 80)

Wherein X₁ is G, or A

Wherein X₅ is R, or L

Wherein X₆ is A, or Q

Wherein X₇ is T, or S

TABLE 21
CDRL3 consensus sequence

| | | SEQ ID |
|---|---|---|
| Clone | Amino acid sequence | NO. |
| GDF11 | MQATHWPYT | 27 |
| Inh-1 | | |
| GDF11 | QKYSTAPLT | 33 |
| Inh-2 | | |
| GDF11 | QQYGSAPLT | 39 |
| Inh-3 | | |
| GDF11 | QQSYTTPIT | 45 |
| Inh-4 | | |
| GDF11 | QHYG_P_{—} | 51 |
| Inh-5 | | |
| GDF11 | QQYQSYPIT | 57 |
| Inh-6 | | |
| GDF11 | QQSYSTPPT | 63 |
| Inh-7 | | |

CDRL3 Consensus sequence X₁ X₂ X₃ X₄ X₅ X₆ P X₈ X₉ (SEQ ID NO: 69)

Wherein X₁ is M, or Q

Wherein X₂ is Q, K, or H

Wherein X₃ is A, Y, or S

Wherein X₄ is T, S, G, Y, or Q

Wherein X₅ is H, T, S, or absent

Wherein X₆ is W, A, T, Y, or absent

Wherein X₈ is Y, L, I, P, or absent

Wherein X₉ is T, or absent

CDRL3 Consensus sequence of Bin1, including GDF11 Inh-1, GDF11 Inh-2, GDF11 Inh-3, and GDF11 Inh-4, having the amino acid sequence X₁ X₂ X₃ X₄ X₅ X₆ P X₈ T (SEQ ID NO: 75)

Wherein X₁ is M, or Q

Wherein X₂ is Q, or K

Wherein X₃ is A, Y, or S

Wherein X₄ is T, S, G, or Y

Wherein X₅ is H, T, or S

Wherein X₆ is W, A, or T

Wherein X₈ is Y, L, or I

CDRL3 Consensus sequence of Bin2, including GDF11 Inh-5, and GDF11 Inh-7, having the amino acid sequence Q X₂ X₃ X₄ X₅ X₆ P X₈ X₉ (SEQ ID NO: 81)

Wherein X₂ is H, or Q

Wherein X₃ is Y, or S

Wherein X₄ is G, or Y

Wherein X₅ is S, or absent

Wherein X₆ is T, or absent

Wherein X₈ is P, or absent

Wherein X₉ is T, or absent

The foregoing isolated anti-GDF11 prodomain complex antibody CDR sequences establish a novel family and motif of GDF11 prodomain complex binding proteins, isolated in accordance with this disclosure, and comprising polypeptides that include the CDR sequences listed in Tables 3-15.

### GDF11 Modulatory Binding Proteins

Some binding proteins (e.g., antibodies or antigen binding portions thereof), presented herein, are GDF11-modulatory antibodies. Such antibodies may bind GDF11, a GDF11 fragment or one or more protein complexes comprising GDF11. In some cases, these antibodies may be releasing antibodies or stabilizing antibodies with regard to GDF11 growth factor release and/or activity. GDF11-modulating antibodies of the disclosure may comprise or be developed using any of the scFv sequences listed in Table 7 or fragments thereof. The scFv sequences listed comprise a VH domain joined to a VL domain via a linker comprising the sequence ASAPTLGGGGSGGGGSAAA (SEQ ID NO: 150). Some recombinant GDF-modulating antibodies of the disclosure may be designed to include at least one variable domain pair (VH and VL) present in any of the scFvs listed in Table 7 or variants thereof with at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5% or at least about 99.9% sequence identity to any of the scFv sequences listed. Recombinant GDF11-modulatory antibodies may, in some cases, comprise alternative combinations of the VH and VL domains present in the scFvs presented. Further recombinant GDF11-modulatory antibodies may, in some cases, comprise VH and/or VL domains presented, but with different combinations of CDRs. Some recombinant GDF-modulating antibodies of the disclosure may comprise or be developed using scFv sequences that comprise at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5% or at least about 99.9% sequence identity to any of the scFv sequences listed in Table 7 (SEQ ID NOs: 1-7). In some cases, GDF11-modulatory antibodies constructed using one or more of the scFv sequences presented in Table 7 may interact with one or more of the recombinant proteins listed in Tables 1-6.

Recombinant GDF11-modulating antibodies of the disclosure may comprise or be developed using any of the VH sequences listed in Table 8 (SEQ ID NOs: 8, 10, 12, 14, 16, 18 and 20). Some recombinant GDF-modulating antibodies of the disclosure may comprise or be developed using VH sequences that comprise at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5% or at least about 99.9% sequence identity to any of the VH sequences listed in Table 8. Recombinant GDF11-modulating antibodies may, in some cases, comprise VH domains presented, but with different combinations of CDRs (e.g. CDR-H1, CDR-H2 or CDR-H3). In some cases, such antibodies may interact with one or more of the recombinant proteins listed in Tables 1-6.

Recombinant GDF11-modulating antibodies of the disclosure may comprise or be developed using any of the CDR-H sequences (CDR-H1, CDR-H2 and/or CDR-H3) listed in Tables 10-12, or any of the CDR-H consensus sequences provided herein. Some recombinant GDF11-modulating antibodies of the disclosure may comprise or be developed using CDR-H sequences that comprise at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5% or at least about 99.9% sequence identity to any of the CDR-H sequences listed in Tables 10-12, or any of the CDR-H consensus sequences provided herein. Recombinant GDF11-modulating antibodies may, in some cases, comprise CDR-H domains presented, but with different combinations of CDRs from other clones listed. In some cases, such antibodies may interact with one or more of the recombinant proteins listed in Tables 1-6.

Recombinant GDF11-modulating antibodies of the disclosure may comprise or be developed using any of the VL sequences listed in Table 8 (SEQ ID NOs: 9, 11, 13, 15, 17, 19 and 21). Some recombinant GDF11-modulating antibodies of the disclosure may comprise or be developed using VL sequences that comprise at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5% or at least about 99.9% sequence identity to any of the VL sequences listed in Table 8. Recombinant GDF11-modulating antibodies may, in some cases, comprise VL domains presented, but with different combinations of CDRs (e.g. CDR-L1, CDR-L2 or CDR-L3). In some cases, such antibodies may interact with one or more of the recombinant proteins listed in Tables 1-6.

Recombinant GDF11-modulating antibodies of the disclosure may comprise or be developed using any of the CDR-L sequences (CDR-L1, CDR-L2 and/or CDR-L3) listed in Tables 13-15, or any of the CDR-L consensus sequences provided herein. Some recombinant GDF11-modulating antibodies of the disclosure may comprise or be developed using CDR-L sequences that comprise at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5% or at least about 99.9% sequence identity to any of the CDR-L sequences listed in Tables 13-15, or any of the CDR-L consensus sequences provided herein. Recombinant GDF11-modulating antibodies may, in some cases, comprise CDR-L domains presented, but with different combinations of CDRs from other clones listed. In some cases, such antibodies may interact with one or more of the recombinant proteins listed in Tables 1-6.

GDF8 and GDF11 share considerable homology. While the prodomains only share 48% homology, GDF8 and GDF11 growth factor domains share 90% homology (60% homology when prodomains and growth factors are taken together). Given the high degree of sequence similarity, it is not surprising that GDF11 and 8 bind and signal through the same receptors consisting of a Type I receptor (ALK4/5) in association with a type II receptor (ACTRIIA/B). The high degree of conservation in the mature growth factors has made it challenging to identify reagents and monoclonal antibodies that can differentiate between mature GDF11 and 8. Consequently, there are no therapies in clinical trials today that are specific for GDF11.

In some embodiments, the present disclosure provides GDF11-modulatory antibodies as well as methods of developing and identifying such antibodies. In some cases, GDF-modulatory antibodies specifically recognize the prodomain of GDF11, but do not cross-react with the GDF8 prodomain. Such antibodies may be functionally assessed to determine if they block or activate the release of the GDF11 mature growth factor and can be further characterized in animal models to evaluate the effects of modulating specific growth factor levels in disease-relevant models.

Like other members of the TGF-β superfamily, GDF11 and 8 are both initially expressed as inactive precursor polypeptides (termed proGDF8 and proGDF11). For GDF11 and 8, activation and release of the mature growth factor is accomplished by discrete protease cleavage events. The first cleavage step of proGDF8 and proGDF11 is carried out by a proprotein convertase, which cuts at a conserved RXXR site between the prodomain and mature growth factor. This cleavage produces a latent complex, in which the mature growth factor is shielded from binding to its receptors by the prodomain. Activation and release of the mature, active GDF11 growth factor is accomplished after cleavage by an additional protease from the BMP/Tolloid family.

In contrast to myostatin that has a well-defined role as a negative regulator of skeletal muscle mass, much less is known about the physiological roles of GDF11. Recently, several groups have revealed exciting new biology for GDF11. Using parabiotic surgical techniques, blood systems of old and young mice were connected to look at the effects of circulating factors (Loffredo et al., 2013. Cell. 153:828-39). After 4 weeks of shared blood circulation, old mice had dramatically improved reversal of age-related cardiac hypertrophy. Additional studies suggested GDF11 was the factor that was responsible for the rejuvenating effects. Subsequently, follow up studies showed that GDF11 levels decrease with age and if systemic levels are restored, functional impairments in skeletal muscle due to the aging process can be reversed (Sinha, M. et al., 2014. Science Express. 10.1126/science.1251152, p 2-6). Additional improvements in the vasculature of the neurogenic niche were improved by the addition of GDF11 suggesting that increasing GDF11 levels could be beneficial in treating neurodegenerative and neurovascular diseases (Katsimpardi, L. et al., 2014. Science Express. 10.1126/science.1251141). It should be noted that some of these studies have been contradicted (see Brun, C. E. et al., 2015. Cell Metabolism. 22(1):54-6 and Egerman, M. A. et al., 2015. Cell Metabolism. 22(1): 164-74).

Other groups have recently published convincing data that GDF11 is a novel negative regulator of late stage erythropoiesis (Carrancio, S. et al., 2014. Br J Haematol. 165(6):870-82 and Suragani, R.N.V.S. et al. 2014. Blood. 123(25): 3864-72, the contents of each of which are herein incorporated by reference in their entirety). These studies have led to the testing of two TGF-β superfamily ligand traps, ACE-011, an ACTRIIa-Fc fusion protein, and ACE-536, an ACTRIIb-Fc fusion protein, in the clinic for the treatment of anemias such as anemia associated with cancer and β-thalassemia. Although GDF11 was implicated as the growth factor responsible for inhibiting late-stage erythropoiesis, neither of these fusion proteins is specific for GDF11 as they bind to multiple other members of the TGF-β superfamily.

The new data implicating GDF11 in reversing aging contradicts what has previously been established in the literature for GDF8 and GDF11. Moreover, the studies implicating GDF11 in erythropoiesis are still in very early stages. None of the current experimental approaches differentiate GDF11 from GDF8 leaving the question how GDF11 can be distinguished from GDF8 at the level of receptor binding and downstream signaling unanswered. There are no molecular tools that can differentiate among different forms of circulating GDF11 precursor, latent and mature forms so it is unknown what the relative circulating and tissue-bound levels of GDF11 are in the body.

In some embodiments, GDF11-modulatory antibodies of the disclosure may target the GDF11 prodomain, and therefore the activation mechanism of GDF11. Such antibodies may achieve specificity and safety that cannot be obtained by currently available methods. In some cases, GDF11 modulatory antibodies may also be useful as specific tools to interrogate the role of GDF11 in vivo and importantly allow for the development of therapeutic antibodies that may either block or activate GDF11 release and that may be evaluated in clinical trials.

### Anti GDF11 Prodomain Complex Chimeric Antibodies

A chimeric antibody is a molecule in which different portions of the antibody are derived from different origins or animal species. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entireties. In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454 which are incorporated herein by reference in their entireties) by splicing genes from a human antibody molecule of appropriate antigen specificity together with genes from a mouse antibody molecule of appropriate biological activity can be used.

In one embodiment, the chimeric antibodies of the disclosure are produced by inserting the CDRs of the anti-GDF11 prodomain complex binding proteins described herein with a human IgG1 constant region. In another embodiment, the chimeric antibody of the disclosure may comprise a heavy chain variable region (V_{H}) comprising the amino acid sequence of SEQ ID NOs: 8, 10, 12, 14, 16, 18 and 20 and a light chain variable region (V_{L}) comprising the amino acid sequence of SEQ ID NOs:9, 11, 13, 15, 17, 19 and 21.

### Anti GDF11 Prodomain Complex Humanized Antibodies

Humanized antibodies are antibody molecules from non-human species antibody that bind the desired antigen having one or more complementarity determining regions (CDRs) from the non-human species and framework regions from a human immunoglobulin molecule. Known human Ig sequences are disclosed, e.g., www.ncbi.nlm.nih.gov/entrez-/query.fcgi; www.atcc.org/phage/hdb.html; www.sciquest.com/; www.abcam.com/; www.antibodyresource.com/onlinecomp.html; www.public.iastate.edu/.about.pedro/researchtools.html; www.mgen.uni-heidelberg.de/SD/IT/IT.html; www.whfreeman.com/immunology/CH-05/kuby05.htm; www.library.thinkquest.org/12429/Immune/Antibody.html; www.hhmi.org/grants/lectures/1996/vlab/; www.path.cam. ac.uk/.about.mrc7/m-ikeimages.html; www.antibodyresource.com/; mcb.harvard.edu/BioLinks/Immunology.html.www.immunologylink.com/; pathbox.wustl.edu/.about.hcenter/index.-html; www.biotech.ufl.edu/.about.hcl/; www.pebio.com/pa/340913/340913.html-; www.nal.usda.gov/awic/pubs/antibody/; www.m.ehime-u.acjp/.about.yasuhito-/Elisa.html; www.biodesign.con/table.asp; www.icnet.uk/axp/facs/davies/links.html; www.biotech.ufl.edu/.about.fccl/protocol.html; www.isac-net.org/sites_geo.html; aximtl.imt.uni-marburg.de/.about.rek/AEP-Start.html; baserv.uci.kun.nl/.aboutjraats/linksl.html; www.recab.uni-hd.de/immuno.bme.nwu.edu/; www.mrc-cpe.cam.ac.uk/imt-doc/public/INTRO.html; www.ibt.unam.mx/vir/V_mice.html; imgt.cnusc.fr:8104/; www.biochem.uct.ac.uk/.about.martin/abs/index.html; antibody.bath.ac.uk/; abgen.cvm.tamu.edu/lab/wwwabgen.html; www.unizh.ch/.about.honegger/AHOseminar/Slide01.html; www.cryst.bbk.ac.uk/.about.ubcg07s/; www.nimr.mrc.ac.uk/CC/ccaewg/ccaewg.htm; www.path.cam.ac.uk/.about.mrc7/humanisation/TAHHP.html; www.ibt.unam.mx/vir/structure/stat_aim.html; www.biosci.missouri.edu/smithgp/index.html; www.cryst.bioc.cam.ac.uk/.abo-ut.fmolina/Web-pages/Pept/spottech.html; www.jerini.de/fr roducts.htm; www.patents.ibm.com/ibm.html.Kabat et al., Sequences of Proteins of Immunological Interest, U.S. Dept. Health (1983), each entirely incorporated herein by reference. Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, or improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as but not limited to those described in Jones et al., Nature 321:522 (1986); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28(4/5):489-498 (1991); Studnicka et al., Protein Engineering 7(6):805-814 (1994), Roguska. et al., PNAS 91:969-973 (1994); PCT publication WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, each entirely incorporated herein by reference, included references cited therein.

Antibodies with Particular Binding Profiles

Some aspects of the disclosure relate to antibodies having particular binding profiles. In some embodiments, antibodies are selected for use (e.g., inhibiting GDF11 activation) based on the fact that they have a known and/or desired binding profile (a selected binding profile). As used herein the term “binding profile” refers to a set of one or more parameters (e.g., symbols, quantities, measurements, etc.) indicative of the extent to which an antibody specifically binds to one or more antigens. In some embodiments, a parameter indicative of the binding of an antibody for a target antigen is an IC50 or EC50 value. However, in some embodiments, a parameter indicative of the binding of an antibody for a target antigen is an equilibrium dissociation constant (Kd). In some embodiments, a parameter indicative of the binding is an equilibrium association constant (Ka). Other suitable parameters indicative of binding may be used in some embodiments. In some embodiments, the disclosure relates to antibodies having a selected GDF11-related binding profile, which comprises a set of one or more parameters indicative of the extent to which an antibody specifically binds to one or more antigens, at least one of which antigens is a GDF11-related protein (e.g., proGDF11 or latent GDF11).

In some embodiments, a binding profile comprises one or more parameters indicative of whether or not an antibody exhibits a threshold level of binding (e.g., specific binding) to one or more antigens. In some embodiments, a threshold level of binding is a level of binding that above (or below, depending on the parameter) a control or reference level of binding (e.g., background or non-specific binding). In some embodiments, a threshold level of binding is a level of binding that is at least 1, at least 2, at least 3, at least 4, at least 5, at least 6, at least 7 or more standard deviations greater than (or less than, depending on the assay and/or parameter) a control or reference level of binding (e.g., background or non-specific binding), as measured by an appropriate immunoassay. In some embodiments, a threshold level of binding is a level of binding that is in a range of 1 to 10 standard deviations, 2 to 10 standard deviations, or 4 to 6 standard deviations greater than (or less than, depending on the assay and/or parameter) a control or reference level of binding (e.g., background or non-specific binding), as measured by an appropriate immunoassay.

In some embodiments, a threshold level of binding is determined through an appropriate immunoassay. In some embodiments, an appropriate immunoassay assesses the binding affinity of an antibody for a target antigen. In some embodiments, an appropriate immunoassay is an enzyme linked immune-sorbent assay. In some embodiments, an appropriate immunoassay is an assay that determines a kinetic measurement (e.g., on rate, off rate) indicative of binding between an antibody and antigen. In some embodiments, an appropriate immunoassay is an assay, such as an Octet assay, that determines one or more a kinetic parameters indicative of binding between an antibody and antigen. In some embodiments, an appropriate immunoassay is a cell-based assay that determines one or more a parameters indicative of specific binding between an antibody and antigen based on cellular activity, e.g., growth factor signaling (such as SMAD signaling), cell growth, cell survival, gene expression, reporter expression, protein production, protein secretion, etc. In some embodiments, an appropriate immunoassay is an in vivo assay that determines one or more a parameters indicative of specific binding between an antibody and antigen based on cellular, tissue or other physiological activity.

In some embodiments, a binding profile comprises a set of one or more parameters indicative of the extent to which an antibody specifically binds to one or more one or more TGFβ family member proteins (e.g., GDF11) or forms thereof, one or more portions or domains of TGFβ family member proteins and/or one or more chimeras of TGFβ family member proteins. In some embodiments, a binding profile comprises a set of one or more parameters indicative of the extent to which an antibody specifically binds or does not specifically bind to one or more different antigens. In some embodiments, a binding profile relates to the extent to which an antibody specifically binds or does not specifically bind to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more different antigens. In some embodiments, a binding profile relates to the extent to which an antibody specifically binds or does not specifically bind to 4, 5, 6, 7, or 8 different antigens.

In some embodiments, a particular binding profile comprises a set of one or more parameters indicative of the extent to which an antibody specifically binds to one or more TGFβ family member proteins or forms thereof. In some embodiments, a TGFβ family member proteins is selected from the group consisting of AMH, ARTN, BMP10, BMP15, BMP2, BMP3, BMP4, BMPS, BMP6, BMP7, BMP8A, BMP8B, GDF1, GDF10, GDF11, GDF15, GDF2, GDF3, GDF3A, GDFS, GDF6, GDF7, GDF8, GDF9, GDNF, INHA, INHBA, INHBB, INHBC, INHBE, LEFTY1, LEFTY2, NODAL, NRTN, PSPN, TGFβ1, TGFβ2, and TGFβ3 protein. In some embodiments, TGFβ family member proteins or forms thereof are from a vertebrate organism. In some embodiments, TGFβ family member proteins or forms thereof are from a human, a monkey, a mouse or a rat. In some embodiments, TGFβ family member proteins or forms thereof are from a human or a mouse. In some embodiments, TGFβ family member proteins or forms thereof are from a human. Examples of sequences of human and non-human TGFβ family member proteins are shown in Tables 1 and 3, provided herein. In some embodiments, TGFβ family member proteins or forms thereof may include any naturally-occurring isoforms or variants of TGFβ family member proteins (e.g., GDF11). In some embodiments, TGFβ family member proteins comprise an amino acid sequence that is at least 70%, 75%, 80%, 85%, 90%, 95%, 98%, or 99% identical to any of the amino acid sequences as set forth in any one of SEQ ID NOs: 82-84 and 93-98. In some embodiments, TGFβ family member proteins comprise an amino acid sequence as set forth in any one of SEQ ID NOs: 82-84 and 93-98. In some embodiments, TGFβ family member proteins consist of an amino acid sequence as set forth in any one of SEQ ID NOs: 82-84 and 93-98.

In some embodiments, a particular binding profile may also comprise one or more parameters indicative of to the extent to which antibodies specifically bind to or do not specifically bind to a portion or domain of one or more TGFβ family member proteins. In some embodiments, the portion or domain of a TGFβ family member protein is a prodomain, a straight jacket region, a growth factor domain, a fastener region, a furin cleavage site region, a bmp/tolloid cleavage site, an arm region, a fingers region 1, a fingers region 2, a latency loop, an alpha 1 helical region, and/or a bowtie region. Exemplary portions or domains of TGFβ family member proteins are shown in Tables 2, and 4, provided herein. In some embodiments, portions or domains of TGFβ family member proteins are portions or domains of GDF proteins. In some embodiments, portions or domains of TGFβ family member proteins are portions or domains of GDF8 and/or GDF11. In some embodiments, portions or domains of TGFβ family member proteins are portions or domains of Inhibin beta A. However, it should be appreciated that the portions or domains of TGFβ family member proteins may be from any TGFβ family member protein provided herein. In some embodiments, the portion or domain of a TGFβ family member protein comprises an amino acid sequence that is at least 70%, 75%, 80%, 85%, 90%, 95%, 98%, or 99% identical to any of the amino acid sequences as set forth in any one of SEQ ID NOs: 85-92 and 99-110. In some embodiments, portions or domains of TGFβ family member proteins comprise an amino acid sequence as set forth in any one of SEQ ID NOs: 85-92 and 99-110. In some embodiments, portions or domains of TGFβ family member proteins consist of an amino acid sequence as set forth in any one of SEQ ID NOs: 85-92 and 99-110.

In some embodiments, forms of TGFβ family member proteins refer to complexes of TGFβ family member proteins. For example, forms of TGFβ family member proteins may include pro-forms, latent-forms, primed-forms, or mature forms of dimeric TGFβ family member proteins, such as, for example, proGDF8, proGDF11, latent GDF8, latent GDF11, primed GDF8, or primed GDF11. In some embodiments, TGFβ family member proteins form dimeric complexes. In some embodiments, TGFβ family member proteins form homodimeric complexes. In some embodiments, TGFβ family member proteins form heterodimeric complexes. Dimeric forms of TGFβ family member proteins may include TGFβ family member proteins that are full-length or TGFβ family member proteins that have been cleaved (e.g., by a proprotein convertase and/or a tolloid protease). In some embodiments, forms of TGFβ family member proteins are pro forms of TGFβ family member proteins (e.g., proGDF11). In some embodiments, forms of TGFβ family member proteins include full-length TGFβ family member proteins. For example, pro forms of TGFβ family member proteins include, without limitation, proGDF8 and proGDF11 that have not been cleaved at a proprotein convertase cleavage site (e.g., by a proprotein convertase such as PCSK5) or a tolloid protease cleavage site (e.g., by a tolloid protease such as BMP-1). In some embodiments, forms of TGFβ family member proteins are latent forms of TGFβ family member proteins (e.g., latent GDF8 or latent GDF11). In some embodiments, forms of TGFβ family member proteins include TGFβ family member proteins that have been cleaved (e.g., by a proprotein convertase). For example, latent forms of TGFβ family member proteins include, without limitation, latent GDF8 and latent GDF11 that have been cleaved at a proprotein convertase cleavage site (e.g., by a proprotein convertase such as furin or PCSK5) but not at a tolloid protease cleavage site. In some embodiments, forms of TGFβ family member proteins are primed forms of TGFβ family member proteins (e.g., primed GDF8 or primed GDF11). In some embodiments, forms of TGFβ family member proteins include TGFβ family member proteins that have been cleaved (e.g., by a proprotein convertase and/or a tolloid protease). For example, primed forms of TGFβ family member proteins include, without limitation, primed GDF8 and primed GDF11 that have been cleaved at a proprotein convertase cleavage site (e.g., by a proprotein convertase such as furin) and a tolloid protease cleavage site (e.g., by a tolloid protease such as BMP-1). In some embodiments, forms of TGFβ family member proteins are mature forms of TGFβ family member proteins (e.g., mature GDF8 or mature GDF11). In some embodiments, forms of TGFβ family member proteins include TGFβ family member proteins that have been cleaved (e.g., by a proprotein convertase and/or a tolloid protease) and are not in complex with one or more portions of a prodomain of a TGFβ family member protein. For example, mature forms of TGFβ family member proteins include, without limitation, mature GDF8 and mature GDF11 that have been cleaved at a proprotein convertase cleavage site (e.g., by a proprotein convertase such as furin or PCSK5), a tolloid protease cleavage site (e.g., by a tolloid protease such as BMP-1), and are not in complex with a prodomain of a TGFβ family member protein.

It is noted that some prodomains may be cleaved by proprotein convertase enzymes. As used herein, the term “proprotein convertase” refers to an enzyme that cleaves a prodomain from a translated protein to facilitate protein maturation. Some proprotein convertases of the present disclosure include the subtilisin-like proprotein convertase (SPC) family member enzymes. The SPC family comprises calcium-dependent serine endoproteases that include, but are not limited to furin/PACE, PC1/3, PC2, PC4, PC5/6, PACE4 and PC7 (Fuller et al., 2009. Invest Ophthalmol Vis Sci. 50(12):5759-68, the contents of which are herein incorporated by reference in their entirety). GDF11 may in some cases, be cleaved by PC5/6. In some cases, proprotein convertases may cleave proproteins at additional sites, other than those indicated in Table 1. In some embodiments, pro-proteins may be cleaved at a first cleavage site (the first site being the site closest to the N-terminus). In other embodiments, pro-proteins may be cleaved at a cleavage site other than a first cleavage site. In some cases, proprotein convertase cleavage may occur intracellularly. In some cases, proprotein convertase cleavage may occur extracellularly.

In some embodiments, a particular binding profile comprises a set of one or more parameters indicative of the extent to which antibodies specifically bind to or do not specifically bind to chimeras of TGFβ family member proteins. In some embodiments, chimeras of TGFβ family member proteins can be used to provide information relating to particular epitopes to which any of the antibodies provided herein specifically bind or do not specifically bind. In some embodiments, chimeric TGFβ family member proteins comprise at least 1, 2, 3, 4, 5, 6, 7, 8, 9, 10, 11, 12, 13, 14 or 15 portions or domains of different TGFβ family member protein. For example, a chimeric TGFβ family member protein may comprise a prodomain, a straight jacket region, a growth factor domain, a fastener region, a furin cleavage site region, a bmp/tolloid cleavage site, an arm region, a fingers region 1, a fingers region 2, a latency loop, an alpha 1 helical region, and/or a bowtie region of one TGFβ family member protein and at least 1, 2, 3, 4, 5, 6, 7, 8, 9, 10, 11, 12, 13, 14 or 15 of a prodomain, a straight jacket region, a growth factor domain, a fastener region, a furin cleavage site region, a bmp/tolloid cleavage site, an arm region, a fingers region 1, a fingers region 2, a latency loop, an alpha 1 helical region, and/or a bowtie region from one or more different TGFβ family member proteins. Exemplary chimeras of TGFβ family member proteins are shown in Tables 5 and 6, provided herein. In some embodiments, chimeras of TGFβ family member proteins comprise an amino acid sequence that is at least 70%, 75%, 80%, 85%, 90%, 95%, 98%, or 99% identical to any of the amino acid sequences as set forth in any one of SEQ ID NOs: 111-138. In some embodiments, chimeras of TGFβ family member proteins comprise an amino acid sequence as set forth in any one of SEQ ID NOs: 111-138. In some embodiments, chimeras of TGFβ family member proteins consist of an amino acid sequence as set forth in any one of SEQ ID NOs: 111-138.

In some embodiments, a particular binding profile comprises a set of one or more parameters indicative of the extent to which antibodies specifically bind to or do not specifically bind to one or more of a human proGDF11, a murine proGDF11, a human latent GDF11, a murine latent GDF11, a human proGDF11 ARM8, a human proGDF8, a human prodomain GDF11 ARM8, and/or a human mature GDF11. However, in certain embodiments, parameters indicative of the extent of binding to one or more murine antigens can be removed from a binding profile. For example, in some embodiments, a particular binding profile comprises a set of one or more parameters indicative of the extent to which antibodies specifically bind to or do not specifically bind to one or more of a human proGDF11, a human latent GDF11, a human proGDF11 ARM8, a human proGDF8, a human prodomain GDF11 ARM8, and/or a human mature GDF11. In some embodiments, a particular binding profile relates to an extent to which antibodies specifically bind to or do not specifically bind to one or more of a protein that comprises an amino acid sequence that is at least 70%, 75%, 80%, 85%, 90%, 95%, 98%, or 99% identical to a human proGDF11, a murine proGDF11, a human latent GDF11, a murine latent GDF11, a human proGDF11 ARM8, a human proGDF8, a human prodomain GDF11 ARM8, and/or a human mature GDF11. In some embodiments, the human proGDF11 comprises an amino acid sequence as set forth in SEQ ID NO: 82. In some embodiments, the murine proGDF11 comprises an amino acid sequence as set forth in SEQ ID NO: 97. In some embodiments, the murine latent GDF11 comprises an amino acid sequence as set forth in SEQ ID NO: 82. In some embodiments, the human GDF8 prodomain comprises an amino acid sequence as set forth in SEQ ID NO: 97. In some embodiments, the human proGDF11 ARM8 comprises an amino acid sequence as set forth in SEQ ID NO: 122. In some embodiments, the human proGDF8 comprises an amino acid sequence as set forth in SEQ ID NO: 83. In some embodiments, the human prodomain GDF11 ARM8 comprises an amino acid sequence as set forth in SEQ ID NO: 124. In some embodiments, the human mature GDF11 comprises an amino acid sequence as set forth in SEQ ID NO: 90. In some embodiments, antibodies provided herein have a binding profile as set forth in Table 22.

In some embodiments, a binding profile may comprise one or more symbols (e.g., +, −, +/−) indicative of the extent to which an antibody binds to an antigen. For example, in some embodiments, binding of an antibody to an antigen at a level detectable beyond a threshold level (e.g., 5 standard deviations beyond a reference level) may be indicated by a “+”. In some embodiments, a “−” indicates that the antibody does not bind the antigen at level detectable beyond a threshold in a particular assay (e.g., is less than 2, 3, 4 or 5 standard deviations beyond a reference level, e.g., an assay background level). In some embodiments, a “+/−” indicates that an antibody is at or near a threshold of binding the antigen as determined by a particular assay (e.g., within 2 to 5, 3 to 5, or 4 to 5 standard deviations of a reference level).

In some embodiments, an antibody that “specifically binds” to a target antigen, binds to the target antigen with greater affinity, avidity, more readily, and/or with greater duration than it binds to non-target antigens. In some embodiments, antibodies provided herein have particular binding profiles, e.g., based on whether they specifically bind or do not specifically bind to one or more TGFβ family member proteins or forms thereof, one or more portions or domains of TGFβ family member proteins and/or one or more chimeras of TGFβ family member proteins. In some embodiments an antibody specifically binds an antigen if binding to that antigen is detected above a background level (e.g., of a control antigen) using an in vitro binding assay (e.g., an ELISA). In some embodiments, an antibody specifically binds an antigen if binding to that antigen is detected at least one, at least 2, at least 3, at least 4, at least 5, at least 6, at least 7, at least 8, at least 9, or at least 10 standard deviations above a background level (e.g., of a control antigen) using an in vitro binding assay. In some embodiments an antibody specifically binds an antigen if binding to that antigen is detected at least one, at least 5 standard deviations above a background level (e.g., of a control antigen) using an in vitro binding assay. In some embodiments, the in vitro binding assay is an enzyme linked immunosorbent assay (ELISA). In some embodiments, the ELISA is performed as described in Example 2, provided herein. However, it should be appreciated that additional methods for determining the binding affinity of a protein to an antigen are also within the scope of this disclosure

In some embodiments, an antibody specifically binds to an antigen (e.g., proGDF11) if it binds that antigen with a higher affinity as compared to another antigen (e.g., latent GDF11). In some embodiments, an antibody specifically binds to an antigen if it binds to that antigen by at least 2-fold, 5-fold, 10-fold, 50-fold, 100-fold, 200-fold, 500-fold, or 1,000-fold higher than another antigen.

In some embodiments, an antibody specifically binds to an antigen (e.g., proGDF11) if it binds that antigen with a higher affinity as compared to another antigen (e.g., latent GDF11). In some embodiments, an antibody specifically binds to an antigen if it binds to that antigen with a dissociation constant (Kd) that is less than 10⁻³ M, 10⁻⁴ M, 10⁻⁵ M, 10⁻⁶ M, 10⁻⁷ M, 10⁻⁸ M.

Some aspects of the invention provide antibodies that specifically bind to human proGDF11. In some embodiments, the human proGDF11 comprises the amino acid sequence as set forth in (SEQ ID NO: 82). In some embodiments, the antibody that specifically binds to a human proGDF11 does not specifically bind to any combination of 1, 2, 3, 4, 5, 6, 7, 8 or 9 of the following: a human proGDF8, a human latent GDF8, a murine proGDF8, a murine latent GDF8, a human proActivin A, a human latent ActivinA, a mature GDF11, a mature GDF8, or a mature Activin A. In some embodiments, the human proGDF8 comprises the amino acid sequence as set forth in (SEQ ID NO: 83). In some embodiments, the human latent GDF8 comprises the amino acid sequence as set forth in (SEQ ID NO: 83). In some embodiments, the murine proGDF8 comprises the amino acid sequence as set forth in (SEQ ID NO: 93). In some embodiments, the murine latent GDF8 comprises the amino acid sequence as set forth in (SEQ ID NO: 93). In some embodiments, the human proActivin A comprises the amino acid sequence as set forth in (SEQ ID NO: 84). In some embodiments, the human latent ActivinA comprises the amino acid sequence as set forth in (SEQ ID NO: 84). In some embodiments, the mature GDF11 comprises the amino acid sequence as set forth in (SEQ ID NO: 90). In some embodiments, the mature GDF8 comprises the amino acid sequence as set forth in (SEQ ID NO: 89). In some embodiments, the mature Activin A comprises the amino acid sequence as set forth in (SEQ ID NO: 120).

In some embodiments, any of the antibodies that specifically bind to a human pro GDF11 may also specifically bind to any combination of 1, 2, or 3 of the following: a human latent GDF11, a mouse proGDF11, a mouse latent GDF11. In some embodiments, the human latent GDF11 comprises the amino acid sequence as set forth in (SEQ ID NO: 82). In some embodiments, the murine proGDF11 comprises the amino acid sequence as set forth in (SEQ ID NO: 97). In some embodiments, the murine latent GDF11 comprises the amino acid sequence as set forth in (SEQ ID NO: 97).

### Sweeping Antibodies

In some embodiments, binding protein bind an antigen but cannot effectively eliminate the antigen from the plasma. Thus, in some embodiments, the concentration of the antigen in the plasma may be increased by reducing the clearance of the antigen. However, in some embodiments, binding proteins (e.g., sweeping antibodies or antigen binding portions) provided herein have an affinity to an antigen that is sensitive to pH. Such pH sensitive binding protein may bind to the antigen in plasma at neutral pH and dissociate from the antigen in an acidic endosome, thus reducing binding protein-mediated antigen accumulation and/or promoting antigen clearance from the plasma. Aspects of the disclosure relate to sweeping antibodies. As used herein “sweeping antibodies” refer to antibodies having both pH-sensitive antigen binding and at least a threshold level of binding to cell surface neonatal Fc receptor (FcRn) at neutral or physiological pH. In some embodiments, sweeping antibodies bind to the neonatal Fc receptor FcRn at neutral pH. For example sweeping antibodies may bind to the FcRn at a pH ranging from 7.0 to 7.6. In some embodiments, sweeping antibodies can bind to an antigen at an antigen binding site and bind to a cellular FcRn via an Fc portion of the antibody. In some embodiments, sweeping antibodies may then be internalized, releasing antigen in an acidic endosome, which may be degraded. In some embodiments, a sweeping antibody, no longer bound to the antigen, may then be released (e.g., by exocytosis) by the cell back into the serum.

In some embodiments, FcRn in the vascular endothelia (e.g., of a subject) extends the half-life of a sweeping antibody. In some embodiments, vascular endothelial cells internalize sweeping antibodies, which in some embodiments are bound to an antigen such as GDF11 (e.g., pro GDF11, latent GDF11 or primed GDF11). In some embodiments, a sweeping antibody is recycled back into the bloodstream. In some embodiments, a sweeping antibody has an increased half-life (e.g., in the serum of a subject) as compared to its conventional counterpart. In some embodiments, a conventional counterpart of a sweeping antibody refers the antibody from which the sweeping antibody was derived (e.g., prior to engineering the Fc portion of the conventional antibody to bind FcRn with greater affinity at pH 7). In some embodiments, a sweeping antibody has a half-life in the serum of a subject that is at least 1%, 5%, 10%, 15%, 20%, 25%, 35%, 50%, 75%, 100%, 150%, 200% or 250% longer as compared to its conventional counterpart.

In some embodiments, an Fc portion of a sweeping antibody binds FcRn. In some embodiments, the Fc portion of a sweeping antibody binds to FcRn at a pH of 7.4 with a Kd ranging from 10⁻³ M to 10⁻⁸ M. In some embodiments, a sweeping antibody binds to FcRn at a pH of 7.4 with a Kd ranging from10⁻³ M to 10⁻⁷ M, from 10⁻³ M to 10⁻⁶ M, from 10⁻³ M to 10⁻⁵ M, from 10⁻³ M to 10⁻⁴ M, from 10⁻⁴ M to 10⁻⁸ M, from 10⁴ M to 10⁻⁷ M, from 10⁴ M to 10⁻⁶ M, from 10⁻⁴ M to 10⁻⁵ M, from 10⁻⁵ M to 10⁻⁸ M, from 10⁻⁵ M to 10⁻⁷ M, from 10⁻⁵ M to 10⁻⁶ M, from 10⁻⁶ M to 10⁻⁸ M, from 10⁻⁶ M to 10⁻⁷ M, or from 10⁻⁷ M to 10⁻⁸ M. In some embodiments, FcRn binds to the CH2-CH3 hinge region of a sweeping antibody. In some embodiments, FcRn binds to the same region as proteinA or protein G. In some embodiments, FcRn binds to a different binding site from FcyRs. In some embodiments, the amino acid residues AA of a sweeping antibody Fc region are required for binding to FcRn. In some embodiments, the amino acid residues AA of a sweeping antibody Fc region affect binding to FcRn.

In some embodiments, any of the antibodies provided herein are engineered to bind FcRn with greater affinity. In some embodiments, any of the antibodies provided herein are engineered to bind FcRn with greater affinity at pH 7.4. In some embodiments, the affinity of sweeping antibodies to FcRn is increased to extend their pharmacokinetic (PK) properties as compared to their conventional counterparts. For example, in some embodiments, sweeping antibodies elicit less adverse reactions due to their efficacy at lower doses. In some embodiments, sweeping antibodies are administered less frequently. In some embodiments, transcytosis of sweeping antibodies to certain tissue types are increased. In some embodiments, sweeping antibodies enhance efficiency of trans-placental delivery. In some embodiments, sweeping antibodies are less costly to produce.

In some embodiments, any of the antibodies provided herein are engineered to bind FcRn with lower affinity. In some embodiments, any of the antibodies provided herein are engineered to bind FcRn with lower affinity at pH 7.4. In some embodiments, the affinity of sweeping antibodies to FcRn is decreased to shorten their pharmacokinetic (PK) properties as compared to their conventional counterparts. For example, in some embodiments, sweeping antibodies are more rapidly cleared for imaging and/or radioimmunotherapy. In some embodiments, sweeping antibodies promote clearance of endogenous pathogenic antibodies as a treatment for autoimmune diseases. In some embodiments, sweeping antibodies reduce the risk of adverse pregnancy outcome, which may be caused by trans-placental transport of material fetus-specific antibodies.

In some embodiments, sweeping antibodies have decreased affinity to an antigen at low pH as compared to a neutral or physiological pH (e.g., pH 7.4). In some embodiments, sweeping antibodies have a decreased affinity to an antigen at an acidic pH (e.g. a pH ranging from 5.5 to 6.5) as compared to a physiological pH (e.g., pH 7.4). It should be appreciated that any of the antibodies provided herein can be engineered to dissociate from the antigen depending on changes in pH (e.g., pH sensitive antibodies). In some embodiments, sweeping antibodies provided herein are engineered to bind antigen dependent on pH. In some embodiments, sweeping antibodies provided herein are engineered to bind FcRn dependent on pH. In some embodiments, sweeping antibodies provided herein are internalized by endocytosis. In some embodiments, sweeping antibodies provided here are internalized by FcRn binding. In some embodiments, endocytosed sweeping antibodies release antigen in an endosome. In some embodiments, sweeping antibodies are recycled back to the cell surface. In some embodiments, sweeping antibodies remain attached to cells. In some embodiments, endocytosed sweeping antibodies are recycled back to the plasma. It should be appreciated that the Fc portion of any of the antibodies provided herein may be engineered to have different FcRn binding activity. In some embodiments, FcRn binding activity affects the clearance time of an antigen by a sweeping antibody. In some embodiments, sweeping antibodies may be long-acting or rapid-acting sweeping antibodies.

In some embodiments, converting a conventional therapeutic antibody into a sweeping antibody reduces the efficacious dose. In some embodiments, converting a conventional therapeutic antibody into a sweeping antibody reduces the efficacious dose by at least 1%, 2%, 5%, 10%, 20%, 30%, 40%, 50%, 60%, 70%, 80%, 90%, 95%, or 99%. In some embodiments, converting a conventional therapeutic antibody into a sweeping antibody reduces the efficacious dose by at least 1.5 fold, 2 fold, 3 fold, 4 fold, 5 fold, 6 fold, 8 fold, 10 fold, 15 fold, 20 fold, 50 fold or 100 fold.

In some embodiments, selecting an appropriate dose of a sweeping antibody for therapy may be performed empirically. In some embodiments, a high dose of a sweeping antibody may saturate FcRn, resulting in antibodies which stabilize antigen in serum without being internalized. In some embodiments, a low dose of a sweeping antibody may not be therapeutically effective. In some embodiments, sweeping antibodies are administered once a day, once a week, once every two weeks, once every three weeks, once every four weeks, once every 6 weeks, once every 8 weeks, once every 10 weeks, once every 12 weeks, once every 16 weeks, once every 20 weeks, or once every 24 weeks.

In some embodiments, any of the antibodies provided herein may be modified or engineered to be sweeping antibodies. In some embodiments, any of the antibodies provided herein may be converted into a sweeping antibody using any suitable method. For example, suitable methods for making sweeping antibodies have been previously described in Igawa et al., (2013) “Engineered Monoclonal Antibody with Novel Antigen-Sweeping Activity In Vivo,” PLoS ONE 8(5): e63236; and Igawa et al., “pH-dependent antigen-binding antibodies as a novel

therapeutic modality,” Biochimica et Biophysica Acta 1844 (2014) 1943-1950; the contents of each of which are hereby incorporated by reference. It should be appreciated, however, that the methods for making sweeping antibodies as provided herein are not meant to be limiting. Thus, additional methods for making sweeping antibodies are within the scope of this disclosure.

### Competing and Cross-Competing Binding Proteins

Aspects of the disclosure relate to binding proteins (e.g., antibodies or antigen binding portions thereof) that compete or cross-compete with any of the binding proteins provided herein. The term “compete”, as used herein with regard to a binding protein, means that a first binding protein binds to an epitope of a protein (e.g., latent GDF11) in a manner sufficiently similar to the binding of a second binding protein, such that the result of binding of the first binding protein with its epitope is detectably decreased in the presence of the second binding protein compared to the binding of the first binding protein in the absence of the second binding protein. The alternative, where the binding of the second binding protein to its epitope is also detectably decreased in the presence of the first antibody, can, but need not be the case. That is, a first binding protein can inhibit the binding of a second binding protein to its epitope without that second binding protein inhibiting the binding of the first antibody to its respective epitope. However, where each binding protein detectably inhibits the binding of the other antibody with its epitope or ligand, whether to the same, greater, or lesser extent, the antibodies are said to “cross-compete” with each other for binding of their respective epitope(s). Both competing and cross-competing binding proteins are within the scope of this disclosure. Regardless of the mechanism by which such competition or cross-competition occurs (e.g., steric hindrance, conformational change, or binding to a common epitope, or portion thereof), the skilled artisan would appreciate that such competing and/or cross-competing binding proteins are encompassed and can be useful for the methods and/or compositions provided herein.

In some embodiments, aspects of the disclosure relate to antibodies that compete or cross-compete with any of the antibodies provided herein. In some embodiments, an antibody binds at or near the same epitope as any of the antibodies provided herein. In some embodiments, an antibody binds near an epitope if it binds within 15 or fewer amino acid residues of the epitope. In some embodiments, any of the antibodies provided herein bind within 1, 2, 3, 4, 5, 6, 7, 8, 9, 10, 11, 12, 13, 14 or 15 amino acid residues of an epitope that is bound by any of the antibodies provided herein. For example, in some embodiments any of the antibodies provided herein bind at or near a tolloid cleavage site or at or near a tolloid docking site of a TGFβ family member protein (e.g., proGDF11 or latent GDF11). In some embodiments, an antibody binds near a tolloid cleavage site or near a tolloid docking site if it binds within 15 or fewer amino acid residues of the tolloid cleavage site or tolloid docking site. In some embodiments, any of the antibodies provided herein bind within 1, 2, 3, 4, 5, 6, 7, 8, 9, 10, 11, 12, 13, 14 or 15 amino acid residues of a tolloid cleavage site or tolloid docking site. For example, a tolloid cleavage site comprising the amino acid residues GD corresponding to amino acid residues 97-98 of GDF11 (SEQ ID NO: 82). In some embodiments, an antibody binds at or near a tolloid cleavage site of GDF11. For example, an antibody may bind an amino acid sequence as set forth in SEQ ID NO: 149. KAPPLQQILDLHDFQGDALQPEDFLEEDEYHA (SEQ ID NO: 149). In some embodiments, binding of an antibody at or near a tolloid cleavage site or at or near a tolloid docking site of a TGFβ family member protein (e.g., GDF11) inhibits cleavage of the TGFβ family member protein, for example, by a tolloid protease (e.g., BMP-1).

In other embodiments, any of the antibodies provided herein bind at or near a proprotein convertase cleavage site or at or near a proprotein convertase docking site of a TGFβ family member protein (e.g., proGDF11 or latent GDF11). In some embodiments, an antibody binds near a proprotein convertase cleavage site or near a proprotein convertase docking site if it binds within 15 or fewer amino acid residues of the proprotein convertase cleavage site or proprotein convertase docking site. In some embodiments, any of the antibodies provided herein bind within 1, 2, 3, 4, 5, 6, 7, 8, 9, 10, 11, 12, 13, 14 or 15 amino acid residues of a proprotein convertase cleavage site or proprotein convertase docking site. In some embodiments, an antibody binds at or near a proprotein convertase cleavage site of GDF11. For example, the GDF11 proprotein convertase cleavage site may comprise RSSR (SEQ ID NO: 151), corresponding to amino acid residues 23-26 of GDF11 (SEQ ID NO: 82); RELR (SEQ ID NO: 161), corresponding to amino acid residues 48-51 of GDF11 (SEQ ID NO: 82); or RSRR (SEQ ID NO: 152), corresponding to amino acid residues 271-274 of GDF11 (SEQ ID NO: 82). As another example, an antibody may bind an amino acid sequence GLHPFMELRVLENTKRSRRNLGLDCDEHSSESRC (SEQ ID NO: 153); PEPDGCPVCVWRQHSRELRLESIKSQILSKLRLK (SEQ ID NO: 154); or AAAAAAAAAAGVGGERSSRPAPSVAPEPDGCPVC (SEQ ID NO: 155). In some embodiments, binding of an antibody at or near a proprotein convertase cleavage site or at or near a proprotein convertase docking site of a TGFβ family member protein inhibits cleavage of the TGFβ family member protein, for example, by a proprotein convertase (e.g., PCSKS).

In other embodiments, any of the antibodies provided herein bind at or near a straight jacket region or at or near an ARM region of a TGFβ family member protein (e.g., proGDF11 or latent GDF11) In some embodiments, an antibody binds near a straight jacket region or near an ARM region site if it binds within 15 or fewer amino acid residues of the straight jacket region or ARM region. In some embodiments, any of the antibodies provided herein bind within 1, 2, 3, 4, 5, 6, 7, 8, 9, 10, 11, 12, 13, 14 or 15 amino acid residues of a proprotein convertase cleavage site or proprotein convertase docking site. In some embodiments, an antibody binds at or near a straight jacket region of GDF11. For example, an antibody may bind at or near a straight jacket region of GDF11 comprising the amino acid sequence set forth in SEQ ID NO: 88. As another example, an antibody may bind at or near an ARM region of GDF11 comprising the amino acid sequence set forth in SEQ ID NO: 92. In some embodiments, binding of an antibody at or near a straight jacket region or at or near an ARM region of a TGFβ family member protein inhibits activation of the TGFβ family member protein, for example, by inhibiting the release of the mature growth factor (e.g., mature GDF11).

In another embodiment, an antibody competes or cross-competes for binding to any of the antigens provided hererin (e.g., one or more TGFβ family member proteins or forms thereof, one or more portions or domains of TGFβ family member proteins and/or one or more chimeras of TGFβ family member proteins) with an equilibrium dissociation constant, Kd, between the antibody and the protein of less than 10⁻⁶ M. In other embodiments, an antibody competes or cross-competes for binding to any of the antigens provided herein with a Kd in a range from 10⁻¹¹ M to 10⁻⁶ M.

Any of the antibodies provided herein can be characterized using any suitable methods. For example, one method is to identify the epitope to which the antigen binds, or “epitope mapping.” There are many suitable methods for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screen by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in binding assays. In additional examples, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of TGFβ family member proteins have been replaced (swapped) with sequences from related, but antigenically distinct proteins, such as another member of a TGFβ family member protein. By assessing a binding profile of an antigen, the importance of the particular antigen fragment to antibody binding can be assessed.

Alternatively, competition assays can be performed using other antibodies known to bind to the same antigen to determine whether an antibody binds to the same epitope as the other antibodies. Such competition assays would be apparent to the skilled artisan.

Any of the suitable methods, e.g., the epitope mapping methods as described herein, can be applied to determine whether any of the antibodies provided hererin binds one or more of the specific residues/segments of one or more TGFβ family member proteins as described herein. Further, the interaction of an antibody with one or more of those defined residues in TGFβ family member proteins can be determined by routine technology. For example, a crystal structure can be determined, and the distances between the residues in TGFβ family member proteins and one or more residues in an antibody can be determined accordingly. Based on such distance, whether a specific residue in a TGFβ family member protein interacts with one or more residues in an antibody can be determined. Further, suitable methods, such as competition assays and target mutagenesis assays can be applied to determine the preferential binding of a candidate antibody to a TGFβ family member protein as compared to another TGFβ family member protein.

### Variations of Binding Proteins

Aspects of the disclosure provide variations of any of the polypeptides (e.g., anti-GDF11 antibodies) provided herein. Compounds and/or compositions of the present disclosure may exist as a whole polypeptide, a plurality of polypeptides or fragments of polypeptides, which independently may be encoded by one or more nucleic acids, a plurality of nucleic acids, fragments of nucleic acids or variants of any of the aforementioned. In some embodiments, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

As used herein, the term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Variants may possess at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5% or at least about 99.9% identity (homology) to a native or reference sequence.

In some embodiments “variant mimics” are provided. As used herein, the term “variant mimic” refers to a variant which contains one or more amino acids which would mimic an activated sequence. For example, glutamate may serve as a mimic for phospho-threonine and/or phospho-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of the compounds and/or compositions of the disclosure may comprise naturally occurring amino acids and as such may be considered to be proteins, peptides, polypeptides, or fragments thereof. Alternatively, the compounds and/or compositions may comprise both naturally and non-naturally occurring amino acids.

As used herein, the term “amino acid sequence variant” refers to molecules with some differences in their amino acid sequences as compared to a native or starting sequence. The amino acid sequence variants may possess substitutions, deletions, and/or insertions at certain positions within the amino acid sequence. As used herein, the terms “native” or “starting” when referring to sequences are relative terms referring to an original molecule against which a comparison may be made. Native or starting sequences should not be confused with wild type sequences. Native sequences or molecules may represent the wild-type (that sequence found in nature) but do not have to be identical to the wild-type sequence.

Ordinarily, variants will possess at least about 70% homology to a native sequence, and preferably, they will be at least about 80%, more preferably at least about 90% homologous to a native sequence.

As used herein, the term “homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

As used herein, the term “homolog” as it applies to amino acid sequences is meant the corresponding sequence of other species having substantial identity to a second sequence of a second species.

As used herein, the term “analog” is meant to include polypeptide variants which differ by one or more amino acid alterations, e.g., substitutions, additions or deletions of amino acid residues that still maintain the properties of the parent polypeptide.

As used herein, the term “derivative” is used synonymously with the term “variant” and refers to a molecule that has been modified or changed in any way relative to a reference molecule or starting molecule.

The present disclosure contemplates several types of compounds and/or compositions which are amino acid based including variants and derivatives. These include substitutional, insertional, deletional and covalent variants and derivatives. As such, included within the scope of this disclosure are compounds and/or compositions comprising substitutions, insertions, additions, deletions and/or covalent modifications. For example, sequence tags or amino acids, such as one or more lysines, can be added to peptide sequences of the disclosure (e.g., at the N-terminal or C-terminal ends). Sequence tags can be used for peptide purification or localization. Lysines can be used to increase peptide solubility or to allow for biotinylation. In some cases, amino acid sequences may be included that are targets for biotinylation (e.g. via bacterial ligase). Such sequences may include any of those listed in U.S. Pat. No. 5,723,584, the contents of which are herein incorporated by reference in their entirety. For example, the amino acid sequence GLNDIFEAQKIEWHE (SEQ ID NO: 156) may be used, where the biotin is joined via bacterial ligase to the embedded lysine residue. In addition, antibodies specific for GLNDIFEAQKIEWHE (SEQ ID NO: 156) may be used to target proteins expressing that sequence. In some cases, these sequences are expressed in association with N- and/or C-terminal secretion signal sequences [e.g. human Ig kappa chains with amino acid sequence MDMRVPAQLLGLLLLWFSGVLG (SEQ ID NO: 157)], flag tag sequences [e.g. DYKDDDDK (SEQ ID NO: 158)], one or more 3C protease cleavage site [e.g. LEVLFQGP (SEQ ID NO: 159)], one or more biotinylation site and/or His-tag sequences [e.g. HHHHHH (SEQ ID NO: 160)].

Amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein, the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As used herein, the term “insertional variants” when referring to proteins are those with one or more amino acids inserted immediately adjacent to an amino acid at a particular position in a native or starting sequence. As used herein, the term “immediately adjacent” refers to an adjacent amino acid that is connected to either the alpha-carboxy or alpha-amino functional group of a starting or reference amino acid.

As used herein, the term “deletional variants” when referring to proteins, are those with one or more amino acids in the native or starting amino acid sequence removed. Ordinarily, deletional variants will have one or more amino acids deleted in a particular region of the molecule.

As used herein, the term “derivatives,” as referred to herein includes variants of a native or starting protein comprising one or more modifications with organic proteinaceous or non-proteinaceous derivatizing agents, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the disclosure are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this disclosure, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337.

As used herein, the term “features” when referring to proteins are defined as distinct amino acid sequence-based components of a molecule. Features of the proteins of the present disclosure include surface manifestations, local conformational shape, folds, loops, half-loops, domains, half-domains, sites, termini or any combination thereof.

As used herein, the term “surface manifestation” when referring to proteins refers to a polypeptide based component of a protein appearing on an outermost surface.

As used herein, the term “local conformational shape” when referring to proteins refers to a polypeptide based structural manifestation of a protein which is located within a definable space of the protein.

As used herein, the term “fold”, when referring to proteins, refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein, the term “turn” as it relates to protein conformation, refers to a bend which alters the direction of the backbone of a peptide or polypeptide and may involve one, two, three or more amino acid residues.

As used herein, the term “loop,” when referring to proteins, refers to a structural feature of a peptide or polypeptide which reverses the direction of the backbone of a peptide or polypeptide and comprises four or more amino acid residues. Oliva et al. have identified at least 5 classes of protein loops (Oliva, B. et al., An automated classification of the structure of protein loops. J Mol Biol. 1997. 266(4):814-30).

As used herein, the term “half-loop,” when referring to proteins, refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+1-0.5 being 3 or 4).

As used herein, the term “domain,” when referring to proteins, refers to a motif of a polypeptide having one or more identifiable structural or functional characteristics or properties (e.g., binding capacity, serving as a site for protein-protein interactions).

As used herein, the term “half-domain,” when referring to proteins, refers to a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein, the terms “site,” as it pertains to amino acid based embodiments is used synonymously with “amino acid residue” and “amino acid side chain”. A site represents a position within a peptide or polypeptide that may be modified, manipulated, altered, derivatized or varied within the polypeptide based molecules of the present disclosure.

As used herein, the terms “termini” or “terminus,” when referring to proteins refers to an extremity of a peptide or polypeptide. Such extremity is not limited only to the first or final site of the peptide or polypeptide but may include additional amino acids in the terminal regions. The polypeptide based molecules of the present disclosure may be characterized as having both an N-terminus (terminated by an amino acid with a free amino group (NH2)) and a C-terminus (terminated by an amino acid with a free carboxyl group (COOH)). Proteins of the disclosure are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the disclosure, any of several manipulations and/or modifications of these features may be performed by moving, swapping, inverting, deleting, randomizing or duplicating. Furthermore, it is understood that manipulation of features may result in the same outcome as a modification to the molecules of the disclosure. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

In some embodiments, compounds and/or compositions of the present disclosure may comprise one or more atoms that are isotopes. As used herein, the term “isotope” refers to a chemical element that has one or more additional neutrons. In some embodiments, compounds of the present disclosure may be deuterated. As used herein, the term “deuterate” refers to the process of replacing one or more hydrogen atoms in a substance with deuterium isotopes. Deuterium isotopes are isotopes of hydrogen. The nucleus of hydrogen contains one proton while deuterium nuclei contain both a proton and a neutron. The compounds and/or compositions of the present disclosure may be deuterated in order to change one or more physical property, such as stability, or to allow compounds and/or compositions to be used in diagnostic and/or experimental applications.

In some aspects, the disclosure provide antibodies (e.g., anti-GDF11 antibodies) having a heavy chain variable and/or a light chain variable amino acid sequence homologous to any of those described herein. In some embodiments, the antibody comprises a heavy chain variable sequence or a light chain variable sequence that is at least 75% (e.g., 80%, 85%, 90%, 95%, 98%, or 99%) identical to the heavy chain variable sequence of any of SEQ ID NOs: 8, 10, 12, 14, 16, 18, and 20, or a light chain variable sequence of any one of SEQ ID NOs: 9, 11, 13, 15, 17, 19, or 21. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein.

### Production of Binding Proteins and Binding Protein-Producing Cell Lines

In one embodiment, anti-GDF11 prodomain complex binding proteins of the present disclosure may exhibit a high capacity to reduce or to neutralize GDF11 activity, e.g., as assessed by any one of several in vitro and in vivo assays known in the art.

In one embodiment, the isolated antibody, or antigen-binding portion thereof, binds human GDF11 prodomain complex, wherein the antibody, or antigen-binding portion thereof, dissociates from human GDF11 prodomain complex with a k_{off} rate constant of about 0.1 s⁻¹ or less, as determined by surface biolayer interferometry, or which inhibits human GDF11 activity with an IC₅₀ of about 1×10⁻⁶ M or less. Alternatively, the antibody, or an antigen-binding portion thereof, may dissociate from human GDF11 prodomain complex with a k_{off} rate constant of about 1×10⁻² s⁻¹ or less, as determined by surface biolayer interferometry, or may inhibit human GDF11 activity with an IC₅₀ of about 1×10⁻⁷ M or less. Alternatively, the antibody, or an antigen-binding portion thereof, may dissociate from human GDF11 prodomain complex with a k_{off} rate constant of about 1×10⁻³ s⁻¹ or less, as determined by surface biolayer interferometry, or may inhibit human GDF11 activity with an IC₅₀ of about 1×10⁻⁸ M or less. Alternatively, the antibody, or an antigen-binding portion thereof, may dissociate from human GDF11 prodomain complex with a k_{off} rate constant of about 1×10⁻⁴ s⁻¹ or less, as determined by surface biolayer interferometry, or may inhibit human GDF11 activity with an IC₅₀ of about 1×10⁻⁸ M or less. Alternatively, the antibody, or an antigen-binding portion thereof, may dissociate from human GDF11 prodomain complex with a _{koff} rate constant of about 1×10⁻⁵ s⁻¹ or less, as determined by surface biolayer interferometry, or may inhibit human GDF11 activity with an IC₅₀ of about 1×10 M or less.

In certain embodiments, the antibody comprises a heavy chain constant region, such as an IgG1, IgG2, IgG3, IgG4, IgA, IgE, IgM or IgD constant region. In another embodiment, the heavy chain constant region is an IgG1 heavy chain constant region or an IgG4 heavy chain constant region. Furthermore, the antibody can comprise a light chain constant region, either a kappa light chain constant region or a lambda light chain constant region. In another embodiment, the antibody comprises a kappa light chain constant region. Alternatively, the antibody portion can be, for example, a Fab fragment or a single chain Fv fragment.

Replacements of amino acid residues in the Fc portion to alter antibody effector function are known in the art (Winter, et al. U.S. Pat. Nos. 5,648,260; 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fc.gamma.Rs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment provides a labeled binding protein wherein an antibody or antibody portion of the disclosure is derivatized or linked to another functional molecule (e.g., another peptide or protein). For example, a labeled binding protein of the disclosure can be derived by functionally linking an antibody or antibody portion of the disclosure (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

Useful detectable agents with which an antibody or antibody portion of the disclosure may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

Another embodiment of the disclosure provides a crystallized binding protein. In another embodiment, the disclosure relates to crystals of whole anti-GDF11 prodomain complex antibodies and fragments thereof as disclosed herein, and formulations and compositions comprising such crystals. In another embodiment, the crystallized binding protein has a greater half-life in vivo than the soluble counterpart of the binding protein. In another embodiment the binding protein retains biological activity after crystallization.

Crystallized binding protein of the disclosure may be produced according methods known in the art and as disclosed in WO 02072636, incorporated herein by reference.

Another embodiment of the disclosure provides a glycosylated binding protein wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have important effect on the effector function of the Fc domain, with minimal effect on antigen binding or half-life of the antibody (R. Jefferis, Biotechnol. Prog. 21 (2005), pp. 11-16). In contrast, glycosylation of the variable domain may have an effect on the antigen binding activity of the antibody. Glycosylation in the variable domain may have a negative effect on antibody binding affinity, likely due to steric hindrance (Co, M. S., et al., Mol. Immunol. (1993) 30:1361-1367), or result in increased affinity for the antigen (Wallick, S. C., et al., Exp. Med. (1988) 168:1099-1109; Wright, A., et al., EMBO J. (1991) 10:2717 2723).

One aspect of the present disclosure is directed to generating glycosylation site mutants in which the O- or N-linked glycosylation site of the binding protein has been mutated. One skilled in the art can generate such mutants using standard well-known technologies. Glycosylation site mutants that retain the biological activity, but have increased or decreased binding activity, are another object of the present disclosure.

In another embodiment, the glycosylation of the antibody or antigen-binding portion of the disclosure is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in PCT Publication WO2003016466A2, and U.S. Pat. Nos. 5,714,350 and 6,350,861, each of which is incorporated herein by reference in its entirety.

Additionally or alternatively, a modified antibody of the disclosure can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342 80, each of which is incorporated herein by reference in its entirety.

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the disclosure may include, but are not limited to, glucose, galactose, mannose, fucose, n-acetylglucosamine and sialic acid. In another embodiment, the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. patent applications 20040018590 and 20020137134 and PCT publication WO2005100584 A2).

In addition to the binding proteins, the present disclosure is also directed to an anti-idiotypic (anti-Id) antibody specific for such binding proteins of the disclosure. An anti-Id antibody is an antibody, which recognizes unique determinants generally associated with the antigen-binding region of another antibody. The anti-Id can be prepared by immunizing an animal with the binding protein or a CDR containing region thereof. The immunized animal will recognize, and respond to the idiotypic determinants of the immunizing antibody and produce an anti-Id antibody. The anti-Id antibody may also be used as an “immunogen” to induce an immune response in yet another animal, producing a so-called anti-anti-Id antibody.

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. In another embodiment, the protein having a particularly selected novel glycosylation pattern exhibits improved or altered biological properties.

### Uses of Anti-GDF11 Prodomain Complex Binding Proteins

Given their ability to bind to human GDF11, the anti-human GDF11 prodomain complex antibodies, or portions thereof, of the disclosure can be used to detect human GDF11 prodomain complex (e.g., in a biological sample, such as serum or plasma), using a conventional immunoassay, such as an enzyme linked immunosorbent assays (ELISA), an radioimmunoassay (RIA), western blots, immunoaffinity mass spec, immunoprecipitation, immunofluorescence, MSD or tissue immunohistochemistry. The disclosure provides a method for detecting human GDF11 prodomain complex in a biological sample comprising contacting a biological sample with an antibody, or antibody portion, of the disclosure and detecting either the antibody (or antibody portion) bound to human GDF11 prodomain complex or unbound antibody (or antibody portion), to thereby detect human GDF11 prodomain complex in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol.

Alternative to labeling the binding protein, human GDF11 prodomain complex can be assayed in biological fluids by a competition immunoassay utilizing rhGDF11 prodomain complex standards labeled with a detectable substance and an unlabeled anti-human GDF11 prodomain complex antibody. In this assay, the biological sample, the labeled rhGDF11 prodomain complex standards and the anti-human GDF11 prodomain complex antibody are combined and the amount of labeled rhGDF11 prodomain complex standard bound to the unlabeled antibody is determined. The amount of human GDF11 prodomain complex in the biological sample is inversely proportional to the amount of labeled rhGDF11 prodomain complex standard bound to the anti-GDF11 prodomain complex antibody. Similarly, human GDF11 prodomain complex can also be assayed in biological fluids by a competition immunoassay utilizing rhGDF11 prodomain complex standards labeled with a detectable substance and an unlabeled anti-human GDF11 prodomain complex antibody.

In one embodiment, the antibodies and antibody portions of the disclosure are capable of neutralizing human GDF11 activity both in vitro and in vivo. Accordingly, such antibodies and antibody portions of the disclosure can be used to inhibit hGDF11 activity, e.g., in a cell culture containing hGDF11 prodomain complex, in human subjects or in other mammalian subjects having GDF11 prodomain complex with which an antibody of the disclosure cross-reacts. In another embodiment, the disclosure provides a method for inhibiting hGDF11 activity comprising contacting hGDF11 prodomain complex with an antibody or antibody portion of the disclosure such that hGDF11 activity is inhibited. For example, in a cell culture containing, or suspected of containing hGDF11 prodomain complex, an antibody or antibody portion of the disclosure can be added to the culture medium to inhibit hGDF11 activity in the culture.

In another embodiment, the disclosure provides a method for reducing hGDF11 activity in a subject, advantageously from a subject suffering from a disease or disorder in which GDF11 activity is detrimental. The disclosure provides methods for reducing GDF11 activity in a subject suffering from such a disease or disorder, which method comprises administering to the subject a binding protein, antibody or antibody portion of the disclosure such that GDF11 activity in the subject is reduced. In another embodiment, the GDF11 is human GDF11, and the subject is a human subject. Alternatively, the subject can be a mammal expressing a GDF11 prodomain complex to which an antibody of the disclosure is capable of binding. In another embodiment, the subject can be a mammal into which GDF11 has been introduced (e.g., by administration of GDF11 or by expression of an GDF11 transgene). An antibody of the disclosure can be administered to a human subject for therapeutic purposes. Moreover, an antibody of the disclosure can be administered to a non-human mammal expressing a GDF11 prodomain complex with which the antibody is capable of binding for veterinary purposes or as an animal model of human disease. Regarding the latter, such animal models may be useful for evaluating the therapeutic efficacy of antibodies of the disclosure (e.g., testing of dosages and time courses of administration).

As used herein, the term “a disorder in which GDF11 activity is detrimental” is intended to include diseases and other disorders in which the presence of GDF11 in a subject suffering from the disorder has been shown to be or is suspected of being either responsible for the pathophysiology of the disorder or a factor that contributes to a worsening of the disorder. Accordingly, a disorder in which GDF11 activity is detrimental is a disorder in which reduction of GDF11 activity is expected to alleviate the symptoms and/or progression of the disorder. Such disorders may be evidenced, for example, by an increase in the concentration of GDF11 in a biological fluid of a subject suffering from the disorder (e.g., an increase in the concentration of GDF11 in serum, plasma, synovial fluid, etc. of the subject), which can be detected, for example, using an anti-GDF11 antibody. Non-limiting examples of disorders that can be treated with the antibodies of the disclosure include those disorders discussed in the section below pertaining to pharmaceutical compositions of the antibodies of the disclosure.

In another embodiment, the binding proteins of the disclosure may be incorporated into multispecific binding proteins capable of binding target pairs including, but not limited to, GDF11 prodomain complex and another protein, such as TGF-beta, or other members of the TGF-beta super-family.

### Inhibiting GDF11 Proteolytic Cleavage

Some aspects of the disclosure provide methods of modulating growth factor activity (e.g., GDF11 growth factor activity) using inhibitors of GDF11 proteolytic activation. In some embodiments, the methods comprise delivering to a subject (e.g., a human or a mouse) an inhibitor of GDF11 proteolytic activation. In some embodiments, the the inhibitor of GDF11 proteolytic activation is a binding protein, compound, or small molecule. As used herein, a “small molecule” refers to a low molecular weight organic compound. In some embodiments, a small molecule has a molecular weight of 900 daltons or less. In some embodiments, a small molecule has a molecular weight of 850 daltons or less, 800 daltons or less, 750 daltons or less, 700 daltons or less, 650 daltons or less, 600 daltons or less, 550 daltons or less, 500 daltons or less, 450 daltons or less, 400 daltons or less, 350 daltons or less, 300 daltons or less, 250 daltons or less, 200 daltons or less, 150 daltons or less, 100 daltons or less, 50 daltons or less, or 20 daltons or less. Typically, small molecules may be used to penetrate cells to impact one or more biological functions.

In some embodiments, the inhibitor of GDF11 proteolytic activation is an inhibitor of a proprotein convertase or a furin protease. In some embodiments, the inhibitor of GDF11 proteolytic activation is an inhibitor of a proprotein convertase. In some embodiments, the inhibitor is an inhibitor of furin/PACE, PC1/3, PC2, PC4, PC5/6 (i.e., PCSK5), PACE4 or PC7. In some embodiments, the inhibitor is an inhibitor of PCSK5. Inhibitors of proprotein convertases are known in the art and would be apparent to the skilled artisan. For example, proprotein convertase inhibitors may include, without limitation, chloromethylketone, guanidinylated 2,5-dideoxystreptamine derivatives, (1,1′-(4,6-bis(4-guanidinophenoxy)cyclohexane-1,3-diyl)diguanidine), (1,3-bis(2,4-diguanidinophenoxy) benzene), and (1,1′-(4-((2,4-diguanidino-5-(4-guanidinophenoxy)cyclohexyl)oxy)-1,3-phenylene)diguanidine). Additional proprotein convertase inhibitors that are within the scope of this disclosure include those that have been described in Coppola J. M., et al., “A Small-Molecule Furin Inhibitor Inhibits Cancer Cell Motility and Invasiveness” Neoplasia 2008, vol 10, issue 4, p. 363-370.; Becker G. L., et al., Highly potent inhibitors of the proprotein convertase furin as potential drugs for the treatment of infectious diseases,” JBC, 2012, M111.332643; and Kowalska D., et al., “Synthetic Small-Molecule Prohormone Convertase 2 Inhibitors,” Molecular Pharmacology, 2009, vol. 75 no.3, 617-625; the entire contents of each are incorporated by reference herein.

In some embodiments, the inhibitor of GDF11 proteolytic activation is an inhibitor of a tolloid protease. In some embodiments, the inhibitor is an inhibitor of BMP-1, mammalian tolloid protein (mTLD), mammalian tolloid-like 1 (mTLL1), or mammalian tolloid-like 2 (mTLL2). In some embodiments, the inhibitor is an inhibitor of BMP-1 or mTLL2. Inhibitors of tolloid proteases, such as BMP-1 and mTLL2, would be apparent to the skilled artisan and are within the scope of this disclosure. It should be appreciated that the methods of delivering any of the inhibitors of GDF11 proteolytic activation may further include delivering any of the antibodies provided herein.

### Pharmaceutical Compositions

The disclosure also provides pharmaceutical compositions comprising a binding protein, antibody, or antigen-binding portion thereof, of the disclosure and a pharmaceutically acceptable carrier. In some embodiments, a “pharmaceutical composition” refers to a compound and/or composition of the present disclosure that has been formulated with on or more pharmaceutically acceptable excipients. The pharmaceutical compositions comprising antibodies of the disclosure are for use in, but not limited to, diagnosing, detecting, or monitoring a disorder, in preventing, treating, managing, or ameliorating of a disorder or one or more symptoms thereof, and/or in research. In a specific embodiment, a composition comprises one or more antibodies of the disclosure. In another embodiment, the pharmaceutical composition comprises one or more antibodies of the disclosure and one or more prophylactic or therapeutic agents other than antibodies of the disclosure for treating a disorder in which GDF11 activity is detrimental. In one embodiment, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The antibodies and antibody-portions of the disclosure can be incorporated into pharmaceutical compositions suitable for administration to a subject. Typically, the pharmaceutical composition comprises an antibody or antibody portion of the disclosure and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

Various delivery systems are known and can be used to administer one or more antibodies of the disclosure or the combination of one or more antibodies of the disclosure and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the disclosure include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entireties. In one embodiment, an antibody of the disclosure, combination therapy, or a composition of the disclosure is administered using Alkermes AIR.®. pulmonary drug delivery technology (Alkermes, Inc., Cambridge, Mass.). In a specific embodiment, prophylactic or therapeutic agents of the disclosure are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the disclosure locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices, or collagen matrices. In one embodiment, an effective amount of one or more antibodies of the disclosure antagonists is administered locally to the affected area to a subject to prevent, treat, manage, and/or ameliorate a disorder or a symptom thereof. In another embodiment, an effective amount of one or more antibodies of the disclosure is administered locally to the affected area in combination with an effective amount of one or more therapies (e.g., one or more prophylactic or therapeutic agents) other than an antibody of the disclosure of a subject to prevent, treat, manage, and/or ameliorate a disorder or one or more symptoms thereof.

In another embodiment, the prophylactic or therapeutic agent of the disclosure can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the disclosure (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the disclosure. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy &Oncology 39:179-189, Song et al., 1995, “Antibody Mediated Lung Targeting of Long-Circulating Emulsions,” PDA Journal of Pharmaceutical Science &Technology 50:372-397, Cleek et al., 1997, “Biodegradable Polymeric Carriers for a bFGF Antibody for Cardiovascular Application,” Pro. Intl Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

In a specific embodiment, where the composition of the disclosure is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the disclosure is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

If the compositions of the disclosure are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well known in the art.

If the method of the disclosure comprises intranasal administration of a composition, the composition can be formulated in an aerosol form, spray, mist or in the form of drops. In particular, prophylactic or therapeutic agents for use according to the present disclosure can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the disclosure comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the disclosure may comprise pulmonary administration, e.g., by use of an inhaler or nebulizer, of a composition formulated with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entireties. In a specific embodiment, an antibody of the disclosure, combination therapy, and/or composition of the disclosure is administered using Alkermes AIR.®. pulmonary drug delivery technology (Alkermes, Inc., Cambridge, Mass.).

The method of the disclosure may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the disclosure may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the disclosure encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the disclosure also provides that one or more of the prophylactic or therapeutic agents, or pharmaceutical compositions of the disclosure is packaged in a hermetically sealed container such as an ampoule or sachette indicating the quantity of the agent. In one embodiment, one or more of the prophylactic or therapeutic agents, or pharmaceutical compositions of the disclosure is supplied as a dry sterilized lyophilized powder or water free concentrate in a hermetically sealed container and can be reconstituted (e.g., with water or saline) to the appropriate concentration for administration to a subject. In another embodiment, one or more of the prophylactic or therapeutic agents or pharmaceutical compositions of the disclosure is supplied as a dry sterile lyophilized powder in a hermetically sealed container at a unit dosage of at least 5 mg, at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the disclosure should be stored at between 2. degree. C. and 8. degree. C. in its original container and the prophylactic or therapeutic agents, or pharmaceutical compositions of the disclosure should be administered within 1 week, within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the disclosure is supplied in liquid form in a hermetically sealed container indicating the quantity and concentration of the agent. In another embodiment, the liquid form of the administered composition is supplied in a hermetically sealed container at least 0.25 mg/ml, at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2. degree. C. and 8. degree. C. in its original container.

The antibodies and antibody-portions of the disclosure can be incorporated into a pharmaceutical composition suitable for parenteral administration. In another embodiment, the antibody or antibody-portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 24%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants. The pharmaceutical composition comprising the antibodies and antibody-portions of the disclosure prepared as an injectable solution for parenteral administration, can further comprise an agent useful as an adjuvant, such as those used to increase the absorption, or dispersion of a therapeutic protein (e.g., antibody). A particularly useful adjuvant is hyaluronidase, such as Hylenex.®. (recombinant human hyaluronidase). Addition of hyaluronidase in the injectable solution improves human bioavailability following parenteral administration, particularly subcutaneous administration. It also allows for greater injection site volumes (i.e. greater than 1 ml) with less pain and discomfort, and minimum incidence of injection site reactions. (see WO2004078140, US2006104968 incorporated herein by reference).

The compositions of this disclosure may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. In another embodiment, typical compositions are in the form of injectable or infusible solutions, such as compositions similar to those used for passive immunization of humans with other antibodies. In another embodiment, the mode of administration is parenteral (e.g., intravenous, subcutaneous, intraperitoneal, intramuscular). In another embodiment, the antibody is administered by intravenous infusion or injection. In another embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In another embodiment, in the case of sterile, lyophilized powders for the preparation of sterile injectable solutions, the methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The antibodies and antibody-portions of the present disclosure can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the disclosure may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the disclosure by other than parenteral administration, it may be necessary to coat the compound with, or co-administer the compound with, a material to prevent its inactivation.

Supplementary active compounds can also be incorporated into the compositions. In certain embodiments, an antibody or antibody portion of the disclosure is co-formulated with and/or co-administered with one or more additional therapeutic agents that are useful for treating disorders in which GDF11 activity is detrimental. For example, an anti-hGDF11 antibody or antibody portion of the disclosure may be coformulated and/or coadministered with one or more additional antibodies that bind other targets (e.g., antibodies that bind other cytokines or that bind cell surface molecules). Furthermore, one or more antibodies of the disclosure may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, an antibody to GDF11 prodomain complex or fragment thereof is linked to a half-life extending vehicle known in the art. Such vehicles include, but are not limited to, the Fc domain, polyethylene glycol, and dextran. Such vehicles are described, e.g., in U.S. application Ser. No. 09/428,082 and published PCT Application No. WO 99/25044, which are hereby incorporated by reference for any purpose.

In a specific embodiment, nucleic acid sequences comprising nucleotide sequences encoding an antibody of the disclosure or another prophylactic or therapeutic agent of the disclosure are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the disclosure, the nucleic acids produce their encoded antibody or prophylactic or therapeutic agent of the disclosure that mediates a prophylactic or therapeutic effect.

Any of the methods for gene therapy available in the art can be used according to the present disclosure. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley &Sons, NY (1993); and Kriegler, Gene Transfer and Expression, A Laboratory Manual, Stockton Press, NY (1990). Detailed description of various methods of gene therapy is disclosed in US20050042664 A1 which is incorporated herein by reference.

In another aspect, this application features a method of treating (e.g., curing, suppressing, ameliorating, delaying or preventing the onset of, or preventing recurrence or relapse of) or preventing a GDF11-associated disorder, in a subject. The method includes: administering to the subject a GDF11 prodomain complex binding agent (particularly an antagonist), e.g., an anti-GDF11 prodomain complex antibody or fragment thereof as described herein, in an amount sufficient to treat or prevent the GDF11-associated disorder. The GDF11 antagonist, e.g., the anti-GDF11 prodomain complex antibody or fragment thereof, can be administered to the subject, alone or in combination with other therapeutic modalities as described herein.

In one embodiment, the subject is a mammal, e.g., a human suffering from one or more GDF11-associated disorders, including, e.g., respiratory disorders (e.g., asthma (e.g., allergic and nonallergic asthma), chronic obstructive pulmonary disease (COPD), and other conditions involving airway inflammation, eosinophilia, fibrosis and excess mucus production; atopic disorders (e.g., atopic dermatitis and allergic rhinitis); inflammatory and/or autoimmune conditions of, the skin, gastrointestinal organs (e.g., inflammatory bowel diseases (IBD), such as ulcerative colitis and/or Crohn's disease), and liver (e.g., cirrhosis, fibrosis); scleroderma; tumors or cancers, e.g., Hodgkin's lymphoma as described herein. Accordingly, the disclosure includes the use of a GDF11 prodomain complex binding agent (such as an anti-GDF11 prodomain complex antibody or fragment thereof described herein) for a treatment described herein and the use of an GDF11 prodomain complex binding agent (such as an anti-GDF11 prodomain complex antibody or fragment thereof described herein) for preparing a medicament for a treatment described herein.

In one embodiment, examples of GDF11-associated disorders include, but are not limited to, anemia or erythroid hyperplasia. In another embodiment, examples of GDF11-associated disorders include, but are not limited to, a disorder chosen from one or more of:

respiratory disorders, e.g., asthma (e.g., allergic and nonallergic asthma (e.g., asthma due to infection with, e.g., respiratory syncytial virus (RSV), e.g., in younger children)), chronic obstructive pulmonary disease (COPD), and other conditions involving airway inflammation, eosinophilia, fibrosis and excess mucus production, e.g., cystic fibrosis and pulmonary fibrosis; atopic disorders, e.g., resulting from an increased sensitivity to GDF11 (e.g., atopic dermatitis, urticaria, eczema, allergic rhinitis, and allergic enterogastritis); inflammatory and/or autoimmune conditions of, the skin (e.g., atopic dermatitis), gastrointestinal organs (e.g., inflammatory bowel diseases (IBD), such as ulcerative colitis and/or Crohn's disease), liver (e.g., cirrhosis, hepatocellular carcinoma), and scleroderma; tumors or cancers (e.g., soft tissue or solid tumors), such as leukemia, glioblastoma, and lymphoma, e.g., Hodgkin's lymphoma; viral infections (e.g., from HTLV-1); fibrosis of other organs, e.g., fibrosis of the liver, (e.g., fibrosis caused by a hepatitis B and/or C virus); and suppression of expression of protective type 1 immune responses, (e.g., during vaccination), as described herein.

In other embodiments, this application provides a method of treating (e.g., reducing, ameliorating) or preventing one or more symptoms associated with a respiratory disorder, e.g., asthma (e.g., allergic and nonallergic asthma); allergies; chronic obstructive pulmonary disease (COPD); a condition involving airway inflammation, eosinophilia, fibrosis and excess mucus production, e.g., cystic fibrosis and pulmonary fibrosis. For example, symptoms of asthma include, but are not limited to, wheezing, shortness of breath, bronchoconstriction, airway hyperreactivity, decreased lung capacity, fibrosis, airway inflammation, and mucus production. The method comprises administering to the subject an GDF11 prodomain complex antagonist, e.g., an GDF11 prodomain complex antibody or a fragment thereof, in an amount sufficient to treat (e.g., reduce, ameliorate) or prevent one or more symptoms. The GDF11 prodomain complex antibody can be administered therapeutically or prophylactically, or both. The GDF11 prodomain complex antagonist, e.g., the anti-GDF11 prodomain complex antibody, or fragment thereof, can be administered to the subject, alone or in combination with other therapeutic modalities as described herein. In another embodiment, the subject is a mammal, e.g., a human suffering from a GDF11-associated disorder as described herein.

In another aspect, the binding proteins of the disclosure are useful for treating a disease or disorder associated with myopathy. As used herein, the term “myopathy” refers to a muscular disease in which the muscle fibers do not function properly, typically resulting in muscular weakness. Myopathies include muscular diseases that are neuromuscular or musculoskeletal in nature. In some embodiments, the myopathy is an inherited myopathy. Inherited myopathies include, without limitation, dystrophies, myotonias, congenital myopathies (e.g.,nemaline myopathy, multi/minicore myopathy, and centronuclear myopathy), mitochondrial myopathies, familial periodic myopathies, inflammatory myopathies and metabolic myopathies (e.g., glycogen storage diseases and lipid storage disorder). In some embodiments, the myopathy is an acquired myopathy. Acquired myopathies include, without limitation, external substance induced myopathy (e.g., drug-induced myopathy and glucocorticoid myopathy, alcoholic myopathy, and myopathy due to other toxic agents), myositis (e.g.,dermatomyositis, polymositis and inclusion body myositis), myositis ossificans, rhabdomyolysis, and myoglobinurias, and disuse atrophy. In some embodiments, the myopathy is disuse atrophy, which may be caused by bone fracture (e.g. a hip fracture) or by nerve injury (e.g., spinal cord injury (SCI)). In some embodiments the myopathy is related to a disease or disorder such as amyotrophic lateral sclerosis (ALS), spinal muscular atrophy (SMA),cachexia syndromes due to renal failure, AIDS, cardiac conditions and/or cancer. In some embodiments the myopathy is related to ageing.

An aspect of the disclosure includes a method of treating a subject having a myopathy, the method comprising administering to the subject an effective amount of a binding protein described herein. In some embodiments, the myopathy is a primary myopathy. In another embodiment, the primary myopathy comprises disuse atrophy. In other embodiments, the disuse atrophy is associated with hip fracture, elective joint replacement, critical care myopathy, spinal cord injury or stroke. In some embodiments, the myopathy is a secondary myopathy, in which muscle loss is secondary to a disease pathology. In other embodiments, the secondary myopathy comprises denervation, genetic muscle weakness or cachexia. In another embodiment, the secondary myopathy is a denervation associated with amyotrophic lateral sclerosis or spinal muscular atrophy. In some embodiments, the secondary myopathy is a genetic muscle weakness associated with a muscular dystrophy. In other embodiments, the secondary myopathy is a cachexia associated with renal failure, AIDS, a cardiac condition, cancer or aging.

Another aspect of the disclosure includes a method of treating a subject having a disease or condition related to aging. Exemplary diseases and conditions related to ageing include, without limitation, sarcopenia (age-related muscle loss), frailty, and androgen deficiency.

Another aspect of the disclosure includes a method of treating a subject having a disease or condition related to disuse atrophy/trauma. Exemplary diseases and conditions related to disuse atrophy/trauma include, without limitation, muscle weakness related to time spent in an intensive care unit (ICU), hip/joint replacement, hip fracture, stroke, bed rest, SCI, rotator cuff injury, knee replacement, bone fracture, and burns.

Another aspect of the disclosure includes a method of treating a subject having a neurodegenerative disease or condition. Exemplary neurodegenerative diseases or conditions include, without limitation, spinal muscular atrophy and amyotrophic lateral sclerosis (ALS).

Another aspect of the disclosure includes a method of treating a subject having a disease or condition related to Cachexia. Exemplary diseases and conditions related to cachexia include, without limitation, cancer, chronic heart failure, acquired immune deficiency syndrome (AIDS), chronic obstructive pulmonary disease (COPD), and chronic kidney disease (CKD).

Another aspect of the disclosure includes a method of treating a subject having a disease or condition related to rare diseases. Exemplary rare diseases and conditions include, without limitation, osteogenesis imperfecta, sporadic Inclusion body myositis, and acute lymphoblastic leukemia.

Another aspect of the disclosure includes a method of treating a subject having a disease or condition related to a metabolic disorder and/or body composition. In some embodiments, the disease or condition is obesity (e.g., severe obesity), Prader-Willi, type II diabetes, or anorexia. However, additional diseases or conditions related to metabolic disorders and/or body composition would be apparent to the skilled artisan and are within the scope of this disclosure.

Another aspect of the disclosure includes a method of treating a subject having a disease or condition related to congenital myopathies. Exemplary congenital myopathies include, without limitation, X-linked myotubular myopathy, autosomal dominant centronuclear myopathy, autosomal recessive centronuclear myopathy, nemaline myopathy, and congenital fiber-type disproportion myopathy.

Another aspect of the disclosure includes a method of treating a subject having a disease or condition related to muscular dystrophies. Exemplary muscular dystrophies include, without limitation, Duchenne's, Becker's, facioscapulohumeral (FSH), and Limb-Girdle muscular dystrophies. Another aspect of the disclosure includes a method of treating a subject having a urogynecological related disease or condition, glottic disorders (stenosis), extraocular myopathy, carpel tunnel, Guillain-Barré, or osteosarcoma.

In another aspect, the binding proteins of the disclosure are useful for treating a disorder selected from the group consisting of Thalassemia, beta thalassemia, anemia, iron deficiency anemia, plummer-vinson syndrome, pernicious anemia, megaloblastic anemia, protein deficiency anemia, scurvy, acanthocytosis, alpha-thalassemia, aplastic anemia, congenital dyserythropoietic anemia, hemolytic anemia fanconi anemia, hereditary sperocytosis, hereditary elliptocytosis, hereditary pyropoilikocytosis cold hemagglutinin disease, hemolytic uremic syndrome, hyperanemia, ineffective erythropesis, cacrocytic anemia, myelophthisic anemia, neuroacanthocytosis, chorea, acanthoscytosis, pyruvate kinase deficiency, sickle cell disease, thriosephophosphate isomerase deficiency, arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthropathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjorgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), H is bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignant Lymphoma, malignant histiocytosis, malignant melanoma, meningitis, meningococcemia, metabolic/idiopathic, migraine headache, mitochondrial multi system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, Acute coronary syndromes, Acute Idiopathic Polyneuritis, Acute Inflammatory Demyelinating Polyradiculoneuropathy, Acute ischemia, Adult Still's Disease, Alopecia greata, Anaphylaxis, Anti-Phospholipid Antibody Syndrome, Aplastic anemia, Arteriosclerosis, Atopic eczema, Atopic dermatitis, Autoimmune dermatitis, Autoimmune disorder associated with Streptococcus infection, Autoimmune Enteropathy, Autoimmune hearingloss, Autoimmune Lymphoproliferative Syndrome (ALPS), Autoimmune myocarditis, Autoimmune premature ovarian failure, Blepharitis, Bronchiectasis, Bullous pemphigoid, Cardiovascular Disease, Catastrophic Antiphospholipid Syndrome, Celiac Disease, Cervical Spondylosis, Chronic ischemia, Cicatricial pemphigoid, Clinically isolated Syndrome (CIS) with Risk for Multiple Sclerosis, Conjunctivitis, Childhood Onset Psychiatric Disorder, Chronic obstructive pulmonary disease (COPD), Dacryocystitis, dermatomyositis, Diabetic retinopathy, Diabetes mellitus, Disk herniation, Disk prolaps, Drug induced immune hemolytic anemia, Endocarditis, Endometriosis, endophthalmitis, Episcleritis, Erythema multiforme, erythema multiforme major, Gestational pemphigoid, Guillain-Barre Syndrome (GBS), Hay Fever, Hughes Syndrome, Idiopathic Parkinson's Disease, idiopathic interstitial pneumonia, IgE-mediated Allergy, Immune hemolytic anemia, Inclusion Body Myositis, Infectious ocular inflammatory disease, Inflammatory demyelinating disease, Inflammatory heart disease, Inflammatory kidney disease, IPF/UIP, Iritis, Keratitis, Keratojuntivitis sicca, Kussmaul disease or Kussmaul-Meier Disease, Landry's Paralysis, Langerhan's Cell Histiocytosis, Livedo reticularis, Macular Degeneration, Microscopic Polyangiitis, Morbus Bechterev, Motor Neuron Disorders, Mucous membrane pemphigoid, Multiple Organ failure, Myasthenia Gravis, Myelodysplastic Syndrome, Myocarditis, Nerve Root Disorders, Neuropathy, Non-A Non-B Hepatitis, Optic Neuritis, Osteolysis, Pauciarticular JRA, peripheral artery occlusive disease (PAOD), peripheral vascular disease (PVD), peripheral artery disease (PAD), Phlebitis, Polyarteritis nodosa (or periarteritis nodosa), Polychondritis, Polymyalgia Rheumatica, Poliosis, Polyarticular JRA, Polyendocrine Deficiency Syndrome, Polymyositis, polymyalgia rheumatica (PMR), Post-Pump Syndrome, primary parkinsonism, Prostatitis, Pure red cell aplasia, Primary Adrenal Insufficiency, Recurrent Neuromyelitis Optica, Restenosis, Rheumatic heart disease, SAPHO (synovitis, acne, pustulosis, hyperostosis, and osteitis), Scleroderma, Secondary Amyloidosis, Shock lung, Scleritis, Sciatica, Secondary Adrenal Insufficiency, Silicone associated connective tissue disease, Sneddon-Wilkinson Dermatosis, spondilitis ankylosans, Stevens-Johnson Syndrome (SJS), Systemic inflammatory response syndrome, Temporal arteritis, toxoplasmic retinitis, toxic epidermal necrolysis, Transverse myelitis, TRAPS (Tumor Necrosis Factor Receptor, Type 1 allergic reaction, Type II Diabetes, Urticaria, Usual interstitial pneumonia (UIP), Vasculitis, Vernal conjunctivitis, viral retinitis, Vogt-Koyanagi-Harada syndrome (VKH syndrome), Wet macular degeneration, and Wound healing.

In another aspect, the binding proteins of the disclosure are useful for treating a disorder selected from the group consisting of Acute Lymphoblastic Leukemia, Acute Myeloid Leukemia, Adrenocortical Carcinoma, Anal Cancer, Appendix Cancer, Cerebellar Astrocytoma, Cerebral Astrocytoma, Basal Cell Carcinoma, Bile Duct Cancer, Extrahepatic, Bladder Cancer, Bone Cancer, Osteosarcoma/Malignant Fibrous Histiocytoma Brain Stem Glioma, Brain Tumor, Brain Stem Glioma, Cerebral strocytoma/Malignant Glioma, Ependymoma, Medulloblastoma, Supratentorial Primitive Neuroectodermal Tumors, Visual Pathway and Hypothalamic Glioma, Breast Cancer, Bronchial Adenomas/Carcinoids, Carcinoid Tumor, Carcinoid Tumor, Gastrointestinal Carcinoma of Unknown Primary, Central Nervous System Lymphoma, Primary Cerebellar Astrocytoma, Cervical Cancer, Chronic Lymphocytic Leukemia, Chronic Myelogenous Leukemia Chronic Myeloproliferative Disorders, Colon Cancer, Colorectal Cancer, Cutaneous T-Cell Lymphoma, Endometrial Cancer, Ependymoma, Esophageal Cancer, Ewing Family of Tumors, Extracranial Germ Cell Tumor, Extragonadal Germ Cell Tumor, Extrahepatic Bile Duct Cancer, Eye Cancer, Intraocular Melanoma Retinoblastoma, Gallbladder Cancer, Gastric (Stomach) Cancer, Gastrointestinal Carcinoid Tumor, Gastrointestinal Stromal Tumor (GIST), Extracranial Germ Cell Tumor, Extragonadal Germ Cell Tumor, Ovarian Germ Cell Tumor, Gestational Trophoblastic Tumor, Glioma, Brain Stem Glioma, Cerebral Astrocytoma Glioma, Childhood Visual Pathway and Hypothalamic Glioma, Hairy Cell Leukemia, Head and Neck Cancer, Hepatocellular (Liver) Cancer, Hodgkin Lymphoma, Hypopharyngeal Cancer, Intraocular Melanoma, Islet Cell Carcinoma (Endocrine Pancreas), Kaposi Sarcoma, Kidney (Renal Cell) Cancer, Laryngeal Cancer, Acute Lymphoblastic Leukemia, Acute Myeloid Leukemia, Chronic Lymphocytic Leukemia, Chronic Myelogenous Leukemia, Hairy Cell Leukemia, Lip and Oral Cavity Cancer, Liver Cancer, Non-Small Cell Lung Cancer, Small Cell Lung Cancer, AIDS-Related Lymphoma, Burkitt Lymphoma, Cutaneous T-Cell Lymphoma, Hodgkin Lymphoma, Non-Hodgkin Lymphoma, Primary Central Nervous System Lymphoma, Waldenstrom Macroglobulinemia, Malignant Fibrous Histiocytoma of Bone/Osteosarcoma, Medulloblastoma, Melanoma, Intraocular (Eye) Melanoma, Merkel Cell Carcinoma, Malignant Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia, Chronic Myeloid Leukemia, Multiple Myeloma, Myeloproliferative Disorders, Nasal Cavity and Paranasal Sinus Cancer, Nasopharyngeal Cancer, Neuroblastoma, Oral Cancer, Oral Cavity Cancer, Lip and Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus and Nasal Cavity Cancer, Parathyroid Cancer, Penile Cancer, Pharyngeal Cancer, Pheochromocytoma, Pineoblastoma and Supratentorial Primitive Neuroectodermal Tumors, Pituitary Tumor, Plasma Cell Neoplasmi/Multiple Myeloma, Pleuropulmonary Blastoma, Prostate Cancer, Rectal Cancer, Renal Cell (Kidney) Cancer, Renal Pelvis and Ureter, Transitional Cell Cancer, Retinoblastoma, Salivary Gland Cancer, Sarcoma, Ewing Family of Tumors, Kaposi Sarcoma, Soft Tissue Sarcoma, Uterine Sarcoma, Sezary Syndrome, Skin Cancer (Nonmelanoma), Skin Cancer (Melanoma), Merkel Cell Skin Carcinoma, Small Intestine Cancer, Squamous Cell Carcinoma, Metastatic Squamous Neck Cancer with Occult Primary, Stomach (Gastric) Cancer, Supratentorial Primitive Neuroectodermal Tumors, Cutaneous T-Cell Lymphoma, Testicular Cancer, Throat Cancer, Thymoma, Thymoma and Thymic Carcinoma, Thyroid Cancer, Transitional Cell Cancer of the Renal Pelvis and Ureter, Gestational Trophoblastic Tumor, Ureter and Renal Pelvis, Transitional Cell Cancer, Urethral Cancer, Uterine Cancer, Endometrial Uterine Sarcoma, Vaginal Cancer, Visual Pathway and Hypothalamic Glioma, Vulvar Cancer, Waldenstrom Macroglobulinemia, and Wilms Tumor.

In another aspect the disclosure provides a method of treating a patient suffering from a disorder in which human GDF11 is detrimental comprising the step of administering any one of the binding proteins disclosed above before, concurrent, or after the administration of a second agent, as discussed herein. In an embodiment, the additional therapeutic agent that can be coadministered and/or coformulated with one or more GDF11 antagonists, (e.g., anti-GDF11 prodomain complex antibodies or fragments thereof,) include, but are not limited to, one or more of: inhaled steroids; oral steroids; beta-agonists, e.g., short-acting or long-acting beta-agonists; antagonists of leukotrienes or leukotriene receptors; combination drugs such as ADVAIR; IgE inhibitors, e.g., anti-IgE antibodies (e.g., XOLAIR); phosphodiesterase inhibitors (e.g., PDE4 inhibitors); xanthines; anticholinergic drugs; mast cell-stabilizing agents such as cromolyn; IL-4 inhibitors; IL-5 inhibitors; eotaxin/CCR3 inhibitors; antagonists of histamine or its receptors including H1, H2, H3, and H4, and antagonists of prostaglandin D or its receptors (DP1 and CRTH2). Such combinations can be used to treat asthma and other respiratory disorders. Additional examples of therapeutic agents that can be coadministered and/or coformulated with one or more anti-GDF11 antibodies or fragments thereof include one or more of: TNF antagonists (e.g., a soluble fragment of a TNF receptor, e.g., p55 or p75 human TNF receptor or derivatives thereof, e.g., 75 kD TNFR-IgG (75 kD TNF receptor-IgG fusion protein, ENBREL)); TNF enzyme antagonists, e.g., TNF converting enzyme (TACE) inhibitors; muscarinic receptor antagonists; TGF-beta antagonists; interferon gamma; perfenidone; chemotherapeutic agents, e.g., methotrexate, leflunomide, or a sirolimus (rapamycin) or an analog thereof, e.g., CCI-779; COX2 and cPLA2 inhibitors; NSAIDs; immunomodulators; p38 inhibitors, TPL-2, MK-2 and NFkB inhibitors, among others. Additional second agent is selected from the group consisting of budenoside, epidermal growth factor, corticosteroids, cyclosporin, sulfasalazine, aminosalicylates, 6-mercaptopurine, azathioprine, metronidazole, lipoxygenase inhibitors, mesalamine, olsalazine, balsalazide, antioxidants, thromboxane inhibitors; IL-1 receptor antagonists, anti-IL-1.beta. monoclonal antibodies, anti-IL-6 monoclonal antibodies, growth factors, elastase inhibitors, pyridinyl-imidazole compounds, antibodies or agonists of TNF, LT, IL-1, IL-2, IL-6, IL-7, IL-8, IL-15, IL-16, IL-18, EMAP-II, GM-CSF, FGF, and PDGF, antibodies of CD2, CD3, CD4, CD8, CD25, CD28, CD30, CD40, CD45, CD69, CD90 or their ligands, methotrexate, cyclosporin, FK506, rapamycin, mycophenolate mofetil, leflunomide, NSAIDs, ibuprofen, corticosteroids, prednisolone, phosphodiesterase inhibitors, adensosine agonists, antithrombotic agents, complement inhibitors, adrenergic agents, IRAK, NIK, IKK, p38, MAP kinase inhibitors, IL-1.beta, converting enzyme inhibitors, TNFconverting enzyme inhibitors, T-cell signalling inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1RI, sIL-1RII, sIL-6R, antiinflammatory cytokines, IL-4, IL-10, IL-11, and TGF-beta.

Antibodies of the disclosure, or antigen binding portions thereof can be used alone or in combination to treat such diseases. It should be understood that the antibodies of the disclosure or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present disclosure. The additional agent also can be an agent that imparts a beneficial attribute to the therapeutic composition e.g., an agent which affects the viscosity of the composition.

It should further be understood that the combinations which are to be included within this disclosure are those combinations useful for their intended purpose. The agents set forth below are illustrative for purposes and not intended to be limited. The combinations, which are part of this disclosure, can be the antibodies of the present disclosure and at least one additional agent selected from the lists below. The combination can also include more than one additional agent, e.g., two or three additional agents if the combination is such that the formed composition can perform its intended function.

The combination therapy can include one or more GDF11 prodomain complex antagonists, e.g., anti-GDF11 prodomain complex antibodies or fragments thereof, coformulated with, and/or coadministered with, one or more additional therapeutic agents, e.g., one or more cytokine and growth factor inhibitors, immunosuppressants, anti-inflammatory agents (e.g., systemic anti-inflammatory agents), anti-fibrotic agents, metabolic inhibitors, enzyme inhibitors, and/or cytotoxic or cytostatic agents, as described in more herein.

The pharmaceutical compositions of the disclosure may include a “therapeutically effective amount” or a “prophylactically effective amount” of an antibody or antibody portion of the disclosure. A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the disclosure is 0.1-20 mg/kg, 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In another embodiment, the pharmaceutical compositions disclosed herein are administered to the subject by at least one mode selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

### Recombinant and Chimeric Protein Use in Antibody Generation

In some embodiments, recombinant and/or chimeric proteins described herein may be used as antigens (referred to herein as antigenic proteins) to generate antibodies. Such antigenic proteins may comprise epitopes that may be less accessible for antibody generation in similar wild type proteins. Some antibodies directed to antigenic proteins of the present disclosure may modulate the release of one or more growth factors from one or more GPCs). Some such antibodies may be stabilizing [reducing or preventing dissociation between two agents, (e.g. growth-factor release from GPCs, GPC release from one or more protein interactions)] and/or releasing [enhancing the dissociation between two agents (e.g. growth-factor release from GPCs, GPC release from one or more protein interactions)] antibodies. Antigenic proteins of the present disclosure may comprise TGF-β-related proteins as well as components and/or protein modules thereof. In some cases, antigenic proteins of the present disclosure may comprise prodomains without associated growth factors, furin cleavage-deficient mutants, mutants deficient in extracellular protein associations and/or combinations thereof.

In some embodiments, antigenic proteins may comprise TGF-β-related proteins and/or modules thereof. Such antigenic proteins may comprise epitopes from regions where growth factors associate with or comprise stereological proximity with prodomain regions. Antibodies of the present disclosure directed to such epitopes may bind overlapping regions between growth factors and prodomains. Such antibodies may stereologically inhibit the dissociation of growth factors from GPCs.

In some embodiments, antigenic proteins comprise only the prodomain or only the growth factor from a particular GPC. Epitopes present on such antigenic proteins may be shielded or unexposed in intact GPCs. Some antibodies of the present disclosure may be directed to such epitopes. Such antibodies may be releasing antibodies, promoting growth factor dissociation from GPCs. Further antibodies may compete with free growth factor for prodomain binding, thereby promoting growth factor dissociation from GPCs.

In some embodiments, antigenic proteins may comprise proprotein convertase (e.g. furin) cleavage site mutations. Such mutations may prevent enzymatic cleavage of growth factors from their prodomains. Some antibodies of the present disclosure may be directed to epitopes present on such mutant proteins. Such antibodies may stabilize the association between prodomains and growth factors. In some embodiments, furin cleavage site mutants comprise D2G mutants as described herein.

In some embodiments, antigenic proteins comprising prodomains may comprise N-terminal mutations that lead to decreased prodomain association with extracellular proteins and therefore may present epitopes in the N-terminal region that may otherwise be shielded by those associations. Some antibodies of the present disclosure may be directed to such epitopes.

In some embodiments, antigenic proteins of the present disclosure may comprise one or more protein modules from GDFs (e.g. GDF11 and/or GDF8). In some embodiments, antibodies of the present disclosure may be directed toward antigenic proteins comprising GDF11 protein modules. In some embodiments, such antibodies may modulate GDF11 levels and/or activity in one or more niches. In some embodiments, antibodies of the present disclosure may prevent the release of GDF11 growth factors from GPCs. In some embodiments, antibodies of the present disclosure may be used to repair and/or enhance muscle tissues.

In some embodiments, recombinant proteins (including, but not limited to chimeric proteins) described herein may be used in studies to identify and map epitopes that may be important targets for antibody development. Such studies may be used to identify epitopes that may promote growth factor release or stabilization of GPCs upon antibody binding.

In some cases, recombinant proteins of the disclosure may comprise recombinant binding proteins, including, but not limited to antibodies, antibody fragments and fusion proteins comprising one or more antibodies or antibody fragments. Such recombinant binding proteins may comprise one or more regions from one or more antibodies developed using one or more recombinant antigens described herein.

### Releasing Antibodies

Some aspects of the disclosure provide releasing antibodies. As used herein, the term “releasing antibody” refers to an antibody that increases the ratio of active and/or free growth factor relative to inactive and/or prodomain-associated growth factor upon the introduction of the antibody to a GPC, cell, niche, natural depot or any other site of growth factor sequestration. In this context, releasing antibodies may be characterized as agonists. As used herein, the term “natural depot” refers to a location within a cell, tissue or organ where increased levels of a biomolecule or ion are stored. For example, the extracellular matrix may act as a natural depot for one or more growth factors.

The contact necessary for growth-factor release may be defined as direct or indirect contact of antibody with a GPC or a component thereof or with a cellular structure such as an extracellular and/or cellular matrix protein and/or protein associated with the extracellular and/or cellular matrix [e.g., fibrillins (e.g. fibrillin-1, fibrillin-2, fibrillin-3 and/or fibrillin-4), perlecan, decorin, elastin, collagen and/or GASPs] for release of growth factor. Release of at least 5%, 10%, 20%, 30%, 40%, 50%, 60%, 70%, 80%, 90% or more of growth factor is sufficient to characterize antibodies of the present disclosure as releasing antibodies. It is understood that growth factor release after antibody administration may be local and may occur over a sustained period of time and may include peaks or spikes of release. Antibodies of the present disclosure may act to release one or more growth factor over minutes, hours, days or longer.

Release profiles may have an initial peak or burst within from about 4 hours to about 7 days of contacting in vivo or shorter periods in vitro. For example, initial peak or burst may occur from about 4 hours to about 5 hours, or from about 4 hours to about 6 hours, or from about 4 hours to about 7 hours, or from about 4 hours to about 8 hours, or from about 4 hours to about 9 hours, or from about 4 hours to about 10 hours, or from about 4 hours to about 11 hours, or from about 4 hours to about 12 hours, or from about 4 hours to about 24 hours, or from about 4 hours to about 36 hours, or from about 4 hours to about 48 hours, or from about 1 day to about 7 days, or from about 1 day to about 2 days, or from about 1 day to about 3 days, or from about 1 day to about 4 days, or from about 4 days to about 5 days, or from about 4 days to about 6 days, or from about 4 days to about 7 days. Compounds and/or compositions of the present disclosure may stimulate the release of 5 to 100% of the growth factor present. For example, the percent of growth factor release may be from about 5% to about 10%, or from about 5% to about 15%, or from about 5% to about 20%, or from about 5% to about 25%, or from about 10% to about 30%, or from about 10% to about 40%, or from about 10% to about 50%, or from about 10% to about 60%, or from about 20% to about 70%, or from about 20% to about 80%, or from about 40% to about 90%, or from about 40% to about 100%.

In some embodiments, releasing antibodies of the disclosure may be characterized according to their half maximal effective concentration (EC₅₀). In some cases, this value may represent the concentration of antibody necessary to produce an increase in growth factor activity equal to half of the maximum amount of activity possible. Such EC₅₀ values may be from about 0.001 nM to about 0.01 nM, from about 0.005 nM to about 0.05 nM, from about 0.01 nM to about 1 nM, from about 0.05 nM to about 5 nM, from about 0.1 nM to about 10 nM, from about 0.5 nM to about 25 nM, from about 1 nM to about 50 nM, from about 5 nM to about 75 nM, from about 10 nM to about 100 nM, from about 25 nM to about 250 nM, from about 200 nM to about 1000 nM or more than 1000 nM.

Releasing antibodies generated according to methods described herein may be generated to release growth factors from GPCs comprising any of the pro-proteins listed in Table 1. In some cases, releasing antibodies are directed to GPCs comprising GDFs and/or one or more modules from GDFs. Some releasing antibodies of the disclosure release GDF11 from GPCs or other protein complexes.

### Stabilizing Binding Proteins

Some aspects of the disclosure provide stabilizing binding proteins (e.g., antibodies or antigen binding portions thereof). For example, as used herein, the term “stabilizing antibody” refers to an antibody that decreases the ratio of active and/or free growth factor relative to inactive and/or prodomain-associated growth factor upon the introduction of the antibody to one or more GPC, cell, niche, natural depot and/or any other site of growth factor sequestration. In this context, antibodies may be characterized as antagonists. As used herein, an “antagonist” is one which interferes with or inhibits the physiological action of another. Antagonist action may even result in stimulation or activation of signaling downstream and hence may act agonistically relative to another pathway, separate from the one being antagonized. Pathways are interrelated, so, in one nonlimiting example, a TGF-β antagonist could act as a BMP agonist and vice versa. In the context of cellular events, as used herein, the term “downstream” refers to any signaling or cellular event that happens after the action, binding or targeting by compounds and/or compositions of the present disclosure.

Contact necessary for inhibition or stabilization may be direct or indirect contact between antibody and GPC or components thereof or with cellular structures such as an extracellular and/or cellular matrix protein and/or protein associated with the extracellular and/or cellular matrix [e.g., fibrillins (e.g. fibrillin-1, fibrillin-2, fibrillin-3 and/or fibrillin-4), perlecan, decorin, elastin, collagen, and/or GASPs] whereby release of growth factor is inhibited. Inhibition of release of at least 5%, 10%, 20%, 30%, 40%, 50%, 60%, 70%, 80%, 90% or more of growth factors may be sufficient, in some cases, to characterize antibodies of the present disclosure as inhibitory or stabilizing. Inhibitory antibodies may stabilize GPCs and trap them as heterodimers.

In some cases, inhibitory antibodies of the disclosure are GDF11 inhibitory antibodies. Such antibodies may block the release of GDF11 growth factors from GPCs or other protein complexes. In some cases, GDF11 inhibitory antibodies otherwise reduce or eliminate GDF11 growth factor activity.

It is understood that inhibition of growth factor release after contact with one or more antibodies of the present disclosure may be local and may occur over a sustained period of time and may include peaks, troughs or spikes. Inhibitory antibodies which may also function to stabilize GPCs may be defined by their release kinetics. Release of growth factor and corresponding release kinetics, even locally, may be directly measured or inferred by downstream signaling events. In some embodiments, changes in protein or nucleic acid concentrations or phenotypic responses may be indicative of the effects of compounds and/or compositions of the present disclosure.

Antibodies of the present disclosure may act to inhibit release of a growth factor over minutes, hours or days. Inhibition and/or stabilization profiles may have an initial trough within from about 4 hours to about 7 days of introduction in vivo or shorter periods in vitro. For example, initial trough of inhibition or stabilization may occur from about 4 hours to about 5 hours, or from about 4 hours to about 6 hours, or from about 4 hours to about 7 hours, or from about 4 hours to about 8 hours, or from about 4 hours to about 9 hours, or from about 4 hours to about 10 hours, or from about 4 hours to about 11 hours, or from about 4 hours to about 12 hours, or from about 4 hours to about 24 hours, or from about 4 hours to about 36 hours, or from about 4 hours to about 48 hours, or from about 1 day to about 7 days, or from about 1 day to about 2 days, or from about 1 day to about 3 days, or from about 1 day to about 4 days, or from about 4 days to about 5 days, or from about 4 days to about 6 days, or from about 4 days to about 7 days. Introduction of compounds and/or compositions of the present disclosure may lead to inhibition and/or stabilization of 5% to 100% of growth factor present. For example, the percent of growth factor inhibition or stabilization may be from about 5% to about 10%, from about 5% to about 15%, from about 5% to about 20%, from about 5% to about 25%, from about 10% to about 30%, from about 10% to about 40%, from about 10% to about 50%, from about 10% to about 60%, from about 20% to about 70%, from about 20% to about 80%, from about 40% to about 90% or from about 40% to about 100%.

In some embodiments, stabilizing antibodies of the disclosure may be characterized according to their half maximal inhibitory concentration (IC₅₀). In some cases, this value may represent the concentration of antibody necessary to produce a decrease in growth factor activity equal to half of the maximum inhibition observed with the highest concentrations of antibody. Such IC₅₀ values may be from about 0.001 nM to about 0.01 nM, from about 0.005 nM to about 0.05 nM, from about 0.01 nM to about 1 nM, from about 0.05 nM to about 5 nM, from about 0.1 nM to about 10 nM, from about 0.5 nM to about 25 nM, from about 1 nM to about 50 nM, from about 5 nM to about 75 nM, from about 10 nM to about 100 nM, from about 25 nM to about 250 nM, from about 200 nM to about 1000 nM or more than 1000 nM.

Stabilizing antibodies generated according to methods described herein may be generated to block the release of growth factors from GPCs comprising any of the pro-proteins listed in Table 1. Such antibodies may physically interact with GPC protease cleavage sites and/or block the interaction of proteolytic enzymes that may target such cleavage sites. In some cases, stabilizing antibodies are directed to GPCs comprising GDFs and/or one or more modules from GDFs.

Stabilizing antibodies directed to GPCs comprising GDF11 may block metalloproteinase cleavage of such complexes. Such agents may bind to GPCs comprising GDF11 in such a way as to physically prevent interactions between such GPCs and metalloproteinases targeting such GPCs. Agents that actually target metalloproteinases themselves have been described previously (see U.S. Pat. No. 7,572,599, the contents of which are herein incorporated by reference in their entirety).

### Binding Protein Selection

A desired binding proteins (e.g., antibodies or antigen binding portions thereof) may be selected from a larger pool of two or more candidates based on the desired binding protein's ability to associate with desired antigens and/or epitopes. Such antigens and/or epitopes may include, but are not limited to any of those described herein, including, but not limited to recombinant proteins, chimeric proteins, GPCs, prodomains, growth factors, protein modules, fibrillins, GASPs, TGF-β-related proteins and/or mutants and/or variants and/or complexes and/or combinations thereof. In some embodiments, selection of desired antibodies may be carried out using an antibody binding assay, such as a surface plasmon resonance-based assay, an enzyme-linked immunosorbent assay (ELISA) or fluorescence flow cytometry-based assay. Such assays may utilize a desired antigen to bind a desired antibody and then use one or more detection methods to detect binding.

In some embodiments, antibodies of the present disclosure may be selected from a larger pool of two or more candidate antibodies based on their ability to associate with desired antigens and/or epitopes from multiple species (referred to herein as “positive selection.”)

In some embodiments, such species may comprise vertebrate species. In some embodiments, such species may comprise mammalian species. In some embodiments, such species may include, but are not limited to mice, rats, rabbits, goats, sheep, pigs, horses, cows and/or humans.

In some embodiments, negative selection is used to remove antibodies from a larger pool of two or more candidate antibodies. As used herein the term “negative selection” refers to the elimination of one or more factors from a group based on their ability to bind to one or more undesired antigens and/or epitopes. In some embodiments, undesired antigens and/or epitopes may include, but are not limited to any of those described herein, including, but not limited to recombinant proteins, chimeric proteins, GPCs, prodomains, growth factors, protein modules, fibrillins, GASPs, TGF-β-related proteins and/or mutants and/or variants and/or combinations and/or complexes thereof.

In some embodiments, antibodies of the present disclosure may be directed to prodomains (e.g. the prodomain portion of a GPC) that decrease growth factor signaling and/or levels (e.g. GDF growth factor signaling and/or levels) in a given niche. In some embodiments, antibodies of the present disclosure directed to prodomains may increase growth factor signaling and/or levels in a given niche. In some embodiments, antibodies of the present disclosure may be directed to prodomains and/or GPCs only when complexed with one or more extracellular protein, such as fibrillins, perlican, decorin and/or GASPs.

In some embodiments, antibodies of the present disclosure may be selected from a larger pool of two or more candidate antibodies based on their ability to modulate growth factor levels and/or activity. In some cases, growth factor activity assays may be used to test the ability of candidate antibodies to modulate growth factor activity. Growth factor activity assays may include, cell-based assays as described herein below. Additional assays that may be used to determine the effect of candidate antibodies on growth factor activity may include, but are not limited to enzyme-linked immunosorbent assay (ELISA), Western blotting, reporter assays (e.g. luciferase-based reporter assays or other enzyme-based reporter assays), PCR analysis, RT-PCR analysis and/or other methods known in the art including any of the methods described in International Patent Application No. WO2014074532, the contents of which are herein incorporated by reference in their entirety.

In some embodiments, one or more recombinant proteins or antibodies disclosed herein may be used in assays to test, develop and/or select antibodies. Recombinant GPCs may be expressed to test releasing and/or stabilizing abilities of one or more antibodies being assayed. In some embodiments, recombinant proteins may be expressed as positive or negative control components of assays. In some embodiments, multiple recombinant proteins may be expressed at once to modulate growth factor release and/or activity, wherein such recombinant proteins may act synergistically or antagonistically in such modulation.

### Recombinant Binding Protein

Recombinant binding proteins (e.g., antibodies or antigen binding portions thereof) of the present disclosure may be generated according to any of the methods disclosed in International Patent Application No. WO2014074532, the contents of which are herein incorporated by reference in their entirety. In some embodiments, recombinant antibodies may be produced using variable domains obtained from hybridoma cell-derived antibodies produced according to methods described herein. Heavy and light chain variable region cDNA sequences of antibodies may be determined using standard biochemical techniques. Total RNA may be extracted from antibody-producing hybridoma cells and converted to cDNA by reverse transcriptase (RT) polymerase chain reaction (PCR). PCR amplification may be carried out on resulting cDNA to amplify variable region genes. Such amplification may comprise the use of primers specific for amplification of heavy and light chain sequences. In other embodiments, recombinant antibodies may be produced using variable domains obtained from other sources. This includes the use of variable domains selected from one or more antibody fragment library, such as an scFv library used in antigen panning. Resulting PCR products may then be subcloned into plasmids for sequence analysis. Once sequenced, antibody coding sequences may be placed into expression vectors. For humanization, coding sequences for human heavy and light chain constant domains may be used to substitute for homologous murine sequences. The resulting constructs may then be transfected into mammalian cells for large scale translation.

In one embodiment, the disclosure provides an antibody construct comprising any one of the binding proteins disclosed above and a linker polypeptide or an immunoglobulin. In another embodiment, the antibody construct is selected from the group consisting of an immunoglobulin molecule, a monoclonal antibody, a fully human antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, and a bispecific antibody. In another embodiment, the antibody construct comprises a heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, and a human IgA constant domain. In another embodiment, the disclosure provides an antibody conjugate comprising the antibody construct disclosed herein and an agent, wherein the agent is selected from the group consisting of; an immunoadhesion molecule, an imaging agent, a therapeutic agent, and a cytotoxic agent. In another embodiment, the imaging agent is selected from the group consisting of a radiolabel, an enzyme, a fluorescent label, a luminescent label, a bioluminescent label, a magnetic label, and biotin. In another embodiment, the imaging agent is a radiolabel selected from the group consisting of ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm. In another embodiment, the therapeutic or cytotoxic agent is selected from the group consisting of; an anti-metabolite, an alkylating agent, an antibiotic, a growth factor, a cytokine, an anti-angiogenic agent, an anti-mitotic agent, an anthracycline, toxin, and an apoptotic agent.

In another embodiment, the antibody construct is glycosylated. In another embodiment, the glycosylation is a human glycosylation pattern.

### Development of Cytotoxic Binding Proteins

In some embodiments, binding proteins (e.g., antibodies or antigen binding portions thereof) of the present disclosure may be capable of inducing antibody-dependent cell-mediated cytotoxicity (ADCC), complement-dependent cytotoxicity (CDC) and/or antibody-dependent cell phagocytosis (ADCP). ADCC is an immune mechanism whereby cells are lysed as a result of immune cell attack. Such immune cells may include CD56+ cells, CD3—natural killer (NK) cells, monocytes and neutrophils (Strohl, W. R. Therapeutic Antibody Engineering. Woodhead Publishing, Philadelphia Pa. 2012. Ch. 8, p 186, the contents of which are herein incorporated by reference in their entirety).

In some cases, binding proteins (e.g., antibodies or antigen binding portions thereof) of the present disclosure may be engineered to comprise a given isotype depending on whether or not ADCC or ADCP is desired upon binding to antigen. Such binding proteins, for example, may be engineered according to any of the methods disclosed by Alderson, K. L. et al., J Biomed Biotechnol. 2011. 2011:379123). In the case of mouse antibodies, different isotypes of antibodies are more effective at promoting ADCC. IgG2a, for example, is more effective at inducing ADCC than is IgG2b. Some antibodies of the present disclosure, comprising mouse IgG2b antibodies may be reengineered to comprise IgG2a antibodies. Such reengineered antibodies may be more effective at inducing ADCC upon binding cell-associated antigens.

In some embodiments, genes encoding variable regions of antibodies developed according to methods of the present disclosure may be cloned into mammalian expression vectors encoding human Fc regions. Such Fc regions may comprise Fc regions from human IgG1κ. IgG1κ Fc regions may comprise amino acid mutations known to enhance Fc-receptor binding and antibody-dependent cell-mediated cytotoxicity ADCC.

In some cases, antibodies may be engineered to reduce ADCC. Antibodies that do not activate ADCC or that are associated with reduced levels of ADCC may be desireable for antibody embodiments of the present disclosure, in some cases due to no or limited immune-mediated clearance, allowing longer half-lives in circulation.

### Antibody Fragment Display Library Screening Techniques

In some embodiments, antibodies of the present disclosure may be produced and/or optimized using high throughput methods of discovery. Such methods may include any of the display techniques (e.g. display library screening techniques) disclosed in International Patent Application No. WO2014074532, the contents of which are herein incorporated by reference in their entirety. In some embodiments, synthetic antibodies may be designed, selected or optimized by screening target antigens using display technologies (e.g. phage display technologies). Phage display libraries may comprise millions to billions of phage particles, each expressing unique antibody fragments on their viral coats. Such libraries may provide richly diverse resources that may be used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of V_{H} and V_{L} antibody domains joined by a flexible linker (e.g. a Ser/Gly-rich linker). In some cases, scFvs may contain the same sequence with the exception of unique sequences encoding variable loops of the complementarity determining regions (CDRs). In some cases, scFvs are expressed as fusion proteins, linked to viral coat proteins (e.g. the N-terminus of the viral pIII coat protein). V_{L} chains may be expressed separately for assembly with V_{H} chains in the periplasm prior to complex incorporation into viral coats.

Phage selection according to the present disclosure may include the use of the antibody display library described in Schofield, D. et al., 2007. Genome Biol. 8, R254 and Pershad, K. et al., 2010. Protein Engineering Design and Selection. 23:279-88, the contents of which are herein incorporated by reference in their entirety. This library included over 10¹⁰ clones and has been validated through the successful generation of antibodies to over 300 antigens, producing more than 7,500 distinct antibody clones. Further, antibody production using this library may be carried out as described in Falk, R. et al., 2012. Methods. 58: 69-78 and/or Melidoni et al., 2013. PNAS 110(44): 17802-7, the contents of each of which are herein incorporated by reference in their entirety.

For selection, target antigens may be incubated, in vitro, with phage display library particles for precipitation of positive binding partners. This process is referred to herein as “phage enrichment.” In some cases, phage enrichment comprises solid-phase phage enrichment. According to such enrichment, target antigens are bound to a substrate (e.g. by passive adsorption) and contacted with one or more solutions comprising phage particles. Phage particles with affinity for such target antigens are precipitated out of solution. In some cases, phage enrichment comprises solution-phase phage enrichment where target antigens are present in a solution that is combined with phage solutions. According to such methods, target antigens may comprise detectable labels (e.g. biotin labels) to facilitate retrieval from solution and recovery of bound phage. In other embodiments, solution-phase phage enrichment may comprise the use of antigens bound to beads (e.g. streptavidin beads). In some cases, such beads may be magnetic beads to facilitate precipitation.

In some embodiments, phage enrichment may comprise solid-phase enrichment where target antigens are immobilized on solid surface. According to such methods, phage solutions may be used to contact the solid surface for enrichment with the immobilized antigens. Solid surfaces may include any surfaces capable of retaining antigens and may include, but are not limited to dishes, plates, flasks and tubes. In some cases, immunotubes may be used wherein the inner surface of such tubes may be coated with antigens. Phage enrichment with immunotubes may be carried out by passage of phage solution through the tubes to enrich bound antigens.

After selection, bound phage may be used to infect E. coli cultures that are co-infected with helper phage, to produce an amplified output library for the next round of enrichment. This process may be repeated producing narrower and narrower clone sets. In some embodiments, rounds of enrichment are limited to improve the diversity of selected phage.

Precipitated library members may be sequenced from the bound phage to obtain cDNA encoding desired scFvs. Such sequences may be directly incorporated into antibody sequences for recombinant antibody production, or mutated and utilized for further optimization through in vitro affinity maturation.

IgG antibodies comprising one or more variable domains from selected scFvs may be synthesized for further testing and/or product development. Such antibodies may be produced by insertion of one or more segments of scFv cDNA into expression vectors suited for IgG production. Expression vectors may comprise mammalian expression vectors suitable for IgG expression in mammalian cells. Mammalian expression of IgGs may be carried out to ensure that antibodies produced comprise modifications (e.g. glycosylation) characteristic of mammalian proteins and/or to ensure that antibody preparations lack endotoxin and/or other contaminants that may be present in protein preparations from bacterial expression systems.

In some embodiments, scFvs developed according to the disclosure may be expressed as scFv-Fc fusion proteins, comprising an antibody Fc domain. Such scFvs may be useful for further screening and analysis of scFv binding and affinity.

In some cases phage display screening may be used to generate broadly diverse panels of antibodies. Such diversity may be measured by diversity of antibody sequences and/or diversity of epitopes targeted.

Affinity binding estimates may be made using cross blocking experiments to bin antibodies. In some cases, affinity analysis instruments may be used. Such instruments may include, but are not limited surface plasmon resonance instrumentation, including, but not limited to Octet® (ForteBio, Menlo Park, Calif.).

In some cases, epitope binning may be carried out to identify groups of antibodies binding distinct epitopes present on the same antigen. Such binning may be informed by data obtained from affinity analysis using cross blocking experiments and/or affinity analysis instrumentation.

Affinity Maturation Techniques

Affinity maturation techniques of the present disclosure may comprise any of those disclosed in International Patent Application No. WO2014074532, the contents of which are herein incorporated by reference in their entirety. After antibody fragments capable of binding target antigens are identified (e.g. through the use of phage display libraries as described above), high affinity mutants may be derived from these through the process of affinity maturation. Affinity maturation technology is used to identify sequences encoding CDRs that have the highest affinity for target antigens. Using such technologies, select CDR sequences (e.g. ones that have been isolated or produced according to processes described herein) may be mutated randomly as a whole or at specific residues to create millions to billions of variants. Such variants may be subjected to repeated rounds of affinity screening (e.g. display library screening) for their ability to bind target antigens. Such repeated rounds of selection, mutation and expression may be carried out to identify antibody fragment sequences with the highest affinity for target antigens. Such sequences may be directly incorporated into antibody sequences for recombinant antibody production.

### Antibody Characterization

Binding proteins (e.g., antibodies or antigen binding portions thereof) of the disclosure may be characterized or grouped according to different structural and/or functional attributes. In some cases, antibodies of the disclosure may be characterized by affinity for one or more epitopes. In some cases, antibody affinity may be determined or described using association (Ka) or dissociation (Kd) constants. The Kd (when referring to antibodies of the disclosure), also referred to as the equilibrium constant, represents the ratio of the concentration of epitope-dissociated antibody divided by the concentration of epitope-associated antibody in a given system. Smaller values are indicative of higher affinity. In some cases, Kd values for antibody epitope binding are determined by ELISA analysis. In some cases, Kd values for antibody epitope binding are determined by surface plasmon resonance analysis (e.g., using an OCTET® instrument, ForteBio, Menlo Park, Calif.).

Compounds and/or compositions of the present disclosure comprising antibodies may act to decrease local concentration of one or more GPC through removal by phagocytosis, pinocytosis, or inhibiting assembly in the extracellular matrix and/or cellular matrix. Introduction of compounds and/or compositions of the present disclosure may lead to the removal of 5% to 100% of the growth factor present in a given area. For example, the percent of growth factor removal may be from about 5% to about 10%, from about 5% to about 15%, from about 5% to about 20%, from about 5% to about 25%, from about 10% to about 30%, from about 10% to about 40%, from about 10% to about 50%, from about 10% to about 60%, from about 20% to about 70%, from about 20% to about 80%, from about 40% to about 90% or from about 40% to about 100%.

Measures of release, inhibition or removal of one or more growth factors may be made relative to a standard or to the natural release or activity of growth factor under normal physiologic conditions, in vitro or in vivo. Measurements may also be made relative to the presence or absence of antibodies. Such methods of measuring growth factor levels, release, inhibition or removal include standard measurement in tissue and/or fluids (e.g. serum or blood) such as Western blot, enzyme-linked immunosorbent assay (ELISA), activity assays, reporter assays, luciferase assays, polymerase chain reaction (PCR) arrays, gene arrays, Real Time reverse transcriptase (RT) PCR and the like.

Antibodies of the present disclosure may bind or interact with any number of epitopes on or along GPCs or their associated structures to either enhance or inhibit growth factor signaling. Such epitopes may include any and all possible sites for altering, enhancing or inhibiting GPC function. In some embodiments, such epitopes include, but are not limited to epitopes on or within growth factors, regulatory elements, GPCs, GPC modulatory factors, growth factor receiving cells or receptors, fastener regions, furin cleavage sites, arm regions, fingers regions, fibrillin binding domains, latency lassos, alpha 1 regions, RGD sequences, bowtie regions, extracellular matrix and/or cellular matrix components and/or epitopes formed by combining regions or portions of any of the foregoing.

Compounds and/or compositions of the present disclosure exert their effects via binding (reversibly or irreversibly) to one or more epitopes and/or regions of antibody recognition. While not wishing to be bound by theory, such binding sites for antibodies, are most often formed by proteins, protein domains or regions. Binding sites may; however, include biomolecules such as sugars, lipids, nucleic acid molecules or any other form of binding epitope.

In some embodiments, antagonist antibodies of the present disclosure may bind to GDF prodomains, stabilizing and preventing growth factor release, for example, by blocking an enzymatic cleavage site or by stabilizing the structure. Such antibodies would be useful in the treatment of GDF-related indications resulting from excessive GDF activity.

Alternatively or additionally, antibodies of the present disclosure may function as ligand mimetics which would induce internalization of GPCs. Such antibodies may act as nontraditional payload carriers, acting to deliver and/or ferry bound or conjugated drug payloads to specific GPC and/or GPC-related sites.

Changes elicited by antibodies of the present disclosure may result in neomorphic changes in the cell. As used herein, the term “neomorphic change” refers to a change or alteration that is new or different. For example, an antibody that elicits the release or stabilization of one or more growth factor not typically associated with a particular GPC targeted by the antibody, would be a neomorphic antibody and the release would be a neomorphic change.

In some embodiments, compounds and/or compositions of the present disclosure may act to alter and/or control proteolytic events. In some embodiments, such proteolytic events may be intracellular or extracellular. In some embodiments, such proteolytic events may include the alteration of furin cleavage and/or other proteolytic processing events. In some embodiments, such proteolytic events may comprise proteolytic processing of growth factor signaling molecules or downstream cascades initiated by growth factor signaling molecules.

In some embodiments, compounds and/or compositions of the present disclosure may induce or inhibit dimerization or multimerization of growth factors (ligands) or their receptors. In some embodiments, such actions may be through stabilization of monomeric, dimeric or multimeric forms or through the disruption of dimeric or multimeric complexes.

In some embodiments, compounds and/or compositions of the present disclosure may act on homo and/or heterodimers of the monomeric units comprising either receptor groups or GPCs or other signaling molecule pairs.

Antibodies of the present disclosure may be internalized into cells prior to binding target antigens. Upon internalization, such antibodies may act to increase or decrease one or more signaling events, release or stabilize one or more GPCs, block or facilitate growth factor release and/or alter one or more cell niche.

In some embodiments, compounds and/or compositions of the present disclosure may also alter the residence time of one or more growth factor in one or more GPC and/or alter the residence time of one or more GPC in the extracellular matrix and/or cellular matrix. Such alterations may result in irreversible localization and/or transient localization.

Antibodies of the present disclosure may be designed, manufactured and/or selected using any methods known to one of skill in the art. In some embodiments, antibodies and/or antibody producing cells of the present disclosure are produced according to any of the methods listed in International Patent Application No. WO2014074532, the contents of which are herein incorporated by reference in their entirety.

### Binding Proteins Generation in Knockout Mice

In some embodiments, binding proteins (e.g., antibodies or antigen binding portions thereof) of the current disclosure may be generated in knockout mice that lack a gene encoding one or more desired antigens. Such mice would not be tolerized to such antigens and therefore may be able to generate antibodies against them that could cross react with human and mouse forms of the antigen. For the production of monoclonal antibodies, host mice are immunized with the target peptide to elicit lymphocytes that specifically bind that peptide. Lymphocytes are collected and fused with an immortalized cell line. The resulting hybridoma cells are cultured in a suitable culture medium with a selection agent to support the growth of only the fused cells.

In some embodiments, knocking out one or more growth factor gene may be lethal and/or produce a fetus or neonate that is non-viable. In some embodiments, neonatal animals may only survive for a matter of weeks (e.g. 1, 2, 3, 4 or 5 weeks). In such embodiments, immunizations may be carried out in neonatal animals shortly after birth. Oida et al (Oida, T. et al., TGF-β induces surface LAP expression on Murine CD4 T cells independent of FoxP3 induction. PLOS One. 2010. 5(11):e15523) demonstrate immunization of neonatal TGF-β knockout mice through the use of galectin-1 injections to prolong survival (typically 3-4 weeks after birth in these mice). Mice were immunized with cells expressing murine TGF-β every other day for 10 days beginning on the 8^{th} day after birth and spleen cells were harvested on day 22 after birth. Harvested spleen cells were fused with myeloma cells and of the resulting hybridoma cells, many were found to successfully produce anti-LAP antibodies. In some embodiments of the present disclosure, these methods may be used to generate antibodies. In some embodiments, such methods may comprise the use of human antigens.

Methods of the present disclosure may also comprise one or more steps of the immunization methods described by Oida et al combined with one or more additional and/or modified steps. Modified steps may include, but are not limited to the use of alternate cell types for fusions, the pooling of varying number of spleen cells when performing fusions, altering the injection regimen, altering the date of spleen cell harvest, altering immunogen and/or altering immunogen dose. Additional steps may include the harvesting of other tissues (e.g. lymph nodes) from immunized mice.

### Modulatory Binding Proteins

In some embodiments, antibodies of the present disclosure may comprise activating or inhibiting antibodies. As used herein, the term “inhibiting antibody” refers to an antibody that reduces growth factor activity. Inhibiting antibodies include antibodies targeting any epitope that reduces growth factor activity when associated with such antibodies. Such epitopes may lie on prodomains, growth factors or other epitopes that lead to reduced growth factor activity when bound by antibody. Inhibiting antibodies of the present disclosure may include, but are not limited to GDF-inhibiting antibodies such as GDF-11-inhibiting antibodies. In contrast, as used herein, the term “activating antibody” refers to an antibody that promotes growth factor activity. Activating antibodies include antibodies targeting any epitope that promotes growth factor activity. Such epitopes may lie on prodomains, growth factors or other epitopes that when bound by antibody, lead to growth factor activity. Activating antibodies of the present disclosure may include GDF-11-activating antibodies.

In some embodiments, inhibiting antibodies of the disclosure may include anti-primed complex antibodies. Such antibodies may target GDF-11 primed complexes and block resulting growth factor activity. In some cases, anti-primed complex antibodies may prevent dissociation of bound prodomain upon receptor binding. In some cases, anti-primed complex antibodies may prevent primed complexes from binding to receptors. In some cases, anti-primed complex antibodies may prevent primed complexes from associating with one or more other factors, leading to modulation of growth factor activity.

Embodiments of the present disclosure include methods of using activating and/or inhibiting antibodies in solution, in cell culture and/or in subjects to modify growth factor signaling.

### Anti-Prodomain Antibodies

In some embodiments, compounds and/or compositions of the present disclosure may comprise one or more antibody targeting a growth factor prodomain. Such antibodies may reduce or elevate growth factor signaling depending on the specific prodomain that is bound and/or depending on the specific epitope targeted by such antibodies. Anti-prodomain antibodies of the disclosure may promote dissociation of free growth factors from GPCs. Such dissociation may be induced upon antibody binding to a GPC or dissociation may be promoted by preventing the reassociation of free growth factor with prodomains. In some cases, anti-GDF prodomain antibodies are provided. Anti-GDF prodomain antibodies may comprise GDF-activating antibodies. Such antibodies may increase GDF activity (e.g. GDF-11 activity), in some cases by releasing GDF free growth factor from latent GPCs and/or preventing the reassociation of free growth factors with prodomains. In some cases, anti-GDF prodomain antibodies may increase GDF activity more favorably when a proGDF is associated with an extracellular protein (e.g., fibrillin or a GASP protein).

### Conjugates and Combinations

It is contemplated by the present invention that the compounds and/or compositions of the present invention may be complexed, conjugated or combined with one or more homologous or heterologous molecules. As used herein, the term “homologous molecule” refers to a molecule which is similar in at least one of structure or function relative to a starting molecule while a “heterologous molecule” is one that differs in at least one of structure or function relative to a starting molecule. Structural homologs are therefore molecules which may be substantially structurally similar. In some embodiments, such homologs may be identical. Functional homologs are molecules which may be substantially functionally similar. In some embodiments, such homologs may be identical.

Compounds and/or compositions of the present invention may comprise conjugates. Such conjugates of the invention may include naturally occurring substances or ligands, such as proteins (e.g., human serum albumin (HSA), low-density lipoprotein (LDL), high-density lipoprotein (HDL), or globulin); carbohydrates (e.g., a dextran, pullulan, chitin, chitosan, inulin, cyclodextrin or hyaluronic acid); or lipids. Conjugates may also be recombinant or synthetic molecules, such as synthetic polymers, e.g., synthetic polyamino acids, an oligonucleotide (e.g. an aptamer). Examples of polyamino acids may include polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

In some embodiments, conjugates may also include targeting groups. As used herein, the term “targeting group” refers to a functional group or moiety attached to an agent that facilitates localization of the agent to a desired region, tissue, cell and/or protein. Such targeting groups may include, but are not limited to cell or tissue targeting agents or groups (e.g. lectins, glycoproteins, lipids, proteins, an antibody that binds to a specified cell type such as a kidney cell or other cell type). In some embodiments, targeting groups may comprise thyrotropins, melanotropins, lectins, glycoproteins, surfactant protein A, mucin carbohydrates, multivalent lactose, multivalent galactose, N-acetyl-galactosamine, N-acetyl-gulucosamine, multivalent mannose, multivalent fucose, glycosylated polyaminoacids, multivalent galactose, transferrin, bisphosphonate, polyglutamate, polyaspartate, lipids, cholesterol, steroids, bile acids, folates, vitamin B12, biotin, an RGD peptide, an RGD peptide mimetic or an aptamer.

In some embodiments, targeting groups may be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also comprise hormones and/or hormone receptors.

In some embodiments, targeting groups may be any ligand capable of targeting specific receptors. Examples include, without limitation, folate, GalNAc, galactose, mannose, mannose-6-phosphate, apatamers, integrin receptor ligands, chemokine receptor ligands, transferrin, biotin, serotonin receptor ligands, PSMA, endothelin, GCPII, somatostatin, LDL, and HDL ligands. In some embodiments, targeting groups are aptamers. Such aptamers may be unmodified or comprise any combination of modifications disclosed herein.

In still other embodiments, compounds and/or compositions of the present invention may be covalently conjugated to cell penetrating polypeptides. In some embodiments, cell-penetrating peptides may also include signal sequences. In some embodiments, conjugates of the invention may be designed to have increased stability, increased cell transfection and/or altered biodistribution (e.g., targeted to specific tissues or cell types).

In some embodiments, conjugating moieties may be added to compounds and/or compositions of the present invention such that they allow the attachment of detectable labels to targets for clearance. Such detectable labels include, but are not limited to biotin labels, ubiquitins, fluorescent molecules, human influenza hemaglutinin (HA), c-myc, histidine (His), flag, glutathione S-transferase (GST), V5 (a paramyxovirus of simian virus 5 epitope), biotin, avidin, streptavidin, horse radish peroxidase (HRP) and digoxigenin.

In some embodiments, compounds of the invention may be conjugated with an antibody Fc domain to create an Fc fusion protein. The formation of an Fc fusion protein with any of the compounds described herein may be carried out according to any method known in the art, including as described in U.S. Pat. Nos. 5,116,964, 5,541,087 and 8,637,637, the contents of each of which are herein incorporated by reference in their entirety. Fc fusion proteins of the invention may comprise a compound of the invention linked to the hinge region of an IgG Fc via cysteine residues in the Fc hinge region. Resulting Fc fusion proteins may comprise an antibody-like structure, but without C_{H1} domains or light chains. In some cases, Fc fusion proteins may comprise pharmacokinetic profiles comparable to native antibodies. In some cases, Fc fusion proteins of the invention may comprise extended half-life in circulation and/or altered biological activity.

In some embodiments, compounds and/or compositions of the present invention may be combined with one another or other molecules in the treatment of diseases and/or conditions.

### Nucleic Acids

In some embodiments, compounds and/or compositions of the present invention may be encoded by nucleic acid molecules. Such nucleic acid molecules include, without limitation, DNA molecules, RNA molecules, polynucleotides, oligonucleotides, mRNA molecules, vectors, plasmids and the like. In some embodiments, the present invention may comprise cells programmed or generated to express nucleic acid molecules encoding compounds and/or compositions of the present invention. In some cases, nucleic acids of the invention include codon-optimized nucleic acids. Methods of generating codon-optimized nucleic acids are known in the art and may include, but are not limited to those described in U.S. Pat. Nos. 5,786,464 and 6,114,148, the contents of each of which are herein incorporated by reference in their entirety.

In another embodiment, the instant disclosure pertains to an isolated nucleic acid encoding any one of the binding proteins, antibody constructs or antibody conjugates provided herein. A further embodiment provides a vector comprising the isolated nucleic acid provided herein wherein said vector is selected from the group consisting of pcDNA; pTT (Durocher et al., Nucleic Acids Research 2002, Vol 30, No. 2); pTT3 (pTT with additional multiple cloning site; pEFBOS (Mizushima, S, and Nagata, S., (1990) Nucleic acids Research Vol 18, No. 17); pBV; pJV; and pBJ.

### Methods of Use

Methods of the present invention include methods of modifying growth factor activity in one or more biological system. Such methods may include contacting one or more biological system with a compound and/or composition of the invention. In some cases, these methods include modifying the level of free growth factor in a biological system (e.g. in a cell niche or subject). Compounds and/or compostions according to such methods may include, but are not limited to biomolecules, including, but not limited to recombinant proteins, protein complexes and/or antibodies described herein.

In some embodiments, methods of the present invention may be used to initiate or increase growth factor activity, termed “activating methods” herein. Some such methods may comprise growth factor release from a GPC and/or inhibition of growth factor reassociation into a latent GPC. In some cases, activating methods may comprise the use of an antibody, a recombinant protein and/or a protein complex. According to some activating methods, one or more activating antibody is provided. In such methods, one or more growth factor may be released or prevented from being drawn back into a GPC. In one, non-limiting example, an anti-prodomain antibody may be provided that enhances dissociation between a growth factor and a GPC and/or prevents reformation of a GPC.

Embodiments of the present invention include methods of using anti-prodomain antibodies to modify growth factor activity. In some cases, such methods may include the use of anti-GDF prodomain antibodies as GDF-activating antibodies.

In some embodiments, methods of the present invention may be used to reduce or eliminate growth factor activity, termed “inhibiting methods” herein. Some such methods may comprise growth factor retention in a GPC and/or promotion of reassociation of growth factor into a latent GPC. In some cases, inhibiting methods may comprise the use of an antibody, a recombinant protein and/or a protein complex. According to some inhibiting methods, one or more inhibiting antibody is provided. In some cases, inhibiting methods comprise the use of inhibiting recombinant proteins or inhibiting protein complexes capable of association with a growth factor, wherein the association prevents growth factor activity.

### Targeting Complexes

In some embodiments methods of the present invention may comprise the use of one or more targeting complex. As used herein, the term “targeting complex” refers to a protein complex wherein at least one protein component acts as a targeting agent. As used herein, the term “targeting agent” refers to an agent that directs cargo or other components complexed with the agent to a target site.

In some cases, targeting complexes may comprise one or more extracellular matrix proteins and/or proteins associated with the extracellular matrix. Such proteins may function as targeting agents in a targeting complex. According to such embodiments, the extracellular matrix component of a targeting complex may direct the complex to target sites comprising extracellular matrix and/or cellular matrix. Extracellular matrix components of targeting complexes may include, but are not limited to LTBPs (e.g. LTBP1, LTBP2, LTBP3 and/or LTBP4), fibrillins (e.g. fibrillin-1, fibrillin-2, fibrillin-3 and/or fibrillin-4), perlecan, decorin, elastin, collagen, GASPs and/or GARPs (e.g. GARP and/or LRRC33).

In some embodiments, LTBP isoforms may be used as targeting agents to direct targeting complexes to areas of extra cellular matrix surrounding different tissues. LTBP1, for example, has been shown to be expressed predominantly in the heart, lung, kidney, placenta, spleen and stomach. As such, targeting complexes may be directed to those organs by incorporation of LTBP1 as a targeting agent. Similarly, LTBP2 is found in the lung, skeletal muscle, liver and placenta while LTBP3 and LTBP4 are both known to be expressed in the skeletal muscle, heart, ovaries and small intestine (Ceco, E. 2013. FEBS J. 280(17):4198-209, the contents of which are herein incorporated by reference in their entirety). These differential regions of expression may be target sites for targeting complexes in which LTBP2, 3 or 4 isoforms may be used as targeting agents.

Some targeting complexes of the invention may comprise one or more prodomain component, such as a prodomain. In some cases, the portion of such targeting complexes may function to bind free growth factors to reduce free growth factor levels and/or activity. In some cases, GDF prodomains may be included in targeting complexes.

### Therapeutics

In some embodiments, compositions and methods of the invention may be used to treat a wide variety of diseases, disorders and/or conditions. In some cases, such diseases, disorders and/or conditions may be TGF-β-related indications. As used herein, the term “TGF-β-related indication” refers to any disease, disorder and/or condition related to expression, activity and/or metabolism of a TGF-β family member protein or any disease, disorder and/or condition that may benefit from modulation of the activity and/or levels of one or more TGF-β family member protein. TGF-β-related indications may include, but are not limited to, fibrosis, anemia of the aging, cancer (including, but not limited to colon, renal, breast, malignant melanoma and glioblastoma), facilitation of rapid hematopoiesis following chemotherapy, bone healing, endothelial proliferation syndromes, asthma and allergy, gastrointestinal disorders, aortic aneurysm, orphan indications (such as Marfan's syndrome and Camurati-Engelmann disease), obesity, diabetes, arthritis, multiple sclerosis, muscular dystrophy, amyotrophic lateral sclerosis (ALS), Parkinson's disease, osteoporosis, osteoarthritis, osteopenia, metabolic syndromes, nutritional disorders, organ atrophy, chronic obstructive pulmonary disease (COPD), and anorexia. Additional indications may include any of those disclosed in US Pub. No. 2013/0122007, U.S. Pat. No. 8,415,459 or International Pub. No. WO 2011/151432, the contents of each of which are herein incorporated by reference in their entirety.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of compositions of the present invention, “effective against” for example a cancer, indicates that administration in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating the particular type of cancer.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given composition or formulation of the present invention can also be judged using an experimental animal model for the given disease as known in the art. When using an experimental animal model, efficacy of treatment is evidenced when a statistically significant change is observed.

### Therapeutics for Anemia, Thrombocytopenia and Neutropenia

During chemotherapy, cell division is temporarily halted to prevent the growth and spread of cancerous cells. An unfortunate side effect is the loss of red blood cells, platelets and white blood cells which depend on active cell division of bone marrow cells. In some embodiments, compounds and/or compositions of the present invention may be designed to treat patients suffering from anemia (the loss of red blood cells), thrombocytopenia (a decrease in the number of platelets) and/or neutropenia (a decrease in the number of neutrophils).

### Therapeutics for Cancer

Various cancers may be treated with compounds and/or compositions of the present invention. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

In cancer, TGF-β may be either growth promoting or growth inhibitory. As an example, in pancreatic cancers, SMAD4 wild type tumors may experience inhibited growth in response to TGF-β, but as the disease progresses, constitutively activated type II receptor is typically present. Additionally, there are SMAD4-null pancreatic cancers. In some embodiments, compounds and/or compositions of the present invention are designed to selectively target components of TGF-β signaling pathways that function uniquely in one or more forms of cancer. Leukemias, or cancers of the blood or bone marrow that are characterized by an abnormal proliferation of white blood cells i.e., leukocytes, can be divided into four major classifications including Acute lymphoblastic leukemia (ALL), Chronic lymphocytic leukemia (CLL), Acute myelogenous leukemia or acute myeloid leukemia (AML) (AML with translocations between chromosome 10 and 11 [010, 11)], chromosome 8 and 21 [t(8;21)], chromosome 15 and 17 [t(15;17)], and inversions in chromosome 16 [inv(16)]; AML with multilineage dysplasia, which includes patients who have had a prior myelodysplastic syndrome (MDS) or myeloproliferative disease that transforms into AML; AML and myelodysplastic syndrome (MDS), therapy-related, which category includes patients who have had prior chemotherapy and/or radiation and subsequently develop AML or MDS; d) AML not otherwise categorized, which includes subtypes of AML that do not fall into the above categories; and e) Acute leukemias of ambiguous lineage, which occur when the leukemic cells cannot be classified as either myeloid or lymphoid cells, or where both types of cells are present); and Chronic myelogenous leukemia (CML).

The types of carcinomas include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

In some embodiments, compositions and methods of the invention may be used to treat one or more types of cancer or cancer-related conditions that may include, but are not limited to colon cancer, renal cancer, breast cancer, malignant melanoma and glioblastomas (Schlingensiepen et al., 2008; Ouhtit et al., 2013).

The invention further relates to the use of compounds and/or compositions of the present invention for treating one or more forms of cancer, in combination with other pharmaceuticals and/or other therapeutic methods, e.g., with known pharmaceuticals and/or known therapeutic methods, such as, for example, those which are currently employed for treating these disorders. For example, the compounds and/or compositions of the present invention can also be administered in conjunction with one or more additional anti-cancer treatments, such as biological, chemotherapy and radiotherapy. Accordingly, a treatment can include, for example, imatinib (Gleevac), all-trans-retinoic acid, a monoclonal antibody treatment (gemtuzumab, ozogamicin), chemotherapy (for example, chlorambucil, prednisone, prednisolone, vincristine, cytarabine, clofarabine, farnesyl transferase inhibitors, decitabine, inhibitors of MDR1), rituximab, interferon-a, anthracycline drugs (such as daunorubicin or idarubicin), L-asparaginase, doxorubicin, cyclophosphamide, doxorubicin, bleomycin, fludarabine, etoposide, pentostatin, or cladribine), bone marrow transplant, stem cell transplant, radiation therapy, anti-metabolite drugs (methotrexate and 6-mercaptopurine), or any combination thereof.

Radiation therapy (also called radiotherapy, X-ray therapy, or irradiation) is the use of ionizing radiation to kill cancer cells and shrink tumors. Radiation therapy can be administered externally via external beam radiotherapy (EBRT) or internally via brachytherapy. The effects of radiation therapy are localized and confined to the region being treated. Radiation therapy may be used to treat almost every type of solid tumor, including cancers of the brain, breast, cervix, larynx, lung, pancreas, prostate, skin, stomach, uterus, or soft tissue sarcomas. Radiation is also used to treat leukemia and lymphoma.

Chemotherapy is the treatment of cancer with drugs that can destroy cancer cells. In current usage, the term “chemotherapy” usually refers to cytotoxic drugs which affect rapidly dividing cells in general, in contrast with targeted therapy. Chemotherapy drugs interfere with cell division in various possible ways, e.g. with the duplication of DNA or the separation of newly formed chromosomes. Most forms of chemotherapy target all rapidly dividing cells and are not specific to cancer cells, although some degree of specificity may come from the inability of many cancer cells to repair DNA damage, while normal cells generally can.

Most chemotherapy regimens are given in combination. Exemplary chemotherapeutic agents include , but are not limited to, 5-FU Enhancer, 9-AC, AG2037, AG3340, Aggrecanase Inhibitor, Aminoglutethimide, Amsacrine (m-AMSA), Asparaginase, Azacitidine, Batimastat (BB94), BAY 12-9566, BCH-4556, Bis-Naphtalimide, Busulfan, Capecitabine, Carboplatin, Carmustaine+Polifepr Osan, cdk4/cdk2 inhibitors, Chlorombucil, CI-994, Cisplatin, Cladribine, CS-682, Cytarabine HCl, D2163, Dactinomycin, Daunorubicin HCl, DepoCyt, Dexifosamide, Docetaxel, Dolastain, Doxifluridine, Doxorubicin, DX8951f, E 7070, EGFR, Epirubicin, Erythropoietin, Estramustine phosphate sodium, Etoposide (VP16-213), Farnesyl Transferase Inhibitor, FK 317, Flavopiridol, Floxuridine, Fludarabine, Fluorouracil (5-FU), Flutamide, Fragyline, Gemcitabine, Hexamethylmelamine (HMM), Hydroxyurea (hydroxycarbamide), Ifosfamide, Interferon Alfa-2a, Interferon Alfa-2b, Interleukin-2, Irinotecan, ISI 641, Krestin, Lemonal DP 2202, Leuprolide acetate (LHRH-releasing factor analogue), Levamisole, LiGLA (lithium-gamma linolenate), Lodine Seeds, Lometexol, Lomustine (CCNU), Marimistat, Mechlorethamine HCl (nitrogen mustard), Megestrol acetate, Meglamine GLA, Mercaptopurine, Mesna, Mitoguazone (methyl-GAG; methyl glyoxal bis-guanylhydrazone; MGBG), Mitotane (o.p′-DDD), Mitoxantrone, Mitoxantrone HCl, MMI 270, MMP, MTA/LY 231514, Octreotide, ODN 698, OK-432, Oral Platinum, Oral Taxoid, Paclitaxel (TAXOL.®.), PARP Inhibitors, PD 183805, Pentostatin (2′ deoxycoformycin), PKC 412, Plicamycin, Procarbazine HCl, PSC 833, Ralitrexed, RAS Farnesyl Transferase Inhibitor, RAS Oncogene Inhibitor, Semustine (methyl-CCNU), Streptozocin, Suramin, Tamoxifen citrate, Taxane Analog, Temozolomide, Teniposide (VM-26), Thioguanine, Thiotepa, Topotecan, Tyrosine Kinase, UFT (Tegafur/Uracil), Valrubicin, Vinblastine sulfate, Vindesine sulfate, VX-710, VX-853, YM 116, ZD 0101, ZD 0473/Anormed, ZD 1839, ZD 9331.

Biological therapies use the body's immune system, either directly or indirectly, to fight cancer or to lessen the side effects that may be caused by some cancer treatments. In some embodiments, compounds and/or compositions of the present invention may be considered biological therapies in that they may stimulate immune system action against one or more tumor, for example. However, this approach may also be considered with other such biological approaches, e.g., immune response modifying therapies such as the administration of interferons, interleukins, colony-stimulating factors, other monoclonal antibodies, vaccines, gene therapy, and nonspecific immunomodulating agents are also envisioned as anti-cancer therapies to be combined with the compounds and/or compositions of the present invention.

Small molecule targeted therapy drugs are generally inhibitors of enzymatic domains on mutated, overexpressed, or otherwise critical proteins within the cancer cell, such as tyrosine kinase inhibitors imatinib (Gleevec/Glivec) and gefitinib (Iressa). Examples of monoclonal antibody therapies that can be used with compounds and/or compositions of the present invention include, but are not limited to, the anti-HER2/neu antibody trastuzumab (Herceptin) used in breast cancer, and the anti-CD20 antibody rituximab, used in a variety of B-cell malignancies. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial.

Cancer immunotherapy refers to a diverse set of therapeutic strategies designed to induce the patient's own immune system to fight the tumor, and include, but are not limited to, intravesical BCG immunotherapy for superficial bladder cancer, vaccines to generate specific immune responses, such as for malignant melanoma and renal cell carcinoma, and the use of Sipuleucel-T for prostate cancer, in which dendritic cells from the patient are loaded with prostatic acid phosphatase peptides to induce a specific immune response against prostate-derived cells.

### Therapeutics for Angiogenic and Endothelial Proliferation Conditions

The compounds and/or compositions of the present invention may be used to treat angiogenic and endothelial proliferation syndromes, diseases or disorders. The term “angiogenesis”, as used herein refers to the formation and/or reorganization of new blood vessels. Angiogenic disease involves the loss of control over angiogenesis in the body. In such cases, blood vessel growth, formation or reorganization may be overactive (including during tumor growth and cancer where uncontrolled cell growth requires increased blood supply) or insufficient to sustain healthy tissues. Such conditions may include, but are not limited to angiomas, angiosarcomas, telangiectasia, lymphangioma, congenital vascular anomalies, tumor angiogenesis and vascular structures after surgery. Excessive angiogenesis is noted in cancer, macular degeneration, diabetic blindness, rheumatoid arthritis, psoriasis as well as many other conditions. Excessive angiogenesis is often promoted by excessive angiogenic growth factor expression. Compounds and/or compositions of the present invention may act to block growth factors involved in excessive angiogenesis. Alternatively, compounds and/or compositions of the present invention may be utilized to promote growth factor signaling to enhance angiogenesis in conditions where angiogenesis is inhibited. Such conditions include, but are not limited to coronary artery disease, stroke, diabetes and chronic wounds.

### Therapeutics for Cardiovascular Indications

In some embodiments, compounds and/or compositions of the present invention may be used to treat one or more cardiovascular indications, including, but not limited to cardiac hypertrophy. Cardiac hypertrophy comprises enlargement of the heart due, typically due to increased cell volume of cardiac cells (Aurigemma 2006. N Engl J Med. 355(3):308-10). Age-related cardiac hypertrophy may be due, in part, to reduced circulating levels of GDF-11. A study by Loffredo et al (Loffredo et al., 2013. Cell. 153:828-39) found that fusion of the circulatory system between young and old mice had a protective effect with regard to cardiac hypertrophy. The study identified GDF-11 as a circulating factor that decreased with age in mice and was able to show that its administration could also reduce cardiac hypertrophy. Some compounds and/or compositions of the present invention may be used to treat and/or prevent cardiac atrophy. Such compounds and/or compositions may comprise GDF-11 agonists that elevate levels of circulating GDF-11, in some cases through enhancing the dissociation of GDF-11 growth factor from latent GPCs.

In some embodiments, animal models may be used to develop and test compounds and/or compositions of the present invention for use in the treatment of cardiovascular diseases, disorders and/or conditions. In some cases, vascular injury models may be used to test compounds in the treatment of atherosclerosis and/or restenosis. Such models may include balloon injury models. In some cases, these may be carried out as described in Smith et al., 1999. Circ Res. 84(10):1212-22, the contents of which are herein incorporated by reference in their entirety.

Therapeutics Related to Muscle Disorders and/or Injuries

In some embodiments, compounds and/or compositions of the present invention may be used to treat one or more muscle disorders and/or injuries. In some cases, such compounds and/or composition may include, but are not limited to antibodies that modulate GDF-11 activity. Muscle comprises about 40-50% of total body weight, making it the largest organ in the body. Muscle disorders may include cachexia (e.g. muscle wasting). Muscle wasting may be associated with a variety of diseases and catabolic disorders (e.g. HIV/AIDS, cancer, cancer cachexia, renal failure, congestive heart failure, muscular dystrophy, disuse atrophy, chronic obstructive pulmonary disease, motor neuron disease, trauma, neurodegenerative disease, infection, rheumatoid arthritis, immobilization, diabetes, etc.). In such disorders, GDF signaling activity may contribute to muscle catabolism (Han et al., 2013. Int J Biochem Cell Biol. 45(10):2333-47; Lee., 2010. Immunol Endocr Metab Agents Med Chem. 10:183-94, the contents of each of which are herein incorporated by reference in their entirety). Other muscle disorders may comprise sarcopenia. Sarcopenia is the progressive loss of muscle and function associated with aging. In the elderly, sarcopenia can cause frailty, weakness, fatigue and loss of mobility (Morely. 2012. Family Practice. 29:i44-i48). With the aged population increasing in numbers, sarcopenia is progressively becoming a more serious public health concern. A study by Hamrick et al (Hamrick et al., 2010. 69(3):579-83) demonstrated that GDF inhibition could repair muscle in a mouse model of fibula osteotomoy comprising lateral compartment muscle damage. Administration of GDF propeptides was sufficient to increase muscle mass by nearly 20% as well as improve fracture healing. Some compounds and/or compositions of the present invention may be used to treat muscle diseases, disorders and/or injuries by modulating GDF-11 activity. In some cases, compounds of the present invention may be GDF-11 signaling antagonists, preventing or reducing GDF-11 signaling activity.

Inclusion body myositis (IBM) is a disease characterized by progressive muscle loss, typically occurring in mid- to late-life. The disease is thought to occur due to an autoimmune response to autoantigens in the muscle causing T-cell invasion of the muscle fiber and resulting in myofiber destruction (Greenberg 2012. Curr Opin Neurol. 25(5):630-9). Therapeutic compounds are being investigated, including Bimagrumab (BYM338; Novartis, Basel, Switzerland), an antibody that targets type II activin receptors, preventing GDF and/or activin signal transduction, thereby stimulating muscle production and strengthening [see clinical trial number NCT01925209 entitled Efficacy and Safety of Bimagrumab/BYM338 at 52 Weeks on Physical Function, Muscle Strength, Mobility in sIBM Patients (RESILIENT)]. Some compounds and/or compositions of the present invention may be used to treat subjects with IBM. In some cases, such compounds and/or compositions may block GDF-11 activity (e.g. through stabilization of GDF-11 GPCs). In addition to IBM, BYM338 is being investigated for treatment of chronic obstructive pulmonary disease (COPD). In some cases, compounds and/or compositions of the present invention utilized for IBM treatment, may be used to treat COPD as well. In some cases, compounds and/or compositions of the present invention may be administered in combination and/or coordination with BYM338.

### Therapeutics for Diabetes

Skeletal muscle uses and stores glucose for fuel. Due to this, skeletal muscle is an important regulator of circulating glucose levels. Uptake of glucose by muscle can be stimulated by either contraction or by insulin stimulation (McPherron et al., 2013. Adipocyte. 2(2):92-8, herein incorporated by reference in its entirety). A recent study by Guo et al (Guo, et al., 2012. Diabetes 61(10):2414-23) found that when GDF receptor-deficient mice were crossed with A-ZIP/F1 mice (a lipodistrophic mouse strain, used as a diabetic model), hybrid off-spring showed reduced levels of blood glucose and improved sensitivity to insulin. Hyperphagia (excessive eating) was also reduced in these mice. As another example, GDF receptor signaling has been implicated in the de-differentiation of pancreatic β cells that occurs in Type II diabetes. See Blum B., et al., “Reversal of β cell de-differentiation by a small molecule inhibitor of the TGFβ pathway” eLife (2014) 3:e02809, 1-17; the entire contents of which are incorporated by reference herein. An inhibitor of the Alk5 receptor was shown to restore mature β cells in a model of severe type II diabetes. Additionally, GDF11 deficient animals have defects in the development of the pancreas, displaying exocrine hypoplasia and an increase in NGN3+endocrine precursor cells, effects on differentiated endocrine cells were not consistent between the two studies. See Dichmann D. S., et al., “Analysis of Pancreatic Endocrine Development in GDF11-Deficient Mice” Developmantal Dynamics (2006) 235:3016-3025; and Harmon E. B., et al., “GDF11 modulates NGN3⁺ islet progenitor cell number and promotes β-cell differentiation in pancreas development” Development (2004) 131, 6163-6174; the entire contents of each are incorporated by reference herein. In some embodiments, compound and/or compositions of the present invention may be used to treat diabetes and/or hyperphagia. Some such treatments may be used to reduce blood glucose and/or improve insulin sensitivity. In some cases, such treatments may comprise GDF-11 signaling antagosists, such as one or more antibodies that prevent dissociation of GDF-11 from its prodomain.

### Therapeutics for GDF-11-Related Indications

In some embodiments, compounds of the invention may be used to treat TGF-β-related indications comprising GDF-11-related indications. As used herein, a GDF-11-related indication is a disease, disorder and/or condition related to GDF-11 activity. GDF-11 expression is systemic and its activity is thought to be involved in multiple processes (Lee et al., 2013. PNAS. 110(39):E3713-22, the contents of which are herein incorporated by reference in their entirety). It is believed to be involved in development of multiple tissues, including, but not limited to the retina, kidney, pancreas and olfactory system. It is also believed to be a circulating factor in the blood. Recent studies indicate that GDF-11 may rejuvenate skeletal muscle, improve cerebral circulation and promote neurogenesis (Sinha, M. et al., 2014. Science Express. 10.1126/science.1251152, p2-6 and Katsimpardi, L. et al., 2014. Science Express. 10.1126/science.1251141, the contents of each of which are herein incorporated by reference in their entirety). In some cases, antibodies of the invention may promote skeletal muscle rejuvenation, improve cerebral circulation and promote neurogenesis by promoting the release of GDF-11 growth factor from latent complexes.

Although its role is somewhat controversial, GDF-11 is thought to be involved in the regulation of erythropoiesis with both positive and negative regulation being described in scientific literature. Carrancio et al and Suragani et al (Carrancio, S. et al., 2014. Br J Haematol. 165(6):870-82 and Suragani, R.N.V.S. et al. 2014. Blood. 123(25): 3864-72, the contents of each of which are herein incorporated by reference in their entirety) demonstrate that a GDF-11 ligand trap, comprising an activin receptor IIA extracellular domain (SOTATERCEPT®), enhances erythropoiesis. A similar agent shown to bind GDF-11 and modified to reduce affinity for activin, ACE-536, was also shown to stimulate erythropoiesis (Suragani, R.N.V.S. et al. 2014. Nature Medicine. 20(4): 408-17, the contents of which are herein incorporated by reference in their entirety). Further studies demonstrate short term increases in hemoglobin levels in patients receiving SOTATERCEPT® (El-Shahawy, M. et al., 2014. Poster #81, National Kidney Foundation (NKF) 2014 Spring Clinical Meeting). In some cases, GDF-11 inhibiting antibodies of the invention may be used according to the methods described in these studies to stimulate erythropoiesis and treat anemia and/or β-thalassemia.

### Veterinary Applications

In some embodiments, it is contemplated that compositions and methods of the invention will find utility in the area of veterinary care including the care and treatment of non-human vertebrates. As described herein, the term “vertebrate” includes all vertebrates including, but not limited to fish, amphibians, birds, reptiles and mammals (including, but not limited to alpaca, banteng, bison, camel, cat, cattle, deer, dog, donkey, gayal, goat, guinea pig, horse, llama, mice, monkeys, mule, pig, rabbit, rats, reindeer, sheep water buffalo, yak and humans). As used herein the term “non-human vertebrate” refers to any vertebrate with the exception of humans (i.e. Homo sapiens). Exemplary non-human vertebrates include wild and domesticated species such as companion animals and livestock. Livestock include domesticated animals raised in an agricultural setting to produce materials such as food, labor, and derived products such as fiber and chemicals. Generally, livestock includes all mammals, avians and fish having potential agricultural significance. In particular, four-legged slaughter animals include steers, heifers, cows, calves, bulls, cattle, swine and sheep.

### Bioprocessing

In some embodiments, the present invention provides methods for producing one or more biological products in host cells by contacting such cells with compounds and/or compositions of the present invention capable of modulating expression of target genes, or altering the level of growth factor signaling molecules wherein such modulation or alteration enhances production of biological products. According to the present invention, bioprocessing methods may be improved by using one or more compounds and/or compositions of the present invention. They may also be improved by supplementing, replacing or adding one or more compounds and/or compositions.

### Pharmaceutical Compositions

In some embodiments, the pharmaceutical compositions described herein may be characterized by one or more of bioavailability, therapeutic window and/or volume of distribution.

### Bioavailability

In some embodiments, pharmaceutical compositions comprise complexes of compounds and/or compositions of the present invention with GPCs. In such embodiments, complexes may be implanted at desired therapeutic sites where steady dissociation of growth factors from complexes may occur over a desired period of time. In some embodiments, implantation complexes may be carried out in association with sponge and/or bone-like matrices. Such implantations may include, but are not limited to dental implant sites and/or sites of bone repair.

In some embodiments, compounds and/or compositions of the present invention are made in furin-deficient cells. GPCs produced in such cells may be useful for treatment in areas where release is slowed due to the fact that furin cleavage in vivo is rate-limiting during GPC processing. In some embodiments, one or more tolloid and/or furin sites in GPCs are mutated, slowing the action of endogenous tolloid and/or furin proteases. In such embodiments, growth factor release may be slowed (e.g. at sites of implantation).

Antibodies of the present invention, when formulated into compositions with delivery/formulation agents or vehicles as described herein, may exhibit increased bioavailability as compared to compositions lacking delivery agents as described herein. As used herein, the term “bioavailability” refers to the systemic availability of a given amount of a particular agent administered to a subject. Bioavailability may be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound may be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in their entirety.

Cₘₐₓ values are maximum concentrations of compounds achieved in serum or plasma of a subject following administration of compounds to the subject. Cₘₐₓ values of particular compounds may be measured using methods known to those of ordinary skill in the art. As used herein, the phrases “increasing bioavailability” or “improving the pharmacokinetics,” refer to actions that may increase the systemic availability of a compounds and/or compositions of the present invention (as measured by AUC, Cₘₐₓ, or Cₘᵢₙ) in a subject. In some embodiments, such actions may comprise co-administration with one or more delivery agents as described herein. In some embodiments, the bioavailability of compounds and/or compositions may increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%.

### Therapeutic Window

Compounds and/or compositions of the present invention, when formulated with one or more delivery agents as described herein, may exhibit increases in the therapeutic window of compound and/or composition administration as compared to the therapeutic window of compounds and/or compositions administered without one or more delivery agents as described herein. As used herein, the term “therapeutic window” refers to the range of plasma concentrations, or the range of levels of therapeutically active substance at the site of action, with a high probability of eliciting a therapeutic effect. In some embodiments, therapeutic windows of compounds and/or compositions when co-administered with one or more delivery agent as described herein may increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%.

### Volume of Distribution

Compounds and/or compositions of the present invention, when formulated with one or more delivery agents as described herein, may exhibit an improved volume of distribution (V_{dist}), e.g., reduced or targeted, relative to formulations lacking one or more delivery agents as described herein. V_{dist} relates the amount of an agent in the body to the concentration of the same agent in the blood or plasma. As used herein, the term “volume of distribution” refers to the fluid volume that would be required to contain the total amount of an agent in the body at the same concentration as in the blood or plasma: V_{dist} equals the amount of an agent in the body/concentration of the agent in blood or plasma. For example, for a 10 mg dose of a given agent and a plasma concentration of 10 mg/L, the volume of distribution would be 1 liter. The volume of distribution reflects the extent to which an agent is present in the extravascular tissue. Large volumes of distribution reflect the tendency of agents to bind to the tissue components as compared with plasma proteins. In clinical settings, V_{dist} may be used to determine loading doses to achieve steady state concentrations. In some embodiments, volumes of distribution of compounds and/or compositions of the present invention when co-administered with one or more delivery agents as described herein may decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Formulation, Administration, Delivery and Dosing

In some embodiments, compounds and/or compositions of the present invention are pharmaceutical compositions. In some embodiments, pharmaceutical compositions may optionally comprise one or more additional active substances, e.g. therapeutically and/or prophylactically active substances. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

In some embodiments, compositions may be administered to humans, human patients or subjects. For the purposes of the present disclosure, the phrase “active ingredient” generally refers to compounds and/or compositions of the present invention to be delivered as described herein.

Although the descriptions of pharmaceutical compositions provided herein are principally directed to pharmaceutical compositions which are suitable for administration to humans, it will be understood by the skilled artisan that such compositions are generally suitable for administration to other subjects, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, formulations of the pharmaceutical compositions described herein may be prepared by any method known or hereafter developed in the art of pharmacology. In general, such preparatory methods include the step of bringing active ingredients into association with excipients and/or one or more other accessory ingredients, and then, if necessary and/or desirable, dividing, shaping and/or packaging products into desired single- or multi-dose units.

In some embodiments, pharmaceutical compositions of the present invention may be prepared, packaged, and/or sold in bulk, as single unit doses, and/or as a plurality of single unit doses. As used herein, the term “unit dose” refers to a discrete amount of the pharmaceutical composition comprising a predetermined amount of active ingredient. Amounts of active ingredient are generally equal to the dosage of active ingredients which would be administered to subjects and/or convenient fractions of such a dosages such as, for example, one-half or one-third of such a dosages.

In some embodiments, relative amounts of active ingredients, pharmaceutically acceptable excipients, and/or any additional ingredients in pharmaceutical compositions of the present invention may vary, depending upon identity, size, and/or condition of subjects to be treated and further depending upon routes by which compositions are to be administered. By way of example, compositions may comprise between about 0.1% and 100%, e.g., from about 0.5% to about 50%, from about 1% to about 30%, from about 5% to about 80% or at least 80% (w/w) active ingredient. In some embodiments, active ingredients are antibodies directed toward regulatory elements and/or GPCs.

### Formulations

Compounds and/or compositions of the present invention may be formulated using one or more excipients to: (1) increase stability; (2) increase cell permeability; (3) permit the sustained or delayed release (e.g., of compounds and/or growth factors from such formulations); and/or (4) alter the biodistribution (e.g., target compounds to specific tissues or cell types). In addition to traditional excipients such as any and all solvents, dispersion media, diluents, liquid vehicles, dispersion aids, suspension aids, surface active agents, isotonic agents, thickening agents, emulsifying agents and preservatives, formulations of the present invention may comprise, without limitation, liposomes, lipid nanoparticles, polymers, lipoplexes, core-shell nanoparticles, peptides, proteins, cells transfected with the compounds and/or compositions of the present invention (e.g., for transplantation into subjects) and combinations thereof.

### Excipients

Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference).

In some embodiments, the use of conventional excipient media are contemplated within the scope of the present disclosure, except insofar as any conventional excipient media may be incompatible with substances and/or their derivatives, such as by producing any undesirable biological effects or otherwise interacting in deleterious manners with any other component(s) of pharmaceutical compositions.

Formulations of pharmaceutical compositions described herein may be prepared by any method known or hereafter developed in the art of pharmacology. In general, such preparatory methods include steps of associating active ingredients with excipients and/or other accessory ingredients.

Pharmaceutical compositions, in accordance with the present disclosure, may be prepared, packaged, and/or sold in bulk, as single unit doses, and/or as a plurality of single unit doses.

Relative amounts of active ingredients, pharmaceutically acceptable excipients, and/or additional ingredients in pharmaceutical compositions of the present disclosure may vary, depending upon identity, size, and/or condition of subjects being treated and further depending upon routes by which pharmaceutical compositions may be administered.

In some embodiments, pharmaceutically acceptable excipient are at least 95%, at least 96%, at least 97%, at least 98%, at least 99% or 100% pure. In some embodiments, excipients are approved for use in humans and/or for veterinary use. In some embodiments, excipients are approved by the United States Food and Drug Administration. In some embodiments, excipients are pharmaceutical grade. In some embodiments, excipients meet the standards of the United States Pharmacopoeia (USP), the European Pharmacopoeia (EP), the British Pharmacopoeia, and/or the International Pharmacopoeia.

In some embodiments, pharmaceutically acceptable excipients of the present invention may include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [Span®60], sorbitan tristearate [Span®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol),; natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Formulation Vehicles: Liposomes, Lipoplexes, and Lipid Nanoparticles

Compounds and/or compositions of the present invention may be formulated using one or more liposomes, lipoplexes and/or lipid nanoparticles. In some embodiments, pharmaceutical compositions comprise liposomes. Liposomes are artificially-prepared vesicles which may primarily be composed of a lipid bilayer and may be used as delivery vehicles for the administration of nutrients and pharmaceutical formulations. Liposomes may be of different sizes such as, but not limited to, multilamellar vesicles (MLVs) which may be hundreds of nanometers in diameter and may contain a series of concentric bilayers separated by narrow aqueous compartments, small unicellular vesicle (SUVs) which may be smaller than 50 nm in diameter and large unilamellar vesicle (LUVs) which may be between 50 and 500 nm in diameter. Liposome components may include, but are not limited to, opsonins or ligands in order to improve the attachment of liposomes to unhealthy tissue or to activate events such as, but not limited to, endocytosis. Liposomes may comprise low or high pH. In some embodiments, liposome pH may be varied in order to improve delivery of pharmaceutical formulations.

In some embodiments, liposome formation may depend on physicochemical characteristics such as, but not limited to, the pharmaceutical formulation entrapped, liposomal ingredients, the nature of the medium in which lipid vesicles are dispersed, the effective concentration of entrapped substances, potential toxicity of entrapped substances, additional processes involved during the application and/or delivery of vesicles, optimization size, polydispersity, shelf-life of vesicles for the intended application, batch-to-batch reproducibility and possibility of large-scale production of safe and efficient liposomal products.

In some embodiments, formulations may be assembled or compositions altered such that they are passively or actively directed to different cell types in vivo.

In some embodiments, formulations may be selectively targeted through expression of different ligands on formulation surfaces as exemplified by, but not limited by, folate, transferrin, N-acetylgalactosamine (GalNAc), and antibody targeted approaches.

In some embodiments, pharmaceutical compositions of the present invention may be formulated with liposomes, lipoplexes and/or lipid nanoparticles to improve efficacy of function. Such formulations may be able to increase cell transfection by pharmaceutical compositions. In some embodiments, liposomes, lipoplexes, or lipid nanoparticles may be used to increase pharmaceutical composition stability.

In some embodiments, liposomes are specifically formulated for pharmaceutical compositions comprising one or more antibodies. Such liposomes may be prepared according to techniques known in the art, such as those described by Eppstein et al. (Eppstein, D. A. et al., Biological activity of liposome-encapsulated murine interferon gamma is mediated by a cell membrane receptor. Proc Natl Acad Sci USA. 1985 June; 82(11):3688-92); Hwang et al. (Hwang, K. J. et al., Hepatic uptake and degradation of unilamellar sphingomyelin/cholesterol liposomes: a kinetic study. Proc Natl Acad Sci USA. 1980 July; 77(7):4030-4); U.S. Pat. No. 4,485,045 and U.S. Pat. No. 4,544,545. Production of liposomes with sustained circulation time are also described in U.S. Pat. No. 5,013,556.

In some embodiments, liposomes of the present invention comprising antibodies may be generated using reverse phase evaporation utilizing lipids such as phosphatidylcholine, cholesterol as well as phosphatidylethanolamine that have been polyethylene glycol-derivatized. Filters with defined pore size are used to extrude liposomes of the desired diameter. In another embodiment, compounds and/or compositions of the present invention may be conjugated to external surfaces of liposomes by disulfide interchange reactions as is described by Martin et al. (Martin, F. J. et al., Irreversible coupling of immunoglobulin fragments to preformed vesicles. An improved method for liposome targeting. J Biol Chem. 1982 Jan. 10; 257(1):286-8).

### Formulation Vehicles: Polymers and Nanoparticles

Compounds and/or compositions of the present invention may be formulated using natural and/or synthetic polymers. Non-limiting examples of polymers which may be used for delivery include, but are not limited to DMRI/DOPE, poloxamer, chitosan, cyclodextrin, and poly(lactic-co-glycolic acid) (PLGA) polymers. In some embodiments, polymers may be biodegradable.

In some embodiments, polymer formulation may permit sustained and/or delayed release of compounds and/or compositions (e.g., following intramuscular and/or subcutaneous injection). Altered release profile for compounds and/or compositions of the present invention may result in, for example, compound release over an extended period of time. Polymer formulations may also be used to increase the stability of compounds and/or compositions of the present invention.

In some embodiments, polymer formulations may be selectively targeted through expression of different ligands as exemplified by, but not limited by, folate, transferrin, and N-acetylgalactosamine (GalNAc) (Benoit, D. S. et al., Synthesis of folate-functionalized RAFT polymers for targeted siRNA delivery. Biomacromolecules. 2011 12:2708-14; Rozema, D. B. et al., Dynamic polyconjugates for targeted in vivo delivery of siRNA to hepatocytes. Proc Natl Acad Sci USA. 2007 104:12982-12887; Davis, M. E. et al., The first targeted delivery of siRNA in humans via a self-assembling, cyclodextrin polymer-based nanoparticle: from concept to clinic. Mol Pharm. 2009 6:659-668; Davis, M. E. et al., Evidence of RNAi in humans from systemically administered siRNA via targeted nanoparticles. Nature. 2010. 464:1067-70; the contents of each of which are herein incorporated by reference in their entirety).

Compounds and/or compositions of the present invention may be formulated as nanoparticles using combinations of polymers, lipids, and/or other biodegradable agents, such as, but not limited to, calcium phosphates. In some embodiments, components may be combined in core-shells, hybrids, and/or layer-by-layer architectures, to allow for fine-tuning of nanoparticle structure, so delivery may be enhanced. For antibodies of the present invention, systems based on poly(2-(methacryloyloxy)ethyl phosphorylcholine)-block-(2-(diisopropylamino)ethyl methacrylate), (PMPC-PDPA), a pH sensitive diblock copolymer that self-assembles to form nanometer-sized vesicles, also known as polymersomes, at physiological pH may be used. These polymersomes have been shown to successfully deliver relatively high antibody payloads within live cells. (Massignani, M. et al., Cellular delivery of antibodies: effective targeted subcellular imaging and new therapeutic tool. Nature Proceedings. 2010. p 1-1′7).

In some embodiments, PEG-charge-conversional polymers (Pitella, F. et al., Enhanced endosomal escape of siRNA-incorporating hybrid nanoparticles from calcium phosphate and PEG-block charge-conversional polymer for efficient gene knockdown with negligible cytotoxicity. Biomaterials. 2011 32:3106-14) may be used to form nanoparticles for delivery of compounds and/or compositions of the present invention. In some embodiments, PEG-charge-conversional polymers may improve upon PEG-polyanion block copolymers by being cleaved into polycations at acidic pH, thus enhancing endosomal escape.

In some embodiments, complexation, delivery and/or internalization of polymeric nanoparticles may be precisely controlled by altering chemical compositions in both core and shell nanoparticle components (Siegwart, D. J. et al., Combinatorial synthesis of chemically diverse core-shell nanoparticles for intracellular delivery. Proc Natl Acad Sci USA. 2011 108:12996-3001).

In some embodiments, matrices of poly(ethylene-co-vinyl acetate), are used to deliver compounds and/or compositions of the invention. Such matrices have bee described by others (Sherwood, J. K. et al., Controlled antibody delivery systems. Nature Biotechnology. 1992. 10:1446-9).

### Binding Protein Formulations

Binding proteins (e.g., antibodies or antigen binding portions thereof) of the present invention may be formulated for intravenous administration or extravascular administration (Daugherty, et al., Formulation and delivery issues for monoclonal antibody therapeutics. Adv Drug Deliv Rev. 2006 Aug. 7; 58(5-6):686-706 and US patent application publication number US2011/0135570, the contents of each of which are herein incorporated by reference in their entirety). Extravascular administration routes may include, but are not limited to subcutaneous administration, intraperitoneal administration, intracerebral administration, intraocular administration, intralesional administration, topical administration and intramuscular administration.

In some embodiments, binding protein structures may be modified to improve effectiveness as therapeutics. Improvements may include, but are not limited to improved thermodynamic stability, reduced Fc receptor binding properties and/or imporved folding efficiency. Modifications may include, but are not limited to amino acid substitutions, glycosylation, palmitoylation and/or protein conjugation.

In some embodiments, binding proteins (e.g., antibodies or antigen binding portions thereof) of the present invention may be formulated with antioxidants to reduce antibody oxidation. Antibodies of the present invention may also be formulated with additives to reduce protein aggregation. Such additives may include, but are not limited to albumin, amino acids, sugars, urea, guanidinium chloride, polyalchohols, polymers (such as polyethylene glycol and dextrans), surfactants (including, but not limited to polysorbate 20 and polysorbate 80) or even other antibodies.

In some embodiments, binding proteins (e.g., antibodies or antigen binding portions thereof) of the present invention may be formulated to reduce the impact of water on binding protein structure and function. Antibody preparartions in such formulations may be may be lyophilized. Formulations subject to lyophilization may include carbohydrates or polyol compounds to protect and/or stabilize antibody structure. Such compounds may include, but are not limited to sucrose, trehalose and mannitol.

In some embodiments, binding proteins (e.g., antibodies or antigen binding portions thereof) of the present invention may be formulated with polymers. In some embodiments, polymer formulations may comprise hydrophobic polymers. Such polymers may be microspheres formulated with polylactide-co-glycolide through solid-in-oil-in-water encapsulation methods. In some embodiments, microspheres comprising ethylene-vinyl acetate copolymer may also be used for antibody delivery and/or to extend the time course of antibody release at sites of delivery. In some embodiments, polymers may be aqueous gels. Such gels may, for example, comprise carboxymethylcellulose. In some embodiments, aqueous gels may also comprise hyaluronic acid hydrogels. In some embodiments, antibodies may be covalently linked to such gels through hydrazone linkages that allow for sustained delivery in tissues, including but not limited to tissues of the central nervous system.

### Formulation Vehicles: Peptides and Proteins

Compounds and/or compositions of the present invention may be formulated with peptides and/or proteins. In some embodiments, peptides such as, but not limited to, cell penetrating peptides and/or proteins/peptides that enable intracellular delivery may be used to deliver pharmaceutical formulations. Non-limiting examples of a cell penetrating peptides which may be used with pharmaceutical formulations of the present invention include cell-penetrating peptide sequences attached to polycations that facilitates delivery to the intracellular space, e.g., HIV-derived TAT peptide, penetratins, transportans, or hCT derived cell-penetrating peptides (see, e.g. Caron, N.J. et al., Intracellular delivery of a Tat-eGFP fusion protein into muscle cells. Mol Ther. 2001. 3(3):310-8; Langel, U., Cell-Penetrating Peptides: Processes and Applications, CRC Press, Boca Raton Fla., 2002; El-Andaloussi, S. et al., Cell-penetrating peptides: mechanisms and applications. Curr Pharm Des. 2003. 11(28):3597-611; and Deshayes, S. et al., Cell-penetrating peptides: tools for intracellular delivery of therapeutics. Cell Mol Life Sci. 2005. 62(16):1839-49, the contents of each of which are herein incorporated by reference in their entirety). Compounds and/or compositions of the present invention may also be formulated to include cell penetrating agents, e.g., liposomes, which enhance delivery of the compositions to intracellular spaces. Compounds and/or compositions of the present invention may be complexed with peptides and/or proteins such as, but not limited to, peptides and/or proteins from Aileron Therapeutics (Cambridge, Mass.) and Permeon Biologics (Cambridge, Mass.) in order to enable intracellular delivery (Cronican, J. J. et al., Potent delivery of functional proteins into mammalian cells in vitro and in vivo using a supercharged protein. ACS Chem Biol. 2010. 5:747-52; McNaughton, B. R. et al., Mammalian cell penetration, siRNA transfection, and DNA transfection by supercharged proteins. Proc Natl Acad Sci, USA. 2009. 106:6111-6; Verdine, G. L. et al., Stapled peptides for intracellular drug targets. Methods Enzymol. 2012. 503:3-33; the contents of each of which are herein incorporated by reference in their entirety).

In some embodiments, the cell-penetrating polypeptides may comprise first and second domains. First domains may comprise supercharged polypeptides. Second domains may comprise protein-binding partner. As used herein, protein-binding partners may include, but are not limited to, antibodies and functional fragments thereof, scaffold proteins and/or peptides. Cell-penetrating polypeptides may further comprise intracellular binding partners for protein-binding partners. In some embodiments, cell-penetrating polypeptides may be capable of being secreted from cells where compounds and/or compositions of the present invention may be introduced.

Compositions of the present invention comprising peptides and/or proteins may be used to increase cell transfection and/or alter compound/composition biodistribution (e.g., by targeting specific tissues or cell types).

### Formulation Vehicles: Cells

Cell-based formulations of compounds and/or compositions of the present invention may be used to ensure cell transfection (e.g., in cellular carriers) or to alter biodistribution (e.g., by targeting cell carriers to specific tissues or cell types).

### Cell Transfer Methods

A variety of methods are known in the art and suitable for introduction of nucleic acids or proteins into cells, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

The technique of sonoporation, or cellular sonication, is the use of sound (e.g., ultrasonic frequencies) for modifying the permeability of cell plasma membranes. Sonoporation methods are known to those in the art and are used to deliver nucleic acids in vivo (Yoon, C. S. et al., Ultrasound-mediated gene delivery. Expert Opin Drug Deliv. 2010 7:321-30; Postema, M. et al., Ultrasound-directed drug delivery. Curr Pharm Biotechnol. 2007 8:355-61; Newman, C. M. et al., Gene therapy progress and prospects: ultrasound for gene transfer. Gene Ther. 2007. 14(6):465-75; the contents of each of which are herein incorporated by reference in their entirety). Sonoporation methods are known in the art and are also taught for example as they relate to bacteria in US Patent application publication US2010/0196983 and as it relates to other cell types in, for example, US Patent application publication US2010/0009424, the contents of each of which are incorporated herein by reference in their entirety.

Electroporation techniques are also well known in the art and are used to deliver nucleic acids in vivo and clinically (Andre, F. M. et al., Nucleic acids electrotransfer in vivo: mechanisms and practical aspects. Curr Gene Ther. 2010 10:267-80; Chiarella, P. et al., Application of electroporation in DNA vaccination protocols. Curr Gene Ther. 2010. 10:281-6; Hojman, P., Basic principles and clinical advancements of muscle electrotransfer. Curr Gene Ther. 2010 10:128-38; the contents of each of which are herein incorporated by reference in their entirety). In some embodiments, compounds and/or compositions of the present invention may be delivered by electroporation.

### Administration and Delivery

Compounds and/or compositions of the present invention may be administered by any of the standard methods or routes known in the art. Such methods may include any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compounds and/or compositions of the present invention may be administered in ways which allow them to cross the blood-brain barrier, vascular barriers, or other epithelial barriers. Methods of formulation and administration may include any of those disclosed in US Pub. No. 2013/0122007, U.S. Pat. No. 8,415,459 or International Pub. No. WO 2011/151432, the contents of each of which are herein incorporated by reference in their entirety. Non-limiting routes of administration for compounds and/or compositions of the present invention are described below.

### Parenteral and Injectible Administration

In some embodiments, compounds and/or compositions of the present invention may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

In some embodiments, compounds and/or compositions of the present invention may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Aadministration

In some embodiments, compounds and/or compositions of the present invention may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, compounds and/or compositions of the present invention may be formulated for administration topically. The skin may be an ideal target site for delivery as it is readily accessible. Three routes are commonly considered to deliver compounds and/or compositions of the present invention to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). Compounds and/or compositions of the present invention can be delivered to the skin by several different approaches known in the art.

In some embodiments, the invention provides for a variety of dressings (e.g., wound dressings) or bandages (e.g., adhesive bandages) for conveniently and/or effectively carrying out methods of the present invention. Typically dressing or bandages may comprise sufficient amounts of compounds and/or compositions of the present invention described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present invention contemplates the use of transdermal patches, which often have the added advantage of providing controlled delivery of compounds and/or compositions of the present invention to the body. Such dosage forms may be prepared, for example, by dissolving and/or dispensing compounds and/or compositions in the proper medium. Alternatively or additionally, rates may be controlled by either providing rate controlling membranes and/or by dispersing compounds and/or compositions in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, compounds and/or compositions of the present invention are formulated in depots for extended release. Generally, specific organs or tissues (“target tissues”) are targeted for administration.

In some aspects of the invention, compounds and/or compositions of the present invention are spatially retained within or proximal to target tissues. Provided are method of providing compounds and/or compositions to target tissues of mammalian subjects by contacting target tissues (which comprise one or more target cells) with compounds and/or compositions under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of compounds and/or compositions that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of compounds and/or compositions administered to subjects are present intracellularly at a period of time following administration. For example, intramuscular injection to mammalian subjects may be performed using aqueous compositions comprising compounds and/or compositions of the present invention and one or more transfection reagent, and retention is determined by measuring the amount of compounds and/or compositions present in muscle cells.

Certain aspects of the invention are directed to methods of providing compounds and/or compositions of the present invention to a target tissues of mammalian subjects, by contacting target tissues (comprising one or more target cells) with compounds and/or compositions under conditions such that they are substantially retained in such target tissues. Compounds and/or compositions comprise enough active ingredient such that the effect of interest is produced in at least one target cell. In some embodiments, compounds and/or compositions generally comprise one or more cell penetration agents, although “naked” formulations (such as without cell penetration agents or other agents) are also contemplated, with or without pharmaceutically acceptable carriers.

In some embodiments, the amount of a growth factor present in cells in a tissue is desirably increased. Preferably, this increase in growth factor is spatially restricted to cells within the target tissue. Thus, provided are methods of increasing the amount of growth factor of interest in tissues of mammalian subjects. In some embodiments, formulations are provided comprising compounds and/or compositions characterized in that the unit quantity provided has been determined to produce a desired level of growth factor of interest in a substantial percentage of cells contained within predetermined volumes of target tissue.

In some embodiments, formulations comprise a plurality of different compounds and/or compositions, where one or more than one targets biomolecules of interest. Optionally, formulations may also comprise cell penetration agents to assist in the intracellular delivery of compounds and/or compositions. In such embodiments, determinations are made of compound and/or composition dose required to target biomolecules of interest in substantial percentages of cells contained within predetermined volumes of the target tissue (generally, without targeting biomolecules of interest in adjacent or distal tissues). Determined doses are then introduced directly into subject tissues. In some embodiments, the invention provides for compounds and/or compositions to be delivered in more than one administration or by split dose administration.

### Pulmonary Administration

In some embodiments, compounds and/or compositions of the present invention may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

In some embodiments, compounds and/or compositions of the present invention may be administered nasaly and/or intranasaly. In some embodiments, formulations described herein as being useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

### Ophthalmic or Otic Administration

In some embodiments, compounds and/or compositions of the present invention may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

### Payload Administration: Detectable Agents and Therapeutic Agents

In some embodiments, compounds and/or compositions of the present invention may be used in a number of different scenarios in which delivery of a substance (the “payload”) to a biological target is desired, for example delivery of detectable substances for detection of the target, or delivery of therapeutic and/or diagnostic agents. Detection methods may include, but are not limited to, both in vitro and in vivo imaging methods, e.g., immunohistochemistry, bioluminescence imaging (BLI), Magnetic Resonance Imaging (MRI), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging/staining/imaging is required.

In some embodiments, compounds and/or compositions may be designed to include both linkers and payloads in any useful orientation. For example, linkers having two ends may be used to attach one end to the payload and the other end to compounds and/or compositions. Compounds and/or compositions of the present invention may include more than one payload. In some embodiments, compounds and/or compositions may comprise one or more cleavable linker. In some embodiments, payloads may be attached to compounds and/or compositions via a linker and may be fluorescently labeled for in vivo tracking, e.g. intracellularly.

In some embodiments, compounds and/or compositions of the present invention may be used in reversible drug delivery into cells.

Compounds and/or compositions of the present invention may be used in intracellular targeting of payloads, e.g., detectable or therapeutic agents, to specific organelles. In addition, compounds and/or compositions of the present invention may be used to deliver therapeutic agents to cells or tissues, e.g., in living animals. For example, the compounds and/or compositions described herein may be used to deliver chemotherapeutic agents to kill cancer cells. Compounds and/or compositions may be attached to therapeutic agents through one or more linkers may facilitate membrane permeation allowing therapeutic agents to travel into cells to reach intracellular targets.

In some embodiments, payloads may be a therapeutic agent such as a cytotoxins, radioactive ions, chemotherapeutics, or other therapeutic agents. Cytotoxins and/or cytotoxic agents may include any agents that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, the contents of each of which are incorporated herein by reference in their entirety), and analogs or homologs thereof. Radioactive ions include, but are not limited to iodine (e.g., ¹²⁵iodine or ¹³¹iodine), ⁸⁹strontium, phosphorous, palladium, cesium, iridium, phosphate, cobalt, ⁹⁰yttrium, ¹⁵³samarium, and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, payloads may be detectable agents, such as various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-l-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DAB ITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and-6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz[e]indolium hydroxide, inner salt, compound with n,n-diethylethanamine(1:1) (IR144); 5-chloro-2-[2-[3-[(5-chloro-3-ethyl-2(3H)-benzothiazol- ylidene)ethylidene]-2-(diphenylamino)-1-cyclopenten-l-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives(e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′tetramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Combinations

In some embodiments, compounds and/or compositions of the present invention may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compounds and/or compositions of the present invention may be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

In some cases, compounds and/or compositions of the present invention may be combined with one or more therapeutic agents known in the art. Such agents may include BYM338 (Novartis, Basel, Switzerland), wherein administration may comprise any of the methods disclosed in clinical trial number NCT01925209 entitled Efficacy and Safety of Bimagrumab/BYM338 at 52 Weeks on Physical Function, Muscle Strength, Mobility in sIBM Patients (RESILIENT). Other agents that may be used in combination with compounds and/or compositions of the present invention may include any of those disclosed in US Pub. No. 2013/0122007, U.S. Pat. No. 8,415,459 or International Pub. No. WO 2011/151432, the contents of each of which are herein incorporated by reference in their entirety.

### Dosing and Dosage Forms

The present disclosure encompasses delivery of compounds and/or compositions of the present invention for any of therapeutic, pharmaceutical, diagnostic or imaging by any appropriate route taking into consideration likely advances in the sciences of drug delivery. Delivery may be naked or formulated.

### Naked Delivery

Compounds and/or compositions of the present invention may be delivered to cells, tissues, organs and/or organisms in naked form. As used herein in, the term “naked” refers to compounds and/or compositions delivered free from agents or modifications which promote transfection or permeability. The naked compounds and/or compositions may be delivered to the cells, tissues, organs and/or organisms using routes of administration known in the art and described herein. In some embodiments, naked delivery may include formulation in a simple buffer such as saline or PBS.

### Formulated Delivery

In some embodiments, compounds and/or compositions of the present invention may be formulated, using methods described herein. Formulations may comprise compounds and/or compositions which may be modified and/or unmodified. Formulations may further include, but are not limited to, cell penetration agents, pharmaceutically acceptable carriers, delivery agents, bioerodible or biocompatible polymers, solvents, and/or sustained-release delivery depots. Formulations of the present invention may be delivered to cells using routes of administration known in the art and described herein.

Compositions may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

### Dosing

The present invention provides methods comprising administering one or more compounds and/or compositions to subjects in need thereof. Compounds and/or compositions of the present invention, or prophylactic compositions thereof, may be administered to subjects using any amount and any route of administration effective for preventing, treating, diagnosing, or imaging diseases, disorders and/or conditions. The exact amount required will vary from subject to subject, depending on species, age and/or general subject condition, severity of disease, particular composition, mode of administration, mode of activity, and the like. Compositions in accordance with the invention are typically formulated in dosage unit form for ease of administration and uniformity of dosage. It will be understood, however, that the total daily usage of compositions of the present invention will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

According to the present invention, compounds and/or compositions of the present invention may be administered in split-dose regimens. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in a 24 hour period. In some embodiments, compounds and/or compositions of the present invention may be administered as a single unit dose. In some embodiments, compounds and/or compositions of the present invention may be administered to subjects in split doses. In some embodiments, compounds and/or compositions of the present invention may be formulated in buffer only or in formulations described herein. Pharmaceutical compositions described herein may be formulated into dosage forms described herein, such as a topical, intranasal, intratracheal, or injectable (e.g., intravenous, intraocular, intravitreal, intramuscular, intracardiac, intraperitoneal, subcutaneous). General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose and/or milk sugar as well as high molecular weight polyethylene glycols and the like.

### Assays

In some embodiments, recombinant proteins (including, but not limited to chimeric proteins) disclosed herein and/or antibodies directed to such proteins may be developed using assays described herein. In some embodiments, recombinant proteins (including, but not limited to chimeric proteins) disclosed herein and/or antibodies directed to such proteins may be used in assays to develop other recombinant proteins and/or antibodies of the present invention.

### Binding Assays

In some embodiments, the present invention provides binding assays. As used herein, the term “binding assay” refers to an assay used to assess the ability of two or more factors to associate. Such assays may assess the ability of a desired antigen to bind a desired antibody and then use one or more detection methods to detect binding. Binding assays of the invention may include, but are not limited to surface Plasmon resonance-based assays, ELISAs and fluorescence flow cytometry-based assays. Binding assays of the invention may comprise the use of one or more recombinant proteins described herein, including, but not limited to any TGF-β family member proteins, any chimeric proteins, any cofactors and any modules, combinations or fragments thereof.

### Cell-Based Assays

In some embodiments, the present invention provides cell-based assays. As used herein, the term “cell-based assay” refers to an assay comprising at least one aspect that involves the use of a living cell or cell culture. In some embodiments, these may be useful for assessing the modulation of growth factor release from GPCs, referred to herein as “growth factor release assays”. In some embodiments, cell-based assays may be useful for assessing the modulation of growth factor activity, referred to herein as “growth factor activity assays”. Cell-based assays of the present invention may comprise expression cells and/or responsive cells. Expression cells, as referred to herein, are cells that express one or more factors being analyzed in a particular assay. Such expression may be natural or may be the result of transfection and/or transduction of a foreign gene. In some embodiments, expression of one or more factors by expression cells may be enhanced or suppressed by the addition of one or more exogenous factors. In some embodiments, expression cells may comprise cell lines (e.g. HEK293 cells, HepG2 cells, CHO cells, TMLC cells, 293T/17 cells, Hs68 cells, CCD1112sk cells, HFF-1 cells, Keloid fibroblasts or Sw-480 cells). In some embodiments, cell lines comprising expression cells may express one or more recombinant proteins of the present invention (e.g. naturally and/or through transfection, stable transfection, and/or transduction).

In some embodiments, growth factor release/activity assays may comprise expression cells that express GPCs. In such embodiments, additional factors may be co-expressed in and/or combined with expression cells to determine their effect on growth factor release from such GPCs. In some embodiments, integrins (including, but not limited to αᵥβ₆ integrin, αᵥβ₈ integrin and/or α₉β₁ integrin) are co-expressed and/or otherwise introduced to GPC-expressing expression cells. In some embodiments, such additional integrin expression may facilitate growth factor release. In some embodiments, extracellular proteins (e.g., fibrillins and/or GASPs) and/or variants thereof are coexpressed and/or otherwise introduced into expression cells.

In some embodiments, one or more genes may be knocked out, knocked down and/or otherwise modulated in expression cells depending on the focus of a particular assay. In some embodiments, one or more gene products may be modulated at the RNA and/or protein level. In some embodiments, gene products may be reduced through the introduction of siRNA molecules to expression cells. In some embodiments, gene products from extracellular protein (e.g., fibrillins and/or GASPs) genes may be reduced and/or eliminated from expression cells of the present invention.

Cell-based assays of the present invention, including, but not limited to growth factor release/activity assays, may comprise responsive cells. As used herein, the term “responsive cell” refers to a cell that undergoes a response to one or more factors introduced into an assay. In some embodiments, such responses may include a change in gene expression, wherein such cells modulate transcription of one or more genes upon contact with one or more factors introduced. In some embodiments, responsive cells may undergo a change in phenotype, behavior and/or viability.

In some embodiments, responsive cells comprise one or more reporter genes. As used herein, the term “reporter gene” refers to a synthetic gene typically comprising a promoter and a protein coding region encoding one or more detectable gene products. Reporter genes are typically designed in a way such that their expression may be modulated in response to one or more factors being analyzed by a particular assay. This may be carried out by manipulating the promoter of reporter genes. As used herein, the term promoter refers to part of a gene that initiates transcription of that gene. Promoters typically comprise nucleotides at the 3′ end of the antisense strand of a given gene and are not transcribed during gene expression. Promoters typically function through interaction with one or more transcription factors as well as RNA polymerase enzymes to initiate transcription of the protein encoding portion of the gene. Segments of the promoter that physically interact with one or more transcription factors and/or polymerase enzymes are referred to herein as response elements. In some embodiments, reporter genes are designed to comprise promoters and/or response elements known to be responsive to one or more factors (including, but not limited to growth factors) being analyzed in a given assay. Changes in responsive cell gene expression may be measured according to any methods available in the art to yield gene expression data. Such gene expression data may be obtained in the form of luciferase activity data [often measured in terms of relative light units (RLUs)].

In some cases, responsive cells undergo a change in viability in response to one or more factors introduced in an assay. Such responsive cells may be used in proliferation assays as described herein. Changes in responsive cell viability may be detected by cell counting and/or other methods known to those skilled the art to yield responsive cell viability data.

Protein encoding regions of reporter genes typically encode one or more detectable proteins. Detectable proteins refer to any proteins capable of detection through one or more methods known in the art. Such detection methods may include, but are not limited to Western blotting, ELISA, assaying for enzymatic activity of detectable proteins (e.g. catalase activity, β-galactosidase activity and/or luciferase activity), immunocytochemical detection, surface plasmon resonance detection and/or detection of fluorescent detectable proteins. When a reporter gene is used in an assay, the expression of detectable proteins correlates with the ability of factors being assayed to activate the promoter present in the reporter gene. In embodiments comprising growth factor release/activity assays, reporter gene promoters typically respond to growth factor signaling. In such embodiments, the level of detectable protein produced correlates with level of growth factor signaling, indicating release and/or activity of a given growth factor.

In some embodiments, reporter genes encode luciferase enzymes. Chemical reactions between luciferase enzymes and substrate molecules are light-emitting reactions. Due to such light-emitting reactions, luciferase enzyme levels can be quantified through the addition of substrate molecules and subsequent photodetection of the emitted light. In some embodiments, reporter genes of the present invention encode firefly luciferase, the sequence of which was cloned from Photinus pyralis. In some embodiments, responsive cells of the present invention comprise reporter genes that express luciferase with promoters that are responsive to growth factors. In such embodiments, luciferase activity may correlate with growth factor activity levels allowing for growth factor activity and/or release from GPCs to be determined.

In some embodiments, reporter genes are inserted into bacterial plasmids to enable replication and/or facilitate introduction into cells. In some embodiments, such plasmids are designed to comprise sequences encoding detectable gene products and may be manipulated to insert promoter sequences that may be responsive to one or more factors of interest. These plasmids are referred to herein as reporter plasmids. In some embodiments of the present invention, promoters that may be responsive to one or more factors of interest may be inserted into reporter plasmids, upstream of sequences encoding detectable gene products to form functional reporter genes within such reporter plasmids. Reporter plasmids that comprise at least one functional reporter gene are referred to herein as reporter constructs. In some embodiments, reporter constructs of the present invention may comprise pGL2 reporter plasmids (Promega BioSciences, LLC, Madison, Wis.), pGL3 reporter plasmids (Promega BioSciences, LLC, Madison, Wis.), pGL4 reporter plasmids (Promega BioSciences, LLC, Madison, Wis.) or variants thereof. Such reporter constructs express firefly luciferase in response to promoter activation.

In some embodiments, reporter constructs may be introduced directly into expression cells or may be introduced into one or more responsive cells. Responsive cells of the present invention comprising one or more reporter genes are referred to herein as reporter cells. In some embodiments, reporter cells may be transiently transfected with reporter constructs or may comprise stable expression of such constructs (e.g. reporter constructs are successfully replicated along with genomic DNA during each round of cell division). Cell lines that stably comprise reporter constructs are referred to herein as reporter cell lines. In some embodiments, reporter cells are mammalian. In some embodiments, reporter cells may comprise mouse cells, rabbit cells, rat cells, monkey cells, hamster cells and human cells. In some embodiments, cell lines useful for transient and/or stable expression of reporter genes may include, but are not limited to HEK293 cells, HepG2 cells, HeLa cells, Sw-480 cells, TMLC cells [as disclosed by Abe et al (Abe, M. et al., An assay for transforming growth factor-β using cells transfected with a plasminogen activator inhibitor-1 promoter-luciferase construct. Analytical Biochemistry. 1994. 216:276-84)], 293T/17 cells, Hs68 cells, CCD1112sk cells, HFF-1 cells, Keloid fibroblasts, A204 cells, L17 RIB cells [as disclosed by Cash et al (Cash, J. N et al., The structure of myostatin:follistatin 288: insights into receptor utilization and heparin binding. The EMBO Journal. 2009. 28:2662-76)], C₂C₁₂ cells, HepG2 cells and EL4 T lymphoma cells.

In embodiments where one or more reporter cells and/or reporter cell lines are utilized, such cells may be cultured with expression cells as part of a co-culture system. In some embodiments reporter cells/reporter cell lines may be cultured separately from expression cells. In such embodiments, lysates and/or media from expression cells may be combined with reporter cell/reporter cell line cultures to assess expressed factors (including, but not limited to growth factors).

In some embodiments, cell-based assays of the present invention may only comprise expression cells and not responsive cells. In such embodiments, expressed proteins, including but not limited to GPCs and/or growth factors, may be detected by one or more methods that are not cell based. Such methods may include, but are not limited to Western Blotting, enzyme-linked immunosorbent assay (ELISA), immunocytochemistry, surface plasmon resonance and other methods known in the art for protein detection. In some embodiments, GDF release in expression cell cultures and/or culture medium may be detected by ELISA. In some embodiments, the GDF-8/myostatin quantikine ELISA kit (R&D Systems, Minneapolis, MN) may be used. Examples of anti-GDF-8/myostatin antibodies that may be used for detection include AF1539, MAB788 and AF788 (R&D Systems, Minneapolis, Minn.).

### Proliferation/Differentiation Assays

In some embodiments, cell-based assays of the present invention may comprise proliferation assays. As used herein, the term “proliferation assay” refers to an assay that determines the effect on one or more agents on cell proliferation.

In some embodiments, cell differentiation assays may be used to assess growth factor activity modulation by activating and/or inhibiting antibodies. Cell differentiation assays may include skeletal muscle differentiation assays. Such assays may be used to test GDF-11 activating and/or inhibiting antibodies. In some cases, skeletal muscle differentiation assays assess myoblast differentiation by looking at changes in the expression level of proteins that change during stages of differentiation. Such proteins may include, but are not limited to myogenin, myosin heavy chain and creatine kinase. In some cases, GDF-11-inhibiting antibodies may be tested by examining their effect on myoblast differentiation.

### Animal Models

In some embodiments, compounds and/or compositions of the present invention may be tested in animal models including mammalian models of muscles growth and development. These models may include but are not limited to mouse models of grip strength. Such models may include testing by determining the force (in grams) generated when a mouse is pulled from a force measuring lever. Additional models may include but are not limited to other strength and endurance tests such as the Morris water maze test for endurance and the wire hang test for motor deficits. Other mouse models may include, but are not limited to assays which determine changes in whole muscle weight and size.

In another embodiment, compounds and/or compositions of the present invention may be tested in non-mouse animal models including models of muscle growth and development where changes in muscle are observed. Such changes may include, but are not limited to changes in muscle weight and size, muscle fiber length and diameter, number of fibers per muscle bundle, number of synaptic contacts per muscle fiber, and fiber strength.

In some embodiments, tissues from animals treated with compounds and/or compositions of the present invention may be analyzed by immunohistochemical analysis. Such analysis may include immunostaining of muscles, muscle fibers and muscle connections to show changes in response to treatment. Complete blood counts may be carried out pre and post study to enable monitoring of compounds and/or compositions of the present invention.

### Kits and Devices

Any of the compounds and/or compositions of the present invention may be comprised in a kit. In a non-limiting example, reagents for generating compounds and/or compositions, including antigen molecules are included in one or more kit. In some embodiments, kits may further include reagents and/or instructions for creating and/or synthesizing compounds and/or compositions of the present invention. In some embodiments, kits may also include one or more buffers. In some embodiments, kits of the invention may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquotted. Where there are more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present invention may also typically include means for containing compounds and/or compositions of the present invention, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. In such embodiments, dye may then be resuspended in any suitable solvent, such as DMSO.

In some embodiments, kits may include instructions for employing kit components as well the use of any other reagent not included in the kit. Instructions may include variations that may be implemented.

In some embodiments, compounds and/or compositions of the present invention may be combined with, coated onto or embedded in a device. Devices may include, but are not limited to, dental implants, stents, bone replacements, artificial joints, valves, pacemakers and/or other implantable therapeutic device.

It will be readily apparent to those skilled in the art that other suitable modifications and adaptations of the methods of the disclosure described herein are obvious and may be made using suitable equivalents without departing from the scope of the disclosure or the embodiments disclosed herein. Having now described the present disclosure in detail, the same will be more clearly understood by reference to the following examples, which are included for purposes of illustration only and are not intended to be limiting of the disclosure.

### EXAMPLES

### Example 1

### Cleavage-Dependent Activation of proGDF-11 Purification of Recombinant proGDF11

To produce precursor forms of GDF11, stable cell lines overexpressing proGDF11 and PCSKS were established. Protein constructs were stably integrated into FLP-IN™ T-REX™ 293 cells (Life Technologies, Carlsbad, CA) and proteins were expressed according to manufacturer's instructions. Filtered supernatant was collected and the protein of interest purified by Ni-NTA chromatography (Qiagen). The protein was further purified by size exclusion chromatography (SEC).

proGDF11 was purified directly from cell culture supernatants. Latent GDF11 was produced by adding stable cells overexpressing PCSK5 (a proprotein convertase) to the GDF11 expressing stable cells and purifying latent GDF11 from the cell supernatants (see above). The cleaved material was >95% latent, with the proteolysis reaction proceeding almost to completion under the conditions used.

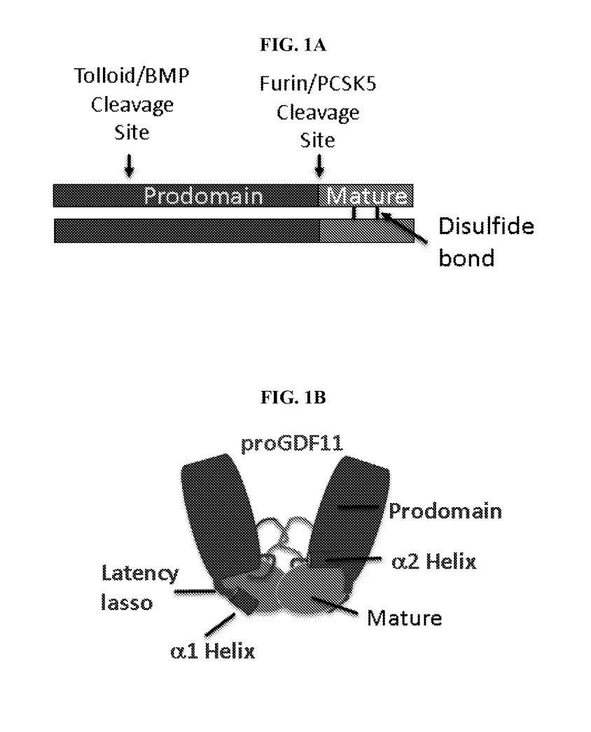

Partially proconvertase-cleaved proGDF-11 was run on an SDS PAGE gel under non-reducing and reducing conditions (see FIG. 1). Under non-reducing conditions, the protein bands consisted of the proGDF-11 dimer (˜100 kD), proGDF-11 monomer (˜60 kD), GDF-11 prodomain (˜38 kD) and GDF-11 growth factor dimer (˜20 kD). Under reducing conditions, the growth factor dimer was reduced to the monomer (˜12 kD).

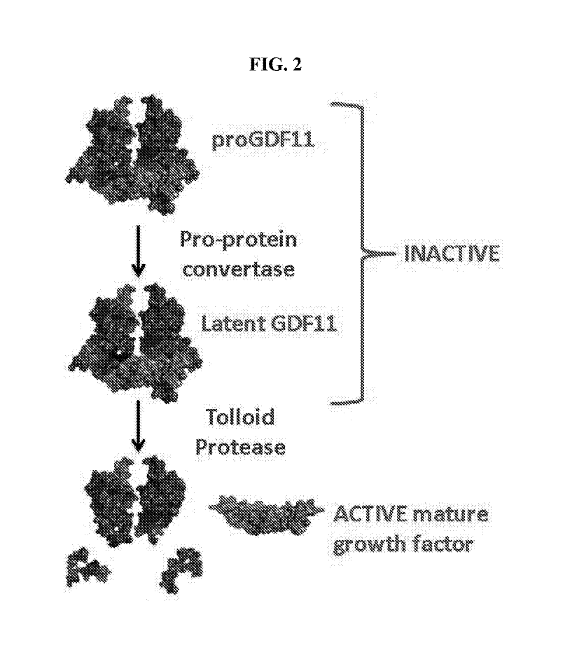

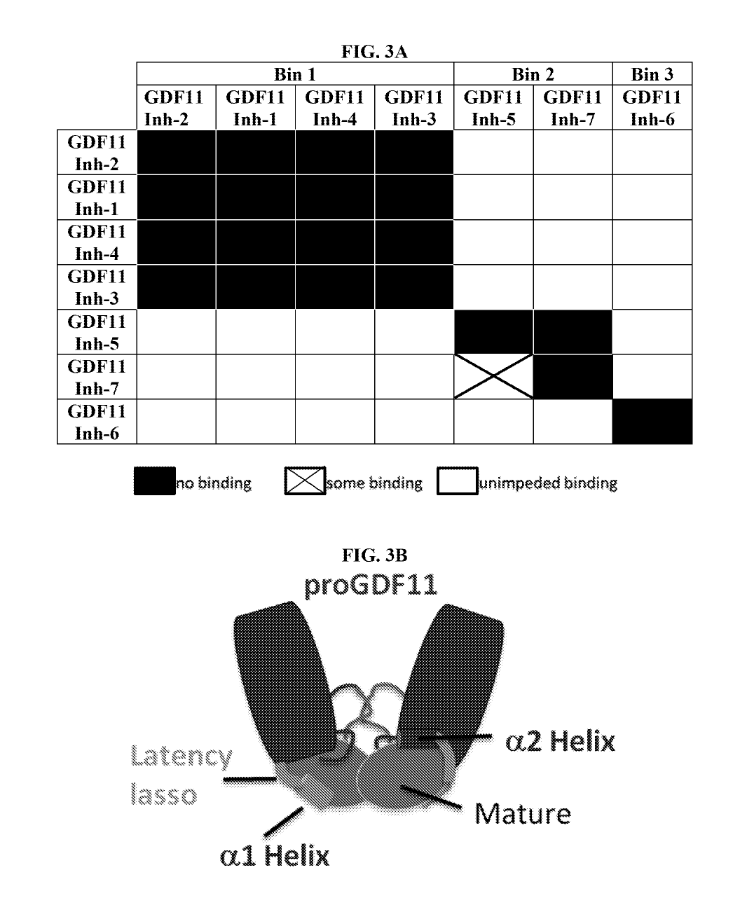

Samples of proGDF-11 were incubated with pro-protein convertase (either Furin/PACE3 or PCSK5), Tolloid proteinase (either BMP-1 or mTLL2), a combination of both proteases or no proteases. The samples were incubated at 37° C. for 16 hrs. Treated samples were analyzed by adding them to 293T or HepG2 cells carrying a CAGA luciferase response plasmid and incubated for six hours. Growth factor activity was assessed by measuring CAGA-dependent luciferase activity in the cell lysates. As shown in FIG. 2, GDF-11 activity resulting from treatment with combined protease lysate (Tolloid proteinase and proprotein convertase) was nearly equal to treatment with recombinant GDF-11 (R&D Systems, Minneapolis, Minn.). The results of further assays are presented in FIG. 3 demonstrating growth factor release from proGDF-11 after treatment with various proteases in both cell lines. Surprisingly, treatment with mTLL-2 and PCSK5 resulted in the highest level of activity in comparison to other protease treatment conditions.

### Example 2

### Generation of Anti-Human GDF11 Prodomain Complex Monoclonal Antibodies

Monoclonal antibodies were identified via selection of a naïve phage display library using proGDF11 (SEQ ID NO: 82) as the primary antigen for selection.

Phage selection and initial screening were performed using a library displaying conventional scFv in a format similar to that described by McCafferty et al. (McCafferty et. al., 1990). Each round of selection consisted of pre-clearing (for removal of nonspecific phage antibodies), incubation with antigen, washing, elution and amplification. Selections were performed via multiple rounds using both solid phase (biotinylated antigens coated on immunotubes) and solution phase (biotinylated antigens, captured using streptavidin coated beads) panning strategies. Antigens were biotinylated with 21329 EZ-Link NHS-PEG4-Biotin (Pierce) according to manufactorers directions. The amount of biotin added was titrated to achieve one biotin molecule per one dimeric antigen complex as assessed by the PierceTM Fluorescence Biotin Quantitation Kit according to manufactorers directions.

In total, 2,304 individual scFv clones were screened for binding to proGDF11. DNA for scFv clones of interest were sequenced and 82 unique clones were identified. Positive binding scFv clones were counterscreened for binding to proMyostatin (SEQ ID NO: 83) as well as to a panel of unrelated proteins (e.g., His-ProTGFβ and His-ICAM1) to confirm specificity for pro/latent GDF11. From the panel of 50 unique scFv clones, 31 were converted to full length IgG (IgG1 isotype) for additional characterization.

Candidate clones were converted to human IgG1 format. Full-length IgG antibodies were further characterized by ELISA for binding to the human and murine pro- and latent-forms of myostatin and GDF11. Antibodies were also screened for binding to the Myostatin prodomain (SEQ ID NO: 85), proTGFDβ1 (human and murine), the mature growth factor of Myostatin (SEQ ID NO: 89), the GDF11 mature growth factor (SEQ ID NO: 90), and proActivin A (SEQ ID NO: 84). Chimeric constructs which swapped portions of the prodomains of Myostatin and GDF11 were designed and produced. For example proGDF11 arm8 (SEQ ID NO: 112) refers to GDF11 having the GDF8 arm domain, and prodomain GDF11 arm8 (SEQ ID NO: 114) refers to the GDF11 prodomain having the GDF8 arm domain. These chimeric proteins were assayed for interaction with screening antibodies by ELISA and allowed for the assessment of binding of antibodies to the ARM or straight jacket portions of the GDF11 prodomain complex. See Table 22. Antibodies were selected based on their cross-reactivity with pro- and latent human and murine GDF11, with no interactions with GDF8, Activin, or TGFβ proteins. Activin A is a homodimer of inhibin-beta A subunits. For example, Inhibin-beta A can heterodimerize with other proteins to produce different Activin proteins.

TABLE 22
ELISA binding data.

| | | | | | | | | prodomain | |
|---|---|---|---|---|---|---|---|---|---|
| | | hu proGDF11 | mu proGDF11 | hu latent | mu latent | proGDF11 | hu proGDF8 | GDF11 ARM8 | mature |
| | | Binding | binding | GDF11 binding | GDF11 binding | ARM8 binding | binding | binding | GDF11 |
| | Epitope | (SEQ ID | (SEQ ID | (SEQ ID | (SEQ ID | (SEQ ID | (SEQ ID | (SEQ ID | (SEQ ID |
| Clone | Bin | NO: 82) | NO: 97) | NO: 82) | NO: 97) | NO: 112) | NO: 83) | NO: 114) | NO: 90) |
| GDF11 | 1 | + | + | + | + | − | − | − | − |
| Inh-2 | | | | | | | | | |
| GDF11 | 1 | + | + | + | + | − | + | + | − |
| Inh-1 | | | | | | | | | |
| GDF11 | 2 | + | + | + | + | + | + | + | − |
| Inh-7 | | | | | | | | | |
| GDF11 | 2 | + | + | + | + | + | + | + | + |
| Inh-5 | | | | | | | | | |
| GDF11 | 1 | + | + | + | + | − | − | − | − |
| Inh-4 | | | | | | | | | |
| GDF11 | 1 | + | + | + | + | − | − | − | − |
| Inh-3 | | | | | | | | | |
| GDF11 | 3 | + | + | + | + | − | − | − | + |
| Inh-6 | | | | | | | | | |

In Table 22, a “+” indicates binding was detected and a “−” indicates that binding was not detected.

In addition to ELISA screening, full length IgG1 clones were screened by using in vitro CAGA luciferase reporter assays. The process of the GDF11 activation assays is as follows: 50 nM proGDF11 was pre-incubated with the antibody to be tested. Conditioned media from cells overexpressing PCSK5 and recombinant BMP1 (R&D systems) were added to the mixture and incubated overnight at 30 C, which led to cleavage of proGDF11 and release of active GDF11. Activation of GDF11 was measured by a CAGA luciferase reporter assay system in which material from the proteolysis reaction was incubated with 293T cells containing a stably integrated pGL4 plasmid (Promega, Madison, WI) with a promoter comprising SMAD-responsive CAGA sequences. Cells were incubated at 37° for 6 hours before detection of luciferase expression using BRIGHT-GLO™ reagent (Promega, Madison, Wis.) according to manufacturer's instructions. Results were compared to control reactions to calculate the fraction of released mature GDF11 growth factor in order to calculate the percent inhibition of GDF11 activation.

### ELISA Analysis

Enzyme-linked immunosorbent assay (ELISA) analysis is carried out to assess antibody binding. 96-well ELISA assay plates are coated with neutravidin, a deglycosylated version of streptavidin with a more neutral pH. Target proteins are expressed with or without histidine (His) tags and subjected to biotinylation. Biotinylated target proteins are incubated with neutravidin-coated ELISA assay plates for two hours at room temperature and unbound proteins are removed by washing three times with wash buffer (either 25 mM Tris, 150 mM NaCl, 0.05% TWEEN®-20, or 20 mM Hepes pH 7.5, 500 mM NaCl, 0.05% TWEEN®-20). Primary antibodies being tested are added to each well and allowed to incubate at room temperature for 1 hour or more. Unbound antibody is then removed by washing three times with wash buffer. Secondary antibodies capable of binding to primary antibodies being tested and conjugated with detectable labels are then incubated in each well for 30 minutes at room temperature. Unbound secondary antibodies are removed by washing three times with wash buffer. Finally, bound secondary antibodies are detected by enzymatic reaction, fluorescence detection and/or luminescence detection, depending on the detectable label present on secondary antibodies being detected.

CAGA Analysis using 293T CAGA-Luciferase Assay

CAGA-luciferase assays are carried out to test antibodies for modulation of GDF-11 activity. 100 μl of 0.01% poly-L-lysine solution is added to each well of a 96-well plate. Plates are incubated for 10 min at room temperature before they are washed with water. 293T cells comprising transient or stable expression of pGL4 (Promega, Madison, Wis.) under the control of a control promoter or promoter comprising SMAD1/2 responsive CAGA sequences are then used to seed poly-L-lysine-coated wells (4×10⁴ cell/well in complete growth medium). The next day, cells are washed with 150 μl/well of cell culture medium with 0.1% bovine serum albumin (BSA) before treatment with proGDF-11 complexes with or without test antibody. Cells are incubated at 37° for 6 hours before detection of luciferase expression using BRIGHT-GLO™ reagent (Promega, Madison, Wis.) according to manufacturer's instructions.

### Example 3

### Characterization of Anti-Human GDF11 Prodomain Complex Monoclonal Antibodies

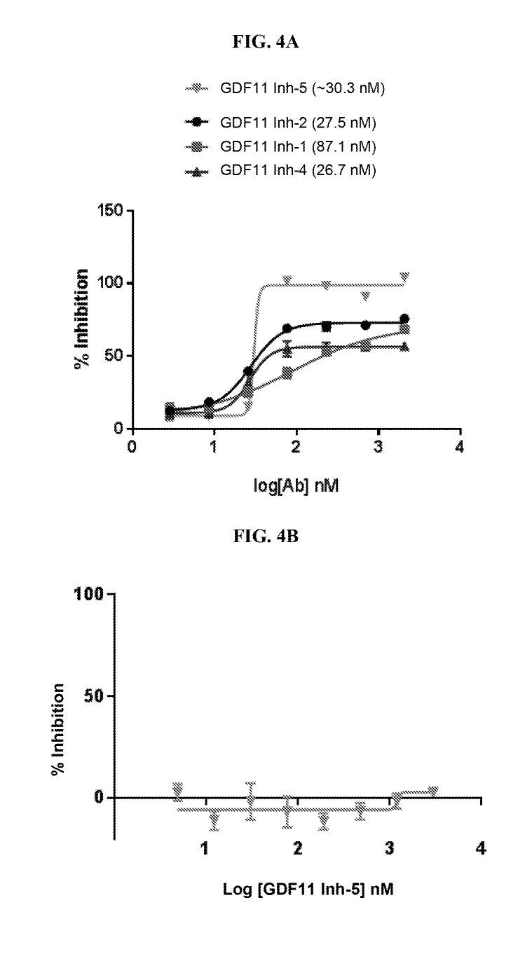

Seven binding proteins achieved >50% inhibition against both human and murine proGDF11 in the activity assay and were further characterized for epitope diversity and EC50 in a dose response activity assay experiment (Table 23 and FIG. 4). In Table 23, maximal percent inhibition of proGDF11 activation for both human and murine GDF11 are reported. Binding affinity for human and murine latent/proGDF11 was also measured for selected clones utilizing surface biolayer interferometry, with affinities reported in pM.

TABLE 23
Binding Specificity of antibody clones. Kd (pM) measured using Octet assay.

| | | | Kd pM human | Kd pM murine | Kd pM human | Human | Human latent | Human |
|---|---|---|---|---|---|---|---|---|
| | Human | Murine | proGDF11 | proGDF11 | latent GDF11 | proGDF8 | GDF8 | proActivin A |
| | proGDF11 | proGDF11 | (SEQ ID | (SEQ ID | (SEQ ID | (SEQ ID | (SEQ ID | (SEQ ID |
| Clone | % Inhibition | % inhibition | NO: 82) | NO: 97) | NO: 82) | NO: 83) | NO: 83) | NO: 84) |
| GDF11 | 91 | 80 | 490 | 848 | − | − | − | − |
| Inh-1 | | | | | | | | |
| GDF11 | 87 | 88 | 710 | 937 | − | − | − | − |
| Inh-3 | | | | | | | | |
| GDF11 | 100 | 95 | 910 | 1730 | 270 | NB | NB | NB |
| Inh-2 | | | | | | | | |
| GDF11 | 94 | 87 | 1080 | − | 310 | NB | NB | NB |
| Inh-4 | | | | | | | | |
| GDF11 | 74 | 53 | 2120 | − | − | − | − | − |
| Inh-7 | | | | | | | | |
| GDF11 | 96 | 90 | <1 | − | <1 | NB | NB | NB |
| Inh-5 | | | | | | | | |
| GDF11 | 77 | 63 | 750 | − | − | − | − | − |
| Inh-6 | | | | | | | | |

In Table 23, NB indicates that no binding was detected and “−” indicates the absence of experimental data.

Epitope mapping was performed to determine the binding site of the anti-GDF11 prodomain complex binding proteins. The binding epitope of these binding proteins are shown in FIG. 3 and Table 22. Epitope binning was carried out by surface biolayer interferometry using a ForteBio BLI instrument, in which the biotinylated proGDF11 was immobilized on a streptavidin coated biosensor chip, and cross-blocking of antibodies was evaluated by sensor response, the results of which are shown in FIG. 3. In a cross-blocking experiment, antibody binding is assessed in a pairwise fashion utilizing two separate antibodies. Initially, saturated binding is achieved for the first antibody, such that all of the epitopes on the proGDF11 complex are occupied by the first antibody. In the second step, a second antibody is then allowed to bind to the saturated proGDF11-antibody 1 complex. If the second antibody displays a sensor response indicating a binding event, then the two antibodies recognize differing epitopes. If no binding event is observed, then it is inferred that the presence of the first antibody blocked the ability of the second to bind to proGDF11 and thus the two antibodies recognize the same epitope. The experiment is then repeated for the same pair in which the order of addition of the two antibodies is switched as the order of antibody addition can provide information on relative affinity and/or potential steric interference. These epitope binning experiments, along with data from the ELISA binding experiments (see Example 2 above), allowed for the segregation of our functionally active lead antibodies into three distinct epitope groups or “Bins” and the mapping of their binding sites on the GDF11 prodomain complex.

Binding affinities of antibody candidates were determined using the ForteBio Octet QKe dip and read label free assay system utilizing bio-layer interferometry. Human pro- and latent GDF8 and human Activin A were immobilized to streptavidin-coated biosensors in each experiment and the antibodies were presented in solution at high concentration (50 μg/mL) to measure binding interactions. Data are shown in Table 23.

FIG. 4 shows dose-dependent inhibition of the GDF11 prodomain complex by some of the disclosed antibodies utilizing the proGDF11 activation assay (described above). At a concentration of 50 nM proGDF11, the EC50s for Clones GDF11 Inh-5, GDF11 Inh-2, GDF11 Inh-1, and GDF11 Inh-4 are 30.13 nM, 27.5 nM, 87.1 nM, and 26.7 nM, respectively. As a control, the GDF11 inhibitory antibody GDF11 Inh-5 was tested and found not to block activation of human proGDF8 (FIG. 4B). In this assay, the human proGDF8 concentration was 400nM.

1.-203. (canceled)

204. A pharmaceutical composition comprising an antibody or antigen-binding portion thereof that specifically binds human pro- or latent GDF11 but does not specifically bind human pro- or latent GDF8, human pro- or latent Activin A, human mature GDF8, or human mature Activin A.

205. The pharmaceutical composition of claim 204, wherein the antibody or antigen-binding portion thereof does not specifically bind human mature GDF11.

206. The pharmaceutical composition of claim 204, wherein the antibody or antigen-binding portion thereof specifically binds murine pro- or latent GDF11.

207. The pharmaceutical composition of claim 204, wherein the antibody or antigen-binding portion thereof is a GDF11 inhibitory antibody.

208. The pharmaceutical composition of claim 207, wherein the antibody or antigen-binding portion thereof is a GDF11 activation inhibitor.

209. The pharmaceutical composition of claim 208, wherein the antibody or antigen-binding portion thereof binds a GDF11 prodomain complex and inhibits the release of mature GDF11 from the GDF11 prodomain complex.

210. The pharmaceutical composition of claim 207, wherein the antibody or antigen-binding portion thereof inhibits proteolytic cleavage of the pro- or latent GDF11.

211. The pharmaceutical composition of claim 207, wherein the antibody or antigen-binding portion thereof is a stabilizing antibody or antigen-binding portion thereof.

212. The pharmaceutical composition of claim 204, wherein the antibody or antigen-binding portion thereof is a monoclonal antibody or antigen-binding portion thereof.

213. The pharmaceutical composition of claim 204, wherein the antibody or antigen-binding portion thereof is a humanized or fully human antibody or antigen-binding portion thereof.

214. The pharmaceutical composition of claim 204, wherein the antibody or antigen-binding portion thereof comprises a CDR-H3 amino acid sequence set forth in SEQ ID NO: 66, 72, 30, 36, or 42.

215. The pharmaceutical composition of claim 204, wherein the antibody or antigen-binding portion thereof comprises a CDR-L3 amino acid sequence as set forth in SEQ ID NO: 69, 75, 33, 39, or 45.

216. The pharmaceutical composition of claim 204, wherein the antibody or antigen-binding portion thereof comprises a CDR-H1 amino acid sequence as set forth in SEQ ID NO: 64, 70, 28, 34, or 40.

217. The pharmaceutical composition of claim 204, wherein the antibody or antigen-binding portion thereof comprises a CDR-L1 amino acid sequence as set forth in SEQ ID NO: 67, 73, 31, 37, or 43.

218. The pharmaceutical composition of claim 204, wherein the antibody or antigen-binding portion thereof comprises a CDR-H2 amino acid sequence as set forth in SEQ ID NO: 65, 71, 29, 35, or 41.

219. The pharmaceutical composition of claim 204, wherein the antibody or antigen-binding portion thereof comprises a CDR-L2 amino acid sequence as set forth in SEQ ID NO: 68, 74, 32, 38, or 44.

220. The pharmaceutical composition of claim 204, wherein the antibody or antigen-binding portion thereof comprises a variable heavy chain amino acid sequence as set forth in SEQ ID NO: 10, 12, or 14.

221. The pharmaceutical composition of claim 204, wherein the antibody or antigen-binding portion thereof comprises a variable heavy chain amino acid sequence as set forth in SEQ ID NO: 11, 13, or 15.

222. A method for treating a disease or condition in a human subject, the method comprising a step of: administering to the human subject a therapeutically effective amount of the pharmaceutical composition of claim 204, wherein the disease or condition is selected from the group consisting of: cancer, angiogenic and endothelial proliferation conditions, cardiovascular indications, muscle disorders and muscle injuries.
